(19) United States
(12) Patent Application Publication
(10) Pub. No.: US 2024/0301073 A1
(43) Pub. Date: Sep. 12, 2024

(54) LTBP COMPLEX-SPECIFIC INHIBITORS OF TGFb1 AND USES THEREOF

(71) Applicant: Scholar Rock, Inc., Cambridge, MA (US)

(72) Inventors: Thomas Schurpf, Cambridge, MA (US); George Coricor, Cambridge, MA (US); Justin W. Jackson, Cambridge, MA (US); Atsuko Polzin, Cambridge, MA (US)

(21) Appl. No.: 18/578,341

(22) PCT Filed: Jul. 14, 2022
(86) PCT No.: PCT/US2022/073740

§ 371 Date: Jan. 11, 2024

Related U.S. Application Data
(60) Provisional application No. 63/319,524, filed on Mar. 14, 2022.
(60) Provisional application No. 63/221,896, filed on Jul. 14, 2021.
(60) Provisional application No. 63/221,910, filed on Jul. 14, 2021.
(60) Provisional application No. 63/298,132, filed on Jan. 10, 2022.

(51) Int. Cl. C07K 16/28; A61K 39/00; A61K 45/06; A61P 11/00; A61P 13/12

(52) U.S. Cl. CPC C07K 16/2863; A61K 45/06; A61P 11/00; A61P 13/12; A61K 2039/505

(57) ABSTRACT

The invention provides method of treating fibrosis, comprising the steps of (a) selecting a TGFβ inhibitor for the treatment of fibrosis, (1) wherein the TGFβ inhibitor (A) specifically binds a LTBP1-proTGFβ complex; (B) does not bind a human GARP-proTGFβ complex; (C) does not bind a human LRRC33-proTGFp complex; and (D) does not bind mature TGFβ1, mature TGFβ2 or mature TGFβ3; and (2) wherein the TGFβ inhibitor is selected using an assay to measure the amount of a marker, wherein the amount of the marker is indicative of the treatment of fibrosis, with the proviso that the marker is not pSmad2; and (b) providing the selected TGFβ inhibitor for administration to a subject in need of treatment for fibrosis. These selected TGFβ inhibitor are isoform-specific, context-selective inhibitors of TGFβ1 that selectively target matrix-associated TGFβ1 activation but not immune cell-associated TGFβ1 activation.

### RELATED APPLICATIONS

This application claims the benefit of and priority to U.S. Provisional Application No. 63/221,896 filed Jul. 14, 2021, U.S. Provisional Application No. 63/221,910 filed Jul. 14, 2021, U.S. Provisional Application No. 63/298,132 filed Jan. 10, 2022, and U.S. Provisional Application No. 63/319,524 filed Mar. 14, 2022, the contents of each of which are expressly incorporated herein by reference in their entireties.

### SEQUENCE LISTING

The instant application contains a Sequence Listing which has been submitted electronically in Sequence Listing XML format and is hereby incorporated by reference in its entirety. Said XML copy, created on Jul. 11, 2022, is named 127036-04720.XML and is 140,245 bytes in size.

### TECHNICAL FIELD

The disclosure generally relates to antibodies or antigen-binding fragments or portions thereof capable of inhibiting a subset of transforming growth factor-beta 1 (TGFβ1) signaling and related methods and uses thereof.

### BACKGROUND

Transforming growth factor beta 1 (TGFβ1) is a member of the TGFβ superfamily of growth factors, along with two other structurally related isoforms, namely, TGFβ2 and TGFβ3, each of which is encoded by a separate gene. TGFβs function as pleiotropic cytokines that regulate cell proliferation, differentiation, immunomodulation (e.g., adaptive immune response), and other diverse biological processes both in homeostasis and in disease contexts. The three TGFβ isoforms signal through the same cell-surface receptors and trigger similar canonical downstream signal transduction events that include the SMAD2/3 pathway.

During protein synthesis, TGFβ polypeptides are produced as precursor forms comprising a prodomain and a growth factor domain. Two chains of the TGFβ polypeptide form a dimer which undergoes proteolysis by a furin convertase that cleaves between the prodomain and the growth factor domain. Upon the furin cleavage, this “latent” proTGFβ remains associated as an inactive complex (“small latent complex” or “SLC”). Furthermore, in the trans Golgi, the proTGFβ SLC becomes covalently attached to an adapter protein referred to as “presenting molecule”, forming a tertiary complex referred to as a large latent complex (“LLC”). To date, four presenting molecules for TGFβ are known: (1) latent TGFβ-binding protein 1 (LTBP1), (2) latent TGFβ-binding protein 3 (LTBP3), (3) glycoprotein A repetitions predominant (GARP, also known as LRRC32), and (4) leucine-rich repeat-containing protein 33 (LRRC33; NRROS). Each of these presenting molecules can form disulfide bonds with a homodimeric pro-protein complex of the TGFβ precursor, i.e., proTGFβ, or SLC. The proTGFβ-presenting molecule complex (LLC) is subsequently secreted into the extracellular space, where it remains “dormant” or inactive (latent) in the respective extracellular niche, e.g., extracellular matrix and immune cell surface, until activation events trigger the release of soluble growth factor from the complex.

TGFβ has been implicated in the pathogenesis and progression of a number of disease conditions, such as fibrosis, cancer and immune disorders. In many cases, such conditions are associated with dysregulation of the extracellular matrix (ECM). For these and other reasons, TGFβ has been an attractive therapeutic target for the treatment of fibrotic conditions as well as various proliferative disorders. However, observations from preclinical studies, including in rats and dogs, have revealed serious toxicities associated with antagonizing TGFβ signaling in vivo, and to date, there are no TGFβ therapeutics available in the market which are deemed both safe and efficacious.

The prevailing view of the field continues to be that it is advantageous to inhibit multiple isoforms of TGFβ to achieve therapeutic effects, and that to manage toxicity by “careful dosing regimen.” See, Brennan et al. (2018) mAbs, 10:1, 1-17. Several groups recently turned to identifying inhibitors that target a subset of the isoforms of TGFβ and still retain sufficient efficacy. Some groups are developing engineered fusion proteins that function as so-called “ligand traps,” at least some of which may be selective or preferential for TGFβ1/3. Another class of inhibitors that suppresses the TGFβ1/3 signaling pathway includes inhibitors of alpha-V (αv) integrins such as antibodies against αvβ6, which is an integrin known to activate both TGFβ1 and TGFβ3, i.e., TGFβ1/3. Still others continue to pursue inhibitors that inhibit all three isoforms of TGFβ, i.e., TGFβ1/2/3, or pan-inhibitors. See, e.g., WO 2018/134681.

Previously, Applicant described a class of monoclonal antibodies that functions with a novel mechanism of action to modulate growth factor signaling (see, for example, WO 2014/182676). These antibodies were designed to exploit the fact that TGFβ1 is expressed as latent pro-protein complex comprised of prodomain and growth factor, which requires an activation step that releases the growth factor from the latent complex. Rather than taking the traditional approach of directly targeting the soluble growth factor itself post-activation (such as neutralizing antibodies), the novel class of inhibitory antibodies specifically targets the inactive pro-proprotein complex itself so as to preemptively block the activation step, upstream of ligand-receptor interaction. Without being bound by theory, this unique mechanism of action may provide advantages for achieving both spatial and temporal benefits in that they act at the source, that is, by targeting the latent proTGFβ1 complex within a disease microenvironment before activation takes place. Indeed, advantages of locally targeting tissue/cell-tethered complex at the source, as opposed to soluble active species (i.e., mature growth factors after being released from the source), are further supported by a recent study. Ishihara et al., (Sci. Transl. Med. 11, eaau3259 (2019) “Targeted antibody and cytokine cancer immunotherapies through collagen affinity”) reported that when systemically administered drugs are targeted to the affected tissue by conjugating with a collagen-binding moiety, they were able to enhance anti-tumor immunity and reduce treatment-related toxicities, as compared to non-targeted counterparts.

Recently, Applicant has taken the novel approach aimed to achieve isoform-selective inhibition of TGFβ1 activation in vivo and demonstrated efficacy and improved safety, as compared to less selective TGFβ inhibitors. See, for example: WO 2017/156500 A8 (Scholar Rock, Inc.), WO 2018/129329 A1 (Scholar Rock, Inc.), WO 2020/014460 (Scholar Rock, Inc.) and WO 2020/014473 (Scholar Rock, Inc.), the contents of all of which are incorporated by reference in their entireties herein. Indeed, this approach has been more recently followed by other groups (see, for example, WO 2021/039945).

Beyond the isoform-selective approach described above, Applicant sought to further fine-tune the pharmacological intervention of TGFβ1 signaling for added safety. To this end, Applicant has identified inhibitory antibodies that specifically target only the LTBP-associated large latent complexes of proTGFβ1, sparing immune cell-associated proTGFβ1. These so-called “LTBP context-specific inhibitors” can be made using the methods previously described e.g., in WO 2019/023661 A1 (Scholar Rock, Inc.); WO 2014/074532 A2 (Scholar Rock Inc.; Children's Medical Center Corporation); and WO 2014/182676 A2 (Scholar Rock, Inc.), the contents of each of which are herein incorporated by reference in their entirety. Early LTBP-selective TGFβ1 inhibitors, which were first disclosed in WO 2019/023661, incorporated by reference in its entirety herein, showed context selectivity and weak inhibitory activities, coupled with suboptimal cross-species reactivity.

Applicant subsequently disclosed more potent TGFβ1 inhibitors that selectively bind to and inhibit LTBP-presented large latent complexes (LLCs) (WO 2020/160291, the contents of which are incorporated by reference in their entirety). When tested at relatively high concentrations (e.g., 30 mg/kg) in preclinical models, these inhibitors were able to suppress phosphorylation of SMAD2/3, demonstrating target engagement and the ability to inhibit the TGFβ pathway in vivo. However, it remained unclear how the observed pharmacodynamics (PD) effects compared to so-called “context-independent” inhibitors, which are antibodies that bind to and inhibit TGFβ1 activation from all four known LLCs (e.g., irrespective of the presenting molecules it is complexed with).

### SUMMARY

The present disclosure provides in vivo effects of TGFβ1 inhibitors that selectively target only a subset of TGFβ1 signaling. More specifically, these TGFβ1 inhibitors selectively bind and thereby inhibit the activation of TGFβ1 associated with the extracellular matrix (ECM) in vivo, where the latent proTGFβ1 complex is tethered to the ECM via LTBP1 and LTBP3, while sparing TGFβ1 associated with immune cells.

Applicant recognized that in many situations, fibrosis is a feature of an underlining disease that results from prolonged/chronic insult or injury to particular tissues or organs involving undesirable inflammation, rather than the root cause of the disease itself, but can create a vicious cycle by further causing added injury and loss of function of the affected tissues/organs. In contrast to cancer immuno-therapy in which TGFβ1 inhibition is aimed to enhance the host's immune response to aid anti-tumor effects (see, for example, Martin et al. (2020) Sci Transl Med., 12(536: eaay8456), therapeutics for fibrosis involving inflammation would benefit from avoiding further immune stimulation.

According to the present disclosure, the LTBP context-selective TGFβ1 inhibitor (such as Ab42 and its derivatives, antibodies that cross-compete with Ab42 for binding, etc.) may be used in the treatment of a disease (e.g., chronic conditions) in which organ injury is driven by inflammation and/or oxidative stress, which can often result in fibrosis of the affected tissue or organ. A body of evidence points to TGFβ1 as a key driver of the pathogenesis or aggravation of fibrotic disorders associated with extracellular matrix dysregulation. Advantageously, LTBP-selective inhibitors of TGFβ1 activation (such as Ab42) are designed to avoid the GARP-TGFβ1 axis associated with regulatory T cell (Treg) function, which may be important in preventing abnormal immune activation that can exacerbate the disease.

To date, relative contributions from the matrix-associated vs immune cell-associated pools of TGFβ1 in these diseases have been unclear. From a safety point of view, Applicant previously postulated that select targeting of the LTBP-associated proTGFβ1 complexes should provide a safer alternative to the context-independent approach by avoiding immune cell-associated proTGFβ1 complexes (see, for example, WO 2020/160291). On the other hand, from an efficacy point of view, a question remained as to whether such narrow intervention of only a subset of TGFβ1 signaling might lead to compromised effectiveness, as compared to context-independent, isoform-selective approach which targets both matrix-associated and immune cell-associated TGFβ1 signaling.

Surprisingly, data presented herein for the first time demonstrate that the select inhibition of the ECM arm of the TGFβ1 pathway alone was sufficient to produce anti-fibrotic effects in multiple preclinical models of fibrosis, including kidney fibrosis models. In addition, in one of the kidney models, the LTBP-selective inhibition approach was just as efficacious in normalizing kidney function, as compared to a context-independent inhibitor as a positive control. Data presented herein reveal the unexpected finding that the degree of anti-fibrotic efficacy produced by LTBP context-selective inhibitors of TGFβ1 was equivalent to that produced by context-independent TGFβ1 inhibitors that bind and inhibit TGFβ1 activation from all four LLCs. Moreover, the LTBP-selective approach did not cause an inflammatory response characterized by disease-associated macrophage accumulation, as compared to less selective approaches.

The inventors of the present disclosure further identified an advantageous epitope within the proTGFβ1 complex to confer LTBP1/3-selective targeting. The epitope comprises one or more residues of the N-terminal stretch of amino acids DMELVKRKRIEAIR (SEQ ID NO:46) within the so-called alpha-1 helix region of the prodomain of the latent proTGFβ1 complex. Advantageously, targeting this region of the antigen complex allows context selective binding because the epitope is available for binding by the antibody when proTGFβ1 is associated with LTBP1 or LTBP3, but not easily available for binding when proTGFβ1 is associated with GARP or LRRC33 on cell surface due to physical hindrance. In preferred embodiments, such epitope further includes one or more amino acid residues within the growth factor domain, which is/are in a close vicinity to the alpha-1 helix region in the three-dimensional structure of proTGFβ1. In further embodiments, the epitope comprises one or more residues of the stretch of amino acids YIDFRKDLGWK (SEQ ID NO: 93) within the growth factor domain.

Preferably, such LTBP-selective TGFβ1 inhibitors are “context-selective” such that the antibody or an antigen-binding fragment thereof can bind recombinant human LTBP1-proTGFβ1 and/or LTBP3-proTGFβ1 with at least 50-fold greater affinities than to recombinant human GARP-proTGFβ1 and/or LRRC33-proTGFβ1. More preferably, such antibody or an antigen-binding fragment thereof can bind recombinant human LTBP1-proTGFβ1 and/or LTBP3-proTGFβ1 with at least 100-fold greater affinities than to recombinant human GARP-proTGFβ1 and/or LRRC33-proTGFβ1. Furthermore, such antibody preferably has an IC50 value (e.g., monovalent IC50 value) of 1 nM or less as measured in a cell-based potency assay, such as CAGA reporter assays.

Accordingly, disclosed herein are isoform-selective, context-selective inhibitors of TGFβ1 activation with advantageous features that can be used for the treatment of diseases characterized by dysregulation of the TGFβ pathway, such as fibrosis. The methods disclosed and claimed herein are based on in vivo data from multiple preclinical disease models, which demonstrate surprisingly effective therapeutic results, such as reduction in the amount of collagen present in a fibrotic tissue, reduction in the amount of new collagen synthesis, and/or reduction in the amount of phosphorylated Smad2 in a fibrotic tissue. Moreover, the disclosure provides novel therapeutic dosing strategies, including a loading/maintenance dosing strategy demonstrated to be surprisingly therapeutically effective in vivo.

More specifically, the TGFβ1 inhibitors include monoclonal antibodies (including immunoglobulins and antigen-binding fragments or portions thereof) that exhibit slow dissociation rates (i.e., off-rates, k_{OFF}). Thus, the disclosure is based at least on the recognition that treatment of chronic and progressive disease such as fibrosis, may require inhibitors with superior durability, which may be reflected in the dissociation rate of such antibody.

The affinity of an antibody to its antigen is typically measured as the equilibrium dissociation constant, or K_{D}. The ratio of the experimentally measured off- and on-rates (k_{OFF}/k_{ON}) can be used to calculate the K_{D} value. The k_{OFF} value represents the antibody dissociation rate, which indicates how quickly it dissociates from its bound antigen, whilst the k_{ON} value represents the antibody association rate which provides how quickly it binds to its antigen. The latter is typically concentration-dependent, while the former is concentration-independent. The K_{D} value relates to the concentration of antibody (the amount of antibody needed for a particular experiment) and so the lower the K_{D} value (lower concentration) and thus the higher the affinity of the antibody. With respect to a reference antibody, a higher affinity antibody may have a lower k_{OFF} rate, a higher k_{ON} rate, or both.

Both the k_{OFF} and k_{ON} rates contribute to the overall affinity of a particular antibody to its antigen, and relative importance or impact of each component may depend on the mechanism of action of the antibody. For example, neutralizing antibodies, which bind mature growth factors (e.g., soluble, transient TGFβ1 ligand liberated from a latent complex), must compete with the endogenous high-affinity receptors for ligand binding in vivo. Because the ligand-receptor interaction is a local event and because the ligand is short-lived, such antibodies must be capable of rapidly targeting and sequestering the soluble growth factor before the ligand finds its cellular receptor—thereby activating the TGFβ1 signaling pathway—in the tissue. Therefore, for ligand-targeting neutralizing antibodies to be potent, the ability to bind the target growth factor fast, i.e., high association rates (k_{ON}), may be especially important.

By contrast, Applicant reasoned that antibodies that inhibit the TGFβ1 signaling by preventing the release of mature growth factor from the latent complex (“activation inhibitors”) may preferentially benefit from having slow dissociation rates once the antibody is engaged with the target antigen (e.g., proTGFβ1 complexes). Unlike neutralizing antibodies, such antibodies do not directly compete with cellular receptors; rather, they work upstream of the signaling by targeting inactive precursor forms (e.g., latent proTGFβ1 complexes) that remain dormant within a tissue environment thereby preemptively preventing the activation of TGFβ1. Such antibodies may exert their inhibitory activity by preventing mature growth factor from being liberated from the latent complex. For example, such antibodies may function like a “clamp” to lock the active growth factor in the prodomain cage structure to keep it in an inactive (e.g., “latent”) state. Indeed, structural analyses, including epitope mapping, provided insight into the molecular mechanism underlining the ability of these antibodies to block TGFβ1 activation. In this regard, the Latency Lasso region of the prodomain may be a particularly useful target.

Upon target engagement, antibodies that are able to remain bound to the target (e.g., dissociate very slowly from the latent complex) are expected to be advantageous in achieving superior in vivo potency, due to enhanced durability of effects and/or avidity. Based on this recognition, Applicant of the present disclosure sought to identify isoform-selective activation inhibitors of TGFβ1 with particularly low k_{OFF} values as compared to previously described antibodies. Thus, according to the disclosure, preferred antibodies have high affinities (e.g., a K_{D} of sub-nanomolar to picomolar range) primarily attributable to a slow dissociation rate (k_{OFF}), as opposed to fast association rate (k_{ON}). In some embodiments, such antibodies bind an epitope that comprises at least a portion of Latency Lasso.

In some embodiments, the antibody comprises a CDRH1 sequence comprising FTFRSYVMH (SEQ ID NO: 1); a CDRH2 sequence comprising VISHEGSLKYYADSVKG (SEQ ID NO: 2); a CDRH3 sequence comprising ARPRIAARRGGFGY (SEQ ID NO:3); a CDRL1 sequence comprising TRSSGNIDNNYVQ (SEQ ID NO: 4); a CDRL2 sequence comprising EDNQRPS (SEQ ID NO: 5) and a CDRL3 sequence comprising QSYDYDTQGVV (SEQ ID NO: 6). In some embodiments, the antibody comprises a CDRH1 sequence comprising an amino acid sequence at least 85%, 90%, 95%, 96%, 97%, 98% or 99% identical to FTFRSYVMH (SEQ ID NO: 1); a CDRH2 sequence comprising an amino acid sequence at least 85%, 90%, 95%, 96%, 97%, 98% or 99% identical to VISHEGSLKYYADSVKG (SEQ ID NO: 2); a CDRH3 sequence comprising an amino acid sequence at least 85%, 90%, 95%, 96%, 97%, 98% or 99% identical to ARPRIAARRGGFGY (SEQ ID NO:3); a CDRL1 sequence comprising an amino acid sequence at least 85%, 90%, 95%, 96%, 97%, 98% or 99% identical to TRSSGNIDNNYVQ (SEQ ID NO: 4); a CDRL2 sequence comprising an amino acid sequence at least 85%, 90%, 95%, 96%, 97%, 98% or 99% identical to EDNQRPS (SEQ ID NO: 5) and a CDRL3 sequence comprising an amino acid sequence at least 85%, 90%, 95%, 96%, 97%, 98% or 99% identical to QSYDYDTQGVV (SEQ ID NO: 6). In some embodiments, the antibody comprises a CDRH1 sequence consisting of FTFRSYVMH (SEQ ID NO: 1); a CDRH2 sequence consisting of VISHEGSLKYYADSVKG (SEQ ID NO: 2); a CDRH3 sequence consisting of ARPRIAARRGGFGY (SEQ ID NO:3); a CDRL1 sequence consisting of TRSSGNIDNNYVQ (SEQ ID NO: 4); a CDRL2 sequence consisting of EDNQRPS (SEQ ID NO: 5) and a CDRL3 sequence consisting of QSYDYDTQGVV (SEQ ID NO: 6). In particularly preferred embodiments, the antibody comprises the six CDR sequences of Ab42. In particularly preferred embodiments, the antibody consists of the six CDR sequences of Ab42.

The disclosure includes compositions, such as pharmaceutical compositions (e.g., formulations, medicament) that are suitable for administration to human patients, comprising at least one of the antibodies or fragment thereof in accordance with the present disclosure, and an excipient. Thus, the antibodies or fragment thereof in accordance with the present disclosure can be used in the manufacture of such medicament.

The disclosure further provides therapeutic use of such antibodies. Thus, the TGFβ1-selective inhibitors (e.g., monoclonal antibodies or antigen-binding fragments thereof) of the present disclosure may be used in the treatment of TGFβ1-related indications in a subject. The TGFβ1-selective inhibitors may be particularly advantageous for treating such disease or disorders involving dysregulation of the extracellular matrix (ECM), including, for example, fibrotic disorders (such as organ fibrosis, and fibrosis involving chronic inflammation), proliferative disorders (such as cancer, e.g., solid tumors and myelofibrosis), disease involving endothelial-to-mesenchymal transition (EndMT), disease involving epithelial-to-mesenchymal transition (EMT), disease involving proteases, disease with aberrant gene expression of certain markers described herein. The TGFβ31-selective inhibitors may be used in conjunction with another therapy as combination therapies (e.g., add-on therapies). Methods for treating such disease or disorders comprising administration of the TGFβ1-selective inhibitor in a subject, either as monotherapy or combination therapy, are encompassed by the disclosure.

According to one aspect, the disclosure provides a method of treating fibrosis in a subject, the method comprising steps of administering a therapeutically effective amount of a TGFβ inhibitor to the subject as a loading dose/maintenance dose regimen, wherein the TGFβ inhibitor inhibits TGFβ1 but does not inhibit one or both of TGFβ2 and/or TGFβ3, thereby treating fibrosis in the subject.

This disclosure provides evidence showing that selective inhibition of extracellular TGFβ1 (e.g., LTBP1-proTGFβ1 and LTBP3-proTGFβ1) is sufficient to reduce fibrosis in multiple preclinical models, including diet-induced liver fibrosis and kidney injury models. Surprisingly, anti-fibrotic effects were observed at doses not sufficient to show reduced SMAD2 phosphorylation. More specifically, pharmacodynamic effects as measured by p-SMAD2 or p-SMAD2/3 were evident at relatively higher doses of Ab42, such as 30 mg/kg, but not at lower doses. This observation led to the notion that Ab42 was efficacious at 30 mg/kg or higher doses. Unexpectedly, antifibrotic efficacy was achieved at lower doses (such as 10 mg/kg) of Ab42 in Alport and adenine-induced kidney injury models. In the liver fibrosis model, LTBP-selective inhibition of TGFβ1 can reduce new collagen synthesis as measured by hydroxyproline content. In the kidney models, LTBP-selective inhibition of TGFβ1 can reduce new collagen synthesis as measured by hydroxyproline contents, reduced collagen deposits; collagen accumulation as measured by PSR staining, and normalize kidney function as measured by plasma concentrations of blood urea nitrogen (BUN) and creatinine. These data suggest that selective targeting of the extracellular TGFβ1 can achieve anti-fibrotic effects in multiple organ fibrosis models in vivo. Moreover, data presented herein suggest that use of SMAD2/3 phosphorylation as a pharmacodynamic marker may not be informative in determining exposures (e.g., serum concentrations) of a test drug (e.g., antibodies, or fragments thereof) required to achieve efficacy. Collectively, data indicate that serum exposure of approximately 200 μg/mL is sufficient to reduce tissue collagen accumulation as measured by PSR staining with Ab42. This contrasts with the previous observation in rat liver fibrosis model that approximately 340 μg/mL serum exposure was required to observe target engagement as determined by reduced p-SMAD2. Indeed, despite higher serum exposure than reference antibody (a context-independent inhibitor of TGFβ1 activation), Ab42 demonstrated little to no pSMAD suppression at twenty-four hours. These data also suggest that a loading dose approach may provide consistent serum concentrations over treatment duration.

Accordingly, the disclosure provides an LTBP-selective inhibitor of TGFβ1 activation for use in the treatment of fibrosis in a subject, wherein the treatment comprises administration of an effective amount of the LTBP-selective inhibitor of TGFβ1 activation.

In some embodiments, the LTBP-selective inhibitor of TGFβ1 activation is Ab42 or a variant thereof. In some embodiments, the LTBP-selective inhibitor of TGFβ1 activation is an engineered construct comprising an antigen-binding fragment of Ab42 or its variant. The variant of Ab42 or the engineered construct preferably binds an epitope that comprises one or more amino acid residues of VKRKRIEA (SEQ ID NO: 9), which is in the α1-helix region of the prodomain of proTGFβ1. In another embodiment, the variant of Ab42 or the engineered construct preferably binds an epitope that comprises one or more amino acid residues of DMELVKRKRIEAIR (SEQ ID NO:46). Additionally or alternatively, in some embodiments, the inhibitor is an antibody which is not Ab42 and competes binding with Ab42 and binds an epitope comprising one or more residues of the amino acid stretch YIDFRKDLGWK (SEQ ID NO: 93) within the TGFβ1 growth factor domain (e.g., the “finger-1” domain). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42. In some embodiments, the antibody or fragment thereof contacts one or more of the following residues: Tyr289 (Y); Lys294 (K); Asp295 (D) (shown in bold above).

In some embodiments, the effective amount of the LTBP-selective inhibitor of TGFβ1 activation (such as Ab42) is an amount sufficient to reduce tissue collagen deposits or accumulation, as measured by, for example, PSR staining and/or hydroxyproline contents. In some embodiments, the effective amount is an amount sufficient to normalize gene expression of certain genes as described herein. In some embodiments, the effective amount is an amount that is below the amount required to reduce p-SMAD2 or p-SMAD2/3 levels as measured by ELISA. In some embodiments, the effective amount is below 30 mg/kg of Ab42 or a fragment or a variant thereof. In some embodiments, the treatment comprises administration of Ab42 or a fragment or a variant thereof at 1-25 mg/kg, e.g., 1, 2, 3, 5, 7, 7.5, 8, 10, 12, 12.5, 14, 15, 16, 17, 18, 19, 20, 21, 22, 23, 24 or 25 mg/kg. In some embodiments, the effective amount is an amount sufficient to achieve serum exposure of between 180-220 μg/mL (e.g., 180, 185, 190, 195, 200, 205, 210, 215 or 220 μg/mL). In some embodiments, the effective amount is an amount sufficient to achieve serum exposure of 200 μg/mL. In some embodiments the treatment comprises administration of a loading dose followed by a maintenance dose of the LTBP-selective inhibitor of TGFβ1 activation (e.g., Ab42, or a fragment or a variant thereof). In some embodiments, the loading dose is administered intravenously and the maintenance dose is administered subcutaneously.

In some embodiments, the treatment comprises a subcutaneous injection of a composition comprising the LTBP-selective inhibitor of TGFβ1 activation, such as Ab42, or a fragment or a variant thereof.

In some embodiments, combination therapy is provided. For example, the treatment further comprises one or more additional therapeutic agents. Examples of the additional therapeutic agents which can be used with an anti-TGFβ antibody of the disclosure include, but are not limited to, a myostatin inhibitor, a VEGF agonist, an IGF1 agonist, an FXR agonist, a CCR2 inhibitor, a CCR5 inhibitor, a dual CCR2/CCR5 inhibitor, a lysyl oxidase-like-2 inhibitor, an ASK1 inhibitor, an Acetyl-CoA Carboxylase (ACC) inhibitor, a β8 kinase inhibitor, pirfenidone, nintedanib, selonsertib, cilofexor, firsocostat, obeticholic acid, elafibranor, an anti-CD147 antibody, an anti-GP73 antibody, a galactin-1 inhibitor, selonsertib, a caspase inhibitor (EMRICASAN®, IDN-6556, PF-03491390), a GDF11 inhibitor, a GDF8/myostatin inhibitor, a GLP-1 receptor agonist, and the like. The GDF8/myostatin inhibitor is preferably a myostatin-selective inhibitor (e.g., an antibody or an antigen-biding fragment). The myostatin-selective inhibitor may bind latent myostatin. Non-limiting examples of myostatin-selective inhibitors include apitegromab (also known as SRK-015) (e.g., see WO 2017/218592 A1) and trevogrumab (also known as REGN1033), or any variant thereof, or an antibody according to WO 2016/098357, GYM329 (RO7204239) and variants thereof.

In some embodiments, the additional agent is a checkpoint inhibitor. In some embodiments, the additional agent is selected from the group consisting of a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an oncolytic virus, and a PARP inhibitor. In some embodiments, the additional therapy is radiation. In some embodiments, the additional agent is a chemotherapeutic agent. In some embodiments, the chemotherapeutic agent is Taxol. In some embodiments, the additional agent is an anti-inflammatory agent. In some embodiments, the additional agent inhibits the process of monocyte/macrophage recruitment and/or tissue infiltration. In some embodiments, the additional agent is an inhibitor of hepatic stellate cell activation. In some embodiments, the additional agent is a chemokine receptor antagonist, e.g., CCR2 antagonists and CCR5 antagonists. In some embodiments such chemokine receptor antagonist is a dual specific antagonist, such as a CCR2/CCR5 antagonist. In some embodiments, the additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that binds a pro/latent myostatin complex and blocks activation of myostatin. In some embodiments, the monoclonal antibody that binds a pro/latent myostatin complex and blocks activation of myostatin does not bind free, mature myostatin.

A combination therapy that includes a TGFβ inhibitor (such as TGFβ1-selective inhibitors disclosed herein), in conjunction with one or more additional therapies, may be considered for treating a variety of liver diseases. Non-limiting examples of liver diseases include: non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and primary sclerosing cholangitis (PSC).

In some embodiments, one or more of the following therapies may be used in conjunction with the TGFβ inhibitor (such as TGFβ1-selective inhibitors disclosed herein) for the treatment of a liver disease such as those listed above: Pioglitazone (PPARγ agonist); Elafibranor (PPARα/δ agonist); Saroglitazar (PPARα/γ agonist); Obeticholic acid (FXR agonist); Liraglutide (GLP-1 receptor agonist); semaglutide (GLP-1 receptor agonist); Aramchol (SCD inhibitor); Volixibat (SHP-626) (ASBT inhibitor); BMS-986036 (FGF-21 analogue); NGM-282 (FGF-19 analogue); Tesamorelin (GHRH analogue); NDI-010976 (ACC inhibitor); GS-9674 (FXR agonist); Dur-928 (Sulfated oxysterol); AZD4076 (miR-103/107 antagonist); Rosuvastatin (HMG-CoA reductase inhibitor); INT-767 (FXR/TGR5 agonist); Sevelamer (Bile acid sequestrant); Vitamin E (Antioxidant); Pentoxifylline (PDE inhibitor); Cenicriviroc (CCR2/CCR5 antagonist); Emricasan (Caspase inhibitors); GS-4997 (ASK1 inhibitor); Amlexanox (IKKε/TBK1 inhibitor); PXS-4728A (VAP-1 inhibitor); Orlistat (Intestinal lipase inhibitor); IMM-124e (IgG-rich bovine colostrum); Solithromycin (Antibiotic); Faecal microbial transplant (Modulation of gut microbiome); Simtuzumab (LOXL2 antibody); GR-MD-02 (Galectin-3 inhibitor); Trevogrumab (myostatin inhibitor); Garetosmab (activin A inhibitor); GYM329 (myostatin inhibitor), and apitegromab/SRK-015 (myostatin inhibitor).

Such combination therapies may advantageously utilize lower dosages of the administered therapeutic agents, thus avoiding possible toxicities or complications associated with the various monotherapies. In some embodiments, the combination therapy may comprise administration of a composition comprising two or more of the therapeutic agents formulated in a single formulation. Alternatively, in some embodiments, the combination therapy may comprise administration of two or more compositions in which two or more of the therapeutic agents are formulated separately. The composition or compositions may be formulated for intravenous infusion or subcutaneous injection.

In some embodiments, add-on or adjunct therapies are contemplated. The LTBP-selective inhibitor of TGFβ1 activation (such as Ab42) is used in the treatment of fibrosis in a subject who has received or is on a background therapy. The background therapy may comprise any one of the following: a myostatin inhibitor, a VEGF agonist, an IGF1 agonist, an FXR agonist, a CCR2 inhibitor, a CCR5 inhibitor, a dual CCR2/CCR5 inhibitor, a lysyl oxidase-like-2 inhibitor, an ASK1 inhibitor, an Acetyl-CoA Carboxylase (ACC) inhibitor, a β8 kinase inhibitor, Pirfenidone, Nintedanib, selonsertib, cilofexor, firsocostat, Pirfenidone, obeticholic acid, elafibranor, an anti-CD147 antibody, an anti-GP73 antibody, a Galactin-1 inhibitor, selonsertib, a caspase inhibitor (Emricasan, IDN-6556, PF-03491390), a GDF11 inhibitor, a GDF8/myostatin inhibitor (e.g., anti-myostatin neutralizing antibodies, anti-ActRII antibodies such as bimagromab, GDF8/GDF11 ligand traps, anti-myostatin Adnectins such as BMS-986089/Taldefgrobep alpha, anti-pro/latent myostatin antibodies, Follistatin or analogs thereof, etc.), a GLP-1 receptor agonist, a biguanide (e.g., metformin), a DPP-4 inhibitor, and the like. In some embodiments, the GDF8/myostatin inhibitor is preferably a myostatin-selective inhibitor (e.g., an antibody or an antigen-biding fragment). The myostatin-selective inhibitor may bind latent myostatin. Non-limiting examples of myostatin-selective inhibitors include SRK-015 (e.g., see WO2017/218592A1) and trevogrumab, or any variant thereof, or an antibody according to WO 2016/098357, GYM329 and variants thereof; Pioglitazone (PPARγ agonist); Elafibranor (PPARα/δ agonist); Saroglitazar (PPARα/γ agonist); Obeticholic acid (FXR agonist); Liraglutide (GLP-1 receptor agonist); semaglutide (GLP-1 receptor agonist); Aramchol (SCD inhibitor); Volixibat (SHP-626) (ASBT inhibitor); BMS-986036 (FGF-21 analogue); NGM-282 (FGF-19 analogue); Tesamorelin (GHRH analogue); NDI-010976 (ACC inhibitor); GS-9674 (FXR agonist); Dur-928 (Sulfated oxysterol); AZD4076 (miR-103/107 antagonist); Rosuvastatin (HMG-CoA reductase inhibitor); INT-767 (FXR/TGR5 agonist); Sevelamer (Bile acid sequestrant); Vitamin E (Antioxidant); Pentoxifylline (PDE inhibitor); Cenicriviroc (CCR2/CCR5 antagonist); Emricasan (Caspase inhibitors); GS-4997 (ASK1 inhibitor); Amlexanox (IKKε/TBK1 inhibitor); PXS-4728A (VAP-1 inhibitor); Orlistat (Intestinal lipase inhibitor); IMM-124e (IgG-rich bovine colostrum); Solithromycin (Antibiotic); Faecal microbial transplant (Modulation of gut microbiome); Simtuzumab (LOXL2 antibody); GR-MD-02 (Galectin-3 inhibitor); Trevogrumab (myostatin inhibitor); Garetosmab (activin A inhibitor); GYM329 (myostatin inhibitor), and SRK-015 (myostatin inhibitor).

In some embodiments, the invention provides a method for treating fibrosis, comprising the steps of (1) selecting a TGFβ inhibitor for the treatment of fibrosis, (a) wherein the TGFβ inhibitor (i) specifically binds a LTBP1-proTGFβ complex; (ii) does not bind a human GARP-proTGFβ complex; (iii) does not bind a human LRRC33-proTGFβ complex; and (iv) does not bind mature TGFβ1, mature TGFβ2 or mature TGFβ3; and (b) wherein the TGFβ inhibitor is selected using an assay to measure the amount of a marker, wherein the amount of the marker is indicative of the treatment of fibrosis, with the proviso that the marker is not pSMAD2; and (2) providing the selected TGFβ inhibitor for administration to a subject in need of treatment for fibrosis. These selected TGFβ inhibitors are isoform-specific, context-selective inhibitors of TGFβ1 that selectively target matrix-associated TGFβ1 activation but not immune cell-associated TGFβ1 activation. In some embodiments, when the TGFβ inhibitor is an antibody, or a fragment thereof, comprising six CDRs: (1) CDR-H1 comprising the amino acid sequence FTFRSYVMH (SEQ ID NO: 1); (2) CDR-H2 comprising the amino acid sequence VISHEGSLKYYADSVKG (SEQ ID NO: 2); (3) CDR-H3 comprising the amino acid sequence ARPRIAARRGGFGY (SEQ ID NO: 3); (4) CDR-L1 comprising the amino acid sequence TRSSGNIDNNYVQ (SEQ ID NO: 4); (5) CDR-L2 comprising the amino acid sequence EDNQRPS (SEQ ID NO: 5); and (6) CDR-L3 comprising the amino acid sequence QSYDYDTQGVV (SEQ ID NO: 6). When the TGFβ inhibitor is an antibody, the selected antibody can be a fully human or humanized antibody, or antigen binding fragment thereof, wherein the antibody comprises the six recited CDR sequences.

In some embodiments, when the TGFβ inhibitor is an antibody, the selected antibody comprises an antibody or antigen-binding fragment thereof that competes or cross-competes with an antibody having a heavy chain variable region sequence as set forth in SEQ ID NO: 7 and light chain variable region sequence as set forth in SEQ ID NO: 8, e.g., Ab42.

In some embodiments, when the TGFβ inhibitor is an antibody, the selected antibody comprises the LTBP-specific antibody Ab42, which is highly selective, with picomolar monovalent affinities. Ab42 is described in WO 2020/160291 A2, incorporated by reference in its entirety herein. Ab42 was shown to have greater than 100-fold selectivity for LTBP-complexed TGFβ1 as compared to GARP- and LRRC33-proTGFβ1 complexes. As demonstrated herein, no detectable binding to cell lines overexpressing human or murine LRRC33-proTGFβ1 or detectable binding to activated human Tregs was observed. As further demonstrated herein, Ab42 was a potent inhibitor of LTBP complexed TGFβ1 activation in cell-based assays.

In some embodiments, when the TGFβ inhibitor is an antibody, the selected antibody comprises an isoform-specific, context-selective inhibitor of TGFβ1 that selectively targets matrix-associated TGFβ1 activation but not immune cell-associated TGFβ1 activation. Such inhibitors specifically bind a particular isoform of TGFβ, e.g., proTGFβ1, proTGFβ2, or proTGFβ3, associated with LTBP1 or LTBP3, thus also providing TGFβ isoform specificity. In some embodiments, such inhibitors specifically bind to LTBP-proTGFβ1.

In some embodiments, the disclosure provides isoform-selective inhibitors of TGFβ1 activation for use in the treatment of fibrosis, wherein the dosage is determined based upon pharmacodynamics markers that can be used to demonstrate consistent target engagement. These pharmacodynamic markers do not include standard measurements of SMAD phosphorylation. Accordingly, the dosages incorporate the dose limitations provided in this specification.

In some embodiments, the disclosure provides isoform-selective inhibitors of TGFβ1 activation for use in the treatment of fibrosis, wherein the dosage comprises a loading dose followed by maintenance dose. In some embodiments, the loading dose is administered intravenously and the maintenance dose is administered subcutaneously.

The present disclosure is based upon the surprising finding that there was a discrepancy between pharmacokinetics/pharmacodynamics results and the efficacy results when Ab42 was tested in the adenine rat model described in the EXAMPLES, e.g., EXAMPLE 1. Although the inventors had previously observed a small pSMAD2 suppression with Ab42 at twenty-four hours in a pharmacokinetics/pharmacodynamics (PK/PD) analysis, the signal was not observed at forty-eight or ninety-six hours, despite sustained serum exposure. By contrast, in a corresponding Ab42 efficacy analysis, the inventors observed a reduction of plasma blood urea nitrogen and creatinine. See the EXAMPLES.

Persons having ordinary skill in the immunological art generally determine the dose of the therapeutic to be administered based upon a pharmacokinetic or pharmacodynamic marker that predicts therapeutic efficacy, e.g., the treatment of fibrosis. For therapeutic treatments involving TGFβ, a pharmacodynamic marker known to persons having ordinary skill in the immunological art is the phosphorylation of SMAD protein (p-SMAD), as measured by standard assay, such as ELISAs. In the case of Ab42, it initially escaped notice that Ab42 was an effective inhibitor of fibrosis, because there were clear pharmacodynamic effects observed (e.g., Smad phosphorylation) with lower doses. In earlier results, Ab42 was used at a dose of 30 mg/kg, which tentatively showed that downstream phospho-smad2/3 was reduced by Ab42. The use of lower Ab42 concentrations did not result in PD effects when lower doses were used (by comparison, the context-independent antibody 37021 showed effects). The conclusion could have been that Ab42 would be effective only at quite high concentrations (e.g., 30 mg/kg or higher). Nevertheless, the results of this specification provided guidance for the unexpected efficacy of Ab42 treatment for treatment of fibrotic conditions that involve LTBP and the extracellular matrix. The results of the liver fibrosis (CDHFD) efficacy analysis in the EXAMPLES below showed effectively reduced picrosirius red staining and reduced hydroxyproline contents by Ab42 at doses lower than 30 mg/kg.

The results of the disclosure demonstrated, in part, the surprising finding that p-SMAD2 or pSMAD2/3 was not a preferred measure of the effectiveness of LTBP-selective inhibitors, because the phosphorylation did not correlate with anti-fibrotic efficacy. It was found that LTBP-mediated signaling could be highly localized, such that measurements of Smad phosphorylation at a whole tissue level, by ELISA for example, would not be detected. In summary, P-SMAD2 or P-SMAD2/3 was not a suitable marker for the use of LTBP-selective inhibitors, although it is a widely accepted marker of TGFβ signaling.

In one aspect, the disclosure provides the concept of loading dose followed by maintenance dose. In some embodiments, the loading dose is administered intravenously and the maintenance dose is administered subcutaneously.

The pharmacokinetics simulation results described herein provided a rationale for a loading dose approach in rat adenine efficacy analysis. A loading dose approach thus resulted in consistent antibody exposure levels over treatment period and was in line with PK simulation. The loading dose strategy resulted in consistent serum exposure over treatment duration.

Rodent latent TGFβ1 binding (K_{D}) was determined to be within ten-fold of humans, which allows for proper toxicology assessment.

In some embodiments the disclosure provides a combination therapy for the treatment of fibrosis, e.g., lung fibrosis (e.g., idiopathic pulmonary fibrosis (IPF)), kidney fibrosis (e.g., Alport syndrome), or muscle fibrosis (e.g., muscle fibrosis in Duchene's muscular dystrophy (DMD), which has clinical and preclinical evidence for LTBP involvement). The combination treatment comprised administration of pirfenidone, nintedanib, and Ab42.

These results of the assays using these small molecules are disclosed in this specification. The positive control small molecule inhibitors of TGFβ type I (ALK5) receptor (ALK5i), pirfenidone, and nintedanib did not reduce picrosirius red staining when compared to the vehicle control.

In some embodiments, the disclosure provides for the administration of Ab42 to a subject who is currently receiving pirfenidone and nintedanib treatment.

In some embodiments, the disclosure provides for the administration of Ab42 to a subject who had previous been receiving pirfenidone and nintedanib treatment, i.e., an add-on or adjunct therapy.

In another aspect, the disclosure provides for the selection of a target patient or patient population who has received or is on a background therapy, such as approved standard-of-care.

In some embodiments, the LTBP-selective inhibitor of TGFβ1 activation for use in the treatment of a fibrotic condition in a subject, is an antibody or antigen-binding fragment thereof that binds an epitope in the α-1-helix region of proTGFβ1. In further embodiments, the epitope comprises one or more amino acid residues of VKRKRIEA (SEQ ID NO: 9). In other further embodiments, the epitope comprises one or more amino acid residues of DMELVKRKRIEAIR (SEQ ID NO: 46). In other further embodiments, the antibody is not Ab42.

In some embodiments, the fibrotic condition is NASH, NAFLD, liver fibrosis, chronic kidney disease (CKD), kidney fibrosis, lung disease, lung fibrosis, COPD, IPF, scleroderma, Alport syndrome, bone marrow disorders (e.g., myeloproliferative disorder, myelofibrosis, myelodysplasic syndrome (MDS)), and/or a muscular dystrophy (e.g., DMD).

The disclosure also provides in other aspects an LTBP-selective inhibitor of TGFβ1 activation for use in the treatment of chronic kidney disease (CKD) in a subject, wherein the treatment comprises administration of an LTBP-selective inhibitor which is an antibody that does not bind GARP-proTGFβ1, in an amount effective to treat CKD, wherein optionally the subject is further treated with a myostatin-selective inhibitor.

The disclosure also provides in other aspects a myostatin-selective inhibitor for use in the treatment of CKD in a subject, wherein the treatment comprises administration of a myostatin-selective inhibitor to the subject in an amount effective to treat CKD, wherein optionally the subject is further treated with an LTBP-selective inhibitor of TGFβ1 activation.

In some embodiments, an LTBP-selective inhibitor and a myostatin-selective inhibitor are used in the treatment of CKD in a subject, wherein the treatment comprises administration of an LTBP-selective inhibitor and a myostatin-selective inhibitor to the subject in amounts effective to treat CKD. In some embodiments, the CKD is associated with iron deficiency, wherein optionally the subject is treated with an iron-enhancing therapy.

In some embodiments, the treatment comprises subcutaneous administration of a composition comprising the LTBP-selective inhibitor of TGFβ1 activation.

In another embodiment, the LTBP-selective inhibitor of TGFβ1 activation is a variant of Ab42. In some embodiments, the variant of Ab42 is referred to as Ab42-YTE. In some embodiments, the variant of Ab42 comprises the six CDRs as Ab42, and further comprising a set of three amino acid mutations (YTE) in the Fc region, which increases its affinity for FcRn, resulting in longer serum half-life. In some embodiments, Ab42-YTE retains its affinity for LTBP1/3-proTGFβ1. In some embodiments, the affinity is equivalent to Ab42.

According to one aspect, the disclosure provides a LTBP-selective TGFβ1 inhibitor for use in the treatment of an inflammatory fibrotic disease in a subject, wherein the treatment comprises administration of an effective amount of a LTBP-selective TGFβ1 inhibitor to the subject, wherein the LTBP-selective TGFβ1 inhibitor is a) an antibody or antigen-binding fragment thereof comprising the following six CDRs: (1) CDR-H1 comprising the amino acid sequence FTFRSYVMH (SEQ ID NO: 1); (2) CDR-H2 comprising the amino acid sequence VISHEGSLKYYADSVKG (SEQ ID NO: 2); (3) CDR-H3 comprising the amino acid sequence ARPRIAARRGGFGY (SEQ ID NO: 3); (4) CDR-L1 comprising the amino acid sequence TRSSGNIDNNYVQ (SEQ ID NO: 4); (5) CDR-L2 comprising the amino acid sequence EDNQRPS (SEQ ID NO: 5); and (6) CDR-L3 comprising the amino acid sequence QSYDYDTQGVV (SEQ ID NO: 6); b) an antibody that is not Ab42 and comprises a VH and a VL each of which has at least 90% sequence identity to SEQ ID NO: 7 and SEQ ID NO: 8, respectively, and binds human LTBP1-proTGFβ1 and human LTBP3-proTGFβ1 with a K_{D} of 10 nM or less as measured by a SPR-based assay (such as Biacore); c) an antibody that is not Ab42 and competes for antigen binding with SR42 and binds an epitope comprising one or more amino acid residues of the sequence DMELVKRKRIEAIR (SEQ ID NO: 46), wherein optionally the antibody contacts one or more of the residues: Asp27 (D); Leu30 (L); Arg33 (R); Ile36 (I); Glu37 (E); or, d) an antibody that is not Ab42 and competes for antigen binding with SR42 and binds an epitope comprising one or more amino acid residues of the sequence YIDFRKDLGWK (SEQ ID NO: 93), wherein optionally the antibody contacts one or more of the residues: Tyr289 (Y); Lys294 (K); Asp295 (D); wherein the antibody binds recombinant human LTBP1-proTGFβ1 and/or recombinant human LTBP3-proTGFβ1 with at least 50-fold greater affinities over recombinant human GARP-proTGFβ1 as measured in an in vitro binding assay.

According to some embodiments, the inflammatory fibrotic disease is a viral infection, wherein optionally the viral infection is a severe acute respiratory syndrome. According to further embodiments, the severe acute respiratory syndrome is SARS-CoV (e.g., SARS-CoV1) or COVID19 (SARS-CoV2).

According to some embodiments, the inflammatory fibrotic disease is a chronic kidney disease (CKD), a chronic lung disease, a chronic liver disease, diabetes, muscle disease, systemic sclerosis, cancer, and/or genotoxic therapy-induced inflammation. According to some embodiments of the above aspects and embodiments, the chronic kidney disease is diabetic nephropathy, renal fibrosis, and/or Alport syndrome. According to some embodiments of the above aspects and embodiments, the e chronic liver disease is non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and/or primary sclerosing cholangitis (PSC), wherein optionally the subject has or is at risk of developing obesity, metabolic syndrome, and/or type 2 diabetes. According to some embodiments of the above aspects and embodiments, the LTBP-selective TGFβ1 inhibitor is used in conjunction with another therapy comprising a myostatin inhibitor and/or a GLP-1 receptor agonist. According to some embodiments of the above aspects and embodiments, the myostatin inhibitor is a myostatin-selective inhibitor, wherein optionally the myostatin-selective inhibitor is apitegromab, GYM329, trevogromab, or a variant thereof. According to some embodiments of the above aspects and embodiments, the myostatin inhibitor is a non-selective myostatin inhibitor, wherein optionally the non-selective myostatin inhibitor is an antibody that binds ActRII (e.g., bimagromab), a ligand trap, or an anti-myostatin Adnectin (e.g., BMS-986089). According to some embodiments of the above aspects and embodiments, the diabetes is type 1 diabetes or type 2 diabetes. According to some embodiments of the above aspects and embodiments, the chronic lung disease is IPF, infection, and/or radiation-induced pulmonary fibrosis. According to some embodiments of the above aspects and embodiments, the muscle disease is a dystrophy, wherein optionally the dystrophy is DMD, wherein further optionally the subject is treated with a dystrophin-directed therapy. According to some embodiments of the above aspects and embodiments, the cancer comprises a fibrotic solid tumor. According to further embodiments, the fibrotic solid tumor is a desmoplasia. According to some embodiments of the above aspects and embodiments, the e subject is treated with a genotoxic therapy, wherein the genotoxic therapy-induced inflammation is radiation therapy-induced inflammation or chemotherapy-induced inflammation, wherein optionally, the subject has cancer selected from uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), head and neck squamous cell carcinoma (HNSC), glioblastoma multiforme (GMB), esophageal carcinoma (ESCA), colon adenocarcinoma (COAD), breast invasive carcinoma (BRCA), bladder urothelial carcinoma (BLCA), myelofibrosis, melanoma, adjuvant melanoma, renal cell carcinoma (RCC) (e.g., clear cell RCC, papillary RCC, chromophobe RCC, collecting duct RCC, and unclassified RCC), bladder cancer, colorectal cancer (CRC) (e.g., microsatellite-stable CRC, mismatch repair deficient colorectal cancer), colon cancer, rectal cancer, anal cancer, breast cancer, triple-negative breast cancer (TNBC), HER2-negative breast cancer, HER2-positive breast cancer, BRCA-mutated breast cancer, hematologic malignancies, non-small cell carcinoma, non-small cell lung cancer/carcinoma (NSCLC), small cell lung cancer/carcinoma (SCLC), extensive-stage small cell lung cancer (ES-SCLC), lymphoma (classical Hodgkin's and non-Hodgkin's), primary mediastinal large B-cell lymphoma (PMBCL), T-cell lymphoma, diffuse large B-cell lymphoma, histiocytic sarcoma, follicular dendritic cell sarcoma, interdigitating dendritic cell sarcoma, myeloma, chronic lymphocytic leukemia (CLL), acute myeloid leukemia (AML), small lymphocytic lymphoma (SLL), head and neck cancer (e.g., head and neck squamous cell cancer), urothelial cancer e.g., metastatic urothelial carcinoma), merkel cell carcinoma (e.g., metastatic merkel cell carcinoma), merkel cell skin cancer, cancer with high microsatellite instability (MSI-H), cancer with mismatch repair deficiency (dMMR), tumor mutation burden high cancer, mesothelioma (e.g., malignant pleural mesothelioma), gastric cancer, gastroesophageal junction cancer (GEJ), gastric adenocarcinoma, neuroendocrine tumors, gastrointestinal stromal tumors (GIST), gastric cardia adenocarcinoma, renal cancer, biliary cancer, cholangiocarcinoma, pancreatic cancer, prostate cancer, adenocarcinoma, squamous cell carcinoma, non-squamous cell carcinoma, cutaneous squamous cell carcinoma (CSCC), ovarian cancer, endometrial cancer, fallopian tube cancer, cervical cancer, peritoneal cancer, stomach cancer, brain cancers, malignant glioma, glioblastoma, gliosarcoma, neuroblastoma, thyroid cancer, adrenocortical carcinoma, oral intra-epithelial neoplasia, esophageal cancer, nasal cavity and paranasal sinus squamous cell carcinoma, nasopharynx carcinoma, salivary gland cancer, liver cancer, basal cell carcinoma, and hepatocellular cancer (HCC).

According to some embodiments of the above aspects and embodiments, the subject is treated with a Treg-enhancing agent, wherein optionally the Treg-enhancing agent comprises all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

According to some embodiments of the above aspects and embodiments, the LTBP-selective TGFβ1 inhibitor is administered in an amount sufficient to achieve a serum concentration of about 100-300 μg/mL. According to some embodiments, the LTBP-selective TGFβ1 inhibitor is administered in an amount sufficient to achieve a serum concentration of about 100-200 μg/mL. According to some embodiments, the LTBP-selective TGFβ1 inhibitor is administered in an amount sufficient to achieve a serum concentration of about 200-300 μg/mL. According to some embodiments, the LTBP-selective TGFβ1 inhibitor is administered in an amount sufficient to achieve a serum concentration of about 100, about 125, about 150, about 175, about 200, about 225, about 250, about 275, or about 300 μg/mL.

According to another aspect, the disclosure provides a pharmaceutical composition comprising an antibody or an antigen-binding fragment thereof that binds recombinant human LTBP1-proTGFβ1 and/or recombinant human LTBP3-proTGFβ1 with a K_{D} of 10 nM or less as measured by a SPR-based assay (such as Biacore), wherein the antibody is not Ab42, wherein the antibody competes for antigen binding with SR42 and binds an epitope comprising one or more amino acid residues of the sequence DMELVKRKRIEAIR (SEQ ID NO: 46), wherein optionally the antibody contacts one or more of the residues: Asp27 (D); Leu30 (L); Arg33 (R); Ile36 (I); Glu37 (E).

According to another aspect, the disclosure provides a pharmaceutical composition comprising an antibody or an antigen-binding fragment thereof that binds recombinant human LTBP1-proTGFβ1 and/or recombinant human LTBP3-proTGFβ1 with a K_{D} of 10 nM or less as measured by a SPR-based assay (such as Biacore), wherein the antibody is not Ab42; wherein the antibody competes for antigen binding with SR42 and binds an epitope comprising one or more amino acid residues of the sequence YIDFRKDLGWK (SEQ ID NO: 93), wherein optionally the antibody contacts one or more of the residues: Tyr289 (Y); Lys294 (K); Asp295 (D).

### BRIEF DESCRIPTION OF THE FIGURES

For illustration, some embodiments of the disclosure are shown in the drawings described below. Like numerals in the drawings indicate like elements throughout. The disclosure is not limited to the precise arrangements, dimensions, and instruments shown.

FIG. 1(A) and FIG. 1(B) demonstrate that targeting of the latent form of TGFβ1 provided isoform and context specificity. FIG. 1(A) shows how LTBP presents TGFβ in connective tissue, i.e., extracellular matrix (ECM). As shown in the drawing, TGFβ is expressed as part of a large latent complex (LLC). The activation is by integrin-induced traction force. LTBP-presented TGFβ1 is a major driver of fibrosis. FIG. 1(B) is a drawing that demonstrates functional assays (potency assays) to detect the inhibition of activated recombinant latent TGFβ1. FIG. 1(B) depicts the activation of latent TGFβ1 deposited in the extracellular matrix (ECM). In this assay, presenting molecules were co-transfected with TGFβ1 in integrin-expressing cells. Transiently transfected cells were seeded in assay plates in the presence of inhibitors. Then, latent LTBP-TGFβ1 complex was embedded in the ECM. TGFβ reporter cells were added to the system. Free growth factor (released by integrin) signals and was detected by luciferase assay. See WO 2020/160291 A2. Isoform-specific monoclonal antibodies that bind the latent TGFβ1 prodomain, with no detectable binding to latent TGFβ2 or TGFβ3, and that inhibit integrin-mediated activation of latent TGFβ1 in vitro with the context-dependency as described therein.

FIG. 2 is a set of four graphs showing Ab42 human Fab binding to human TGFβ1 complexes: (1) LTBP1-proTGFβ, (2) LTBP3-proTGFβ, (3) GARP-proTGFβ, or (4) LRRC33-proTGFβ). As shown in FIG. 2, LTBP-complex antibodies were highly specific and had picomolar monovalent affinities as measured by BIACORE®. Note the low level of binding of Ab42 against both GARP and LRRC33 complexed TGFβ1 as shown by the RU values, the Y axis of each graph, and the low responses shown in the bottom two graphs.

FIG. 3 is a graph showing that Ab42 was a more potent inhibitor of LTBP TGFβ1 activation than a context-independent reference antibody. FIG. 3 shows the results from a CAGA assay with endogenous expression of LTBP, and demonstrated the potent inhibition of Ab42. For a description of a similar assay, see Itoh et al. (2019) “A comparative analysis of Smad-responsive motifs identifies multiple regulatory inputs for TGF-β transcriptional activation. Properties of Smad-binding motifs” J. Biol. Chem., 294(42), 15466-15479 (Oct. 18, 2019).

FIG. 4(A) and FIG. 4(B) are two graphs showing Sprague Dawley Rat serum results after Ab42 exposure at 10 mg/kg administration.

FIG. 5(A) and FIG. 5(B) are graphs showing Sprague Dawley Rat serum results after Ab42 exposure at 30 mg/kg administration.

FIG. 6 is a dot plot showing Sprague Dawley rat picrosirius red/serum exposure correlation results after Ab42 exposure at 10 mg/kg and 30 mg/kg administration.

FIG. 7 is a graph showing individual serum exposures of Ab42 in a previous rat choline-deficient high fat diet (CDHFD) pharmacokinetics (PK)/pharmacodynamics (PD) analysis. A serum exposure of about 340 μg/ml was required to reduce pSMAD2 in CDHFD livers.

FIG. 8 is a bar graph showing that a single dose of Ab42 reduced pSmad2 in a rat choline-deficient high fat diet (CDHFD) PK/PD analysis.

FIG. 9 is a graph showing the serum exposure (μg/ml) of Ab42 at two different doses over time.

FIG. 10 shows the percent suppression of hydroxyproline. HuNEG=100%, Pre-treatment=0%. * P<0.05. ** P<0.01. *** P<0.001. **** P<0.0001. FIG. 10 shows the results of the correlation of t-test vs. IgG group and t-test versus vehicle (Alk5i).

FIG. 11 is a graph showing the results of plasma blood urea nitrogen assay and creatinine assays. Measurements were made through day 56.

FIG. 12 is a graph showing the results of a creatinine assay. Measurements were made through day 56.

FIG. 13 is a graph showing HYP levels in the liver after treatment in the CDHFD study.

Results demonstrated that inhibition of the LTBP arm is just as efficacious as the inhibition of all context-independent inhibitors (see Reference Antibody).

FIG. 14 (A)-FIG. 14(C) is a set of graphs showing the ratios of phosphorylated SMAD protein versus total (phosphorylated and unphospohrylated) Smad. FIG. 14(A) is a graph showing relative ratios of phosphorylated versus total (phosphorylated and unphospohrylated) SMAD2/3 (pSMAD2/3:tSMAD2/3) in an Alport mouse model. A single dose of Ab42 was sufficient to significantly inhibit pSmad2/3 signaling in whole kidney lysates. FIG. 14(B) is a graph showing the amount of phosphorylated Smad2/3 (pSmad2/3) as determined by ELISA. FIG. 14(C) is a graph showing the amount of total Smad2/3 (tSmad2/3) protein as determined by ELISA. As shown by FIG. 14(B) and FIG. 14(C), reduction of pSmad was contributing to the change in ratio shown in FIG. 14(A).

FIG. 15 shows the results from a Alport syndrome (mouse) kidney fibrosis PK/PD analysis.

Eleven-week-old mice were treated with a single dose of antibody. Mouse tissue was collected after forty-eight hours. Treatment with Ab42 significantly reduced pSmad2 in Alport syndrome mouse kidneys.

FIG. 16 is a graph showing the serum concentration by hour after administration of Ab42.

FIG. 17 is a pair of graphs showing the results of an rat adenine kidney fibrosis PK/PD analysis.

FIG. 18 is a pair of graphs showing that the YTE mutation enhanced binding of Ab42 to Hu FcRn. Ab42-YTE HuG4—120 nM. Ab42 HuG4—860 nM. The YTE mutation enhanced binding to human FcRn dramatically at acidic pH and slightly at neutral pH, as measured by OCTET©.

FIG. 19 shows that the introduction of the YTE mutations in Ab42 resulted in a reduced melting temperature. The YTE mutation resulted in a decreased melting temperature.

FIG. 20 is a graph showing a similar potency of Ab42 YTE compared to Ab42. The Ab42 and Ab42-YTE lots showed comparable activity compared with a DS210209B positive control.

FIG. 21 is a panel of graphs that show treatment with Ab42 reduced plasma BUN and creatinine in adenine fed rats.

FIG. 22 is a graph that shows the results of an immunohistochemical (IHC) analysis that was carried out to determine the amount of phosphorylated Smad2 (active Smad2) in fibrotic kidney samples after treatment with Ab42.

FIG. 23A shows the average serum exposure for Ab42 using a loading dose strategy. FIG. 23B shows the PSR/serum exposure correlation for Ab42 using a loading dose strategy.

FIG. 24 is a graph that shows exposure with Ab42 was dose-proportional and maintained across all time points.

FIGS. 25A and 25B show the results of immunohistochemical (IHC) analysis to determine the amount of phosphorylated Smad2 (active Smad2) in fibrotic kidney samples after treatment with Ab42.

FIG. 26 is a graph that shows treatment with Ab42 reduced expression of certain TGFβ related genes in the adenine model.

FIG. 27 is a graph that shows a strong induction of profibrotic gene expression in the adenine model, and a reduction of pro-fibrotic genes with treatment with LTBP-TGFβ1 specific inhibitor Ab42.

FIG. 28 is a graph that shows relative gene expression of certain pro-inflammatory genes in the adenine model.

FIG. 29 is a graph that shows relative gene expression of TNFα and TGFβ ligands.

FIG. 30 is a graph that shows relative gene expression of LTBP1, LRRC33 and Col3A1.

FIG. 31 is a graph that shows relative gene expression of KIM1 and LNC2.

FIG. 32 is a graph that shows relative gene expression of FN1 over time.

FIG. 33 is a graph that shows relative gene expression of Col3A1 over time.

FIG. 34 is a graph that shows relative gene expression of IL-1β over time.

FIG. 35 is a graph that shows relative gene expression of IL-6 over time.

FIG. 36 is a graph that shows relative gene expression of GARP over time.

FIG. 37 is a graph that shows relative gene expression of LTBP1 over time.

FIG. 38 is a graph that shows relative gene expression of THBS over time.

FIG. 39 is a graph that shows relative gene expression of col1a11 over time.

FIG. 40 is a graph that shows relative gene expression of CD68 over time.

FIG. 41 is a graph that shows relative gene expression of Loxl2 over time.

FIG. 42 is a graph that shows relative gene expression of MMP2 over time.

FIG. 43 is a graph that shows relative gene expression of MMP9 over time.

FIG. 44 is a graph that shows relative gene expression of KIM1 over time.

FIG. 45 is a graph that shows relative gene expression of LRRC33 over time.

FIG. 46 is a graph that shows relative gene expression of TNFα over time.

FIG. 47 is a graph that shows cell viability using the resazurin assay at endpoint.

FIG. 48 is a graph measuring lactate dehydrogenase levels in supernatant at each timepoint.

FIG. 49 is a graph that shows results from the study described in Example 15. Group averaged PK data up to day 72 is shown.

FIG. 50 is a graph that shows PK data for individual animals from the study described in Example 15.

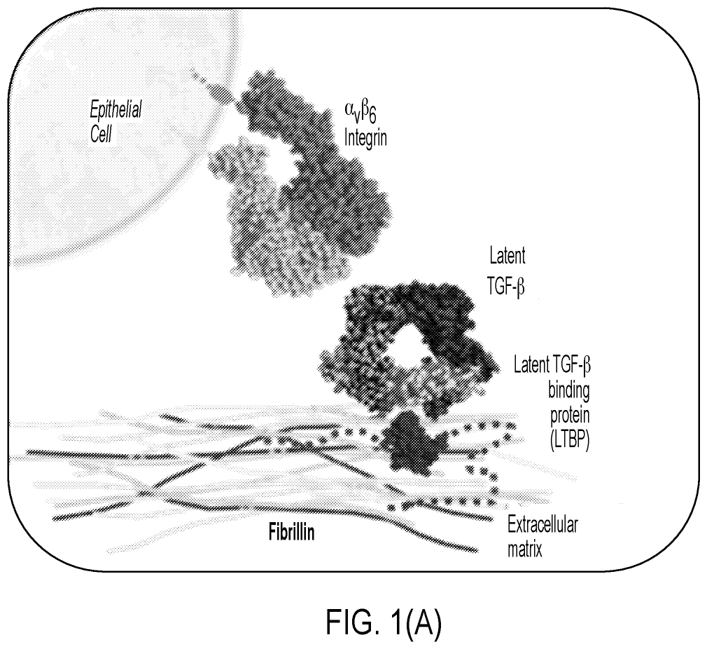

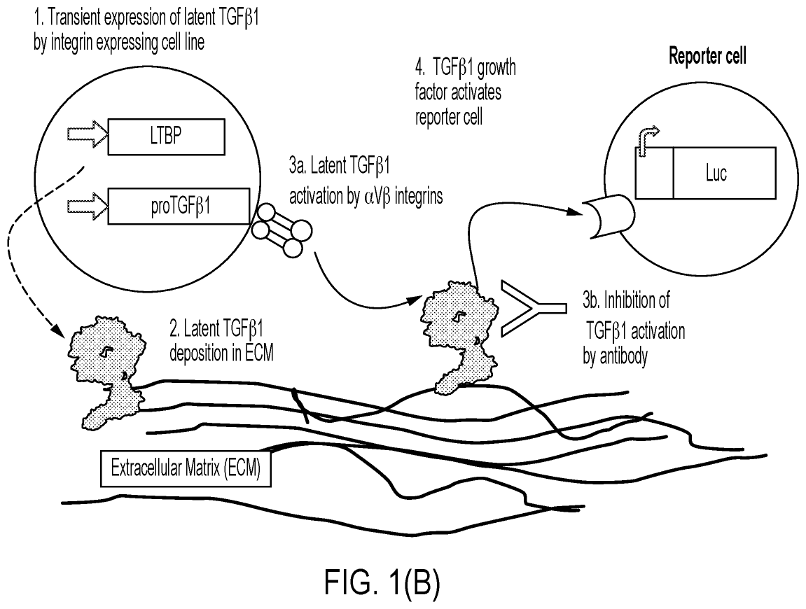

### DETAILED DESCRIPTION OF THE DISCLOSURE

The present disclosure provides compositions and methods that are useful for reducing activation of a subset of TGFβ1. More specifically, potent antibodies or fragments thereof capable of selectively targeting and inhibiting LTBP1/3-associated proTGFβ1 are used in the treatment of TGFβ-associated disease in a human subject. Inhibitors that target latent proTGFβ1 complexes, upstream of growth factor-receptor interaction, are generally referred to as activation inhibitors of TGFβ1.

To date, four presenting molecules for TGFβ have been identified: latent TGF beta-binding protein 1 (“LTBP1”), latent TGF beta-binding protein 3 (“LTBP3”), glycoprotein A repetitions predominant (“GARP”) and leucine-rich repeat-containing protein 33 (“LRRC33”). Each of these presenting molecules can form disulfide bonds with a homodimeric pro-protein complex of the TGFβ1 precursor, i.e., proTGFβ1. The proTGFβ1 complex remains dormant (latent) in the respective extracellular niche (e.g., ECM and immune cell surface) until activation events trigger the release of soluble growth factor from the complex.

As compared to the TGFβ growth factors and the receptors, which are expressed broadly, the presenting molecules show more restricted or selective (e.g., tissue-specific) expression patterns, giving rise to functional compartmentalization of TGFβ1 activities by virtue of association. The four presenting molecule-proTGFβ1 complexes, namely, LTBP1-proTGFβ1, LTBP3-proTGFβ1, GARP-proTGFβ1 and LRRC33-proTGFβ1, therefore, provide discrete “contexts” of TGFβ1 signaling within the tissue in which the presenting molecules are expressed. These contexts may be divided into two broad categories: i) TGFβ1 signaling associated with the ECM (e.g., matrix-associated TGFβ1 function); and ii) TGFβ1 signaling associated with cells (particularly certain immune cell function). The LTBP1-proTGFβ1 and LTBP3-proTGFβ1 complexes fall under the first category, while GARP-proTGFβ1 and LRRC33-proTGFβ1 complexes fall under the second category. Thus, disclosed herein are isoform-selective inhibitors of TGFβ1 that are capable of selectively inhibiting the activation of TGFβ1 that is associated with the ECM.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In exemplary embodiments, the compositions described herein are useful for selectively reducing activation of TGFβ1 in the context of an LTBP protein, e.g., a LTBP1 and/or a LTBP3 protein. Such compositions advantageously inhibit activation of extracellular matrix-associated TGFβ31, without inhibiting TGFβ1 in the context of the immune-associated TGFβ1 presenting molecules GARP and LRRC33. The compositions described herein are useful for treating disorders associated with TGFβ1 activation, particularly fibrotic disorders, such as scleroderma, Alport syndrome, IPF and DMD. Accordingly, in embodiments, the disclosure provides compositions for reducing activation of TGFβ1, methods of use thereof, methods of manufacture, and treatment methods. Methods of selecting a TGFβ1 inhibitor for subjects exhibiting symptoms of a fibrotic disorder are also provided.

In some embodiments, the disclosure provides compositions for reducing activation of TGFβ1, in a subject with scleroderma.

In some embodiments, the disclosure provides compositions for reducing activation of TGFβ1, in a subject with Alport syndrome.

The rationale for the therapeutic use of a TGFβ1 inhibitor that does not target the GARP-proTGFβ1 complex on regulatory T cells is at least threefold:

First, regulatory T cells play a crucial role in maintaining immune tolerance to self-antigens and in preventing autoimmune disease. Since Tregs generally suppress, dampen or downregulate induction and proliferation of effector T cells, systemic inhibition of this function may lead to overactive or exaggerated immune responses in the host by disabling the “break” that is normally provided by Treg cells. The approach of TGFβ1 inhibition without disabling Treg function is aimed to avoid the risk of eliciting autoimmunity. Furthermore, patients who already have a propensity for developing over-sensitive immune responses or autoimmunity may be particularly at risk of triggering or exacerbating such conditions, without the availability of normal Treg function; and therefore, the inhibitors that selectively target the matrix TGFβ1 may advantageously minimize such risk.

Second, evidence suggests that an alteration in the Th17/Treg ratio leads to an imbalance in pro-fibrotic Th17 cytokines, which correlate with severity of fibrosis, such as liver fibrosis. The perturbation of the GARP arm of TGFβ1 function may directly or indirectly exacerbate fibrotic conditions.

Third, regulatory T cells are indispensable for immune homeostasis and the prevention of autoimmunity. Particularly for a TGFβ1 inhibition therapy intended for a long-term or chronic administration, it would be desirable to avoid potential side effects stemming from perturbation of normal Treg function in maintaining immune homeostasis. This strategy preserves normal immune function, which is required, inter alia, for combatting infections.

The disclosure described herein does not concern a process for cloning humans, processes for modifying the germ line genetic identity of humans, uses of human embryos for industrial or commercial purposes or processes for modifying the genetic identity of animals which are likely to cause them suffering with no substantial medical benefit to man or animal, and also animals resulting from such processes.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

Advanced fibrosis: the term “advanced fibrosis” as used herein has the meaning understood in the pertinent art, e.g., as understood by a physician in the context of diagnosing or treating subjects/patients with a fibrotic disease or disorder. As used herein, the term has the medical art-recognized meaning. Subjects suffer from advanced fibrosis if they have an advanced stage of a fibrotic disorder, particularly organ fibrosis, which renders the patients a candidate for receiving, or in need of, an allograft transplant.

Affinity maturation: has the immunological art-recognized meaning of a type of antibody optimization and is a process of improving the affinity of an antibody or a fragment to its antigen and typically involves making one or more changes to the amino acid sequence of the antibody or the fragment to achieve greater affinity. Typically, a parental antibody and an affinity-matured counterpart retain the same epitope. Affinity maturation may include diversification or mutagenesis of one or more CDR sequences.

Affinity: Affinity is the strength of binding of a molecule (such as an antibody) to its ligand (such as an antigen). It is typically measured and reported by the equilibrium dissociation constant (KD). In the context of antibody-antigen interactions, KD is the ratio of the antibody dissociation rate (“off rate” or Koff or Kdis), how quickly it dissociates from its antigen, to the antibody association rate (“on rate” or Kon) of the antibody, how quickly it binds to its antigen. For example, an antibody with an affinity of ≤5 nM has a KD value that is 5 nM or lower (i.e., 5 nM or higher affinity) determined by a suitable in vitro binding assay. Suitable in vitro assays can be used to measure KD values of an antibody for its antigen, such as Biolayer Interferometry (BLI) and Solution Equilibrium Titration (e.g., MSD-SET). The Ab42 in vitro binding assay results provided in the EXAMPLES below showed high-affinity (low K_{D} value) binding.

Alport syndrome: has the medical art-recognized meaning of a genetic disorder characterized by glomerulonephritis, end-stage kidney disease, and hearing loss. Blood in urine is universal. Proteinuria is a feature as kidney disease progresses. Alport syndrome is caused by an inherited defect in type IV collagen—a structural material that is needed for the normal function of different parts of the body. Mouse models exist that have genetic mutations characteristic of Alport syndrome.

Antibody: The term “antibody” encompasses any naturally-occurring, recombinant, modified or engineered immunoglobulin or immunoglobulin-like structure or antigen-binding fragment or portion thereof, or derivative thereof, as further described elsewhere herein. Unless specified to the contrary, the term “antigen” as used herein shall encompass antigen-binding fragments and functional variants thereof. Thus, the term refers to an immunoglobulin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen-binding portions thereof, can be produced in hybridomas, by recombinant DNA techniques, or by enzymatic or chemical cleavage of intact antibodies. The term antibodies, as used herein, includes monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively. In some embodiments, the term also encompasses peptibodies.

Antigen: The term “antigen” broadly includes any molecules comprising an antigenic determinant within a binding region(s) to which an antibody or a binding-fragment specifically binds. An antigen can be a single-unit molecule (such as a protein monomer or a fragment) or a complex comprised of multiple components. An antigen provides an epitope, e.g., a molecule or a portion of a molecule, or a complex of molecules or portions of molecules, capable of being bound by a selective binding agent, such as an antigen-binding protein (including, e.g., an antibody). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen-binding proteins, e.g., antibodies. In the context of the present disclosure, a suitable antigen is a complex (e.g., multimeric complex comprised of multiple components in association) containing a proTGF dimer in association with a presenting molecule. Each monomer of the proTGF dimer comprises a prodomain and a growth factor domain, separated by a furin cleavage sequence. Two such monomers form the proTGF dimer complex. This in turn is covalently associated with a presenting molecule via disulfide bonds, which involve a cysteine residue present near the N-terminus of each of the proTGF monomer. This multi-complex formed by a proTGF dimer bound to a presenting molecule is generally referred to as a large latent complex. An antigen complex suitable for screening antibodies or antigen-binding fragments, for example, includes a presenting molecule component of a large latent complex. Such presenting molecule component may be a full-length presenting molecule or a fragment(s) thereof. Minimum required portions of the presenting molecule typically contain at least 50 amino acids, but more preferably at least 100 amino acids of the presenting molecule polypeptide, which comprises two cysteine residues capable of forming covalent bonds with the proTGFβ1 dimer.

Antigen-binding portion/fragment: The terms “antigen-binding portion” or “antigen-binding fragment” of an antibody, as used herein, refers to one or more fragments of an antibody that retain the ability to specifically bind to an antigen (e.g., TGFβ1). Antigen-binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CH1 domains; (ii) F(ab′)2 fragments, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules (see, e.g., Bird et al., (1988) Science 242:423-426; and Huston et al., (1988) Proc. Nat'l. Acad. Sci. USA 85:5879-5883); (vi) dAb fragments (see, e.g., Ward et al., (1989) Nature 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen-binding portion of an antibody includes a “single chain Fab fragment” otherwise known as an “scFab,” comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

As needed: in the context of pharmaceutical dosing regimens, means that the dosing regimen is not based on a predetermined dosing schedule but instead based on one or more parameters or markers measured or monitored periodically during treatment, which provides information or guidance as to whether additional doses should be beneficial to the subject/patient. For instance, a pharmaceutical composition comprising a TGFβ inhibitor such as TGFβ1/2/3 inhibitors (“pan” inhibitors), TGFβ1/2 inhibitors and TGFβ1/3 inhibitors, may be administered, intermittently, on an “as needed” basis in a therapeutically effective amount sufficient to achieve or maintain clinical benefit, e.g., reduction of one or more clinical markers of fibrosis. In forty-first embodiment, administration of a LTBP-complex selective TGFβ inhibitor, such as Ab42, can be used in combination with a method of determining or monitoring therapeutic efficacy. The LTBP-complex selective TGFβ inhibitor is administered in patients only when clinical benefit from additional doses of the TGFβ inhibitor is expected. It is contemplated that, in order to manage toxicities, intermittent or “as-needed” dosing regimen may be required more frequently with isoform-non-selective inhibitors of TGFβ, as compared to TGFβ1-selective inhibitors, such as those disclosed herein.

Autoimmune disease: has the immunological art-recognized meaning of a condition arising from an abnormal or overactive immune response to a normal body part. Immunostimulating agents administered to such patients with autoimmune conditions may exacerbate the condition.

Bias: In the context of the present disclosure, the term “bias” refers to skewed or uneven affinity towards or against a subset of antigens to which an antibody is capable of specifically binding. For example, an antibody is said to have bias when the affinity for one antigen complex and the affinity for another antigen complex are not equivalent (e.g., more than five-fold difference in affinity). Preferred antibodies of the present disclosure include “matrix-biased” (or “LTBP-biased”) antibodies, which preferentially bind EMC-associated complexes (LTBP1-proTGFβ1 and LTBP3-proTGFβ), such that relative affinities between at least one of the matrix-associated complexes and at least one of the cell-associated complexes (GARP-proTGFβ1 and/or LRRC33-proTGFβ1 complexes) is greater than five-fold. By comparison, antibodies characterized as “unbiased” have approximately equivalent affinities towards such antigen complexes (e.g., less than five-fold difference in affinity).

Binding region: As used herein, a “binding region” is a portion of an antigen (e.g., an antigen complex) that, when bound to an antibody or a fragment thereof, can form an interface of the antibody-antigen interaction. Upon antibody binding, a binding region becomes “protected” from surface exposure, which can be detected by suitable techniques, such as HDX-MS. Antibody-antigen interaction may be mediated via multiple (e.g., two or more) binding regions. A binding region can comprise an antigenic determinant, or epitope.

Bio-Layer Interferometry (BLI): is a label-free technology for optically measuring biomolecular interactions, e.g., between a ligand immobilized on the biosensor tip surface and an analyte in solution. Bio-layer interferometry provides the ability to monitor binding specificity, rates of association and dissociation, or concentration, with precision and accuracy. Bio-layer interferometry platform instruments are commercially available, e.g., from ForteBio and are known as the OCTET® System. Bio-layer interferometry can carry out in vitro binding assays as described herein.

Cell-associated TGFβ1/proTGFβ1: The term refers to TGFβ1 or its signaling complex (e.g., pro/latent TGFβ1) that is membrane-bound (e.g., tethered to cell surface). Typically, such cell is an immune cell. TGFβ1 that is presented by GARP or LRRC33 is a cell-associated TGFβ1. GARP and LRRC33 are transmembrane presenting molecules that are expressed on cell surface of certain cells. GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 activation/signaling. The term also includes recombinant, purified GARP-proTGFβ1 and LRRC33-proTGFβ1 complexes in solution (e.g., in vitro assays) which are not physically attached to cell membranes. Average K_{D} values of an antibody (or its fragment) to a GARP-proTGFβ1 complex and an LRRC33-proTGFβ1 complex may be calculated to collectively represent affinities for cell-associated (e.g., immune cell-associated) proTGFβ1 complexes. See, for example, Table, column (G). Human counterpart of a presenting molecule or presenting molecule complex may be indicated by an “h” preceding the protein or protein complex, e.g., “hGARP,” “hGARP-proTGFβ1,” hLRRC33” and “hLRRC33-proTGFβ1.”

Chronic inflammation: The term “chronic inflammation” as used herein has the medical art-recognized meaning. Fibrotic disorders that involve chronic inflammation are characterized by continuous or persistent injury to a tissue such that it does not resolve in normal healing after an initial injury. Chronic inflammation refers to a prolonged inflammatory response that involves a progressive change in the type of cells present at the site of inflammation, e.g., fibrotic tissues. Chronic inflammation is characterized by the simultaneous destruction and repair of the tissue from the inflammatory process and can follow an acute form of inflammation or be a prolonged low-grade form.

Clinical benefit: As used herein, the term “clinical benefits” is intended to include both efficacy and safety of a therapy. Thus, therapeutic treatment that achieves a desirable clinical benefit is both efficacious and safe (e.g., with tolerable or acceptable toxicities or adverse events).

Combination therapy: “Combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies. When a subject who has been treated with a first therapy (e.g., background therapy) for the treatment of a disease is administered with a second therapy to treat the same disease, the second therapy may be referred to as an “add-on therapy” or “adjunct therapy.” In preferred embodiments, combination therapy or adjunct therapy can produce a synergistic effect such that clinical benefits from both therapies are greater than a sum of clinical benefits from each of the therapies alone,

Combinatory or combinatorial epitope: A combinatorial epitope is an epitope that is recognized and bound by a combinatorial antibody at a site (i.e., antigenic determinant) formed by non-contiguous portions of a component or components of an antigen, which, in a three-dimensional structure, come together in close proximity to form the epitope. Thus, antibodies of the disclosure may bind an epitope formed by two or more components (e.g., portions or segments) of a pro/latent TGFβ1 complex. A combinatory epitope may comprise amino acid residue(s) from a first component of the complex, and amino acid residue(s) from a second component of the complex, and so on. Each component may be of a single protein or of two or more proteins of an antigenic complex. A combinatory epitope is formed with structural contributions from two or more components (e.g., portions or segments, such as amino acid residues) of an antigen or antigen complex.

Compete or cross-compete: The term “compete” when used in the context of antigen-binding proteins (e.g., an antibody or antigen-binding portion thereof) that compete for the same epitope means competition between antigen-binding proteins as determined by an assay in which the antigen-binding protein being tested prevents or inhibits (e.g., reduces) specific binding of a reference antigen-binding protein to a common antigen (e.g., TGFβ1 or a fragment thereof). Numerous types of competitive binding assays can be used to determine if one antigen-binding protein competes with another, for example: solid phase direct or indirect radioimmunoassay (RIA), solid phase direct or indirect enzyme immunoassay (EIA), sandwich competition assay; solid phase direct biotin-avidin EIA; solid phase direct labeled assay, and solid phase direct labeled sandwich assay. Usually, when a competing antigen-binding protein is present in excess, it will inhibit (e.g., reduce) specific binding of a reference antigen-binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more. In some instances, binding is inhibited by at least 80-90%, at least 85%-95%, at least 95-99%. In some instances, binding is inhibited by at least 90%, 91%, 92%, 93%, 94%, 95%, 96%, 97%, 98%, 99% or more. In some embodiments, a first antibody or antigen-binding portion thereof and a second antibody or antigen-binding portion thereof cross-block with each other with respect to the same antigen, for example, as assayed by BLI (such as BIACORE® or OCTET®), using standard test conditions, e.g., according to the manufacturer's instructions (e.g., binding assayed at room temperature, ˜20-25° C.). In some embodiments, the first antibody or fragment thereof and the second antibody or fragment thereof may have the same epitope. In other embodiments, the first antibody or fragment thereof and the second antibody or fragment thereof may have non-identical but overlapping epitopes. In yet further embodiments, the first antibody or fragment thereof and the second antibody or fragment thereof may have separate (different) epitopes which are in close proximity in a three-dimensional space, such that antibody binding is cross-blocked via steric hindrance. “Cross-block” means that binding of the first antibody to an antigen prevents binding of the second antibody to the same antigen, and similarly, binding of the second antibody to an antigen prevents binding of the first antibody to the same antigen.

Complementary determining region (CDR): As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al., (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al., (1989) Nature 342: 877-883) found that certain sub-portions within Kabat CDRs adopt nearly identical peptide backbone conformations, despite having great diversity at the level of amino acid sequence. These sub-portions were designated as L-CDR1, L-CDR2 and L-CDR3 or H-CDR1, H-CDR2 and H-CDR3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen-binding (see, for example: Lu X et al., MAbs. 2019 January; 11(1):45-57). The methods used herein may utilize CDRs defined according to any of these systems, although certain embodiments use Kabat- or Chothia-defined CDRs.

Conformational epitope: A conformational epitope is an epitope that is recognized and bound by a conformational antibody in a three-dimensional conformation, but not in an unfolded peptide of the same amino acid sequence. A conformational epitope may be referred to as a conformation-specific epitope, conformation-dependent epitope, or conformation-sensitive epitope. A corresponding antibody or fragment thereof that specifically binds such an epitope may be referred to as conformation-specific antibody, conformation-selective antibody, or conformation-dependent antibody. Binding of an antigen to a conformational epitope depends on the three-dimensional structure (conformation) of the antigen or antigen complex.

Constant region/domain: An immunoglobulin constant domain refers to a heavy or light chain constant domain. Human IgG heavy chain and light chain constant domain amino acid sequences are known in the art.

Context-independent: According to the present disclosure, “a context-independent antibody” that binds proTGFβ1 has equivalent affinities across the four known presenting molecule-proTGFβ1 complexes, namely, LTBP1-proTGFβ1, LTBP3-proTGFβ1, GARP-proTGFβ1 and LRRC33-proTGFβ1. Any suitable in vitro binding assay, such as surface plasmon resonance, Biolayer Interferometry (BLI), and/or solution equilibrium titration (e.g., MSD-SET), may be employed to measure binding activities of antibodies or fragments thereof.

Context-specific antibodies or context-selective antibodies: as contrasted with “context-independent” antibodies, are those antibodies that can bind selectively to a subset, but not all, of proTGFβ1 complexes associated with a particular biological context. Matrix-selective targeting enables specific inhibition of TGFβ1 function associated with the extracellular matrix. ECM-selective inhibition can be achieved by the use of antibodies or fragments thereof that selectively target the extracellular matrix components, LTBP1-proTGFβ1 or LTBP3-proTGFβ1. Antibodies and fragments disclosed herein therefore represent a class of context-specific antibodies. LTBP1-specific and LTBP3-specific inhibitors of TGFβ1 activation are also context-specific antibodies. In some embodiments, context selective or context specific can include antibodies that bind both LTBP-1 and LTBP-3 complexes but do not bind GARP or LRRC33. In some embodiments, “LTBP context-selectivity” refers to a class of LTBP1-specific and LTBP-3 specific inhibitors of TGFβ1 activation that are have at least 50× more selectivity over GARP or LRRC33 complexes.

Corticosteroid: PREDNISONE© is an example of a known corticosteroid which can reduce inflammation in tissues, such as lungs by suppressing the immune system. Corticosteroids are only prescribed in patients with idiopathic pulmonary fibrosis who have an acute exacerbation of their lung fibrosis and can be harmful in patients with idiopathic pulmonary fibrosis that have scarring that is stable or slowly worsening.

Cross-blocking antibody’ has the immunological art-recognized meaning of a first antibody or antigen-binding portion thereof and a second antibody or antigen-binding portion thereof cross-block with each other with respect to the same antigen, e.g., as assayed by as measured by Biolayer Interferometry, such as OCTET®, or surface plasmon resonance (SPR), such as BIACORE® Systems, using standard test conditions, e.g., according to the manufacturer's instructions, e.g., binding assayed at room temperature, ˜20-25° C. The first antibody or fragment thereof and the second antibody or fragment thereof may have the same epitope; may have non-identical but overlapping epitopes; or, may have separate (different) epitopes which are in close proximity in a three-dimensional space, such that antibody binding is cross-blocked via steric hindrance. The term “cross-block” means that binding of the first antibody to an antigen prevents binding of the second antibody to the same antigen, and similarly, binding of the second antibody to an antigen prevents binding of the first antibody to the same antigen.

Dissociation rate: has the immunological art-recognized meaning of a kinetics parameter measured by how fast/slow a ligand, e.g., antibody or fragment, dissociates from its binding target, e.g., antigen. Dissociation rate is also referred to as the “off” rate (“k_{OFF}”). Relative on/off rates between an antibody and its antigen, i.e., k_{ON} and k_{OFF}) determine the overall strength of the interaction, or affinity, typically expressed as a dissociation constant, or K_{D}. Therefore, equivalent affinities, e.g., K_{D} values) may be achieved by having fast association (high k_{ON}), slow dissociation (low k_{OFF}), or contribution from both factors. Monovalent interactions may be measured by the use of monovalent antigen-binding molecules/fragments, such as fAb (Fab), while divalent interactions may be measured by the use of divalent antigen-binding molecules such as whole immunoglobulins, e.g., IgGs. Dissociation kinetics may be expressed in terms of dissociation half-time (sometimes referred to as half binding time), or t_{1/2}, defined as a duration of time it takes for one half the number of antibody molecules, e.g., mAb, Fab, etc.) to dissociate from bound antigen. Thus, antibodies with slow dissociation rates have long dissociation half-time, and antibodies with fast dissociation rates have short dissociation half-time. Ab42 can remain bound to the antigen for a much longer duration of time (e.g., greater t_{1/2}) than an antibody with much higher “OFF” rate, which “falls off” (e.g., dissociates from) the antigen relatively quickly. Thus, the difference in the dissociation kinetics predominantly attributes to the notable difference in their overall affinities (K_{D}), which may result in enhanced potency. Therefore, characterization of binding kinetics provides useful information as to potential durability of effects and resulting in vivo potency. The Ab42 in vitro binding assay results provided in the EXAMPLES below showed low dissociation rate (OFF-rate).

Dosage: has the medical art-recognized meaning. The typical therapeutic dosage of an antibody of the disclosure ranges between about 1-30 mg/kg per dose. A typical dosing regimen may include once a week, every two weeks, every three weeks, every four weeks, once a month, every six weeks, etc.

ECM-associated TGFβ1/proTGFβ1: The term refers to TGFβ1 or its signaling complex (e.g., pro/latent TGFβ1) that is a component of (e.g., deposited into) the extracellular matrix. TGFβ1 that is presented by LTBP1 or LTBP3 is an ECM-associated TGFβ1. LTBPs are critical for correct deposition and subsequent bioavailability of TGFβ in the ECM, where fibrillin (Fbn) and fibronectin (FN) are believed to be the main matrix proteins responsible for the association of LTBPs with the ECM. Average K_{D} values of an antibody (or its fragment) to an LTBP1-proTGFβ1 complex and an LTBP3-proTGFβ1 complex may be calculated to collectively represent affinities for ECM-associated (or matrix-associated) proTGFβ1 complexes. Human counterpart of a presenting molecule or presenting molecule complex may be indicated by an “h” preceding the protein or protein complex, e.g., “hLTBP1,” “hLTBP1-proTGFβ1,” hLTBP3” and “hLTBP3-proTGFβ1.”

Effective amount: The terms “effective amount” and “therapeutically effective amount” have the medical art-recognized meaning of a dosage or dosing regimen that achieves statistically significant clinical benefits in a patient population. The terms “effective” and “therapeutically effective” refer to the ability or an amount to sufficiently produce a detectable change in a parameter of a disease, e.g., a slowing, pausing, reversing, diminution, or amelioration in a symptom or downstream effect of the disease. The term encompasses but does not require the use of an amount that completely cures a disease. According to some embodiments, an “effective amount” (or therapeutically effective amount, or therapeutic dose) is a dosage, concentration, or dosing regimen that achieves statistically significant clinical benefits (e.g., efficacy) in a patient population. For example, for an antibody that has been shown to be efficacious at doses between 3 mg/kg and 30 mg/kg in preclinical models, the effective amount can be said to be between about 3-30 mg/kg. Effective amount may be expressed as the level of serum exposure. For example, a therapeutically effective amount of Ab42 may be a dose sufficient to achieve a serum exposure of between about 10-400 μg/mL of the antibody.

Effector T cells: Effector T cells, as used herein, are T lymphocytes that actively respond immediately to a stimulus, such as co-stimulation and include, but are not limited to, CD4+ T cells (also referred to as T helper or Th cells) and CD8+ T cells (also referred to as cytotoxic T cells). Th cells assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including Th1, Th2, Th3, Th17, Th9, or TFh, which secrete different cytokines to facilitate different types of immune responses. Signaling from the APC directs T cells into particular subtypes. Cytotoxic (Killer). Cytotoxic T cells (TC cells, CTLs, T-killer cells, killer T cells), on the other hand, destroy virus-infected cells and cancer cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Cytotoxic effector cell (e.g., CD8+ cells) include, e.g., perforin and granzyme B.

Epitope: The term “epitope” may be also referred to as an antigenic determinant, is a molecular determinant (e.g., polypeptide determinant) that can be specifically bound by a binding agent, immunoglobulin or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody specifically binds an antigen when it recognizes its target antigen in a complex mixture of proteins and/or macromolecules.

Fibrosis: The term “fibrosis” or “fibrotic condition/disorder” refers to the process or manifestation characterized by the pathological accumulation of extracellular matrix (ECM) components, such as collagens, within a tissue or organ. Indeed, collagen accumulation is a hallmark of fibrosis. According to some embodiments, the fibrosis is lung (also referred to as pulmonary) fibrosis. In some embodiments, the fibrosis is scleroderma. In some embodiments, the fibrosis is fibrosis of skeletal muscle, wherein optionally the fibrosis of skeletal muscle is associated with muscular dystrophy, wherein further optionally the muscular dystrophy is MDM. In some embodiments, the fibrosis is a kidney fibrosis, wherein the kidney fibrosis is associated with CKD and/or a genetic disorder such as Alport syndrome.

Pulmonary fibrosis: The term “pulmonary fibrosis” or “lung fibrosis” as used in the context of the present disclosure refers to the formation of excess fibrous connective tissue in the lung. According to some embodiments, pulmonary fibrosis may be a secondary effect of other lung diseases. Examples of such diseases include autoimmune disorders, viral infections and bacterial infections (such as tuberculosis). Pulmonary fibrosis may also be idiopathic, with cigarette smoking, environmental factors (e.g. occupational exposure to gases, smoke, chemicals or dusts) or genetic predisposition thought to be risk factors.

Fibrotic microenvironment: The term “fibrotic microenvironment” refers to a local disease niche within a tissue, in which fibrosis occurs in vivo. The fibrotic microenvironment may comprise disease-associated molecular signature (a set of chemokines, cytokines, etc.), disease-associated cell populations (such as activated macrophages, MDSCs, etc.) as well as disease-associated ECM environments (alterations in ECM components and/or structure). Fibrotic microenvironment is thought to support the transition of fibroblast to α-smooth muscle actin-positive myofibroblast in a TGFβ-dependent manner. Fibrotic microenvironment may be further characterized by the infiltration of certain immune cells (such as macrophages and MDSCs).

GARP-TGFβ1 complex: As used herein, the term “GARP-TGFβ1 complex” (or “GARP-proTGFβ1 complex”) refers to a protein complex comprising a pro-protein form or latent form of a transforming growth factor-31 (TGFβ1) protein and a glycoprotein-A repetitions predominant protein (GARP) or fragment or variant thereof. In some embodiments, a pro-protein form or latent form of TGFβ1 protein may be referred to as “pro/latent TGFβ1 protein”. In some embodiments, a GARP-TGFβ1 complex comprises GARP covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a GARP-TGFβ1 complex comprises GARP non-covalently linked with pro/latent TGFβ1. In some embodiments, a GARP-TGFβ1 complex is a naturally-occurring complex, for example a GARP-TGFβ1 complex in a cell. The term “hGARP” denotes human GARP.

GSK3008348: (3S)-3-[3-(3,5-Dimethyl-1H-pyrazol-1-yl)phenyl]-4-{(3S)-3-[2-(5,6,7,8-tetrahydro-1,8-naphthyridin-2-yl)ethyl]-1-pyrrolidinyl}butanoic acid, CAS 1629249-33-7, is a selective small molecule αvβ6 RGD-mimetic. See John et al., Nature Communications, volume 11, Article number 4659 (2020).

Human antibody: The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Humanized antibody: The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody.

As used herein, the term “substantially” in the context of a CDR refers to a CDR having an amino acid sequence at least 80%, at least 85%, at least 90%, at least 95%, at least 96%, at least 97%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

Hydrogen/deuterium exchange mass spectrometry (HDX-MS): HDX-MS is a well-known technique employed to interrogate protein confirmation and protein-protein interactions in solution by measuring the degree of solvent accessibility. See, for example, Wei et al., (2014) Drug Discov Today 19(1): 95-102. “Hydrogen/deuterium exchange mass spectrometry for probing higher order structure of protein therapeutics: methodology and applications.” The HDX-MS technique may be employed to determine a region or regions of an antigen bound by an antibody (i.e., “binding region(s)”). Thus, such binding region(s) may contain or form an epitope.

In the results provided in the EXAMPLES below, epitope mapping by H/DX-MS identified the segment of the antigen that is important in Ab42 binding. This was later confirmed by x-ray crystallography which identified the epitope for Ab42 within the alpha-1 helix region of the prodomain and the finger-1 domain of the growth factor domain.

Immune suppression or immunosuppression: has the immunological art-recognized meaning of suppression or reduction of the strength of the body's immune system. The terms refer to the ability to suppress immune cells, such as T cells, NK cells and B cells. The gold standard for evaluating immunosuppressive function is the inhibition of T cell activity, which may include antigen-specific suppression and non-specific suppression. Regulatory T cells (Tregs) and MDSCs may be considered immunosuppressive cells. M2-polarized macrophages (e.g., TAMs) may also be characterized as immunosuppressive. Patients who “benefit from immunosuppression” include those who have advanced stages of organ fibrosis and are candidates for, being considered for, or have undergone transplantation.

Isoform-specific/selective: has the immunological art-recognized meaning of an agent's ability to discriminate one isoform over other structurally related isoforms (i.e., selectivity). An isoform-specific TGFβ inhibitor exerts its inhibitory activity towards one isoform of TGFβ but not the other isoforms of TGFβ at a given concentration. An isoform-specific TGFβ1 antibody selectively binds TGFβ1. A TGFβ1-specific inhibitor (antibody) preferentially targets (binds, thereby inhibits) the TGFβ1 isoform over TGFβ2 or TGFβ3 with substantially greater affinity. Context-specific inhibitors of this specification are also isoform-specific. For example, the selectivity in this context may refer to at least a 500-1000-fold difference in respective affinities as measured by an in vitro binding assay such as OCTET® and BIACORE®. In some embodiments, the selectivity is such that the inhibitor when used at a dosage effective to inhibit TGFβ1 in vivo does not inhibit TGFβ2 and TGFβ3. For instance, an antibody may preferentially bind TGFβ1 at affinity of ˜1 μM, while the same antibody may bind TGFβ2 and/or TGFβ3 at ˜0.5-50 nM. For such an inhibitor to be useful as a therapeutic, dosage to achieve desirable effects (e.g., therapeutically effective amounts) must fall within the window within which the inhibitor can effectively inhibit the TGFβ1 isoform without inhibiting TGFβ2 or TGFβ3. The terms “isoform-specific” and “isoform-selective” are used interchangeably herein.

Isolated: An “isolated” antibody as used herein, refers to an antibody that is substantially free of other antibodies having different antigenic specificities. In some embodiments, an isolated antibody is substantially free of other unintended cellular material and/or chemicals.

Large Latent Complex: The term “large latent complex” (“LLC”) in the context of the present disclosure refers to a complex comprised of a proTGFβ1 dimer bound to so-called a presenting molecule. Thus, a large latent complex is a presenting molecule-proTGFβ1 complex, such as LTBP1-proTGFβ1, LTBP3-proTGFβ1, GARP-proTGFβ1 and LRRC33-proTGFβ1. Such complexes may be formed in vitro using recombinant, purified components capable of forming the complex. For screening purposes, presenting molecules used for forming such LLCs need not be full length polypeptides; however, the portion of the protein capable of forming disulfide bonds with the proTGFβ1 dimer complex via the cysteine residues near its N-terminal regions is typically required.

Latency associated peptide (LAP): LAP is so-called the “prodomain” of proTGFβ1. As described in more detail herein, LAP is comprised of the “Straight Jacket” domain and the “Arm” domain. Straight Jacket itself is further divided into the Alpha-1 Helix and Latency Lasso domains.

Long-term administration or chronic administration: has the immunological art-recognized meaning of a therapeutic regimen that involves over six months of treatment is considered long-term. In some patient populations, long-term therapeutic regimens involve administration of a drug (such as context-specific TGFβ1 inhibitors) for an indefinite duration of time.

Localized: In the context of the present disclosure, the term “localized” (as in “localized tumor”) refers to anatomically isolated or isolatable abnormalities, such as solid malignancies, as opposed to systemic disease. Certain leukemia, for example, may have both a localized component (for instance the bone marrow) and a systemic component (for instance circulating blood cells) to the disease.

LRRC33-TGFβ1 complex: As used herein, the term “LRRC33-TGFβ1 complex” (or “LRRC33-proTGFβ1 complex”) refers to a complex between a pro-protein form or latent form of transforming growth factor-31 (TGFβ1) protein and a Leucine-Rich Repeat-Containing Protein 33 (LRRC33; also known as Negative Regulator Of Reactive Oxygen Species or NRROS) or fragment or variant thereof. In some embodiments, a LRRC33-TGFβ1 complex comprises LRRC33 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LRRC33-TGFβ1 complex comprises LRRC33 non-covalently linked with pro/latent TGFβ1. In some embodiments, a LRRC33-TGFβ1 complex is a naturally-occurring complex, for example a LRRC33-TGFβ1 complex in a cell. The term “hLRRC33” denotes human LRRC33.

LTBP1-TGFβ1 complex: As used herein, the term “LTBP1-TGFβ1 complex” (or “LTBP1-proTGFβ1 complex”) refers to a protein complex comprising a pro-protein form or latent form of transforming growth factor-31 (TGFβ1) protein and a latent TGF-beta binding protein 1 (LTBP1) or fragment or variant thereof. In some embodiments, a LTBP1-TGFβ1 complex comprises LTBP1 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LTBP1-TGFβ1 complex comprises LTBP1 non-covalently linked with pro/latent TGFβ1. In some embodiments, a LTBP1-TGFβ1 complex is a naturally-occurring complex, for example a LTBP1-TGFβ1 complex in a cell. The term “hLTBP1” denotes human LTBP1.

LTBP3-TGFβ1 complex: As used herein, the term “LTBP3-TGFβ1 complex” (or “LTBP3-proTGFβ1 complex”) refers to a protein complex comprising a pro-protein form or latent form of transforming growth factor-31 (TGFβ1) protein and a latent TGF-beta binding protein 3 (LTBP3) or fragment or variant thereof. In some embodiments, a LTBP3-TGFβ1 complex comprises LTBP3 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LTBP3-TGFβ1 complex comprises LTBP1 non-covalently linked with pro/latent TGFβ1. In some embodiments, a LTBP3-TGFβ1 complex is a naturally-occurring complex, for example a LTBP3-TGFβ1 complex in a cell. The term “hLTBP3” denotes human LTBP3.

Macrophage: has the immunological art-recognized meaning of a type of white blood cells of the immune system and includes heterogeneous, phenotypically diverse subpopulations of myeloid cells. Some macrophages differentiate from bone marrow-derived, circulating monocytes, while others are tissue-specific macrophages that reside within particular anatomical or tissue locations (“resident” macrophages). Tissue-specific macrophages include but are not limited to: adipose tissue macrophages; Kupffer cells (liver); sinus histiocytes (lymph nodes); alveolar macrophages (or dust cells, pulmonary alveoli of lungs); tissue macrophages (histiocytes) leading to giant cells (connective tissue); Langerhans cells (skin and mucosa); microglia (central nervous system); Hofbauer cells (placenta); intraglomerular mesangial cells (kidney); osteoclasts (bone); epithelioid cells (granulomas); red pulp macrophages (or sinusoidal lining cells, red pulp of spleen); peritoneal macrophages (peritoneal cavity); and LysoMac (Peyer's patch). Macrophages, e.g., bone-marrow derived monocytes, can be activated by certain stimuli (such as cytokines) resulting in polarized phenotypes, e.g., M1 and M2. M2-biased activated macrophages are further classified into several phenotypically distinct subtypes, such as M2a, M2b, M2c, e.g., pro-fibrotic) and M2d (pro-tumor or TAM-like).

Marker or Biomarker: encompasses the definition of biomarkers provided by the by the World Health Organization. The International Programme on Chemical Safety, led by the World Health Organization (WHO) in coordination with the United Nations and the International Labor Organization, has defined a biomarker as ‘any substance, structure, or process that can be measured in the body or its products and influence or predict the incidence of outcome or disease.’ WHO International Programme on Chemical Safety Biomarkers in Risk Assessment: Validity and Validation (2001). See also Strimbu & Tavel, “What are Biomarkers?” Curr. Opin. HIV AIDS, 5(6): 463-466 (November 2011). Predictive biomarkers provide information on the probability or likelihood of response to a particular therapy. Typically, a predictive biomarker is measured before and after treatment, and the changes or relative levels of the marker in samples collected from the subject indicates or predicts therapeutic benefit.

Matrix-Associated pro TGFβ1: has the immunological art-recognized meaning. LTBP1 and LTBP3 are presenting molecules that are components of the extracellular matrix. LTBP1-proTGFβ1 and LTBP3-proTGFβ1 may be collectively referred to as “ECM-associated” (or “matrix-associated”) proTGFβ1 complexes, that mediate ECM-associated TGFβ1 activation or signaling. LTBP1 and LTBP3 are components of the extracellular matrix. LTBP1-proTGFβ1 and LTBP3-proTGFβ1 may be collectively referred to as “ECM-associated” (or “matrix-associated”) proTGFβ1 complexes, that mediate ECM-associated TGFβ1 signaling/activities.

Maximally tolerated dose (MTD): has the immunological art-recognized meaning, in the context of safety or toxicology considerations, of the highest amount of a test article (such as a TGFβ1 inhibitor) evaluated with no observed adverse effect level (NOAEL). The NOAEL for a context-independent reference antibody in rats was the highest dose evaluated (100 mg/kg), suggesting that the maximally tolerated dose for a context-independent reference antibody is greater than 100 mg/kg, based on a four-week toxicology analysis.

Meso-Scale Discovery or MSD: has the immunological art-recognized meaning of a type of immunoassay that employs high binding carbon electrodes to capture proteins, e.g., antibodies. The antibodies can be incubated with particular antigens, which binding can be detected with secondary antibodies that are conjugated to electrochemiluminescent labels. Upon an electrical signal, light intensity can be measured to quantify analytes in the sample.

Myelofibrosis or osteomyelofibrosis: is a rare bone marrow proliferative disorder, which belongs to a group of diseases called myeloproliferative disorders and includes primary myelofibrosis and secondary myelofibrosis. Myelofibrosis characterized by the proliferation of an abnormal clone of hematopoietic stem cells in the bone marrow and other sites results in fibrosis, or the replacement of the marrow with scar tissue. Myelofibrosis is characterized by mutations that cause upregulation or overactivation of the downstream JAK pathway.

Myeloid-derived suppressor cells (MDSCs): have the immunological art-recognized meaning of a heterogeneous population of cells generated during various pathologic conditions and thought to represent a pathologic state of activation of monocytes and relatively immature neutrophils. MDSCs include at least two categories of cells termed (i) “granulocytic” (G-MDSC) or polymorphonuclear (PMN-MDSC), which are phenotypically and morphologically like neutrophils; and (ii) monocytic (M-MDSC) which are phenotypically and morphologically similar to monocytes. MDSCs are characterized by a distinct set of genomic and biochemical features and can be distinguished by specific surface molecules. Human G-MDSCs/PMN-MDSCs typically express the cell-surface markers CD11 b, CD33, CD15 and CD66. In addition, human G-MDSCs/PMN-MDSCs may also express HLA-DR or Arginase. By comparison, human M-MDSCs typically express the cell surface markers CD11 b, CD33 and CD14. The MDSCs may also express CD39 and CD73 to mediate adenosine signaling involved in organ fibrosis (such as liver fibrosis, and lung fibrosis), cancer and myelofibrosis. Human M-MDSCs may also express HLA-DR. In addition to such cell-surface markers, MDSCs are characterized by the ability to suppress immune cells, such as T cells, NK cells and B cells. Immune suppressive functions of MDSCs may include inhibition of antigen-non-specific function and inhibition of antigen-specific function. MDSCs can express cell surface LRRC33 or LRRC33-proTGFβ1.

Myofibroblasts: have the immunological art-recognized meaning of cells with certain phenotypes of fibroblasts and smooth muscle cells and generally express vimentin, alpha-smooth muscle actin (α-SMA; human gene ACTA2) and paladin. In many disease conditions involving extracellular matrix dysregulations (such as increased matrix stiffness), normal fibroblast cells become de-differentiated into myofibroblasts in a TGFβ3-dependent manner. Aberrant overexpression of TGFβ is common among myofibroblast-driven pathologies. TGFβ is known to promote myofibroblast differentiation, cell proliferation, and matrix production. Myofibroblasts or myofibroblast-like cells within the fibrotic microenvironment can be called fibrosis-associated fibroblasts (or “FAFs”).

Nintedanib: is an anti-scarring (anti-fibrotic) medication that slows progression of idiopathic pulmonary fibrosis. Some patients taking nintedanib have side effects, most commonly including diarrhea. There are no medications that cure idiopathic pulmonary fibrosis or decrease the amount of scarring in the lungs. Some medications help slow the progression of pulmonary fibrosis.

Off rate (k_{OFF}): has the immunological art-recognized meaning of a kinetic parameter of how fast or how slowly an antibody (such as mAb) or antigen-binding fragment (such as fAb) dissociates from its antigen and may be also referred to as the dissociation rate. Dissociation rates can be experimentally measured in suitable in vitro binding assays, such as bio-layer interferometry (Octet®) or surface plasmon resonance (BIACORE®)-based systems. In the context of antibody-antigen binding kinetics, the term “half-binding-time” (t_{1/2}) or “dissociation half-time” refers to the duration of time required for half the number of antibody molecules, e.g., mAb, Fab) to dissociate from the bound antigen, e.g., LTBP1-proTGFβ1, LTBP3-proTGFβ1. Thus, an antibody that dissociates slowly, i.e., low off rates) from its antigen has a long t_{1/2}. Conversely, an antibody that dissociates rapidly, i.e., high off rates) from its antigen has a short t_{1/2}.

Pan-TGFβ inhibitor or pan-inhibition of TGFβ: As used herein, the term pan inhibitor of TGFβ has the art-recognized meaning. A pan-TGFβ inhibitor is any agent that is capable of inhibiting or antagonizing all three isoforms of TGFβ. Such an inhibitor may be a small molecule inhibitor of TGFβ isoforms. The term includes pan-TGFβ antibody which refers to any antibody capable of binding to each of TGFβ isoforms, i.e., TGFβ1, TGFβ2, and TGFβ3. In a fifty-second embodiment, a pan-TGFβ antibody binds to and neutralizes activities of all three isoforms, i.e., TGFβ1, TGFβ2, and TGFβ3 activities.

Picrosirius red: Also known as Sirius Red F 3B or Direct Red 80), Picosirius red is an azo dye primarily used in staining methods for collagen and amyloid. Picrosirius red (PSR) has the molecular formula C₄₅H₂₆N₁₀Na₆O₂₁S₆.

Pirfenidone: Pirfenidone is an anti-scarring (anti-fibrotic) medication that slows the progression of idiopathic pulmonary fibrosis. Some patients taking pirfenidone have side effects, most commonly stomach upset and skin rash, particularly with exposure to sun. Pirfenidone was approved by Health Canada for the treatment of mild to moderate idiopathic pulmonary fibrosis. To date, there are no medications that cure idiopathic pulmonary fibrosis or decrease the amount of scarring in the lungs. Some medications help slow the progression of pulmonary fibrosis.

Potency: The term potency has the immunological art-recognized meaning of the activity of a drug, such as a functional antibody (or fragment) having inhibitory activity, with respect to concentration or amount of the drug to produce a defined effect. For example, an antibody capable of producing certain effects at a given dosage is more potent than another antibody that requires twice the amount (dosage) to produce equivalent effects. Potency may be measured in cell-based assays, such as TGFβ activation/inhibition assays, whereby the degree of TGFβ activation, such as activation triggered by integrin binding, can be measured in the presence or absence of test article (e.g., inhibitory antibodies) in a cell-based system. Typically, among those capable of binding to the same or overlapping binding regions of an antigen (e.g., cross-blocking antibodies), antibodies with higher affinities (lower K_{D} values) tend to show higher potency than antibodies with lower affinities (greater K_{D} values).

Predictive biomarkers: provide information on the probability or likelihood of response to a particular therapy. Typically, a predictive biomarker is measured before and after treatment, and the changes or relative levels of the marker in samples collected from the subject indicates or predicts therapeutic benefit.

Presenting molecule: As used herein, the term presenting molecule refers to proteins that form covalent bonds with proTGFβ and present the inactive complex in an extracellular niche (such as extracellular matrix or immune cell surface) thereby maintaining its latency until an activation event occurs. Known presenting molecules for proTGFβ1 include: LTBP1, LTBP3, GARP and LRRC33, which can form presenting molecule-proTGFβ1 complexes, namely, LTBP1-proTGFβ1, LTBP3-proTGFβ1, GARP-proTGFβ1 and LRRC33-proTGFβ1, respectively. Presenting molecules in the context of the present disclosure refer to anchoring proteins that can form covalent bonds with latent pro-proteins (e.g., proTGFβ1) and “present” the inactive complex in an extracellular niche (such as ECM or immune cell surface) thereby maintaining its latency until an activation event occurs. Known presenting molecules for proTGFβ1 include: LTBP1, LTBP3, GARP (also known as LRRC32) and LRRC33, which can form presenting molecule-proTGFβ1 complexes (LLCs), namely, LTBP1-proTGFβ1, LTBP3-proTGFβ1, GARP-proTGFβ1 and LRRC33-proTGFβ1, respectively. In nature, LTBP1 and LTBP3 are components of the extracellular matrix (ECM); therefore, LTBP1-proTGFβ1 and LTBP3-proTGFβ1 may be collectively referred to as “ECM-associated” (or “matrix-associated”) proTGFβ1 complexes that mediate ECM-associated TGFβ1 signaling/activities. GARP and LRRC33, on the other hand, are transmembrane proteins expressed on cell surface of certain cells; therefore, GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 signaling/activities.

proTGFβ1: As used herein, the term proTGFβ1 refers to and encompasses precursor forms of inactive TGFβ1 complex that comprises a prodomain sequence of TGFβ1 within the complex. The term can include the pro-forms, as well as the latent-forms of TGFβ1. The expression “pro/latent TGFβ1” may be used interchangeably. The “pro” form of TGFβ1 exists prior to proteolytic cleavage at the furin site. Once cleaved, the resulting form is said to be the “latent” form of TGFβ1. The “latent” complex remains associated until further activation trigger, such as integrin-driven activation event. The proTGFβ1 complex is comprised of dimeric TGFβ1 pro-protein polypeptides, linked with disulfide bonds. The latent dimer complex is covalently linked to a single presenting molecule via the cysteine residue at position 4 (Cys4) of each of the proTGFβ1 polypeptides. The adjective “latent” may be used generally to describe the “inactive” state of TGFβ1, prior to integrin-mediated or other activation events. The proTGFβ1 polypeptide contains a prodomain (LAP) and a growth factor domain.

Regulatory T cells, or Tregs: have the immunological art-recognized meaning of a type of immune cells characterized by the expression of the biomarkers, CD4, forkhead box P3 (FOXP3), and CD25, as well as STATS. Tregs are sometimes referred to as suppressor T cells and represent a subpopulation of T cells that modulate the immune system, maintain tolerance to self-antigens, and prevent autoimmune disease. Tregs are immunosuppressive and generally suppress or downregulate induction and proliferation of effector T (Teff) cells. Tregs can develop in the thymus (so-called CD4+ Foxp3+“natural” Tregs) or differentiate in the periphery upon priming of naïve CD4+ T cells by antigen-presenting cells (APCs), e.g., following exposure to TGFβ or retinoic acid. Treg cells produce and secrete cytokines including IL-10 and TGFβ1. Generally, differentiation of Treg and Th17 cells is negatively correlated.

Selection or selecting: has the plain meaning of the act of choosing from a number or group by fitness or preference. See Merriam-Webster's Unabridged Dictionary (2021).

Selectivity: has immunological art-recognized meaning. While antibody specificity is defined by the epitope the antibody binds to, antibody is defined by the antibody's binding to a unique epitope. Selectivity in this context may refer to at least a 500-1000-fold difference in respective affinities as measured by an in vitro binding assay such as OCTET® and BIACORE®. The selectivity is such that the inhibitor when used at a dosage effective to inhibit TGFβ1 in vivo does not inhibit TGFβ2 and TGFβ3. The Ab42 in vitro binding assay results provided in the EXAMPLES below show selectivity towards the LTBP complexes.

Specific binding or specifically binds: has the immunological art-recognized meaning that the interaction of the antibody or antigen-binding portion thereof with an antigen or amino acid residue is dependent upon the presence of a particular structure, e.g., an antigenic determinant or epitope. The antibody or antigen-binding portion thereof binds to a specific protein rather than to proteins generally.

The term “specific binding to an epitope of proTGFβ1,” “specifically binds to an epitope of proTGFβ1,” “specific binding to proTGFβ1,” or “specifically binds to proTGFβ1” as used herein, refers to an antibody or an antigen-binding portion thereof that binds to proTGFβ1 and has a dissociation constant (K_{D}) of 1.0×10⁻⁸ M or less, as determined by suitable in vitro binding assays. In some embodiments, an antibody or antigen-binding portion thereof can specifically bind to both human and a non-human, (e.g., mouse) orthologues of proTGFβ1. In some embodiments, an antibody or an antigen-binding portion thereof specifically binds to a target, e.g., TGFβ1, if the antibody has a K_{D} for the target of at least about 10-6 M. Preferably, the measured K_{D} values of such antibody range between 10-100 nM. More preferably, the measured K_{D} values of such antibody range between 0.1-10 nM. By in vitro pharmacology as measured by bio-layer interferometry, Ab 42 was measured to have a K_{D} of less than or equal to 10 nM binding to human LTBP1/3 large latent TGFβ1 complexes. See WO 2020/160291, incorporated by reference in its entirety herein.

Subject: The term “subject” in the context of therapeutic applications refers to an individual who receives clinical care or intervention, such as treatment, diagnosis, etc. Suitable subjects include vertebrates, including but not limited to mammals (e.g., human and non-human mammals). Where the subject is a human subject, the term “patient” may be used interchangeably. In a clinical context, the term “a patient population” or “patient subpopulation” is used to refer to a group of individuals that falls within a set of criteria, such as clinical criteria (e.g., disease presentations, disease stages, susceptibility to certain conditions, responsiveness to therapy, etc.), medical history, health status, gender, age group, genetic criteria (e.g., carrier of certain mutation, polymorphism, gene duplications, DNA sequence repeats, etc.) and lifestyle factors (e.g., diet, smoking, alcohol consumption, exercise, etc.).

Target engagement: As used herein, the term target engagement refers to the ability of a molecule (e.g., TGFβ inhibitor) to bind to its intended target in vivo (e.g., endogenous TGFβ). In case of activation inhibitors, the intended target can be a large latent complex.

T helper 17 cells (Th17): is meant to refer to a subset of pro-inflammatory T helper cells characterized by the markers STAT3 and RORγt and the production of cytokines including interleukin 17 (IL-17A/F) and IL-22. Th17 cells are differentiated when naive T cells are exposed to TGFβ and IL-6. Th17 cells are generally associated with tissue inflammation, autoimmunity and clearance of certain pathogens. The differentiation of Th17 cells and Treg cells is generally inversely related. Imbalance in Th17-to-Treg ratios, (e.g., “Th17/Treg”) has been implicated in a number of pathologies, such as fibrotic conditions and autoimmune conditions.

TGFβ inhibitor: The term “TGFβ inhibitor” refers broadly to any agent capable of antagonizing biological activities, signaling or function of TGFβ growth factor (e.g., TGFβ1, TGFβ2 and/or TGFβ3).

The term is not intended to limit its mechanism of action and includes, for example, neutralizing antibodies, receptor antagonists (e.g., kinase inhibitors), soluble ligand traps, and activation inhibitors of TGFβ. Non-selective TGFβ inhibitors are commonly referred to as “pan-inhibitors” of TGFβ. TGFβ inhibitors also include antibodies that are capable of reducing the availability of latent proTGFβ which can be activated in the niche, for example, by inducing antibody-dependent cell mediated cytotoxicity (ADCC), and/or antibody-dependent cellular phagocytosis (ADPC), as well as antibodies that result in internalization of cell-surface complex comprising latent proTGFβ, thereby removing the precursor from the plasma membrane without depleting the cells themselves.

The “TGFβ family” is a class within the TGFβ superfamily and contains three members in human: TGFβ1, TGFβ2, and TGFβ3, which are structurally similar and are encoded by separate genes. The three growth factors are known to signal via the same receptors.

TGFβ1 activation: has the immunological art-recognized meaning. See, e.g., WO 2017/156500, incorporated by reference in its entirety herein. Unlike other cytokines, TGFβ superfamily members are not secreted as active growth factors, but as dimeric pro-proteins which consist of an N-terminal prodomain and a C-terminal growth factor domain. Cleavage of proTGFβ1 by furin proteases separates the homodimeric growth factor domain from its prodomain, also referred to as latency associated peptide (LAP). However, the growth factor and LAP remain noncovalently associated, forming a latent complex which is unable to bind its receptors and induce signaling. During translation, latent TGFβ1, also called the small latent complex (SLC), becomes linked to “presenting molecules” via disulfide bridges, forming the large latent complex (LLC). These molecules allow proTGFβ1 to be presented in specific cellular or tissue contexts. Two cysteines near the N-terminus of the latent TGFβ1 link to appropriately positioned cysteines on the presenting molecule. The identity of the presenting molecule depends on the environment and cell type producing latent TGFβ1. Fibroblasts secrete latent TGFβ1 tethered to latent TGFβ-binding proteins (LTBPs), which then associate with proteins in the extracellular matrix (ECM), i.e., fibronectin, fibrillin-1) to link latent TGFβ to the extracellular matrix.

TGFβ1-related indication: A “TGFβ1-related indication” means any disease, disorder and/or condition related to expression, activity and/or metabolism of a TGFβ1 or any disease, disorder and/or condition that may benefit from inhibition of the activity and/or levels TGFβ1. Certain TGFβ1-related indications are driven predominantly by the TGFβ1 isoform. TGFβ1-related indications include, but are not limited to: fibrotic conditions (such as organ fibrosis, and fibrosis of tissues involving chronic inflammation), proliferative disorders (such as cancer, e.g., solid tumors and myelofibrosis), disease associated with ECM dysregulation (such as conditions involving matrix stiffening and remodeling), disease involving endothelial-to-mesenchymal transition (EndMT), disease involving epithelial-to-mesenchymal transition (EMT), disease involving proteases, disease with aberrant gene expression of certain markers described herein. These disease categories are not intended to be mutually exclusive. According to some embodiments, the TGFβ1-related indication is fibrosis.

TGFβ1 superfamily: in mammals, the transforming growth factor-beta (TGFβ) superfamily is comprised of at least 33 gene products. These include the bone morphogenetic proteins (BMPs), activins, growth and differentiation factors (GDFs), and the three isoforms of the TGFβ family: TGFβ1, TGFβ2, and TGFβ3. The TGFβs have key functions in several processes, such as inhibition of cell proliferation, extracellular matrix (ECM) remodeling, and immune homeostasis. The roles of TGFβ2 and TGFβ3 are less clear to persons having ordinary skill in the immunological art. The three TGFβ isoforms have distinct temporal and spatial expression patterns. The three TGFβ isoforms all through the same receptors, TGFβRI and TGFβRII, although sometimes type III receptors such as betaglycan are also required. The biological importance of the TGFβ pathway in humans was implicated and validated genetic diseases. Several drugs that target the TGFβ pathway were developed and tested in patients, but with limited success.

Th17-to-Treg ratios: have the immunological art-recognized meaning of measured ratios (relative proportions) of the number of Th17 cells versus the number of Treg cells in a tissue or sample of interest. Typically, known cell markers are used to identify, sort or isolate the cell types. Such markers include cell-surface molecules expressed on the particular cell type; a cytokine or a panel of cytokines produced, e.g., secreted) by the particular cell type, or mRNA expression of certain gene markers that serve as a signature/profile of the particular cell type. A Th17/Treg ratio of one (1) means that there is an equal or equivalent number of each of the cell types within the tissue or sample being evaluated. A Th17/Treg ratio of two (2) means that there is approximately twice the number of Th17 cells as compared to Treg cells in the tissue or sample. An elevated Th17/Treg ratio may arise from an increased number of Th17 cells, a decreased number of Treg cells, or combination thereof.

Therapeutic window: has the medical art-recognized meaning a range of doses or concentrations that produces therapeutic response without causing significant, observable, or unacceptable adverse effect in subjects, e.g., within adverse effects that are acceptable or tolerable. Therapeutic window may be calculated as a ratio between minimum effective concentrations (MEC) to the minimum toxic concentrations (MTC). A TGFβ1 inhibitor that achieves in vivo efficacy at 10 mg/kg and shows tolerability or acceptable toxicities at 100 mg/kg provides at least a 10-fold, e.g., 10× therapeutic window. By contrast, a pan-inhibitor of TGFβ that is efficacious at 10 mg/kg but causes adverse effects at 5 mg/kg is said to have “dose-limiting toxicities.” A context-independent TGFβ1 inhibitor antibody is efficacious at dosage ranging between about <3 and 30 mg/kg/week and is free of observable toxicities associated with pan-inhibition of TGFβ at least 100 mg/kg/week for four weeks in preclinical models such as rats. Based on this, the context-independent TGFβ1 inhibitor antibody shows at minimum a 3.3-fold and up to 33-fold therapeutic window.

Toxicity or toxicities: have the immunological art-recognized meaning of unwanted in vivo effects in patients associated with a therapy administered to the patients, such as undesirable side effects and adverse events. “Tolerability” refers to a level of toxicities associated with a therapy or therapeutic regimen, which can be reasonably tolerated by patients, without discontinuing the therapy due to the toxicities, i.e., acceptable level of toxicities. Typically, toxicity/toxicology studies are carried out in one or more preclinical models prior to clinical development to assess safety profiles of a drug candidate, e.g., monoclonal antibody therapy. Toxicity or toxicology studies may help determine the “no observed adverse effect level (NOAEL)” and the “maximally tolerated dose (MTD)” of a test article, based on which a therapeutic window may be deduced. Preferably, a species that is shown to be sensitive to the particular intervention should be chosen as a preclinical animal model in which the safety or toxicity analysis is to be carried out. For TGFβ inhibition, suitable species include rats, dogs, and cynomologus monkeys. Mice are reported to be less sensitive to pharmacological inhibition of TGFβ and may not reveal toxicities that are potentially dangerous in other species, including human, although certain studies report toxicities observed with pan-inhibition of TGFβ in mice. To illustrate, the NOAEF for a context-independent TGFβ1 inhibitor antibody in rats was the highest dose evaluated (100 mg/kg), suggesting that the MTD is greater than 100 mg/kg per week, based on a four-week toxicology analysis.

Treat/treatment: have the medical art-recognized meaning and include therapeutic treatments, prophylactic treatments, and applications in which one reduces the risk that a subject will develop a disorder or other risk factor. The term “treat” or “treatment” includes therapeutic treatments, prophylactic treatments, and applications in which one reduces the risk that a subject will develop a disorder or other risk factor. Thus the term is intended to broadly mean: causing therapeutic benefits in a patient by, for example, slowing disease progression, reversing certain disease features, normalizing gene expression, enhancing or boosting the body's immunity; reducing or reversing immune suppression; reducing, removing or eradicating harmful cells or substances from the body; reducing disease burden (e.g., fibrosis and tumor burden); preventing recurrence or relapse; prolonging a refractory period, and/or otherwise improving survival. The term includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In the context of combination therapy, the term may also refer to: i) the ability of a second therapeutic to reduce the effective dosage of a first therapeutic so as to reduce side effects and increase tolerability; ii) the ability of a second therapy to render the patient more responsive to a first therapy; and/or iii) the ability to effectuate additive or synergistic clinical benefits.

Variable region: The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

GARP and LRRC33, on the other hand, are transmembrane proteins expressed on cell surface of certain cells; therefore, GARP-proTGFβ1 and LRRC33-proTGFβ1 may be collectively referred to as “cell-associated” (or “cell-surface”) proTGFβ1 complexes, that mediate cell-associated, e.g., immune cell-associated) TGFβ1 signaling/activities.

Other than in the operating examples, or where otherwise indicated, all numbers expressing quantities of ingredients or reaction conditions used herein should be understood as modified in all instances by the term “about.” The term “about” when used in connection with percentages can mean±1%.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, e.g., 10-20, 1-10, 30-40, etc.

The disclosure described herein does not concern a process for cloning humans, processes for modifying the germ line genetic identity of humans, uses of human embryos for industrial or commercial purposes or processes for modifying the genetic identity of animals which are likely to cause them suffering with no substantial medical benefit to man or animal, and also animals resulting from such processes.

### Transforming Growth Factor-Beta (TGFβ)

In mammals, the transforming growth factor-beta (TGFβ) superfamily is comprised of at least 33 gene products. These include the bone morphogenetic proteins (BMPs), activins, growth and differentiation factors (GDFs), and the three isoforms of the TGFβ family: TGFβ1, TGFβ2, and TGFβ3. The TGFβs are thought to play key roles in diverse processes, such as inhibition of cell proliferation, extracellular matrix (ECM) remodeling, and immune homeostasis. The importance of TGFβ1 for T cell homeostasis is demonstrated by the observation that TGFβ1−/− mice survive only 3-4 weeks, succumbing to multiorgan failure due to massive immune activation (Kulkarni, A. B., et al., Proc Natl Acad Sci USA, 1993. 90(2): p. 770-4; Shull, M. M., et al., Nature, 1992. 359(6397): p. 693-9). The roles of TGFβ2 and TGFβ3 are less clear. Whilst the three TGFβ isoforms have distinct temporal and spatial expression patterns, they signal through the same receptors, TGFβRI and TGFβRII, although in some cases, for example for TGFβ2 signaling, type III receptors such as betaglycan are also required (Feng, X. H. and R. Derynck, Annu Rev Cell Dev Biol, 2005. 21: p. 659-93; Massague, J., Annu Rev Biochem, 1998. 67: p. 753-91). Ligand-induced oligomerization of TGFβRI/II triggers the phosphorylation of SMAD transcription factors, resulting in the transcription of target genes, such as Coil a1, Col3a1, ACTA2, and SERPINE1 (Massague, J., J. Seoane, and D. Wotton, Genes Dev, 2005. 19(23): p. 2783-810). SMAD-independent TGFβ signaling pathways have also been described, for example in cancer or in the aortic lesions of Marfan mice (Derynck, R. and Y. E. Zhang, Nature, 2003. 425(6958): p. 577-84; Holm, T. M., et al., Science, 2011. 332(6027): p. 358-61).

The biological importance of the TGFβ pathway in humans has been validated by genetic diseases. Camurati-Engelman disease results in bone dysplasia due to an autosomal dominant mutation in the TGF #81 gene, leading to constitutive activation of TGFβ1 signaling (Janssens, K., et al., J Med Genet, 2006. 43(1): p. 1-11). Patients with Loeys/Dietz syndrome carry autosomal dominant mutations in components of the TGFβ signaling pathway, which cause aortic aneurism, hypertelorism, and bifid uvula (Van Laer, L., H. Dietz, and B. Loeys, Adv Exp Med Biol, 2014. 802: p. 95-105). As TGFβ pathway dysregulation has been implicated in multiple diseases, several drugs that target the TGFβ pathway have been developed and tested in patients, but with limited success. Most TGFβ inhibitors described to date lack isoform specificity as briefly summarized below.

Fresolimumab, a humanized monoclonal antibody that binds and inhibits all three isoforms of TGFβ has been tested clinically in patients with focal segmental glomerulosclerosis, malignant melanoma, renal cell carcinoma, and systemic sclerosis (Rice, L. M., et al., J Clin Invest, 2015. 125(7): p. 2795-807; Trachtman, H., et al., Kidney Int, 2011. 79(11): p. 1236-43; Morris, J. C., et al., PLoS One, 2014. 9(3): p. e90353). Additional companies have developed monoclonal antibodies against the TGFβ growth factors with varying degrees of selectivity for TGFβ isoforms. Such agents likely elicit toxicities in vivo through residual activity against other TGFβ family members besides TGFβ1. This lack of isoform specificity may be due to the high degree of sequence identity between isoforms.

Other approaches to target the TGFβ pathway include ACE-1332, a soluble TGFβRII-Fc ligand trap from Acceleron (Yung, L. M., et al., A Am J Respir Crit Care Med, 2016. 194(9): p. 1140-1151), or small molecule inhibitors of the ALK5 kinase, such as Eli Lilly's galunisertib. ACE-1332 binds TGFβ1 and TGFβ3 with equally high affinity (Yung, L. M., et al., Am J Respir Crit Care Med, 2016. 194(9): p. 1140-1151), and ALK5 inhibitors block the activity of all growth factors that signal through TGFR1. Substantial toxicities have been found in preclinical studies using ALK5 inhibitors (Anderton, M. J., et al., Toxicol Pathol, 2011. 39(6): p. 916-24; Stauber, A., et al., Clinical Toxicology, 2014. 4(3): p. 1-10), and sophisticated clinical dosing schemes are required to maintain efficacy while reducing adverse events (Herbertz, S., et al., Drug Des Devel Ther, 2015. 9: p. 4479-99). In fact, the question of TGFβ signaling specificity and its possible effect on toxicity observed with the known TGFβ inhibitors has not been raised in most, if not all, of the candidate drugs that attempted to block TGFβ. For example, how much of the toxicities are due to inhibition of TGFβ1 versus TGFβ2 and/or TGFβ3 has not been addressed. Similarly, modes of TGFβ activation have not been taken into account in designing or developing ways to antagonize TGFβ signaling.

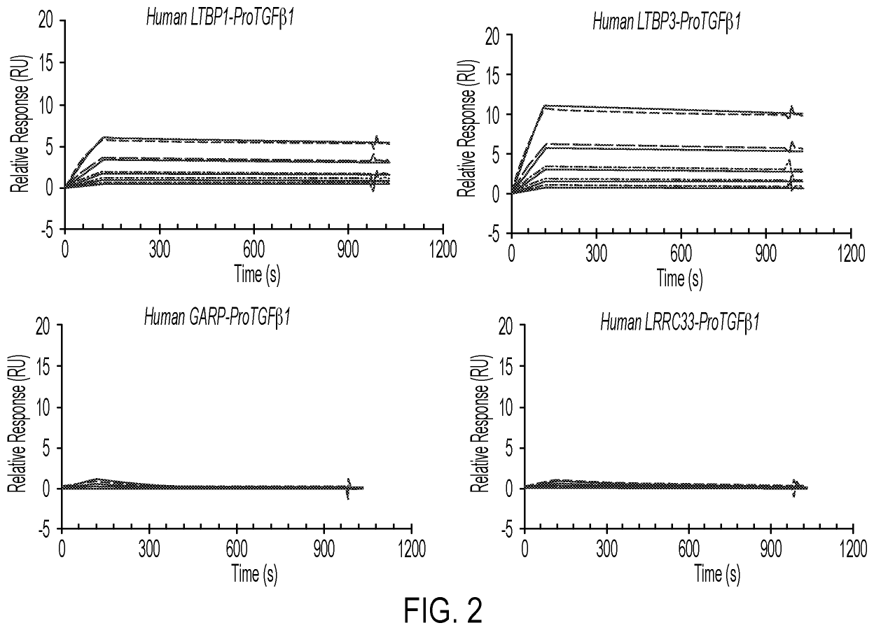

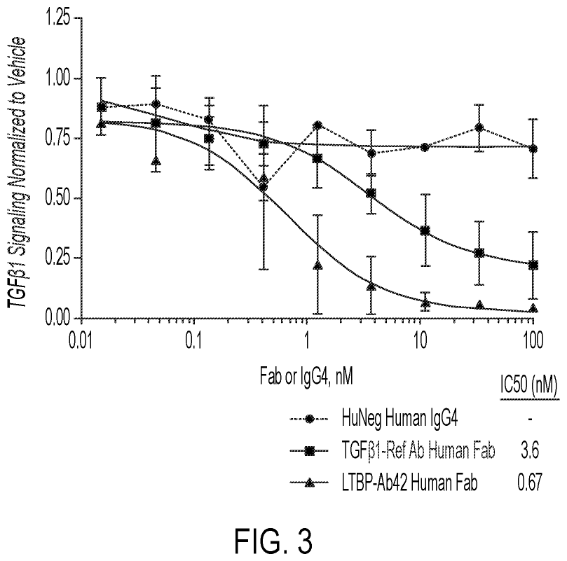

Recent structural insights into the activation mechanism of TGFβ1 (Shi, M., et al., Nature, 2011. 474(7351): p. 343-9) have enabled more specific approaches to TGFβ inhibition (see, e.g., PCT/US2017/21972, the entire contents of which are incorporated herein by reference). Unlike other cytokines, TGFβ superfamily members are not secreted as active growth factors, but as dimeric pro-proteins which consist of an N-terminal prodomain and a C-terminal growth factor domain. Cleavage of proTGFβ1 by furin proteases separates the homodimeric growth factor domain from its prodomain, also referred to as latency associated peptide (LAP). However, the growth factor and LAP remain noncovalently associated, forming a latent complex which is unable to bind its receptors and induce signaling. During translation, latent TGFβ1, also called the small latent complex (SLC), becomes linked to “presenting molecules” via disulfide bridges, forming the large latent complex (LLC). These molecules allow proTGFβ1 to be presented in specific cellular or tissue contexts. Two cysteines near the N-terminus of the latent TGFβ1 link to appropriately positioned cysteines on the presenting molecule. The identity of the presenting molecule depends on the environment and cell type producing latent TGFβ1. For example, fibroblasts secrete latent TGFβ1 tethered to latent TGFβ-binding proteins (LTBPs), which then associate with proteins in the extracellular matrix (ECM) (i.e., fibronectin, fibrillin-1) to link latent TGFβ to the ECM (Robertson et al. Matrix Biol 47: 44-53 (2015) (FIG. 2A). On the surface of activated regulatory T cells latent TGFβ1 is covalently linked to the transmembrane protein GARP (glycoprotein-A repetitions predominant protein (GARP), and a protein closely related to GARP, LRRC33 (leucine-rich repeat-containing protein 33), serves as a presenting molecule for TGFβ1 on the surface of monocytes, macrophages and microglia (Wang, R., et al., Mol Biol Cell, 2012. 23(6): p. 1129-39 and T. A. Springer, Int. BMP Conference 2016).

A number of studies have shed light on the mechanisms of TGFβ1 activation. Three integrins, αVβ6, αVβ8, and αVβ1 have been demonstrated to be key activators of latent TGFβ1 (Reed, N. I., et al., Sci Transl Med, 2015. 7(288): p. 288ra79; Travis, M. A. and D. Sheppard, Annu Rev Immunol, 2014. 32: p. 51-82; Munger, J. S., et al., Cell, 1999. 96(3): p. 319-28). αV integrins bind the RGD sequence present in TGFβ1 and TGFβ1 LAPs with high affinity (Dong, X., et al., Nat Struct Mol Biol, 2014. 21(12): p. 1091-6). Transgenic mice with a mutation in the TGFβ1 RGD site that prevents integrin binding, but not secretion, phenocopy the TGFβ1−/− mouse (Yang, Z., et al., J Cell Biol, 2007. 176(6): p. 787-93). Mice that lack both β6 and β8 integrins recapitulate all essential phenotypes of TGFβ1 and TGFβ3 knockout mice, including multiorgan inflammation and cleft palate, confirming the essential role of these two integrins for TGFβ1 activation in development and homeostasis (Aluwihare, P., et al., J Cell Sci, 2009. 122(Pt 2): p. 227-32). Key for integrin-dependent activation of latent TGFβ1 is the covalent tether to presenting molecules; disruption of the disulfide bonds between GARP and TGFβ1 LAP by mutagenesis does not impair complex formation, but completely abolishes TGFβ1 activation by αVβ6 (Wang, R., et al., Mol Biol Cell, 2012. 23(6): p. 1129-39). The recent structure of latent TGFβ1 illuminates how integrins enable release of active TGFβ1 from the latent complex: the covalent link of latent TGFβ1 to its presenting molecule anchors latent TGFβ1, either to the ECM through LTBPs, or to the cytoskeleton through GARP or LRRC33. Integrin binding to the RGD sequence results in a force-dependent change in the structure of LAP, allowing active TGFβ1 to be released and bind nearby receptors (Shi, M., et al., Nature, 2011. 474(7351): p. 343-9). The importance of integrin-dependent TGFβ1 activation in disease has also been well validated. A small molecular inhibitor of αVβ1 protects against bleomycin-induced lung fibrosis and carbon tetrachloride-induced liver fibrosis (Reed, N. I., et al., Sci Transl Med, 2015. 7(288): p. 288ra79), and αVβ6 blockade with an antibody or loss of integrin 136 expression suppresses bleomycin-induced lung fibrosis and radiation-induced fibrosis (Munger, J. S., et al., Cell, 1999. 96(3): p. 319-28); Horan, G. S., et al., Am J Respir Crit Care Med, 2008. 177(1): p. 56-65). In addition to integrins, other mechanisms of TGFβ1 activation have been implicated, including thrombospondin-1 and activation by proteases such as matrix metalloproteinases (MMPs), cathepsin D or kallikrein. However, the majority of these studies were performed in vitro using purified proteins; there is less evidence for the role of these molecules from in vivo studies. Knockout of thrombospondin-1 recapitulates some aspects of the TGFβ1−/− phenotype in some tissues, but is not protective in bleomycin-induced lung fibrosis, known to be TGFβ3-dependent (Ezzie, M. E., et al., Am J Respir Cell Mol Biol, 2011. 44(4): p. 556-61). Additionally, knockout of candidate proteases did not result in a TGFβ1 phenotype (Worthington, J. J., J. E. Klementowicz, and M. A. Travis, Trends Biochem Sci, 2011. 36(1): p. 47-54). This could be explained by redundancies or by these mechanisms being critical in specific diseases rather than development and homeostasis.

TGFβ has been implicated in a number of biological processes, including fibrosis, immune-modulation and cancer progression. TGFβ1 was the first identified member of the TGFβ superfamily of proteins. Like other members of the TGFβ superfamily, TGFβ1 and the isoforms TGFβ2 and TGFβ3, are initially expressed as inactive precursor pro-protein forms (termed proTGFβ). TGFβ proteins (e.g., TGFβ1, TGFβ2 and TGFβ3) are proteolytically cleaved by proprotein convertases (e.g., furin) to yield the latent form (termed latent TGFβ). In some embodiments, a pro-protein form or latent form of a TGFβ protein (e.g., TGFβ1, TGFβ2 and TGFβ3) may be referred to as “pro/latent TGFβ protein”. TGFβ1 may be presented to other molecules in complex with multiple molecules including, for example, GARP (to form a GARP-TGFβ1 complex), LRRC33 (to form a LRRC33-TGFβ1 complex), LTBP1 (to form a LTBP1-TGFβ1 complex), and/or LTBP3 (to form a LTBP3-TGFβ1 complex). The TGFβ1 present in these complexes may be in either latent form (latent TGFβ1) or in precursor form (proTGFβ1).

### Binding Regions and Epitopes

In the context of the present disclosure, the “binding region” of an antigen provides a structural basis for the antibody-antigen interaction. As used herein, a “binding region” refers to the areas of interface between the antibody and the antigen, such that, when bound to the proTGFβ1 complex (“antigen”) in a physiological solution, the antibody or the fragment protects the binding region from solvent exposure, as determined by suitable techniques, such as hydrogen-deuterium exchange mass spectrometry (HDX-MS).

In some embodiments, the first binding region and/or the second binding region confers the isoform selectivity of the antibody or the fragment.

Advantageously, preferred inhibitory antibodies of the present disclosure are capable of inhibiting the release of mature growth factor from a latent complex associated with LTBP1 or LTBP3, thereby reducing growth factor signaling. Such antibodies may target any epitope that results in a reduction of growth factor release or activity when associated with such antibodies. In some embodiments, the antibodies of the present disclosure specifically bind a combinatorial epitope, i.e., an epitope formed by two or more components/portions of an antigen or antigen complex. For example, a combinatorial epitope may be formed by contributions from multiple portions of a single protein, i.e., amino acid residues from more than one non-contiguous segments of the same protein. Alternatively, a combinatorial epitope may be formed by contributions from multiple protein components of an antigen complex. In some embodiments, the antibodies of the present disclosure specifically bind a conformational epitope (or conformation-specific epitope), e.g., an epitope that is sensitive to the three-dimensional structure (i.e., conformation) of an antigen or antigen complex. In preferred embodiments, the combinatorial epitope comprises an amino acid residue within Alpha-1 Helix and optionally an amino acid residue within the growth factor domain. In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called alpha-1 helix” region of the prodomain of the latent proTGFβ1 complex. In some embodiments, the antibody or the fragment contacts one or more of the following residues within the prodomain: Asp27 (D); Leu3 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

TABLE 1
Select protein domains/modules of human TGFB1 polypeptide

| Human TGFß1 | | SEQ ID |
|---|---|---|
| domain/module | Amino Acid Sequence | NO |
| Latency | LSTCKTIDMELVKRKRIEAIRGQILSKLRLASPPSQGEVPPGPLP | 47 |
| Associated | EAVLALYNSTRDRVAGESAEPEPEPEADYYAKEVTRVLMVETH | |
| Peptide (LAP) | NEIYDKFKQSTHSIYMFFNTSELREAVPEPVLLSRAELRLLRLKL | |
| (prodomain) | KVEQHVELYQKYSNNSWRYLSNRLLAPSDSPEWLSFDVTGVV | |
| | RQWLSRGGEIEGFRLSAHCSCDSRDNTLQVDINGFTTGRRGD | |
| | LATIHGMNRPFLLLMATPLERAQHLQSSRHRR | |
| Straight Jacket | LSTCKTIDMELVKRKRIEAIRGQILSKLRLASPPSQGEVPPGPLP | 48 |
| Growth Factor | ALDTNYCFSSTEKNCCVRQLYIDFRKDLGWKWIHEPKGYHANF | 49 |
| Domain | CLGPCPYIWSLDTQYSKVLALYNQHNPGASAAPCCVPQALEPL | |
| | PIVYYVGRKPKVEQLSNMIVRSCKCS | |
| Fastener | residues 74-76, YYA | |
| Furin cleavage | RHRR | 50 |
| site | | |
| Arm | EAVLALYNSTRDRVAGESAEPEPEPEADYYAKEVTRVLMVETH | 51 |
| | NEIYDKFKQSTHSIYMFFNTSELREAVPEPVLLSRAELRLLRLKL | |
| | KVEQHVELYQKYSNNSWRYLSNRLLAPSDSPEWLSFDVTGVV | |
| | RQWLSRGGEIEGFRLSAHCSCDSRDNTLQVDINGFTTGRRGD | |
| | LATIHGMNRPFLLLMATPLERAQHLQSSRHRR | |
| Finger-1 | CVRQLYIDFRKDLGWKWIHEPKGYHANFC | 52 |
| Finger-2 | CVPQALEPLPIVYYVGRKPKVEQLSNMIVRSCKCS | 53 |
| Residue for | Cys 4 | |
| presenting | | |
| molecule | | |
| association | | |
| Latency Lasso | LASPPSQGEVPPGPL | 54 |
| Extended Latency | LASPPSQGEVPPGPLPEAVLALYNSTR | 55 |
| Lasso | | |
| Alpha-1 Helix | LSTCKTIDMELVKRKRIEAIRGQILSKLR | 56 |
| | (Preferred binding region underlined) | |
| | VKRKRIEA | 9 |
| | DMELVKRKRIEAIR | 46 |
| Alpha-2 Helix | AVLALYNSTR | 57 |
| Trigger Loop | NGFTTGRRGDLATIHGMNRP | 58 |
| Integrin binding | residue 215-217, RGD | |
| Bowtie | CSCDSRDNTLQVD | 59 |

### Latent TGFβ-Binding Proteins (LTBPs)

In mammals there are four known LTBPs, LTBP1-4, each with multiple splice variants (Robertson, I. B., et al., Matrix Biol, 2015. 47: p. 44-53). LTBP2 is the only LTBP that does not associate with latent TGFβ (Saharinen, J. and J. Keski-Oja, Mol Biol Cell, 2000. 11(8): p. 2691-704). While the association between LTBP1 or LTBP3 and latent TGFβ1 has been well validated, the role of LTBP4 in TGFβ presentation is less clear. The complex with LTBP4 and latent TGFβ1 appears to form much less efficiently, potentially due to the absence of several negatively charged residues in the TGFβ-binding domain of LTBP4 (Saharinen, J. and J. Keski-Oja, Mol Biol Cell, 2000. 11(8): p. 2691-704; Chen, Y., et al., J Mol Biol, 2005. 345(1): p. 175-86). Both LTBP4S−/− mice and Urban-Rifkin-Davis syndrome patients, who have null mutations in LTBP4, suffer from disrupted elastic fiber assembly (Urban, Z., et al., Am J Hum Genet, 2009. 85(5): p. 593-605; Dabovic, B., et al., J Cell Physiol, 2015. 230(1): p. 226-36). Additionally, while LTBP4S−/− mice have a lung septation and an elastogenesis defect, transgenic mice with an LTBP4 that cannot form a complex with latent TGFβ1 have no obvious phenotype (Dabovic, B., et al., J Cell Physiol, 2015. 230(1): p. 226-36). Whether LTBP4 is directly involved in regulation of latent TGFβ1 by functioning as a presenting molecule is unclear; LTBP4 may instead be required for proper formation of elastic fibrils in the ECM and its loss indirectly affect latent TGFβ1 activation through defects in the ECM.

In one aspect, the present disclosure is directed to inhibitors, e.g., immunoglobulins, e.g., antibodies, or antigen binding portions thereof, that selectively bind to a complex containing a TGFβ pro-protein and a LTBP protein (e.g., LTBP1 or LTBP3). In a preferred embodiment, the TGFβ protein is TGFβ1. In some embodiments, the binding molecules disclosed herein bind selectively to a complex containing pro/latent TGFβ1 and LTBP1 or LTBP3. Such binding molecules can allow TGFβ1 activity to be selectively modulated in a context-dependent manner, i.e., by modulating TGFβ1 in the context of a LTPB protein, without modulating the activity of TGFβ1 complexed with other presenting molecules (e.g., GARP and/or LRRC33).

Inhibitors that Selectively Bind to a LTBP-TGFβ1 Complex

The present disclosure provides novel, TGFβ1 inhibitors that selectively target matrix- or ECM-associated TGFβ1 activities. More specifically, such inhibitors include isoform-specific, context-selective inhibitors of TGFβ1 activation that specifically bind latent forms of TGFβ1 (e.g., proTGFβ1 complex) within the ECM environment and prevent release of mature growth factor from the complex at the niche. Such matrix-targeting inhibitors are context-specific in that they selectively bind proTGFβ1 associated with ECM presenting molecules, namely, LTBP1 and/or LTBP3. Thus, disclosed herein are monoclonal antibodies and fragments thereof capable of binding an epitope present in an LTBP1-proTGFβ1 complex and/or LTBP3-proTGFβ1 complex, whereas the epitope is not present in a GARP-proTGFβ1 complex and/or LRRC33-proTGFβ1 complex.

In some embodiments, the context-selective inhibitors of the present disclosure are capable of specifically binding both a human LTBP1-proTGFβ1 complex and a human LTBP3-proTGFβ1 complex, with affinities of at least 10 nM (measured K_{D} values) in a suitable in vitro binding assay, such as Octet. On the other hand, these context-specific antibodies show significantly less binding (e.g., at least 50-fold, preferably 100-fold reduced affinities) to a human GARP-proTGFβ1 complex or a human LRRC33-proTGFβ1 complex under the same assay conditions.

The TGFβ1 present in these complexes may be in either latent form (latent TGFβ1) or in precursor form (proTGFβ1). In one embodiment, the inhibitors do not significantly bind to LTBP1 alone (e.g., when not complexed with TGFβ1). In another embodiment, the inhibitors do not significantly bind to LTBP3 alone (e.g., when not complexed with TGFβ1). In another embodiment, the inhibitors do not significantly bind to TGFβ1 alone (e.g., pro or latent TGFβ1 not complexed with LTBP1 or LTBP3, or mature TGFβ1). In another embodiment, the inhibitors that selectively bind a LTBP1-TGFβ1 complex and/or a LTBP3-TGFβ1 complex do not significantly bind to a complex containing TGFβ1 and another presenting molecule, e.g., a GARP-TGFβ1 complex (e.g., GARP complexed to pro- or latent TGFβ1) and/or a LRRC33-TGFβ1 complex (e.g., LRRC33 complexed to pro- or latent TGFβ1). In one embodiment, the inhibitors that selectively bind LTBP1/3-TGFβ1 do not significantly bind one or more (e.g., two or more, three or more, or all four) of the following: LTBP1 alone, TGFβ1 alone, a GARP-TGFβ1 complex, and a LRRC33-TGFβ1 complex. In addition, in some embodiments, the inhibitors do not significantly bind LTBP3 alone.

As used herein, the term “inhibitor” refers to any agent capable of blocking or antagonizing TGFβ1 signaling. Such agents may include small molecule antagonists of TGFβ1 and biologic antagonists of TGFβ1 (e.g., protein fragments and antibodies). In some embodiments, the inhibitor may be an antibody (including fragments thereof, such as Domain Antibodies (dAbs) as described in, for example, U.S. Pat. Nos. 6,291,158; 6,582,915; 6,593,081; 6,172,197; and 6,696,245), a small molecule inhibitor, an Adnectin, an Affibody, a DARPin, an Anticalin, an Avimer, a Versabody or a gene therapy. Use of inhibitors encompassed by the present disclosure also includes antibody mimetics, such as monobodies and single-domain antibodies. Monobodies are synthetic binding proteins that typically employ a fibronectin type III domain (FN3) as a molecular scaffold. Monobodies include Adnectins™ which are based on the 10th fibronectin type III domain.

In some aspects, the inhibitors, e.g., antibodies, or antigen binding portions thereof, selectively bind to an epitope present on a LTBP1/3-TGFβ1 complex, that is not present on a GARP-TGFβ1 complex and/or a LRRC33-TGFβ1 complex. In some embodiments, the epitope is available due to a conformational change in LTBP1/3 and/or TGFβ1 that occurs when LTBP1/3 and TGFβ1 form a complex. In this embodiment, the epitope is not present in LTBP1/3 or TGFβ1 when the proteins are not associated in a complex. In one embodiment, the epitope is present on TGFβ1, when TGFβ1 is in a complex with LTBP1 or LTBP3. In another embodiment, the epitope is present on LTBP1, when LTBP1 is in a complex with TGFβ1. In another embodiment, the epitope is present on LTBP3, when LTBP3 is in a complex with TGFβ1. In another embodiment, the epitope comprises residues from both LTBP1 and TGFβ1. In another embodiment, the epitope comprises residues from both LTBP3 and TGFβ1. In some embodiments, the epitope is not accessible for binding by the antibody or a fragment thereof when the proTGFβ1 complex is associated with a cell via a cell-surface presenting molecule such as GARP and LRRC33.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In one aspect, the disclosure provides functional inhibitors, e.g., antibodies, that modulate TGFβ1 activity. In exemplary embodiments, the antibodies described herein are inhibitory antibodies, which inhibit the function or activity of TGFβ1. In some embodiments, the antibodies, or antigen binding portions thereof, inhibit the activation (release) of TGFβ1 from a LTBP1-TGFβ1 complex and/or a LTBP3-TGFβ1 complex. The present disclosure provides, in exemplary embodiments, “context-specific” or “context-selective” inhibitors of TGFβ1 activation. Such inhibitors can bind a LTBP1/3-TGFβ1 complex and inhibit activation of TGFβ1 that is presented by LTBP1 or LTBP3, without inhibiting the activation of TGFβ1 presented by GARP and/or LRRC33. Accordingly, in some embodiments, the antibodies, or antigen binding portions thereof, described herein inhibit the release of mature TGFβ1 from a LTBP1-TGFβ1 complex and/or a LTBP3-TGFβ1 complex, but do not inhibit the release of mature TGFβ1 from a GARP-TGFβ1 complex and/or a LRRC33-TGFβ1 complex. Due to the differential localization of LTBP, GARP, and LRRC33, the context-specific inhibitors of TGFβ1 provided by the present disclosure can block a particular subset of TGFβ1 activity in vivo. In one embodiment, the context-specific antibodies provided herein that inhibit LTBP1/3-TGFβ1 but do not inhibit GARP-TGFβ1 or LRRC33-TGFβ1 can be used to inhibit TGFβ1 localized to the extracellular matrix. In another embodiment, the context-specific antibodies can inhibit TGFβ1 without modulating TGFβ1-associated immune activity or immune response. In another embodiment, the context-specific antibodies can be used to inhibit TGFβ1 activity associated with the extracellular matrix without modulating TGFβ1 activity associated with hematopoietic cells. Accordingly, the context-specific antibodies can be used to inhibit LTBP1/3-associated TGFβ1 activity in applications in which TGFβ1 activation in the context of GARP and/or LRRC33 is undesirable, as described herein.

In some embodiments, the TGFβ1 comprises a naturally occurring mammalian amino acid sequence. In some embodiment, the TGFβ1 comprises a naturally occurring human amino acid sequence. In some embodiments, the TGFβ1 comprises a human, a monkey, a rat or a mouse amino acid sequence.

In some embodiments, an antibody, or antigen binding portion thereof, described herein selectively binds to a complex comprising a TGFβ1 protein comprising the amino acid sequence set forth in SEQ ID NO: 9, and LTBP1 or LTBP3. In some embodiments, an antibody, or antigen binding portion thereof, described herein selectively binds to a LTBP1/3-TGFβ1 complex which comprises a non-naturally-occurring TGFβ1 amino acid sequence (otherwise referred to herein as a non-naturally-occurring TGFβ1). For example, a non-naturally-occurring TGFβ1 may comprise one or more recombinantly generated mutations relative to a naturally-occurring TGFβ1 amino acid sequence.

In some embodiments, an antibody, or antigen binding portion thereof, described herein does not bind TGFβ2 and/or TGFβ3, or to protein complexes containing TGFβ2 and/or TGFβ3. Exemplary TGFβ2 and TGFβ3 amino acid sequences are set forth in SEQ ID NOs: 10 and 11, respectively. In some embodiments, a TGFβ1, TGFβ2, or TGFβ3 amino acid sequence comprises an amino acid sequence as set forth in SEQ ID NOs: 12-23, as shown in Table 2. In some embodiments, a TGFβ1 amino acid sequence comprises an amino acid sequence as set forth in SEQ ID NOs: 24-31, as shown in Table 3.

TGFß1
(SEQ ID NO: 60)
LSTCKTIDMELVKRKRIEAIRGQILSKLRLASPPSQGEVPPGPLPEAVL
ALYNSTRDRVAGESAEPEPEPEADYYAKEVTRVLMVETHNEIYDKFKQS
THSIYMFFNTSELREAVPEPVLLSRAELRLLRLKLKVEQHVELYQKYSN
NSWRYLSNRLLAPSDSPEWLSFDVTGVVRQWLSRGGEIEGFRLSAHCSC
DSRDNTLQVDINGFTTGRRGDLATIHGMNRPFLLLMATPLERAQHLQSS
RHRRALDTNYCFSSTEKNCCVRQLYIDFRKDLGWKWIHEPKGYHANFCL
GPCPYIWSLDTQYSKVLALYNQHNPGASAAPCCVPQALEPLPIVYYVGR
KPKVEQLSNMIVRSCKCS
TGFß2
(SEQ ID NO: 61)
SLSTCSTLDMDQFMRKRIEAIRGQILSKLKLTSPPEDYPEPEEVPPEVI
SIYNSTRDLLQEKASRRAAACERERSDEEYYAKEVYKIDMPPFFPSENA
IPPTFYRPYFRIVRFDVSAMEKNASNLVKAEFRVFRLQNPKARVPEQRI
ELYQILKSKDLTSPTQRYIDSKVVKTRAEGEWLSFDVTDAVHEWLHHKD
RNLGFKISLHCPCCTFVPSNNYIIPNKSEELEARFAGIDGTSTYTSGDQ
KTIKSTRKKNSGKTPHLLLMLLPSYRLESQQTNRRKKRALDAAYCFRNV
QDNCCLRPLYIDFKRDLGWKWIHEPKGYNANFCAGACPYLWSSDTQHSR
VLSLYNTINPEASASPCCVSQDLEPLTILYYIGKTPKIEQLSNMIVKSC
KCS
TGFß3
(SEQ ID NO: 62)
SLSLSTCTTLDFGHIKKKRVEAIRGQILSKLRLTSPPEPTVMTHVPYQV
LALYNSTRELLEEMHGEREEGCTQENTESEYYAKEIHKFDMIQGLAEHN
ELAVCPKGITSKVFRFNVSSVEKNRTNLFRAEFRVLRVPNPSSKRNEQR
IELFQILRPDEHIAKQRYIGGKNLPTRGTAEWLSFDVTDTVREWLLRRE
SNLGLEISIHCPCHTFQPNGDILENIHEVMEIKFKGVDNEDDHGRGDLG
RLKKQKDHHNPHLILMMIPPHRLDNPGQGGQRKKRALDTNYCFRNLEEN
CCVRPLYIDFRQDLGWKWVHEPKGYYANFCSGPCPYLRSADTTHSTVLG
LYNTLNPEASASPCCVPQDLEPLTILYYVGRTPKVEQLSNMVVKSCKCS

TABLE 2
Exemplary TGFß1, TGFß2, and TGFß3 amino acid sequences

| | | SEQ |
|---|---|---|
| | | ID |
| Protein | Sequence | NO |
| proTGFß1 | LSTCKTIDMELVKRKRIEAIRGQILSKLRLASPPSQGEVPPGPLPE | 63 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHRRALDTNYCFSSTEKNC | |
| | CVRQLYIDFRKDLGWKWIHEPKGYHANFCLGPCPYIWSLDTQY | |
| | SKVLALYNQHNPGASAAPCCVPQALEPLPIVYYVGRKPKVEQLS | |
| | NMIVRSCKCS | |
| proTGFß1 C4S | LSTSKTIDMELVKRKRIEAIRGQILSKLRLASPPSQGEVPPGPLPE | 64 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHRRALDTNYCFSSTEKNC | |
| | CVRQLYIDFRKDLGWKWIHEPKGYHANFCLGPCPYIWSLDTQY | |
| | SKVLALYNQHNPGASAAPCCVPQALEPLPIVYYVGRKPKVEQLS | |
| | NMIVRSCKCS | |
| proTGFß1 D2G | LSTCKTIDMELVKRKRIEAIRGQILSKLRLASPPSQGEVPPGPLPE | 14 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHGALDTNYCFSSTEKNCC | |
| | VRQLYIDFRKDLGWKWIHEPKGYHANFCLGPCPYIWSLDTQYSK | |
| | VLALYNQHNPGASAAPCCVPQALEPLPIVYYVGRKPKVEQLSNM | |
| | IVRSCKCS | |
| proTGFß1 C4S D2G | LSTSKTIDMELVKRKRIEAIRGQILSKLRLASPPSQGEVPPGPLPE | 15 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHGALDTNYCFSSTEKNCC | |
| | VRQLYIDFRKDLGWKWIHEPKGYHANFCLGPCPYIWSLDTQYSK | |
| | VLALYNQHNPGASAAPCCVPQALEPLPIVYYVGRKPKVEQLSNM | |
| | IVRSCKCS | |
| proTGFß2 | SLSTCSTLDMDQFMRKRIEAIRGQILSKLKLTSPPEDYPEPEEVP | 16 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKKRALDAAYCFRNVQDNCCLRPLYIDFKRDLGW | |
| | KWIHEPKGYNANFCAGACPYLWSSDTQHSRVLSLYNTINPEASA | |
| | SPCCVSQDLEPLTILYYIGKTPKIEQLSNMIVKSCKCS | |
| proTGFß2 C5S | SLSTSSTLDMDQFMRKRIEAIRGQILSKLKLTSPPEDYPEPEEVP | 17 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKKRALDAAYCFRNVQDNCCLRPLYIDFKRDLGW | |
| | KWIHEPKGYNANFCAGACPYLWSSDTQHSRVLSLYNTINPEASA | |
| | SPCCVSQDLEPLTILYYIGKTPKIEQLSNMIVKSCKCS | |
| proTGFß2 C5S D2G | SLSTSSTLDMDQFMRKRIEAIRGQILSKLKLTSPPEDYPEPEEVP | 18 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKGALDAAYCFRNVQDNCCLRPLYIDFKRDLGWK | |
| | WIHEPKGYNANFCAGACPYLWSSDTQHSRVLSLYNTINPEASAS | |
| | PCCVSQDLEPLTILYYIGKTPKIEQLSNMIVKSCKCS | |
| proTGFß2 D2G | SLSTCSTLDMDQFMRKRIEAIRGQILSKLKLTSPPEDYPEPEEVP | 19 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKGALDAAYCFRNVQDNCCLRPLYIDFKRDLGWK | |
| | WIHEPKGYNANFCAGACPYLWSSDTQHSRVLSLYNTINPEASAS | |
| | PCCVSQDLEPLTILYYIGKTPKIEQLSNMIVKSCKCS | |
| proTGFß3 | SLSLSTCTTLDFGHIKKKRVEAIRGQILSKLRLTSPPEPTVMTHVP | 20 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKKRALDTNYCFRNLEENCCVRPLYIDFRQDLGWK | |
| | WVHEPKGYYANFCSGPCPYLRSADTTHSTVLGLYNTLNPEASA | |
| | SPCCVPQDLEPLTILYYVGRTPKVEQLSNMVVKSCKCS | |
| proTGFß3 C7S | SLSLSTSTTLDFGHIKKKRVEAIRGQILSKLRLTSPPEPTVMTHVP | 21 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKKRALDTNYCFRNLEENCCVRPLYIDFRQDLGWK | |
| | WVHEPKGYYANFCSGPCPYLRSADTTHSTVLGLYNTLNPEASA | |
| | SPCCVPQDLEPLTILYYVGRTPKVEQLSNMVVKSCKCS | |
| proTGFß3 C7S D2G | SLSLSTSTTLDFGHIKKKRVEAIRGQILSKLRLTSPPEPTVMTHVP | 22 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKGALDTNYCFRNLEENCCVRPLYIDFRQDLGWKW | |
| | VHEPKGYYANFCSGPCPYLRSADTTHSTVLGLYNTLNPEASASP | |
| | CCVPQDLEPLTILYYVGRTPKVEQLSNMVVKSCKCS | |
| proTGFß3 D2G | SLSLSTCTTLDFGHIKKKRVEAIRGQILSKLRLTSPPEPTVMTHVP | 23 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKGALDTNYCFRNLEENCCVRPLYIDFRQDLGWKW | |
| | VHEPKGYYANFCSGPCPYLRSADTTHSTVLGLYNTLNPEASASP | |
| | CCVPQDLEPLTILYYVGRTPKVEQLSNMVVKSCKCS | |

TABLE 3
Exemplary non-human TGFß1 amino acid sequences

| | | | SEQ |
|---|---|---|---|
| | | | ID |
| Protein | Species | Sequence | NO |
| proTGFß1 | Mouse | LSTCKTIDMELVKRKRIEAIRGQILSKLRLASPPSQGEVPPGPL | 24 |
| | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRRALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFß1 | Cyno | LSTCKTIDMELVKRKRIEAIRGQILSKLRLASPPSQGEVPPGPL | 25 |
| | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRRAL | |
| | | DTNYCFSSTEKNCCVRQLYIDFRKDLGWKWIHEPKGYHANF | |
| | | CLGPCPYIWSLDTQYSKVLALYNQHNPGASAAPCCVPQALE | |
| | | PLPIVYYVGRKPKVEQLSNMIVRSCKCS | |
| TGFß1 LAP | Mouse | LSTSKTIDMELVKRKRIEAIRGQILSKLRLASPPSQGEVPPGPL | 26 |
| C4S | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRR | |
| TGFß1 LAP | Cyno | LSTSKTIDMELVKRKRIEAIRGQILSKLRLASPPSQGEVPPGPL | 27 |
| C4S | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRR | |
| proTGFß1 | Mouse | LSTSKTIDMELVKRKRIEAIRGQILSKLRLASPPSQGEVPPGPL | 28 |
| C4S D2G | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHGALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFß1 | Mouse | LSTSKTIDMELVKRKRIEAIRGQILSKLRLASPPSQGEVPPGPL | 29 |
| C4S | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRRALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFß1 | Cyno | LSTSKTIDMELVKRKRIEAIRGQILSKLRLASPPSQGEVPPGPL | 30 |
| C4S | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRRAL | |
| | | DTNYCFSSTEKNCCVRQLYIDFRKDLGWKWIHEPKGYHANF | |
| | | CLGPCPYIWSLDTQYSKVLALYNQHNPGASAAPCCVPQALE | |
| | | PLPIVYYVGRKPKVEQLSNMIVRSCKCS | |
| proTGFß1 | Cyno | LSTSKTIDMELVKRKRIEAIRGQILSKLRLASPPSQGEVPPGPL | 31 |
| C4S D2G | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHGALDT | |
| | | NYCFSSTEKNCCVRQLYIDFRKDLGWKWIHEPKGYHANFCL | |
| | | GPCPYIWSLDTQYSKVLALYNQHNPGASAAPCCVPQALEPL | |
| | | PIVYYVGRKPKVEQLSNMIVRSCKCS | |

In some embodiments, an antibody, or antigen binding portion thereof, as described herein, is capable of selectively binding to a LTBP-TGFβ1 complex. In some embodiments, antigenic protein complexes (e.g., a LTBP-TGFβ1 complex) may comprise one or more LTBP proteins (e.g., LTBP1, LTBP2, LTBP3, and LTBP4).

In some embodiments, the antibody, or antigen binding portion thereof, selectively binds a LTBP1-TGFβ1 complex. In some embodiments, the LTBP1 protein is a naturally-occurring protein. In some embodiments, the LTBP1 protein is a non-naturally occurring protein. In some embodiments, the LTBP1 protein is a recombinant protein. Such recombinant LTBP1 protein may comprise LTBP1, alternatively spliced variants thereof, and/or fragments thereof. Recombinant LTBP1 proteins may also be modified to comprise one or more detectable labels. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). Such detectable labels may include, but are not limited to biotin labels, polyhistidine tags, myc tags, HA tags and/or fluorescent tags. In some embodiments, the LTBP1 protein is a mammalian LTBP1 protein. In some embodiments, the LTBP1 protein is a human, a monkey, a mouse, or a rat LTBP1 protein. In some embodiments, the LTBP1 protein comprises an amino acid sequence as set forth in SEQ ID NO: 32 in Table 4. In some embodiments, the LTBP1 protein comprises an amino acid sequence as set forth in SEQ ID NOs: 33 or SEQ ID NO: 34 in Table 3.

In some embodiments, an antibody, or antigen binding portion thereof, as described herein, is capable of binding to a LTBP3-TGFβ1 complex. In some embodiments, the LTBP3 protein is a naturally-occurring protein. In some embodiments, the LTBP3 protein is a non-naturally occurring protein. In some embodiments, the LTBP3 protein is a recombinant protein. Such recombinant LTBP3 protein may comprise LTBP3, alternatively spliced variants thereof and/or fragments thereof. In some embodiments, the LTBP3 protein comprises a leader sequence (e.g., a native or non-native leader sequence). In some embodiments, the LTBP3 protein does not comprise a leader sequence (i.e., the leader sequence has been processed or cleaved). Recombinant LTBP3 proteins may also be modified to comprise one or more detectable labels. Such detectable labels may include, but are not limited to biotin labels, polyhistidine tags, myc tags, HA tags and/or fluorescent tags. In some embodiments, the LTBP3 protein is a mammalian LTBP3 protein. In some embodiments, the LTBP3 protein is a human, a monkey, a mouse, or a rat LTBP3 protein. In some embodiments, the LTBP3 protein comprises an amino acid sequence as set forth in SEQ ID NO: 35. In some embodiments, the LTBP3 protein comprises an amino acid sequence as set forth in SEQ ID NOs: 36 or 37.

In some embodiments, antigenic protein complexes may be so-called a small latent complex (or SLC), comprised of a dimeric complex of the LAP (or prodomain) and a growth factor domain. In other embodiments, antigenic protein complexes may be so-called a large latent complex (or LLC) which further comprises a presenting molecule bound to a SLC. Presenting molecules include LTBP proteins (e.g., LTBP1, LTBP2, LTBP3, and LTBP4), CARP proteins, LRRC33 proteins, or fragment(s) thereof. When LLCs are used as antigenic protein complexes, typically, a minimum required fragment suitable for carrying out the embodiments disclosed herein includes at least 50 amino acids, preferably at least 100 amino acids, of a presenting molecule protein, comprising at least two cysteine residues capable of forming disulfide bonds with a proTGFβ1 complex. Specifically, these Cys residues form covalent bonds with Cysteine resides present near the N-terminus of each monomer of the proTGFβ1 complex.

TABLE 4A
Exemplary LTBP amino acid sequences

| | | | SEQ ID |
|---|---|---|---|
| Protein | Species | Sequence | NO |
| LTBP1S | Human | NHTGRIKVVFTPSICKVTCTKGSCQNSCEKGNTTTLISE | 32 |
| | | NGHAADTLTATNFRVVICHLPCMNGGQCSSRDKCQCP | |
| | | PNFTGKLCQIPVHGASVPKLYQHSQQPGKALGTHVIHS | |
| | | THTLPLTVTSQQGVKVKFPPNIVNIHVKHPPEASVQIHQ | |
| | | VSRIDGPTGQKTKEAQPGQSQVSYQGLPVQKTQTIHST | |
| | | YSHQQVIPHVYPVAAKTQLGRCFQETIGSQCGKALPGL | |
| | | SKQEDCCGTVGTSWGFNKCQKCPKKPSYHGYNQMM | |
| | | ECLPGYKRVNNTFCQDINECQLQGVCPNGECLNTMGS | |
| | | YRCTCKIGFGPDPTFSSCVPDPPVISEEKGPCYRLVSS | |
| | | GRQCMHPLSVHLTKQLCCCSVGKAWGPHCEKCPLPG | |
| | | TAAFKEICPGGMGYTVSGVHRRRPIHHHVGKGPVFVK | |
| | | PKNTQPVAKSTHPPPLPAKEEPVEALTFSREHGPGVAE | |
| | | PEVATAPPEKEIPSLDQEKTKLEPGQPQLSPGISTIHLH | |
| | | PQFPVVIEKTSPPVPVEVAPEASTSSASQVIAPTQVTEI | |
| | | NECTVNPDICGAGHCINLPVRYTCICYEGYRFSEQQRK | |
| | | CVDIDECTQVQHLCSQGRCENTEGSFLCICPAGFMASE | |
| | | EGTNCIDVDECLRPDVCGEGHCVNTVGAFRCEYCDSG | |
| | | YRMTQRGRCEDIDECLNPSTCPDEQCVNSPGSYQCVP | |
| | | CTEGFRGWNGQCLDVDECLEPNVCANGDCSNLEGSY | |
| | | MCSCHKGYTRTPDHKHCRDIDECQQGNLCVNGQCKN | |
| | | TEGSFRCTCGQGYQLSAAKDQCEDIDECQHRHLCAHG | |
| | | QCRNTEGSFQCVCDQGYRASGLGDHCEDINECLEDKS | |
| | | VCQRGDCINTAGSYDCTCPDGFQLDDNKTCQDINECE | |
| | | HPGLCGPQGECLNTEGSFHCVCQQGFSISADGRTCED | |
| | | IDECVNNTVCDSHGFCDNTAGSFRCLCYQGFQAPQDG | |
| | | QGCVDVNECELLSGVCGEAFCENVEGSFLCVCADENQ | |
| | | EYSPMTGQCRSRTSTDLDVDVDQPKEEKKECYYNLND | |
| | | ASLCDNVLAPNVTKQECCCTSGVGWGDNCEIFPCPVL | |
| | | GTAEFTEMCPKGKGFVPAGESSSEAGGENYKDADECL | |
| | | LFGQEICKNGFCLNTRPGYECYCKQGTYYDPVKLQCF | |
| | | DMDECQDPSSCIDGQCVNTEGSYNCFCTHPMVLDASE | |
| | | KRCIRPAESNEQIEETDVYQDLCWEHLSDEYVCSRPLV | |
| | | GKQTTYTECCCLYGEAWGMQCALCPLKDSDDYAQLC | |
| | | NIPVTGRRQPYGRDALVDFSEQYTPEADPYFIQDRFLN | |
| | | SFEELQAEECGILNGCENGRCVRVQEGYTCDCFDGYH | |
| | | LDTAKMTCVDVNECDELNNRMSLCKNAKCINTDGSYK | |
| | | CLCLPGYVPSDKPNYCTPLNTALNLEKDSDLE | |
| LTBP1S | Cyno | NHTGRIKVVFTPSICKVTCTKGSCQNSCEKGNTTTLISE | 33 |
| | | NGHAADTLTATNFRVVLCHLPCMNGGQCSSRDKCQC | |
| | | PPNFTGKLCQIPVHGASVPKLYQHSQQPGKALGTHVIH | |
| | | STHTLPLTVTSQQGVKVKFPPNIVNIHVKHPPEASVQIH | |
| | | QVSRIDGPTGQKTKEAQPGQSQVSYQGLPVQKTQTIH | |
| | | STYSHQQVIPHVYPVAAKTQLGRCFQETIGSQCGKALP | |
| | | GLSKQEDCCGTVGTSWGFNKCQKCPKKPSYHGYNQM | |
| | | MECLPGYKRVNNTFCQDINECQLQGVCPNGECLNTMG | |
| | | SYRCTCKIGFGPDPTFSSCVPDPPVISEEKGPCYRLVS | |
| | | SGRQCMHPLSVHLTKQLCCCSVGKAWGPHCEKCPLP | |
| | | GTAAFKEICPGGMGYTVSGVHRRRPIHHHVGKGPVFV | |
| | | KPKNTQPVAKSTHPPPLPAKEEPVEALTFSREHGPGVA | |
| | | EPEVATAPPEKEIPSLDQEKTKLEPGQPQLSPGISTIHL | |
| | | HPQFPVVIEKTSPPVPVEVAPEASTSSASQVIAPTQVTE | |
| | | INECTVNPDICGAGHCINLPVRYTCICYEGYKFSEQQRK | |
| | | CVDIDECTQVQHLCSQGRCENTEGSFLCICPAGFMASE | |
| | | EGTNCIDVDECLRPDVCGEGHCVNTVGAFRCEYCDSG | |
| | | YRMTQRGRCEDIDECLNPSTCPDEQCVNSPGSYQCVP | |
| | | CTEGFRGWNGQCLDVDECLEPNVCTNGDCSNLEGSY | |
| | | MCSCHKGYTRTPDHKHCKDIDECQQGNLCVNGQCKN | |
| | | TEGSFRCTCGQGYQLSAAKDQCEDIDECQHHHLCAHG | |
| | | QCRNTEGSFQCVCDQGYRASGLGDHCEDINECLEDKS | |
| | | VCQRGDCINTAGSYDCTCPDGFQLDDNKTCQDINECE | |
| | | HPGLCGPQGECLNTEGSFHCVCQQGFSISADGRTCED | |
| | | IDECVNNTVCDSHGFCDNTAGSFRCLCYQGFQAPQDG | |
| | | QGCVDVNECELLSGVCGEAFCENVEGSFLCVCADENQ | |
| | | EYSPMTGQCRSRTSTDLDVEQPKEEKKECYYNLNDAS | |
| | | LCDNVLAPNVTKQECCCTSGAGWGDNCEIFPCPVLGT | |
| | | AEFTEMCPKGKGFVPAGESSSEAGGENYKDADECLLF | |
| | | GQEICKNGFCLNTRPGYECYCKQGTYYDPVKLQCFDM | |
| | | DECQDPSSCIDGQCVNTEGSYNCFCTHPMVLDASEKR | |
| | | CIRPAESNEQIEETDVYQDLCWEHLSDEYVCSRPLVGK | |
| | | QTTYTECCCLYGEAWGMQCALCPMKDSDDYAQLCNIP | |
| | | VTGRRQPYGRDALVDFSEQYAPEADPYFIQDRFLNSFE | |
| | | ELQAEECGILNGCENGRCVRVQEGYTCDCFDGYHLDT | |
| | | AKMTCVDVNECDELNNRMSLCKNAKCINTEGSYKCLC | |
| | | LPGYVPSDKPNYCTPLNTALNLEKDSDLE | |
| LTBP1S | mouse | NHTGRIKVVFTPSICKVTCTKGNCQNSCQKGNTTTLISE | 34 |
| | | NGHAADTLTATNFRVVICHLPCMNGGQCSSRDKCQCP | |
| | | PNFTGKLCQIPVLGASMPKLYQHAQQQGKALGSHVIHS | |
| | | THTLPLTMTSQQGVKVKFPPNIVNIHVKHPPEASVQIHQ | |
| | | VSRIDSPGGQKVKEAQPGQSQVSYQGLPVQKTQTVHS | |
| | | TYSHQQLIPHVYPVAAKTQLGRCFQETIGSQCGKALPG | |
| | | LSKQEDCCGTVGTSWGFNKCQKCPKKQSYHGYTQMM | |
| | | ECLQGYKRVNNTFCQDINECQLQGVCPNGECLNTMGS | |
| | | YRCSCKMGFGPDPTFSSCVPDPPVISEEKGPCYRLVS | |
| | | PGRHCMHPLSVHLTKQICCCSVGKAWGPHCEKCPLPG | |
| | | TAAFKEICPGGMGYTVSGVHRRRPIHQHIGKEAVYVKP | |
| | | KNTQPVAKSTHPPPLPAKEEPVEALTSSWEHGPRGAE | |
| | | PEVVTAPPEKEIPSLDQEKTRLEPGQPQLSPGVSTIHLH | |
| | | PQFPVVVEKTSPPVPVEVAPEASTSSASQVIAPTQVTEI | |
| | | NECTVNPDICGAGHCINLPVRYTCICYEGYKFSEQLRK | |
| | | CVDIDECAQVRHLCSQGRCENTEGSFLCVCPAGFMAS | |
| | | EEGTNCIDVDECLRPDMCRDGRCINTAGAFRCEYCDS | |
| | | GYRMSRRGYCEDIDECLKPSTCPEEQCVNTPGSYQCV | |
| | | PCTEGFRGWNGQCLDVDECLQPKVCTNGSCTNLEGS | |
| | | YMCSCHRGYSPTPDHRHCQDIDECQQGNLCMNGQCR | |
| | | NTDGSFRCTCGQGYQLSAAKDQCEDIDECEHHHLCSH | |
| | | GQCRNTEGSFQCVCNQGYRASVLGDHCEDINECLEDS | |
| | | SVCQGGDCINTAGSYDCTCPDGFQLNDNKGCQDINEC | |
| | | AQPGLCGSHGECLNTQGSFHCVCEQGFSISADGRTCE | |
| | | DIDECVNNTVCDSHGFCDNTAGSFRCLCYQGFQAPQD | |
| | | GQGCVDVNECELLSGVCGEAFCENVEGSFLCVCADEN | |
| | | QEYSPMTGQCRSRVTEDSGVDRQPREEKKECYYNLN | |
| | | DASLCDNVLAPNVTKQECCCTSGAGWGDNCEIFPCPV | |
| | | QGTAEFTEMCPRGKGLVPAGESSYDTGGENYKDADE | |
| | | CLLFGEEICKNGYCLNTQPGYECYCKQGTYYDPVKLQ | |
| | | CFDMDECQDPNSCIDGQCVNTEGSYNCFCTHPMVLDA | |
| | | SEKRCVQPTESNEQIEETDVYQDLCWEHLSEEYVCSR | |
| | | PLVGKQTTYTECCCLYGEAWGMQCALCPMKDSDDYA | |
| | | QLCNIPVTGRRRPYGRDALVDFSEQYGPETDPYFIQDR | |
| | | FLNSFEELQAEECGILNGCENGRCVRVQEGYTCDCFD | |
| | | GYHLDMAKMTCVDVNECSELNNRMSLCKNAKCINTEG | |
| | | SYKCLCLPGYIPSDKPNYCTPLNSALNLDKESDLE | |
| LTBP3 | Human | GPAGERGAGGGGALARERFKVVFAPVICKRTCLKGQC | 35 |
| | | RDSCQQGSNMTLIGENGHSTDTLTGSGFRVVVCPLPC | |
| | | MNGGQCSSRNQCLCPPDFTGRFCQVPAGGAGGGTG | |
| | | GSGPGLSRTGALSTGALPPLAPEGDSVASKHAIYAVQV | |
| | | IADPPGPGEGPPAQHAAFLVPLGPGQISAEVQAPPPVV | |
| | | NVRVHHPPEASVQVHRIESSNAESAAPSQHLLPHPKPS | |
| | | HPRPPTQKPLGRCFQDTLPKQPCGSNPLPGLTKQEDC | |
| | | CGSIGTAWGQSKCHKCPQLQYTGVQKPGPVRGEVGA | |
| | | DCPQGYKRLNSTHCQDINECAMPGVCRHGDCLNNPG | |
| | | SYRCVCPPGHSLGPSRTQCIADKPEEKSLCFRLVSPEH | |
| | | QCQHPLTTRLTRQLCCCSVGKAWGARCQRCPTDGTA | |
| | | AFKEICPAGKGYHILTSHQTLTIQGESDFSLFLHPDGPP | |
| | | KPQQLPESPSQAPPPEDTEEERGVTTDSPVSEERSVQ | |
| | | QSHPTATTTPARPYPELISRPSPPTMRWFLPDLPPSRS | |
| | | AVEIAPTQVTETDECRLNQNICGHGECVPGPPDYSCHC | |
| | | NPGYRSHPQHRYCVDVNECEAEPCGPGRGICMNTGG | |
| | | SYNCHCNRGYRLHVGAGGRSCVDLNECAKPHLCGDG | |
| | | GFCINFPGHYKCNCYPGYRLKASRPPVCEDIDECRDPS | |
| | | SCPDGKCENKPGSFKCIACQPGYRSQGGGACRDVNE | |
| | | CAEGSPCSPGWCENLPGSFRCTCAQGYAPAPDGRSC | |
| | | LDVDECEAGDVCDNGICSNTPGSFQCQCLSGYHLSRD | |
| | | RSHCEDIDECDFPAACIGGDCINTNGSYRCLCPQGHRL | |
| | | VGGRKCQDIDECSQDPSLCLPHGACKNLQGSYVCVCD | |
| | | EGFTPTQDQHGCEEVEQPHHKKECYLNFDDTVFCDSV | |
| | | LATNVTQQECCCSLGAGWGDHCEIYPCPVYSSAEFHS | |
| | | LCPDGKGYTQDNNIVNYGIPAHRDIDECMLFGSEICKE | |
| | | GKCVNTQPGYECYCKQGFYYDGNLLECVDVDECLDES | |
| | | NCRNGVCENTRGGYRCACTPPAEYSPAQRQCLSPEE | |
| | | MDVDECQDPAACRPGRCVNLPGSYRCECRPPWVPGP | |
| | | SGRDCQLPESPAERAPERRDVCWSQRGEDGMCAGPL | |
| | | AGPALTFDDCCCRQGRGWGAQCRPCPPRGAGSHCP | |
| | | TSQSESNSFWDTSPLLLGKPPRDEDSSEEDSDECRCV | |
| | | SGRCVPRPGGAVCECPGGFQLDASRARCVDIDECREL | |
| | | NQRGLLCKSERCVNTSGSFRCVCKAGFARSRPHGACV | |
| | | PQRRR | |
| LTBP3 | CYNO | GPAGERGAGGGGALARERFKVVFAPVICKRTCLKGQC | 36 |
| | | RDSCQQGSNMTLIGENGHSTDTLTGSGFRVVVCPLPC | |
| | | MNGGQCSSRNQCLCPPDFTGRFCQVPAGGAGGGTG | |
| | | GSGPGLSRAGALSTGALPPLAPEGDSVASKHAIYAVQV | |
| | | IADPPGPGEGPPAQHAAFLVPLGPGQISAEVQAPPPVV | |
| | | NVRVHHPPEASVQVHRIESSNAEGAAPSQHLLPHPKPS | |
| | | HPRPPTQKPLGRCFQDTLPKQPCGSNPLPGLTKQEDC | |
| | | CGSIGTAWGQSKCHKCPQLQYTGVQKPGPVRGEVGA | |
| | | DCPQGYKRLNSTHCQDINECAMPGVCRHGDCLNNPG | |
| | | SYRCVCPPGHSLGPSRTQCIADKPEEKSLCFRLVSPEH | |
| | | QCQHPLTTRLTRQLCCCSVGKAWGARCQRCPADGTA | |
| | | AFKEICPAGKGYHILTSHQTLTIQGESDFSLFLHPDGPP | |
| | | KPQQLPESPSQAPPPEDTEEERGVTTDSPVSEERSVQ | |
| | | QSHPTATTSPARPYPELISRPSPPTMRWFLPDLPPSRS | |
| | | AVEIAPTQVTETDECRLNQNICGHGECVPGPPDYSCHC | |
| | | NPGYRSHPQHRYCVDVNECEAEPCGPGRGICMNTGG | |
| | | SYNCHCNRGYRLHVGAGGRSCVDLNECAKPHLCGDG | |
| | | GFCINFPGHYKCNCYPGYRLKASRPPVCEDIDECRDPS | |
| | | SCPDGKCENKPGSFKCIACQPGYRSQGGGACRDVNE | |
| | | CAEGSPCSPGWCENLPGSFRCTCAQGYAPAPDGRSC | |
| | | VDVDECEAGDVCDNGICTNTPGSFQCQCLSGYHLSRD | |
| | | RSHCEDIDECDFPAACIGGDCINTNGSYRCLCPQGHRL | |
| | | VGGRKCQDIDECTQDPGLCLPHGACKNLQGSYVCVCD | |
| | | EGFTPTQDQHGCEEVEQPHHKKECYLNFDDTVFCDSV | |
| | | LATNVTQQECCCSLGAGWGDHCEIYPCPVYSSAEFHS | |
| | | LCPDGKGYTQDNNIVNYGIPAHRDIDECMLFGAEICKE | |
| | | GKCVNTQPGYECYCKQGFYYDGNLLECVDVDECLDES | |
| | | NCRNGVCENTRGGYRCACTPPAEYSPAQRQCLSPEE | |
| | | MDVDECQDPAACRPGRCVNLPGSYRCECRPPWVPGP | |
| | | SGRDCQLPESPAERAPERRDVCWSQRGEDGMCAGP | |
| | | QAGPALTFDDCCCRQGRGWGAQCRPCPPRGAGSQC | |
| | | PTSQSESNSFWDTSPLLLGKPRRDEDSSEEDSDECRC | |
| | | VSGRCVPRPGGAVCECPGGFQLDASRARCVDIDECRE | |
| | | LNQRGLLCKSERCVNTSGSFRCVCKAGFARSRPHGAC | |
| | | VPQRRR | |
| LTBP3 | Mouse | GPAGERGTGGGGALARERFKVVFAPVICKRTCLKGQC | 37 |
| | | RDSCQQGSNMTLIGENGHSTDTLTGSAFRVVVCPLPC | |
| | | MNGGQCSSRNQCLCPPDFTGRFCQVPAAGTGAGTGS | |
| | | SGPGLARTGAMSTGPLPPLAPEGESVASKHAIYAVQVI | |
| | | ADPPGPGEGPPAQHAAFLVPLGPGQISAEVQAPPPVV | |
| | | NVRVHHPPEASVQVHRIEGPNAEGPASSQHLLPHPKP | |
| | | PHPRPPTQKPLGRCFQDTLPKQPCGSNPLPGLTKQED | |
| | | CCGSIGTAWGQSKCHKCPQLQYTGVQKPVPVRGEVG | |
| | | ADCPQGYKRLNSTHCQDINECAMPGNVCHGDCLNNP | |
| | | GSYRCVCPPGHSLGPLAAQCIADKPEEKSLCFRLVSTE | |
| | | HQCQHPLTTRLTRQLCCCSVGKAWGARCQRCPADGT | |
| | | AAFKEICPGKGYHILTSHQTLTIQGESDFSLFLHPDGPP | |
| | | KPQQLPESPSRAPPLEDTEEERGVTMDPPVSEERSVQ | |
| | | QSHPTTTTSPPRPYPELISRPSPPTFHRFLPDLPPSRSA | |
| | | VEIAPTQVTETDECRLNQNICGHGQCVPGPSDYSCHC | |
| | | NAGYRSHPQHRYCVDVNECEAEPCGPGKGICMNTGG | |
| | | SYNCHCNRGYRLHVGAGGRSCVDLNECAKPHLCGDG | |
| | | GFCINFPGHYKCNCYPGYRLKASRPPICEDIDECRDPS | |
| | | TCPDGKCENKPGSFKCIACQPGYRSQGGGACRDVNE | |
| | | CSEGTPCSPGWCENLPGSYRCTCAQYEPAQDGLSCID | |
| | | VDECEAGKVCQDGICTNTPGSFQCQCLSGYHLSRDRS | |
| | | RCEDIDECDFPAACIGGDCINTNGSYRCLCPLGHRLVG | |
| | | GRKCKKDIDECSQDPGLCLPHACENLQGSYVCVCDEG | |
| | | FTLTQDQHGCEEVEQPHHKKECYLNFDDTVFCDSVLA | |
| | | TNVTQQECCCSLGAGWGDHCEIYPCPVYSSAEFHSLV | |
| | | PDGKRLHSGQQHCELCIPAHRDIDECILFGAEICKEGKC | |
| | | VNTQPGYECYCKQGFYYDGNLLECVDVDECLDESNCR | |
| | | NGVCENTRGGYRCACTPPAEYSPAQAQCLIPERWSTP | |
| | | QRDVKCAGASEERTACVWGPWAGPALTFDDCCCRQP | |
| | | RLGTQCRPCPPRGTGSQCPTSQSESNSFWDTSPLLLG | |
| | | KSPRDEDSSEEDSDECRCVSGRCVPRPGGAVCECPG | |
| | | GFQLDASRARCVDIDECRELNQRGLLCKSERCVNTSG | |
| | | SFRCVCKAGFTRSRPHGPACLSAAADDAAIAHTSVIDH | |
| | | RGYFH | |

In an exemplary embodiment, inhibitors, e.g., antibodies, and antigen-binding fragments thereof, that selectively bind LTBP1-TGFβ11 and/or LTBP3-TGFβ11 with at least 50-fold, preferably 100-fold affinities, as compared to a complex containing TGFβ1 and CARP or LRRC33.

TABLE 4B
Exemplary GARP and LRRC33 amino acid sequences

| | | SEQ |
|---|---|---|
| | | ID |
| Protein | Sequence | NO |
| GARP | AQHQDKVPCKMVDKKVSCQVLGLLQVPSVLPPDTETLDLSGNQ | 38 |
| | LRSILASPLGFYTALRHLDLSTNEISFLQPGAFQALTHLEHLSLAH | |
| | NRLAMATALSAGGLGPLPRVTSLDLSGNSLYSGLLERLLGEAPS | |
| | LHTLSLAENSLTRLTRHTFRDMPALEQLDLHSNVLMDIEDGAFE | |
| | GLPRLTHLNLSRNSLTCISDFSLQQLRVLDLSCNSIEAFQTASQP | |
| | QAEFQLTWLDLRENKLLHFPDLAALPRLIYLNLSNNLIRLPTGPP | |
| | QDSKGIHAPSEGWSALPLSAPSGNASGRPLSQLLNLDLSYNEIE | |
| | LIPDSFLEHLTSLCFLNLSRNCLRTFEARRLGSLPCLMLLDLSHN | |
| | ALETLELGARALGSLRTLLLQGNALRDLPPYTFANLASLQRLNLQ | |
| | GNRVSPCGGPDEPGPSGCVAFSGITSLRSLSLVDNEIELLRAGA | |
| | FLHTPLTELDLSSNPGLEVATGALGGLEASLEVLALQGNGLMVL | |
| | QVDLPCFICLKRLNLAENRLSHLPAWTQAVSLEVLDLRNNSFSLL | |
| | PGSAMGGLETSLRRLYLQGNPLSCCGNGWLAAQLHQGRVDVD | |
| | ATQDLICRFSSQEEVSLSHVRPEDCEKGGLKNINLIIILTFILVSAIL | |
| | LTTLAACCCVRRQKFNQQYKA | |
| SGARP | AQHQDKVPCKMVDKKVSCQVLGLLQVPSVLPPDTETLDLSGNQ | 39 |
| | LRSILASPLGFYTALRHLDLSTNEISFLQPGAFQALTHLEHLSLAH | |
| | NRLAMATALSAGGLGPLPRVTSLDLSGNSLYSGLLERLLGEAPS | |
| | LHTLSLAENSLTRLTRHTFRDMPALEQLDLHSNVLMDIEDGAFE | |
| | GLPRLTHLNLSRNSLTCISDFSLQQLRVLDLSCNSIEAFQTASQP | |
| | QAEFQLTWLDLRENKLLHFPDLAALPRLIYLNLSNNLIRLPTGPP | |
| | QDSKGIHAPSEGWSALPLSAPSGNASGRPLSQLLNLDLSYNEIE | |
| | LIPDSFLEHLTSLCFLNLSRNCLRTFEARRLGSLPCLMLLDLSHN | |
| | ALETLELGARALGSLRTLLLQGNALRDLPPYTFANLASLQRLNLQ | |
| | GNRVSPCGGPDEPGPSGCVAFSGITSLRSLSLVDNEIELLRAGA | |
| | FLHTPLTELDLSSNPGLEVATGALGGLEASLEVLALQGNGLMVL | |
| | QVDLPCFICLKRLNLAENRLSHLPAWTQAVSLEVLDLRNNSFSLL | |
| | PGSAMGGLETSLRRLYLQGNPLSCCGNGWLAAQLHQGRVDVD | |
| | ATQDLICRFSSQEEVSLSHVRPEDCEKGGLKNIN | |
| GARP | ISQRREQVPCRTVNKEALCHGLGLLQVPSVLSLDIQALYLSGNQ | 40 |
| mouse | LQSILVSPLGFYTALRHLDLSDNQISFLQAGVFQALPYLEHLNLA | |
| | HNRLATGMALNSGGLGRLPLLVSLDLSGNSLHGNLVERLLGETP | |
| | RLRTLSLAENSLTRLARHTFWGMPAVEQLDLHSNVLMDIEDGAF | |
| | EALPHLTHLNLSRNSLTCISDFSLQQLQVLDLSCNSIEAFQTAPE | |
| | PQAQFQLAWLDLRENKLLHFPDLAVFPRLIYLNVSNNLIQLPAGL | |
| | PRGSEDLHAPSEGWSASPLSNPSRNASTHPLSQLLNLDLSYNEI | |
| | ELVPASFLEHLTSLRFLNLSRNCLRSFEARQVDSLPCLVLLDLSH | |
| | NVLEALELGTKVLGSLQTLLLQDNALQELPPYTFASLASLQRLNL | |
| | QGNQVSPCGGPAEPGPPGCVDFSGIPTLHVLNMAGNSMGMLR | |
| | AGSFLHTPLTELDLSTNPGLDVATGALVGLEASLEVLELQGNGLT | |
| | VLRVDLPCFLRLKRLNLAENQLSHLPAWTRAVSLEVLDLRNNSF | |
| | SLLPGNAMGGLETSLRRLYLQGNPLSCCGNGWLAAQLHQGRV | |
| | DVDATQDLICRFGSQEELSLSLVRPEDCEKGGLKNVNLILLLSFT | |
| | LVSAIVLTTLATICFLRRQKLSQQYKA | |
| SGARP | ISQRREQVPCRTVNKEALCHGLGLLQVPSVLSLDIQALYLSGNQ | 41 |
| mouse | LQSILVSPLGFYTALRHLDLSDNQISFLQAGVFQALPYLEHLNLA | |
| | HNRLATGMALNSGGLGRLPLLVSLDLSGNSLHGNLVERLLGETP | |
| | RLRTLSLAENSLTRLARHTFWGMPAVEQLDLHSNVLMDIEDGAF | |
| | EALPHLTHLNLSRNSLTCISDFSLQQLQVLDLSCNSIEAFQTAPE | |
| | PQAQFQLAWLDLRENKLLHFPDLAVFPRLIYLNVSNNLIQLPAGL | |
| | PRGSEDLHAPSEGWSASPLSNPSRNASTHPLSQLLNLDLSYNEI | |
| | ELVPASFLEHLTSLRFLNLSRNCLRSFEARQVDSLPCLVLLDLSH | |
| | NVLEALELGTKVLGSLQTLLLQDNALQELPPYTFASLASLQRLNL | |
| | QGNQVSPCGGPAEPGPPGCVDFSGIPTLHVLNMAGNSMGMLR | |
| | AGSFLHTPLTELDLSTNPGLDVATGALVGLEASLEVLELQGNGLT | |
| | VLRVDLPCFLRLKRLNLAENQLSHLPAWTRAVSLEVLDLRNNSF | |
| | SLLPGNAMGGLETSLRRLYLQGNPLSCCGNGWLAAQLHQGRV | |
| | DVDATQDLICRFGSQEELSLSLVRPEDCEKGGLKNVN | |
| LRRC33 | MELLPLWLCLGFHFLTVGWRNRSGTATAASQGVCKLVGGAAD | 42 |
| (also known | CRGQSLASVPSSLPPHARMLTLDANPLKTLWNHSLQPYPLLESL | |
| as NRROS; | SLHSCHLERISRGAFQEQGHLRSLVLGDNCLSENYEETAAALHA | |
| Uniprot | LPGLRRLDLSGNALTEDMAALMLQNLSSLRSVSLAGNTIMRLDD | |
| Accession | SVFEGLERLRELDLQRNYIFEIEGGAFDGLAELRHLNLAFNNLPCI | |
| No. Q86YC3) | VDFGLTRLRVLNVSYNVLEWFLATGGEAAFELETLDLSHNQLLF | |
| | FPLLPQYSKLRTLLLRDNNMGFYRDLYNTSSPREMVAQFLLVDG | |
| | NVTNITTVSLWEEFSSSDLADLRFLDMSQNQFQYLPDGFLRKMP | |
| | SLSHLNLHQNCLMTLHIREHEPPGALTELDLSHNQLSELHLAPGL | |
| | ASCLGSLRLFNLSSNQLLGVPPGLFANARNITTLDMSHNQISLCP | |
| | LPAASDRVGPPSCVDFRNMASLRSLSLEGCGLGALPDCPFQGT | |
| | SLTYLDLSSNWGVLNGSLAPLQDVAPMLQVLSLRNMGLHSSFM | |
| | ALDFSGFGNLRDLDLSGNCLTTFPRFGGSLALETLDLRRNSLTA | |
| | LPQKAVSEQLSRGLRTIYLSQNPYDCCGVDGWGALQHGQTVAD | |
| | WAMVTCNLSSKIIRVTELPGGVPRDCKWERLDLGLLYLVLILPSC | |
| | LTLLVACTVIVLTFKKPLLQVIKSRCHWSSVY | |
| | *Native signal peptide is depicted in bold font. | |
| soluble | MDMRVPAQLLGLLLLWFSGVLGWRNRSGTATAASQGVCKLVG | 43 |
| LRRC33 | GAADCRGQSLASVPSSLPPHARMLTLDANPLKTLWNHSLQPYP | |
| (sLRRC33) | LLESLSLHSCHLERISRGAFQEQGHLRSLVLGDNCLSENYEETA | |
| | AALHALPGLRRLDLSGNALTEDMAALMLQNLSSLRSVSLAGNTI | |
| | MRLDDSVFEGLERLRELDLQRNYIFEIEGGAFDGLAELRHLNLAF | |
| | NNLPCIVDFGLTRLRVLNVSYNVLEWFLATGGEAAFELETLDLSH | |
| | NQLLFFPLLPQYSKLRTLLLRDNNMGFYRDLYNTSSPREMVAQF | |
| | LLVDGNVTNITTVSLWEEFSSSDLADLRFLDMSQNQFQYLPDGF | |
| | LRKMPSLSHLNLHQNCLMTLHIREHEPPGALTELDLSHNQLSEL | |
| | HLAPGLASCLGSLRLFNLSSNQLLGVPPGLFANARNITTLDMSH | |
| | NQISLCPLPAASDRVGPPSCVDFRNMASLRSLSLEGCGLGALPD | |
| | CPFQGTSLTYLDLSSNWGVLNGSLAPLQDVAPMLQVLSLRNMG | |
| | LHSSFMALDFSGFGNLRDLDLSGNCLTTFPRFGGSLALETLDLR | |
| | RNSLTALPQKAVSEQLSRGLRTIYLSQNPYDCCGVDGWGALQH | |
| | GQTVADWAMVTCNLSSKIIRVTELPGGVPRDCKWERLDLGLHH | |
| | HHHH | |
| | *Modified human kappa light chain signal peptide | |
| | is depicted in bold font. | |
| | **Histidine tag is underlined. | |
| Human LRRC33- | MDMRVPAQLLGLLLLWFSGVLGWRNRSGTATAASQGVCKLVG | 44 |
| GARP chimera | GAADCRGQSLASVPSSLPPHARMLTLDANPLKTLWNHSLQPYP | |
| | LLESLSLHSCHLERISRGAFQEQGHLRSLVLGDNCLSENYEETA | |
| | AALHALPGLRRLDLSGNALTEDMAALMLQNLSSLRSVSLAGNTI | |
| | MRLDDSVFEGLERLRELDLQRNYIFEIEGGAFDGLAELRHLNLAF | |
| | NNLPCIVDFGLTRLRVLNVSYNVLEWFLATGGEAAFELETLDLSH | |
| | NQLLFFPLLPQYSKLRTLLLRDNNMGFYRDLYNTSSPREMVAQF | |
| | LLVDGNVTNITTVSLWEEFSSSDLADLRFLDMSQNQFQYLPDGF | |
| | LRKMPSLSHLNLHQNCLMTLHIREHEPPGALTELDLSHNQLSEL | |
| | HLAPGLASCLGSLRLFNLSSNQLLGVPPGLFANARNITTLDMSH | |
| | NQISLCPLPAASDRVGPPSCVDFRNMASLRSLSLEGCGLGALPD | |
| | CPFQGTSLTYLDLSSNWGVLNGSLAPLQDVAPMLQVLSLRNMG | |
| | LHSSFMALDFSGFGNLRDLDLSGNCLTTFPRFGGSLALETLDLR | |
| | RNSLTALPQKAVSEQLSRGLRTIYLSQNPYDCCGVDGWGALQH | |
| | GQTVADWAMVTCNLSSKIIRVTELPGGVPRDCKWERLDLGLLIII | |
| | LTFILVSAILLTTLAACCCVRRQKFNQQYKA | |
| | *Modified human kappa light chain signal peptide | |
| | is depicted in bold font. | |
| | **LRRC33 ectodomain is underlined. | |
| | #GARP transmembrane domain is italicized. | |
| | ##GARP intracellular tail is double underlined. | |

In another aspect, the disclosure provides methods of inhibiting TGFβ1 activation in the context of LTBP1 and/or LTBP3. In one embodiment, the method comprises exposing a LTBP1-proTGFβ1 complex or a LTBP3-proTGFβ1 complex an inhibitor, an antibody or antigen-binding fragment thereof, and/or a pharmaceutical composition described herein. For example, in one embodiment, the inhibitor is an inhibitor of extracellular matrix-associated TGFβ1 activation, which selectively binds a LTBP1/3-presented proTGFβ1 latent complex. In one embodiment, the inhibitor does not significantly inhibit immune cell-associated TGFβ1 activation, for example, immune cell-associated TGFβ1 activation that results from activation of a GARP-presented proTGFβ1 latent complex. In another embodiment, the antibody, or antigen-binding portion thereof, selectively binds an LTBP1-proTGFβ1 latent complex and/or an LTBP3-proTGFβ1 latent complex, thereby modulating release of mature TGFβ1 growth factor from the latent complex, wherein the antibody, or antigen-binding portion thereof, does not bind mature TGFβ1 alone or a GARP-proTGFβ1 latent complex. In one embodiment, the antibody, or antigen-binding portion thereof, inhibits the release of mature TGFβ1 from the LTBP1-proTGFβ1 complex and/or the LTBP3-proTGFβ1 complex. In one embodiment, the antibody, or antigen-binding portion thereof, does not inhibit the release of mature TGFβ1 from a GARP-proTGFβ1 complex or a LRRC33-proTGFβ1 complex.

In one embodiment, the method is performed in vitro. In another embodiment, the method is performed in vivo. In one embodiment, the LTBP1-proTGFβ1 complex or the LTBP3-proTGFβ1 complex is in an extracellular matrix. The extracellular matrix can comprise, for example, fibrillin and/or fibronectin. In some embodiments, the extracellular matrix comprises a protein comprising an RGD motif.

In some embodiments of the foregoing aspects, the antibody, or antigen-binding fragment thereof, does not stimulate immune effector cells. In one embodiment, the antibody, or antigen-binding fragment thereof, inhibits the release of mature TGFβ1 from a LTBP1-proTGFβ1 complex and/or a LTBP3-proTGFβ1 complex, and does not inhibit the release of mature TGFβ1 from a GARP-proTGFβ1 complex and/or an LRRC33-proTGFβ1 complex.

In some embodiments, inhibitors, e.g., antibodies (and fragment thereof) of the present disclosure that selectively bind to a LTBP1-TGFβ1 complex and/or a LTBP3-TGFβ1 complex can bind the complex with relatively high affinity, e.g., with a dissociation constant (K_{D}) less than 10₋₈ M, 10₋₉ M, 10₋₁₀ M, 10₋₁₁ M or lower. In one embodiment, an antibody, or an antigen binding fragment thereof, binds a LTBP1-TGFβ1 complex and/or a LTBP3-TGFβ1 complex with a dissociation constant (K_{D}) of about 10-8 M, about 10-9 M, about 10-10 M, about 10-11 M, about 10-12 M, or about 10-13 M. For example, antibodies that selectively bind to a LTBP1-TGFβ1 complex and/or a LTBP3-TGFβ1 complex can bind the complex with an affinity between 5 μM and 10 nM, e.g., between 50 μM and 20 nM, e.g., between 100 μM and 10 nM. In one embodiment, the antibody, or antigen-binding fragment thereof, can bind a LTBP1-TGFβ1 complex and/or a LTBP3-TGFβ1 complex with an affinity of about 20 nm to about 500 nm. For example, the antibody, or antigen-binding fragment thereof, can bind a LTBP1-TGFβ1 complex and/or a LTBP3-TGFβ1 complex with an affinity of about 0.1 nM to about 10 nM.

The disclosure also includes antibodies or antigen binding fragments that compete with any of the antibodies described herein for binding to a LTBP1-TGFβ1 complex and/or a LTBP3-TGFβ1 complex. In some embodiments, such antibodies have an affinity for the complex of 50 nM or lower (e.g., 20 nM or lower, 10 nM or lower, 500 μM or lower, 50 μM or lower, or 5 μM or lower). The affinity and binding kinetics of antibodies (or antigen binding fragments thereof) that selectively bind to a LTBP1-TGFβ1 complex and/or a LTBP3-TGFβ1 complex can be tested using any suitable method, including but not limited to biosensor technology (e.g., OCTET™ or BIACORE™)

In one embodiment, the antibodies, or antigen-binding fragments thereof, of the present disclosure do not compete with antibody SR-Ab42 for binding to a human LTBP1-proTGFβ1 complex.

Aspects of the disclosure relate to antibodies that compete or cross-compete with any of the antibodies provided herein. The term “compete”, as used herein with regard to an antibody, means that a first antibody binds to an epitope (e.g., an epitope of a LTBP1-TGFβ1 complex and/or an epitope of a LTBP3-TGFβ1 complex) in a manner sufficiently similar to the binding of a second antibody, such that the result of binding of the first antibody with its epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its epitope or ligand, whether to the same, greater, or lesser extent, the antibodies are said to “cross-compete” with each other for binding of their respective epitope(s). Both competing and cross-competing antibodies are within the scope of this disclosure. Regardless of the mechanism by which such competition or cross-competition occurs (e.g., steric hindrance, conformational change, or binding to a common epitope, or portion thereof), the skilled artisan would appreciate that such competing and/or cross-competing antibodies are encompassed and can be useful for the methods and/or compositions provided herein.

Aspects of the disclosure relate to antibodies that compete or cross-compete with any of the specific antibodies, or antigen binding fragment thereof, as provided herein, e.g., an antibody having one or more CDR sequences (1, 2, 3, 4, 5, or 6 CDR sequences) set forth in Table 5. In one embodiment, the disclosure provides antibodies, and antigen-binding fragments thereof, that compete or cross-compete with an antibody having heavy chain CDR sequences comprising SEQ ID NO:1, SEQ ID NO:2, and SEQ ID NO:3 as set forth in Table 5, and/or light chain CDR sequences comprising SEQ ID NO:4, SEQ ID NO:5, and SEQ ID NO:6 as set forth in Table 5. In one embodiment, the disclosure provides antibodies that compete or cross-compete with an antibody, or antigen binding portion thereof, having a heavy chain variable region sequence comprising SEQ ID NO:7, and/or a light chain variable region sequence comprising SEQ ID NO:8. In some embodiments, an antibody, or antigen binding portion thereof, binds at or near the same epitope as any of the antibodies provided herein. In some embodiments, an antibody, or antigen binding portion thereof, binds near an epitope if it binds within 15 or fewer amino acid residues of the epitope. In some embodiments, any of the antibody, or antigen binding portion thereof, as provided herein, binds within 1, 2, 3, 4, 5, 6, 7, 8, 9, 10, 11, 12, 13, 14 or 15 amino acid residues of an epitope that is bound by any of the antibodies provided herein.

In another embodiment, provided herein is an antibody, or antigen binding fragment thereof, competes or cross-competes for binding to any of the antigens provided herein (e.g., a LTBP1-TGFβ1 complex and/or a LTBP3-TGFβ1 complex) with an equilibrium dissociation constant, K_{D}, between the antibody and the protein of less than 10-8 M. In other embodiments, an antibody, or an antigen binding fragment thereof, competes or cross-competes for binding to any of the antigens provided herein with a K_{D} in a range from 10⁻¹¹ M to 10⁻⁸ M. In some embodiments, provided herein is an anti-TGFβ1 antibody, or an antigen binding fragment thereof, that competes for binding with an antibody, or antigen binding fragment thereof, described herein. In some embodiments, provided herein is an anti-TGFβ1 antibody, or antigen binding fragment thereof, that binds to the same epitope as an antibody, or antigen binding fragment thereof, described herein.

The antibodies, and antigen binding fragments thereof, provided herein can be characterized using any suitable methods. For example, one method is to identify the epitope to which the antigen binds, or “epitope mapping.” There are many suitable methods for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). In some embodiments, the epitope is a TGFβ1 epitope that is only available for binding by the antibody, or the antigen binding fragment thereof, described herein, when the TGFβ1 is in a LTBP1-TGFβ1 complex and/or a LTBP3-TGFβ1 complex.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In some embodiments, the antibodies, or antigen binding fragment thereof, of the present disclosure that selectively bind to a LTBP1-TGFβ1 complex and/or a LTBP3-TGFβ1 complex include one or more of complementary determining regions (CDRs) shown in Table 5. In some embodiments, the disclosure provides a nucleic acid molecule that encodes an antibody, or antigen binding portion thereof, that selectively binds to a LTBP1-TGFβ1 complex and/or a LTBP3-TGFβ1 complex, as described herein. In one embodiment, the nucleic acid molecules encode one or more of the CDR sequences shown in Table 5.

TABLE 5
Complementary determining regions of the heavy chain (CDRHs) and the light chain
(CDRLs) of Ab42

| H-CDR1 | H-CDR2 | H-CDR3 | L-CDR1 | L-CDR2 | L-CDR3 |
|---|---|---|---|---|---|
| FTFRSYVMH | VISHEGSLK | ARPRIAA | TRSSGNIDNNYVQ | EDNQRPS | QSYDYDTQGVV |
| (SEQ ID | YYADSVKG | RRGGFGY | (SEQ ID NO: 4) | (SEQ ID | (SEQ ID NO: |
| NO: 1) | (SEQ ID | (SEQ ID | | NO: 5) | 6) |
| | NO: 2) | NO: 3) | | | |

In some embodiments, antibodies of the present disclosure that selectively bind to a LTBP1-TGFβ1 complex and/or a LTBP3-TGFβ1 complex include any antibody, or antigen binding portion thereof, comprising a CDRH1, CDRH2, CDRH3, CDRL1, CDRL2, or CDRL3, or combinations thereof, as provided in Table 5. In some embodiments, antibodies that selectively bind to a LTBP1-TGFβ1 complex and/or a LTBP3-TGFβ1 complex include CDRH1, CDRH2, CDRH3, CDRL1, CDRL2, and CDRL3 as provided in Table 5.

The present disclosure also provides a nucleic acid sequence that encodes a molecule comprising CDRH1, CDRH2, CDRH3, CDRL1, CDRL2, or CDRL3, or combinations thereof, as provided in Table 5.

Antibody heavy and light chain CDR3 domains may play a particularly important role in the binding specificity/affinity of an antibody for an antigen. Accordingly, in some embodiments, the antibodies, or antigen binding fragment thereof, that selectively bind to a LTBP1-TGFβ1 complex and/or a LTBP3-TGFβ1 complex, or the nucleic acid molecules that encode these antibodies, or antigen binding fragment thereof, can include at least the heavy and/or light chain CDR3 of the antibody shown in Table 5.

Aspects of the disclosure relate to a monoclonal antibody, or antigen binding portion thereof, that binds selectively to a LTBP1-TGFβ1 complex and/or a LTBP3-TGFβ1 complex, and that comprises six complementarity determining regions (CDRs): CDRH1, CDRH2, CDRH3, CDRL1, CDRL2, and CDRL3.

In some embodiments (e.g., as for Ab42, shown in Table 5), the antibody, or antigen binding portion thereof, that selectively binds to a LTBP1-TGFβ1 complex and/or a LTBP3-TGFβ1 complex comprises: a CDRH1 comprising an amino acid sequence as set forth in SEQ ID NO: 1, a CDRH2 comprising an amino acid sequence as set forth in SEQ ID NO: 2, a CDRH3 comprising an amino acid sequence as set forth in SEQ ID NO: 3, a CDRL1 comprising an amino acid sequence as set forth in SEQ ID NO: 4, a CDRL2 comprising an amino acid sequence as set forth in SEQ ID NO: 5, and a CDRL3 comprising an amino acid sequence as set forth in SEQ ID NO: 6.

According to some embodiments, the antibody, or the antigen binding fragment thereof, comprises a CDRH1 sequence comprising FTFRSYVMH (SEQ ID NO: 1); a CDRH2 sequence comprising VISHEGSLKYYADSVKG (SEQ ID NO: 2); a CDRH3 sequence comprising ARPRIAARRGGFGY (SEQ ID NO:3); a CDRL1 sequence comprising TRSSGNIDNNYVQ (SEQ ID NO: 4); a CDRL2 sequence comprising EDNQRPS (SEQ ID NO: 5) and a CDRL3 sequence comprising QSYDYDTQGVV (SEQ ID NO: 6). According to some embodiments, the antibody, or the antigen-binding fragment thereof comprises a CDRH1 sequence comprising an amino acid sequence at least 85% identical to FTFRSYVMH (SEQ ID NO: 1); a CDRH2 sequence comprising an amino acid sequence at least 85% identical to VISHEGSLKYYADSVKG (SEQ ID NO: 2); a CDRH3 sequence comprising an amino acid sequence at least 85% identical to ARPRIAARRGGFGY (SEQ ID NO: 3); a CDRL1 sequence comprising an amino acid sequence at least 85% identical to TRSSGNIDNNYVQ (SEQ ID NO: 4); a CDRL2 sequence comprising an amino acid sequence at least 85% identical to EDNQRPS (SEQ ID NO: 5) and a CDRL3 sequence comprising an amino acid sequence at least 85% identical to QSYDYDTQGVV (SEQ ID NO: 6). According to some embodiments, the antibody, or the antigen-binding fragment thereof comprises a CDRH1 sequence comprising an amino acid sequence at least 90% identical to FTFRSYVMH (SEQ ID NO: 1); a CDRH2 sequence comprising an amino acid sequence at least 90% identical to VISHEGSLKYYADSVKG (SEQ ID NO: 2); a CDRH3 sequence comprising an amino acid sequence at least 90% identical to ARPRIAARRGGFGY (SEQ ID NO: 3); a CDRL1 sequence comprising an amino acid sequence at least 90% identical to TRSSGNIDNNYVQ (SEQ ID NO: 4); a CDRL2 sequence comprising an amino acid sequence at least 90% identical to EDNQRPS (SEQ ID NO: 5) and a CDRL3 sequence comprising an amino acid sequence at least 90% identical to QSYDYDTQGVV (SEQ ID NO: 6).

According to some embodiments, the antibody, or the antigen-binding fragment thereof comprises a CDRH1 sequence comprising an amino acid sequence at least 95% identical to FTFRSYVMH (SEQ ID NO: 1); a CDRH2 sequence comprising an amino acid sequence at least 95% identical to VISHEGSLKYYADSVKG (SEQ ID NO: 2); a CDRH3 sequence comprising an amino acid sequence at least 95% identical to ARPRIAARRGGFGY (SEQ ID NO: 3); a CDRL1 sequence comprising an amino acid sequence at least 95% identical to TRSSGNIDNNYVQ (SEQ ID NO: 4); a CDRL2 sequence comprising an amino acid sequence at least 95% identical to EDNQRPS (SEQ ID NO: 5) and a CDRL3 sequence comprising an amino acid sequence at least 95% identical to QSYDYDTQGVV (SEQ ID NO: 6). According to some embodiments, the antibody, or the antigen-binding fragment thereof comprises a CDRH1 sequence comprising an amino acid sequence at least 97% identical to FTFRSYVMH (SEQ ID NO: 1); a CDRH2 sequence comprising an amino acid sequence at least 97% identical to VISHEGSLKYYADSVKG (SEQ ID NO: 2); a CDRH3 sequence comprising an amino acid sequence at least 97% identical to ARPRIAARRGGFGY (SEQ ID NO: 3); a CDRL1 sequence comprising an amino acid sequence at least 97% identical to TRSSGNIDNNYVQ (SEQ ID NO: 4); a CDRL2 sequence comprising an amino acid sequence at least 97% identical to EDNQRPS (SEQ ID NO: 5) and a CDRL3 sequence comprising an amino acid sequence at least 97% identical to QSYDYDTQGVV (SEQ ID NO: 6).

According to some embodiments, the antibody, or the antigen-binding fragment thereof comprises a CDRH1 sequence comprising an amino acid sequence at least 98% identical to FTFRSYVMH (SEQ ID NO: 1); a CDRH2 sequence comprising an amino acid sequence at least 98% identical to VISHEGSLKYYADSVKG (SEQ ID NO: 2); a CDRH3 sequence comprising an amino acid sequence at least 98% identical to ARPRIAARRGGFGY (SEQ ID NO: 3); a CDRL1 sequence comprising an amino acid sequence at least 98% identical to TRSSGNIDNNYVQ (SEQ ID NO: 4); a CDRL2 sequence comprising an amino acid sequence at least 98% identical to EDNQRPS (SEQ ID NO: 5) and a CDRL3 sequence comprising an amino acid sequence at least 98% identical to QSYDYDTQGVV (SEQ ID NO: 6). According to some embodiments, the antibody, or the antigen-binding fragment thereof comprises a CDRH1 sequence comprising an amino acid sequence at least 99% identical to FTFRSYVMH (SEQ ID NO: 1); a CDRH2 sequence comprising an amino acid sequence at least 99% identical to VISHEGSLKYYADSVKG (SEQ ID NO: 2); a CDRH3 sequence comprising an amino acid sequence at least 99% identical to ARPRIAARRGGFGY (SEQ ID NO: 3); a CDRL1 sequence comprising an amino acid sequence at least 99% identical to TRSSGNIDNNYVQ (SEQ ID NO: 4); a CDRL2 sequence comprising an amino acid sequence at least 99% identical to EDNQRPS (SEQ ID NO: 5) and a CDRL3 sequence comprising an amino acid sequence at least 99% identical to QSYDYDTQGVV (SEQ ID NO: 6). According to some embodiments, the antibody, or the antigen binding fragment thereof, comprises a CDRH1 sequence consisting of FTFRSYVMH (SEQ ID NO: 1); a CDRH2 sequence consisting of VISHEGSLKYYADSVKG (SEQ ID NO: 2); a CDRH3 sequence consisting of ARPRIAARRGGFGY (SEQ ID NO:3); a CDRL1 sequence consisting of TRSSGNIDNNYVQ (SEQ ID NO: 4); a CDRL2 sequence consisting of EDNQRPS (SEQ ID NO: 5) and a CDRL3 sequence consisting of QSYDYDTQGVV (SEQ ID NO: 6).

Optionally, one or more of the six CDRs may include one or more (e.g., 1 or 2) amino acid change(s).

The amino acid sequences of the heavy chain variable region (HCVR) and the light chain variable region (LCVR) of the antibody AB42 set forth in Table 5 are provided below as SEQ ID NOs 7 and 8, respectively. In some embodiments, the antibody or an antigen-binding fragment thereof comprises a heavy chain variable domain (VH) and a light chain variable domain (VL), wherein, the VH comprises an amino acid sequence having at least 90% (e.g., 90%, 91%, 92%, 93%, 94%, 95%, 96%, 97%, 98%, 99% and 100%) sequence identity to:

(SEQ ID NO: 7)
QVQLVESGGGVVQPGRSLRLSCAASGFTFRSYVMHWVRQAPGKGLEWVA
VISHEGSLKYYADSVKGRFTISRDNSKNTLYLQMNSLRAEDTAVYYCAR
PRIAARRGGFGYWGQGTLVTVSS

and wherein the VL comprises an amino acid sequence having at least 90% (e.g., 90%, 91%, 92%, 93%, 94%, 95%, 96%, 97%, 98%, 99% and 100%) sequence identity to:

(SEQ ID NO: 8)
NFMLTQPHSVSESPGKTVTISCTRSSGNIDNNYVQWYQQRPGSSPTTVI
YEDNQRPSGVPDRFSGSIDSSSNSASLTISGLKTEDEADYYCQSYDYDT
QGVVFGGGTKLTVL

In one embodiment, the antibody, or the antigen-binding fragment thereof, comprises a heavy chain variable region comprising CDR-H1: SEQ ID NO: 1; CDR-H2: SEQ ID NO: 2; and CDR-H3: SEQ ID NO: 3; and a light chain variable region comprising CDR-L1: SEQ ID NO: 4; CDR-L2: SEQ ID NO: 5; and CDR-L3: SEQ ID NO: 6, optionally comprising up to 3 amino acid changes, for example 1, 2 or 3 amino acid changes, for each of the CDRs.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a LTBP1-TGFβ complex and/or a LTBP3-TGFβ complex, as determined based on the crystal structure. In some embodiments, the likely interface (e.g., residues involved in an antigen-antibody interaction) may be deduced from known structural information on another antigens sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgGI-like (CPPCP (SEQ ID NO: 45)) hinge sequence. Accordingly, any of the antibodies may include a stabilizing ‘Adair’ mutation or the amino acid sequence CPPCP (SEQ ID NO: 45). In one embodiment, an antibody described herein comprises a heavy chain immunoglobulin constant domain of a human IgG4 having a backbone substitution of Ser to Pro, that produces an IgG1-like hinge and permits formation of inter-chain disulfide bonds.

According to some embodiments, the antibodies provided herein comprise a M252Y/S254T/T256E (YTE) mutation. Shown below is the sequence of Ab42-YTE-hulgG4, where the CDRs are underlined using IMGT numbering (SEQ ID NO: 10) or using Kabat numbering (SEQ ID NO: 12).

Sequence of Ab42-YTE huG4: IMGT CDRs underlined. M252Y, S254T, T256E.
SEQ ID NO: 10
QVQLVESGGGVVQPGRSLRLSCAASGFTFRSYVMHWVRQAPGKGLEWVAVISHEGSLKYYADSVKGRFTISRDNS
KNTLYLQMNSLRAEDTAVYYCARPRIAARRGGFGYWGQGTLVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGP
PCPPCPAPEFLGGPSVFLFPPKPKDTLYITREPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNVFSCSVMHEALHNHYTQKSLSLSLG
Sequence of Ab42-YTE huG4: Kabat CDRs underlined-M252Y, S254T, T256E
SEQ ID NO: 12
QVQLVESGGGVVQPGRSLRLSCAASGFTFRSYVMHWVRQAPGKGLEWVAVISHEGSLKYYADSVKGRFTISRDNS
KNTLYLQMNSLRAEDTAVYYCARPRIAARRGGFGYWGQGTLVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGP
PCPPCPAPEFLGGPSVFLFPPKPKDTLYITREPEVTCVVVDVSQEDPEVQFNWYVDGVEVHNAKTKPREEQFNST
YRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLG

Shown below is the sequence of Ab42-YTE-huLambda, where the CDRs are underlined using IMGT numbering (SEQ ID NO: 11) or using Kabat numbering (SEQ ID NO: 13).

Sequence of Ab42-YTE huLambda. IMGT CDRs underlined.
SEQ ID NO: 11
NEMLTQPHSVSESPGKTVTISCTRSSGNIDNNYVQWYQQRPGSSPTTVIYEDNQRPSGVPDRFSGSIDSSSNSAS
LTISGLKTEDEADYYCQSYDYDTQGVVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQVTHEGSTVEKTVAPTECS
Sequence of Ab42-YTE huLambda
SEQ ID NO: 13
NEMLTQPHSVSESPGKTVTISCTRSSGNIDNNYVQWYQQRPGSSPTTVIYEDNQRPSGVPDRFSGSIDSSSNSAS
LTISGLKTEDEADYYCQSYDYDTQGVVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQVTHEGSTVEKTVAPTECS

Antibodies (and antigen binding fragments thereof) of this disclosure that selectively bind to a LTBP1-TGFβ1 complex and/or a LTBP3-TGFβ1 complex may optionally comprise antibody constant regions or parts thereof. For example, a VL domain may be attached at its C-terminal end to a light chain constant domain such as CK or C.

Similarly, a VH domain or portion thereof may be attached to all or part of a heavy chain such as IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or antigen binding fragment thereof, combined with any suitable constant region. In some embodiments, the antibodies, or antigen binding fragment thereof, comprise a heavy chain immunoglobulin constant domain containing all or a portion of a human IgGi or a human IgG4 constant domain. In some embodiments, the antibody, or antigen binding portion thereof, comprises a light chain immunoglobulin constant domain containing all or a portion of a human Ig lambda constant domain or a human Ig kappa constant domain.

In some embodiments, antibodies that selectively bind to a LTBP1-TGFβ1 complex and/or a LTBP3-TGFβ1 complex may or may not include the framework region of the antibodies of SEQ ID NOs: 7 and 8. In some embodiments, antibodies that selectively bind to a LTBP1-TGFβ1 complex and/or a LTBP3-TGFβ1 complex are murine antibodies and include murine framework region sequences. In other embodiments, the antibodies are chimeric antibodies, or antigen binding fragments thereof. In another embodiment, the antibodies are humanized antibodies, or antigen binding fragments thereof. In another embodiment, the antibodies are fully human antibodies, or antigen binding fragments thereof. In one embodiment, the antibody comprises a framework region comprising a human germline amino acid sequence.

The antibodies, and antigen-binding fragments thereof, described herein can have any configuration suitable for binding antigen. For example, in one embodiment, the antibody, or antigen binding portion thereof, comprises four polypeptide chains, including two heavy chain variable regions and two light chain variable regions. In another embodiment, the antibody, or antigen binding portion thereof, comprises one heavy chain variable region and one light chain variable region. In exemplary embodiments, the antibody, or antigen binding portion thereof, is a Fab fragment, a F(ab′)2 fragment, a scFab fragment, an scFv, or a diabody.

In one embodiment, the antibody, or antigen-binding fragment thereof, comprises a heavy chain immunoglobulin constant domain of a human Igd constant domain or a human IgG4 constant domain.

In an exemplary embodiment, the heavy chain immunoglobulin constant domain is a human IgG4 constant domain. In one embodiment, the antibody, or antigen-binding portion thereof, binds a conformational epitope. In one embodiment, the antibody, or antigen-binding portion thereof, binds a combinatorial epitope.

In one embodiment, the antibody, or antigen-binding fragment thereof, comprises a heavy chain immunoglobulin constant domain of a human IgG4 constant domain having a backbone substitution of Ser to Pro that produces an Igd-like hinge and permits formation of inter-chain disulfide bonds. In one embodiment, the antibody, or antigen-binding portion thereof, further comprises a light chain immunoglobulin constant domain comprising a human Ig lambda constant domain, or a human Ig kappa constant domain.

In one embodiment, the antibody is an IgG having four polypeptide chains which are two heavy chains and two light chains. In exemplary embodiments, the antibody can be a humanized antibody, a human antibody, or a chimeric antibody. In one embodiment, the antibody comprises a framework having a human germline amino acid sequence.

In one embodiment, the disclosure provides an antibody, or antigen-binding fragment thereof, that competes for binding with an antibody, or antigen-binding portion thereof, described herein. In one embodiment, the disclosure provides an antibody, or antigen-binding portion thereof, that binds to the same epitope as an antibody, or antigen-binding portion thereof, described herein. In one embodiment, the antibody, or antigen-binding fragment thereof, does not compete with Ab42 for binding to a human LTBP1-proTGFβ1 complex.

### Nucleic Acids

The disclosure includes nucleic acid sequences that encode any one of the amino acid sequences provided above. In some embodiments, antibodies, antigen binding fragment thereof, and/or compositions of the present disclosure may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present disclosure may comprise cells programmed or generated to express nucleic acid molecules encoding compounds and/or compositions of the present disclosure.

Encompassed herein are vectors (e.g., DNA plasmids, such as mammalian expression vectors, and related nucleic acid preparations) comprising the nucleic acid sequence; cells transfected with the vector(s); a cell line with stable expression of the nucleic acids; a cell culture comprising the cell, wherein optionally the cell culture comprises mammalian cells capable of large-scale production of the antibody or a protein construct comprising an antigen-binding fragment of the antibody.

In some cases, nucleic acids of the disclosure include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

### Modifications

Non-limiting variations, modifications, and features of any of the antibodies or antigen-binding fragments thereof encompassed by the present disclosure are briefly discussed below. Embodiments of related analytical methods are also provided.

Naturally-occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human antibody light chains are typically classified as kappa and lambda light chains. Heavy chains are typically classified as mu, delta, gamma, alpha, or epsilon, and define the isotype of the antibody. An antibody can be of any type (e.g., IgM, IgD, IgG, IgA, IgY, and IgE) and class (e.g., IgG1, IgG2, IgG3, IgG4, IgM1, IgM2, IgA1, and IgA2). Within full-length light and heavy chains, typically, the variable and constant regions are joined by a “J” region of about 12 or more amino acids, with the heavy chain also including a “D” region of about 10 more amino acids (see, e.g., Fundamental Immunology, Ch. 7 (Paul, W., ed., 2^{nd} ed. Raven Press, N.Y. (1989)) (incorporated by reference in its entirety)). The variable regions of each light/heavy chain pair typically form the antigen-binding site.

In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a LTBP1-TGFβ1 complex, a LTBP3-TGFβ1 complex, as determined based on the crystal structure. In some embodiments, likely interface (e.g., residues involved in an antigen-antibody interaction) may be deduced from known structural information on another antigen sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk (1987) J. Mol. Biol. 196: 901-917; Chothia et al., (1989) Nature 342: 878-883. The CDRs of a light chain can also be referred to as L-CDR1, L-CDR2, and L-CDR3, and the CDRs of a heavy chain can also be referred to as H-CDR1, H-CDR2, and H-CDR3. In some embodiments, an antibody can comprise a small number of amino acid deletions from the carboxy end of the heavy chain(s). In some embodiments, an antibody comprises a heavy chain having 1-5 amino acid deletions in the carboxy end of the heavy chain. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition, and the contact definition.

An “affinity matured” antibody is an antibody with one or more alterations in one or more CDRs thereof, which result an improvement in the affinity of the antibody for antigen compared to a parent antibody, which does not possess those alteration(s). Exemplary affinity matured antibodies will have nanomolar or even picomolar affinities for the target antigen. Affinity matured antibodies are produced by procedures known in the art. Marks et al., (1992) Bio/Technology 10: 779-783 describes affinity maturation by VH and VL domain shuffling. Random mutagenesis of CDR and/or framework residues is described by Barbas, et al., (1994) Proc Nat. Acad. Sci. USA 91: 3809-3813; Schier et al., (1995) Gene 169: 147-155; Yelton et al., (1995) J. Immunol. 155: 1994-2004; Jackson et al., (1995) J. Immunol. 154(7): 3310-9; and Hawkins et al., (1992) J. Mol. Biol. 226: 889-896; and selective mutation at selective mutagenesis positions, contact or hypermutation positions with an activity enhancing amino acid residue is described in U.S. Pat. No. 6,914,128.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “chimeric antibody” refers to antibodies, which comprise heavy and light chain variable region sequences from one species and constant region sequences from another species, such as antibodies having murine heavy and light chain variable regions linked to human constant regions.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (L-CDR1, L-CDR2, and L-CDR3 of light chain and H-CDR1, H-CDR2, and H-CDR3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

In some embodiments, the antibody, or antigen-binding portion thereof, comprises a heavy chain immunoglobulin constant domain of a human IgM constant domain, a human IgG constant domain, a human IgG1 constant domain, a human IgG2 constant domain, a human IgG2A constant domain, a human IgG2B constant domain, a human IgG2 constant domain, a human IgG3 constant domain, a human IgG3 constant domain, a human IgG4 constant domain, a human IgA constant domain, a human IgA1 constant domain, a human IgA2 constant domain, a human IgD constant domain, or a human IgE constant domain. In some embodiments, the antibody, or antigen-binding portion thereof, comprises a heavy chain immunoglobulin constant domain of a human IgG1 constant domain or a human IgG4 constant domain. In some embodiments, the antibody, or antigen-binding portion thereof, comprises a heavy chain immunoglobulin constant domain of a human IgG4 constant domain. In some embodiments, the antibody, or antigen-binding portion thereof, comprises a heavy chain immunoglobulin constant domain of a human IgG4 constant domain having a backbone substitution of Ser to Pro that produces an IgG1-like hinge and permits formation of inter-chain disulfide bonds.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like (CPPCP (SEQ ID NO: 45)) hinge sequence. Accordingly, any of the antibodies may include a stabilizing ‘Adair’ mutation or the amino acid sequence CPPCP (SEQ ID NO: 45).

In some embodiments, the antibody or antigen-binding portion thereof, further comprises a light chain immunoglobulin constant domain comprising a human Ig lambda constant domain or a human Ig kappa constant domain.

In some embodiments, the antibody is an IgG having four polypeptide chains which are two heavy chains and two light chains.

In some embodiments, wherein the antibody is a humanized antibody, a diabody, or a chimeric antibody. In some embodiments, the antibody is a humanized antibody. In some embodiments, the antibody is a human antibody. In some embodiments, the antibody comprises a framework having a human germline amino acid sequence.

In some embodiments, the antigen-binding portion is a Fab fragment, a F(ab′)2 fragment, a scFab fragment, or an scFv fragment.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al., (2002) Crit. Rev. Immunol. 22(3): 183-200; Marchalonis et al., (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologically activity by at least about 20%, 40%, 60%, 80%, 85%, 90%, 95%. 96%, 97%, 98%, 99% or more.

The term “binding protein” as used herein includes any polypeptide that specifically binds to an antigen (e.g., TGFβ1), including, but not limited to, an antibody, or antigen-binding fragment thereof, a DVD-Ig™, a TVD-Ig, a RAb-Ig, a bispecific antibody and a dual specific antibody.

The term “monoclonal antibody” or “mAb” when used in a context of a composition comprising the same may refer to an antibody preparation obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21: 371-378, incorporated herein by reference), antibodies isolated from an animal (e.g., a mouse) that is transgenic for human immunoglobulin genes (see, Taylor, L. D. et al., (1992) Nucl. Acids Res. 20: 6287-6295; Kellermann, S-A. and Green, L. L. (2002) Cur. Opin. In Biotechnol. 13: 593-597; Little, M. et al., (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some embodiments, the antibody or antigen-binding portion, is an antibody fragment, e.g., (i) Fab fragments, a monovalent fragment consisting of the VL, VH, CL and CH1 domains; (ii) F(ab′)2 fragment, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules; (vi) dAb fragments; or (vii) minimal recognition units consisting of the amino acid residues that mimic the hypervariable region of an antibody (e.g., an isolated complementarity determining region (CDR)). In some embodiments, the antibody or antigen-binding portion, is (i) a “Dual Variable Domain Immunoglobulin” or “DVD-Ig™,” (ii) a “Triple Variable Domain Immunoglobulin” or “TVD-Ig”, (iii) a “Receptor-Antibody Immunoglobulin” or “RAb-Ig,” (iv) a “bispecific antibody,” or (v) a “dual-specific antibody,”

As used herein, “Dual Variable Domain Immunoglobulin” or “DVD-Ig™” and the like include binding proteins comprising a paired heavy chain DVD polypeptide and a light chain DVD polypeptide with each paired heavy and light chain providing two antigen-binding sites. Each binding site includes a total of 6 CDRs involved in antigen-binding per antigen-binding site. A DVD-Ig™ is typically has two arms bound to each other at least in part by dimerization of the CH3 domains, with each arm of the DVD being bispecific, providing an immunoglobulin with four binding sites. DVD-Ig™ are provided in US Patent Publication No. 2010/0260668 and U.S. Pat. No. 9,109,026, each of which is incorporated herein by reference including sequence listings.

As used herein, “Triple Variable Domain Immunoglobulin” or “TVD-Ig” and the like are binding proteins comprising a paired heavy chain TVD binding protein polypeptide and a light chain TVD binding protein polypeptide with each paired heavy and light chain providing three antigen-binding sites. Each binding site includes a total of 6 CDRs involved in antigen-binding per antigen-binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are binding proteins comprising a heavy chain RAb polypeptide, and a light chain RAb polypeptide, which together form three antigen-binding sites in total. One antigen-binding site is formed by the pairing of the heavy and light antibody variable domains present in each of the heavy chain RAb polypeptide and the light chain RAb polypeptide to form a single binding site with a total of 6 CDRs providing a first antigen-binding site. Each the heavy chain RAb polypeptide and the light chain RAb polypeptide include a receptor sequence that independently binds a ligand providing the second and third “antigen” binding sites. A RAb-Ig is typically has two arms bound to each other at least in part by dimerization of the CH3 domains, with each arm of the RAb-Ig being trispecific, providing an immunoglobulin with six binding sites. RAb-Igs are described in US Patent Application Publication No. 2002/0127231, the entire contents of which including sequence listings are incorporated herein by reference).

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al., (1985) Nature 314(6012): 628-631), or by knob-into-hole or similar approaches, which introduce mutations in the Fc region that do not inhibit CH3-CH3 dimerization (see Holliger, P. et al., (1993) Proc. Nat. Acad. Sci USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen it binds to.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly, a dual-specific binding protein has two identical antigen-binding arms, with identical specificity and identical CDR sequences, and is bivalent for each antigen to which it binds.

The term “Kon,” as used herein, is intended to refer to the on rate constant for association of a binding protein (e.g., an antibody) to the antigen to form the, e.g., antibody/antigen complex as is known in the art. The “Kon” also is known by the terms “association rate constant,” or “ka,” as used interchangeably herein. This value indicating the binding rate of an antibody to its target antigen or the rate of complex formation between an antibody and antigen also is shown by the equation: Antibody (“Ab”)+Antigen (“Ag”)→Ab−Ag.

The term “Koff” as used herein, is intended to refer to the off rate constant for dissociation of a binding protein (e.g., an antibody) from the, e.g., antibody/antigen complex as is known in the art. The “Koff” also is known by the terms “dissociation rate constant” or “kdis” as used interchangeably herein. This value indicates the dissociation rate of an antibody from its target antigen or separation of Ab−Ag complex over time into free antibody and antigen as shown by the equation: Ab+Ag←Ab−Ag.

The terms “equilibrium dissociation constant” or “K_{D},” as used interchangeably herein, refer to the value obtained in a titration measurement at equilibrium, or by dividing the dissociation rate constant (koff) by the association rate constant (kon). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a BIACORE® (biomolecular interaction analysis) assay, can be used (e.g., instrument available from BIAcore International AB, a GE Healthcare company, Uppsala, Sweden). Additionally, a KINEXA® (Kinetic Exclusion Assay) assay, available from Sapidyne Instruments (Boise, Idaho), can also be used.

The terms “crystal” and “crystallized” as used herein, refer to a binding protein (e.g., an antibody), or antigen-binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 201-16, Oxford University Press, New York, New York, (1999).

The term “linker” is used to denote polypeptides comprising two or more amino acid residues joined by peptide bonds and are used to link one or more antigen-binding fragment. Such linker polypeptides are well known in the art (see, e.g., Holliger, P. et al., (1993) Proc. Natl. Acad. Sci. USA 90: 6444-6448; Poljak, R. J. et al., (1994) Structure 2:1121-1123). Exemplary linkers include, but are not limited to, ASTKGPSVFPLAP (SEQ ID NO: 65), ASTKGP (SEQ ID NO: 66); TVAAPSVFIFPP (SEQ ID NO: 67); TVAAP (SEQ ID NO: 68); AKTTPKLEEGEFSEAR (SEQ ID NO: 69); AKTTPKLEEGEFSEARV (SEQ ID NO: 70); AKTTPKLGG (SEQ ID NO: 71); SAKTTPKLGG (SEQ ID NO: 72); SAKTTP (SEQ ID NO: 73); RADAAP (SEQ ID NO: 74); RADAAPTVS (SEQ ID NO: 75); RADAAAAGGPGS (SEQ ID NO: 76); RADAAAA(G4S)4 (SEQ ID NO: 77); SAKTTPKLEEGEFSEARV (SEQ ID NO: 78); ADAAP (SEQ ID NO: 79); ADAAPTVSIFPP (SEQ ID NO: 80); QPKAAP (SEQ ID NO: 81); QPKAAPSVTLFPP (SEQ ID NO: 82); AKTTPP (SEQ ID NO: 83); AKTTPPSVTPLAP (SEQ ID NO: 84); AKTTAP (SEQ ID NO: 85); AKTTAPSVYPLAP (SEQ ID NO: 86); GGGGSGGGGSGGGGS (SEQ ID NO: 87); GENKVEYAPALMALS (SEQ ID NO: 88); GPAKELTPLKEAKVS (SEQ ID NO: 89); GHEAAAVMQVQYPAS (SEQ ID NO: 90); TVAAPSVFIFPPTVAAPSVFIFPP (SEQ ID NO: 91); and ASTKGPSVFPLAPASTKGPSVFPLAP (SEQ ID NO: 92).

“Label” and “detectable label” or “detectable moiety” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Antibodies, or antigen binding fragment thereof, of the disclosure may be modified with a detectable label or detectable moiety, including, but not limited to, an enzyme, prosthetic group, fluorescent material, luminescent material, bioluminescent material, radioactive material, positron emitting metal, nonradioactive paramagnetic metal ion, and affinity label for detection and/or isolation of a LTBP1-TGFβ1 complex or a LTBP3-TGFβ1 complex. The detectable substance or moiety may be coupled or conjugated either directly to the polypeptides of the disclosure or indirectly, through an intermediate (such as, for example, a linker (e.g., a cleavable linker)) using suitable techniques. Non-limiting examples of suitable enzymes include horseradish peroxidase, alkaline phosphatase, R-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I), carbon (¹⁴C), sulfur (³⁵S), tritium (³H), indium (¹¹⁵mIn, ¹¹³mIn, ¹¹²In, ¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, Lu, ¹⁵⁹Gd, ¹⁴⁹Pm ¹⁴⁰La, ¹⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, and tin (¹¹³Sn, ¹¹⁷Sn). The detectable substance may be coupled or conjugated either directly to the antibodies of the disclosure that bind selectively to a LTBP1-TGFβ1 complex and/or a LTBP3-TGFβ1 complex, or indirectly, through an intermediate (such as, for example, a linker) using suitable techniques. Any of the antibodies provided herein that are conjugated to a detectable substance may be used for any suitable diagnostic assays, such as those described herein.

In addition, antibodies, or antigen binding fragment thereof, of the disclosure may also be modified with a drug to form, e.g., an antibody-drug conjugate. The drug may be coupled or conjugated either directly to the polypeptides of the disclosure, or indirectly, through an intermediate (such as, for example, a linker (e.g., a cleavable linker)) using suitable techniques. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

In some embodiments, the binding affinity of an antibody, or antigen-binding portion thereof, to an antigen (e.g., protein complex), such as presenting molecule-proTGFβ1 complexes, is determined using an Octet assay. In some embodiments, an Octet assay is an assay that determines one or more a kinetic parameters indicative of binding between an antibody and antigen. In some embodiments, an OCTET® system (FORTEBIO®, Menlo Park, CA) is used to determine the binding affinity of an antibody, or antigen-binding portion thereof, to presenting molecule-proTGFβ1 complexes. For example, binding affinities of antibodies may be determined using the FORTEBIO® Octet QKe dip and read label free assay system utilizing bio-layer interferometry. In some embodiments, antigens are immobilized to biosensors (e.g., streptavidin-coated biosensors) and the antibodies and complexes (e.g., biotinylated presenting molecule-proTGFβ1 complexes) are presented in solution at high concentration (50 μg/mL) to measure binding interactions.

The antibodies according to the present disclosure include pH-sensitive antibodies. In some embodiments, such antibodies or fragments thereof bind the target complex in a pH-dependent manner such that relatively high affinity binding occurs at a neutral or physiological pH, but the antibody dissociates from its antigen more rapidly at an acidic pH; or, dissociation rates are higher at acidic pH than at neutral pH. Such antibodies or fragments thereof may function as recycling antibodies. Such antibodies may also be referred to as “pH-sensitive” antibodies.

Thus, the disclosure encompasses pH-sensitive antibodies that selectively bind a proTGFβ1 complex characterized in that the antibodies have lower dissociation rates at a neutral pH (e.g., around pH 7) as compared to at an acidic pH (e.g., around pH 5).

In some embodiments, such “pH sensitive” antibodies have a K_{dis} (a.k.a. K_{off}) of ≥5×10⁻³ s⁻¹ (e.g., ≥5.1×10⁻³, ≥5.2×10⁻³, ≥5.3×10⁻³, ≥5.4×10⁻³, ≥5.5×10⁻³, ≥5.6×10⁻³, ≥5.7×10⁻³, ≥5.8× 10⁻³, ≥5.9×10⁻³, or ≥6.0×10⁻³) at pH 5, as measured by a suitable affinity assay (e.g., biolayer interferometry, surface plasmon resonance, and/or solution equilibrium titration). In a particular embodiment, such “pH-sensitive” antibodies have a K_{dis}≥5.6×10⁻³ at pH 5.

In some embodiments, such “pH-sensitive” antibodies have a pH 5 K_{dis} to pH 7 K_{dis} ratio (i.e., K_{dis} at pH 5: K_{dis} at pH7) of ≥1.5 (e.g., ≥1.6, ≥1.7, ≥1.8, ≥1.9, or ≥2.0), as measured by a suitable affinity assay (e.g., biolayer interferometry, surface plasmon resonance, and/or solution equilibrium titration). In a particular embodiment, such “pH-sensitive” antibodies have a K_{dis} ratio of ≥2.0, as measured by biolayer interferometry.

### Targeting Agents

In some embodiments methods of the present disclosure comprise the use of one or more targeting agents to target an antibody, or antigen binding portion thereof, as disclosed herein, to a particular site in a subject for purposes of modulating mature TGFβ release from a LTBP1-TGFβ1 complex and/or a LTBP3-TGFβ1 complex. For example, LTBP1-TGFβ1 and LTBP3-TGFβ1 complexes are typically localized to extracellular matrix. Thus, in some embodiments, antibodies disclosed herein can be conjugated to extracellular matrix targeting agents for purposes of localizing the antibodies to sites where LTBP1-TGFβ1 and LTBP3-TGFβ1 complexes reside. In such embodiments, selective targeting of antibodies leads to selective modulation of LTBP1-TGFβ1 and/or LTBP3-TGFβ1 complexes. In some embodiments, selective targeting of antibodies leads to selective inhibition of LTBP1-TGFβ1 and/or LTBP3-TGFβ1 complexes (e.g., for purposes of treating fibrosis). In some embodiments, extracellular matrix targeting agents include heparin binding agents, matrix metalloproteinase binding agents, lysyl oxidase binding domains, fibrillin-binding agents, hyaluronic acid binding agents, and others.

In some embodiments, bispecific antibodies may be used having a first portion that selectively binds a LTBP1/3-TGFβ1 complex and a second portion that selectively binds a component of a target site, e.g., a component of the ECM (e.g., fibrillin).

### Binding Kinetics of Antibodies

The antibodies and antigen-binding fragments thereof (e.g., Fabs) disclosed herein are characterized by enhanced binding properties. The antibodies and the antigen-binding fragments are capable of specifically binding to each of the presenting molecule-proTGFβ1 complexes (sometimes referred to as “Large Latency Complex” or LLC, which is a ternary complex comprised of a proTGFβ1 dimer coupled to a single presenting molecule), namely, LTBP1-proTGFβ1, LTBP3-proTGFβ1, GARP-proTGFβ1 and LRRC33-proTGFβ1. Recombinantly produced, purified protein complexes may be used as antigens (e.g., antigen complexes) to screen, evaluate or confirm the ability of an antibody to bind the antigen complexes in suitable in vitro binding assays. Such assays are well known in the art and include but are not limited to: Bio-Layer Interferometry (BLI)-based assays (such as OCTET®) and surface plasmon resonance (SPR)-based assays (such as BIACORE®).

In some embodiments, the inhibitory potency (e.g., IC₅₀) of the novel antibodies of the present disclosure calculated based on cell-based assays (such as LN229 cell assays described elsewhere herein) may be less than 10 nM measured against each of the hLTBP1-proTGFβ1 and hLTBP3-proTGFβ1 complexes. In some embodiments, the antibodies have an IC50 of 5 nM or less (i.e., <5 nM) measured against each of the hLTBP1-proTGFβ1 and hLTBP3-proTGFβ1 complexes. In preferred embodiments, the IC50 of the antibody measured against at least one of the hLTBP1-proTGFβ1 and hLTBP3-proTGFβ1 complexes is less than 1 nM.

In some embodiments, potency may be evaluated in suitable in vivo models as a measure of efficacy and/or pharmacodynamics effects. For example, if the first antibody is efficacious in an in vivo model at a certain concentration, and the second antibody is equally efficacious at a lower concentration than the first in the same in vivo model, then, the second antibody can be said to be more potent than the first antibody. Any suitable disease models known in the art may be used to assess relative potencies of TGFβ1 inhibitors, depending on the particular indication of interest, e.g., fibrosis models. Preferably, multiple doses or concentrations of each test antibody are included in such studies.

Similarly, pharmacodynamics (PD) effects may be measured to determine relative potencies of inhibitory antibodies. Commonly used PD measures for the TGFβ signaling pathway include, without limitation, phosphorylation of SMAD2/3 and expression of downstream effector genes, the transcription of which is sensitive to TGFβ activation, such as those with a TGFβ3-responsive promoter element (e.g., SMAD-binding elements). In some embodiments, the antibodies of the present disclosure are capable of completely blocking disease-induced SMAD2/3 phosphorylation in preclinical fibrosis models when the animals are administered at a dose of 3 mg/kg or less. In some embodiments, the antibodies of the present disclosure are capable of suppressing fibrosis-induced expression of a panel of marker genes including MMP2, Coil a1, Col3a1, Fn1, CTGF, TIMP1, TGFβ1, TGFβ2, TGFβ3, LTBP1, LTBP3, LRRC33, LRRC32/GARP, SERPINE1/PAI-1, THBS1, and Loxl2, when the animals are administered an antibody, or an antigen-binding fragment thereof, at a dose of 10 mg/kg or less in a model of lung fibrosis, for example an idiopathic pulmonary fibrosis (IPF) model as described hereinin the Examples.

Method for measurements of antibody binding. The antibody or antigen-binding fragment thereof can bind a human LTBP1-proTGFβ1 complex or a human LTBP3-TGFβ1 complex with a K_{D} of less than 5 nM as measured by bio-layer interferometry, and has one or more of the following properties: (i) is cross-reactive with mouse LTBP1-proTGFβ1 complex; (ii) is cross-reactive with mouse LTBP3-proTGFβ1 complex; (iii) binds a mouse LTBP1-proTGFβ1 complex with a K_{D} of less than or equal to 10 nM as measured by bio-layer interferometry; (iv) binds a mouse LTBP3-proTGFβ1 complex with a K_{D} of less than or equal to 10 nM as measured by bio-layer interferometry; (v) binds a human LTBP1-proTGFβ1 complex or a human LTBP3-TGFβ1 complex with a K_{D} that is at least fifty times lower than the K_{D} when binding to a human GARP-proTGFβ1 complex under the same assay conditions; and (vi) shows less than 50-fold (preferably less than 100-fold) binding to a human GARP-proTGFβ1 complex, as measured by bio-layer interferometry, under the same assay conditions as used to measure binding to human LTBP1-proTGFβ1 complex or a human LTBP3-TGFβ1 complex; (vii) shows less than 50-fold (preferably less than 100-fold) binding to an LRRC33-proTGFβ1 complex as measured by bio-layer interferometry, under the same assay conditions as used to measure binding to human LTBP1-proTGFβ1 complex or human LTBP3-TGFβ1 complex.

The antibody or antigen-binding fragment may bind a human LTBP1-proTGFβ1 complex and a human LTBP3-TGFβ1 complex with a K_{D} of less than 5 nM as measured by bio-layer interferometry and has all the above properties (i)-(vii).

In some embodiments, binding characteristics (kinetics, affinities, etc.) are measured using surface plasmon resonance-based assay, such as Biacore. As an example, monovalent (Fab) affinities of Ab42 as measured by Biacore according to the manufacturer's instruction are shown below.

| Ab42 monovalent (Fab) affinities | | | |
|---|---|---|---|
| Human antigen | | | |
| complex | Ab42 K_{D} (nM) | ON rate (1/Ms) | OFF rate (1/s) |
| LTBP1-proTGFβ1 | 0.38 | 2.93e5 | 1.11e−4 |
| LTBP3-proTGFβ1 | 0.57 | 1.91e5 | 1.09e−4 |
| GARP-proTGFβ1 | 144.61 | 4.15e4 | 6e−3 |
| LRRC33-proTGFβ1 | 134.25 | 2.02e4 | 2.71e−3 |

Method for measurement of off-rates, k_{OFF}. Advantageous isoform-selective inhibitors of TGFβ1 activation may include monoclonal antibodies (including immunoglobulins and antigen-binding fragments or portions thereof) that exhibit slow dissociation rates, i.e., off-rates, k_{OFF}. The disclosure is further based on the recognition that treatment of chronic and progressive disease such as fibrosis may require inhibitors with superior durability, which may be reflected on the dissociation rate of such antibody.

The affinity of an antibody to its antigen can be measured as the equilibrium dissociation constant, or K_{D}. The ratio of the experimentally measured off- and on-rates (k_{OFF}/k_{ON}) can be used to calculate the K_{D} value. The k_{OFF} value represents the antibody dissociation rate, which indicates how quickly it dissociates from its antigen, whilst the k_{ON} value represents the antibody association rate which provides how quickly it binds to its antigen. The latter can be concentration-dependent, while the former is concentration-independent. The K_{D} value relates to the concentration of antibody (the amount of antibody needed for a particular experiment) and so the lower the K_{D} value (lower concentration) and thus the higher the affinity of the antibody. With respect to a reference antibody, a higher affinity antibody may have a lower k_{OFF} rate, a higher k_{ON} rate, or both.

Both the k_{OFF} and k_{ON} rates contribute to the overall affinity of a particular antibody to its antigen, and relative importance or impact of each component may depend on the mechanism of action of the antibody. Neutralizing antibodies, which bind mature growth factors, e.g., soluble, transient TGFβ1 ligand liberated from a latent complex, must compete with the endogenous high-affinity receptors for ligand binding in vivo. Because the ligand-receptor interaction is a local event and because the ligand is short-lived, such antibodies must be capable of rapidly targeting and sequestering the soluble growth factor before the ligand finds its cellular receptor (thereby activating the TGFβ1 signaling pathway) in the tissue. Therefore, for ligand-targeting neutralizing antibodies to be potent, the ability to bind the target growth factor fast, i.e., high association rates (k_{ON}), may be especially important.

To illustrate, the measured on rates and off rates of Ab42 are provided in the above table for each of the four human LLCs. As can be seen, sub-nanomolar affinities for the LTBP1/3 complexes are achieved by a combination of high association kinetics and slow dissociation kinetics.

### Potency; Inhibitory Activity

Antibodies disclosed herein may be broadly characterized as “functional antibodies” for their ability to inhibit TGFβ1 signaling. As used herein, “a functional antibody” confers one or more biological activities by virtue of its ability to bind an antigen (e.g., antigen complexes). Functional antibodies therefore broadly include those capable of modulating the activity/function of target molecules (i.e., antigen). Such modulating antibodies include inhibiting antibodies (or inhibitory antibodies) and activating antibodies. The present disclosure is drawn to antibodies which can inhibit a biological process mediated by TGFβ1 signaling associated with multiple contexts of TGFβ1. Inhibitory agents used to carry out the present disclosure, such as the antibodies described herein, are intended to be TGFβ31-selective and not to target or interfere with TGFβ2 and TGFβ3 when administered at a therapeutically effective dose (dose at which sufficient efficacy is achieved within acceptable toxicity levels). The novel antibodies of the present disclosure have enhanced inhibitory activities (potency) as compared to previously identified activation inhibitors of TGFβ1.

Pharmacodynamics (PD) effects may be measured to determine relative potencies of inhibitory antibodies. Commonly used PD measures for the TGFβ signaling pathway include, without limitation, phosphorylation of SMAD2/3 and expression of downstream effector genes, the transcription of which is sensitive to TGFβ activation, such as those with a TGFβ3-responsive promoter element (e.g., SMAD-binding elements). In some embodiments, the antibodies of the present disclosure are capable of completely blocking disease-induced SMAD2/3 phosphorylation in preclinical fibrosis models when the animals are administered at a dose of 3 mg/kg or less. In some embodiments, the antibodies of the present disclosure are capable of reducing and/or completely blocking disease-induced SMAD2/3 phosphorylation. In some embodiments, the antibodies of the present disclosure are capable of reducing and/or completely blocking disease-induced SMAD2 phosphorylation (e.g., regardless of any change in SMAD3). In some embodiments, reduction is measured as a ratio of phosphorylated SMAD2/3 over total SMAD2/3. In some embodiments, reduction is measured as a ratio of phosphorylated SMAD2 over total SMAD2. In some embodiments, the antibodies of the present disclosure are capable of reducing nuclear localization of phosphorylated SMAD2, as measured, for example, by IHC. Without being bound by theory, in some embodiments, measuring SMAD2 phosphorylation (without measuring SMAD3) may improve the accurate detection of a treatment-related effect. Denis et al., Development 143: 3481-90 (2016); Liu et al., J. Biol. Chem. 278: 11721-8 (2003); David et al., Oncoimmunology 6: e1349589 (2017). n some embodiments, the antibodies of the present disclosure are capable of suppressing fibrosis-induced expression of a panel of marker genes including MMP2, Col1a1, Col3a1, Fn1, CTGF, TIMP1, TGFβ1, TGFβ2, TGFβ3, LTBP1, LTBP3, LRRC33, LRRC32/GARP, SERPINE1/PAI-1, THBS1, and Loxl2 when the animals are administered an antibody or antigen-binding fragment of the present disclosure, at a dose of 30 mg/kg or less in a model of lung fibrosis, for example an idiopathic pulmonary fibrosis (IPF) model as described hereinin the Examples. In some embodiments, the antibodies of the present disclosure are capable of suppressing fibrosis-induced expression of a panel of marker genes including MMP2, Col1a1, Col3a1, Fn1, CTGF, TIMP1, TGFB1, TGFB2, TGFB3, LTBP1, LTBP3, LRRC33, LRRC32/GARP, SERPINE1/PAI-1, THBS1, and Loxl2 when the animals are administered an antibody or antigen-binding fragment of the present disclosure, at a dose of 10 mg/kg or less in a model of lung fibrosis, for example an idiopathic pulmonary fibrosis (IPF) model as described hereinin the Examples. In some embodiments, the antibodies of the present disclosure are capable of suppressing fibrosis-induced expression of a panel of marker genes including MMP2, Col1a1, Col3a1, Fn1, CTGF, TIMP1, TGFB1, TGFB2, TGFB3, LTBP1, LTBP3, LRRC33, LRRC32/GARP, SERPINE1/PAI-1, THBS1, and Loxl2 when the animals are administered an antibody or antigen-binding fragment of the present disclosure, at a dose of 3 mg/kg or less in a model of lung fibrosis, for example an idiopathic pulmonary fibrosis (IPF) model as described herein.

In some embodiments, potency of an inhibitory antibody may be measured in suitable cell-based assays, such as CAGA reporter assays described herein. Generally, cultured cells, such as heterologous cells and primary cells, may be used for carrying out cell-based potency assays. Cells that express endogenous TGFβ1 and/or a presenting molecule of interest, such as LTBP1, LTBP3, GARP and LRRC33, may be used. Alternatively, exogenous nucleic acids encoding protein(s) of interest, such as TGFβ1 and/or a presenting molecule of interest, such as LTBP1, LTBP3, GARP and LRRC33, may be introduced into such cells, for example by transfection (e.g., stable transfection or transient transfection) or by viral vector-based infection. In some embodiments, LN229 cells are employed for such assays. The cells expressing TGFβ1 and a presenting molecule of interest (e.g., LTBP1, LTBP3, GARP or LRRC33) are grown in culture, which “present” the large latent complex either on cell surface (when associated with GARP or LRRC33) or deposit into the ECM (when associated with an LTBP). Activation of TGFβ1 may be triggered by integrin, expressed on another cell surface.

The integrin-expressing cells may be the same cells co-expressing the large latent complex or a separate cell type. Reporter cells are added to the assay system, which incorporates a TGFβ-responsive element. In this way, the degree of TGFβ activation may be measured by detecting the signal from the reporter cells (e.g., TGFβ3-responsive reporter genes, such as luciferase coupled to a TGFβ3-responsive promoter element) upon TGFβ activation. Using such cell-based assay systems, inhibitory activities of the antibodies can be determined by measuring the change (reduction) or difference in the reporter signal (e.g., luciferase activities as measured by fluorescence readouts) either in the presence or absence of test antibodies.

### Pharmaceutical Compositions

The disclosure further provides pharmaceutical compositions used as a medicament suitable for administration in human and non-human subjects. One or more isoform-specific antibodies encompassed by the disclosure can be formulated or admixed with a pharmaceutically acceptable carrier (excipient), including, for example, a buffer, to form a pharmaceutical composition. Such formulations may be used for the treatment of a disease or disorder that involves TGFβ signaling. In particularly preferred embodiments, such formulations may be used for diseases or disorders involving fibrosis.

The pharmaceutical compositions of the disclosure may be administered to patients for alleviating a TGFβ-related indication (e.g., fibrosis, immune disorders, and/or cancer). “Acceptable” means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20^{th} Ed. (2000) Lippincott Williams and Wilkins, Ed. K. E. Hoover. In one example, a pharmaceutical composition described herein contains more than one antibody that specifically binds a GARP-proTGFβ1 complex, a LTBP1-proTGFβ1 complex, a LTBP3-proTGFβ1 complex, and a LRRC33-proTGFβ1 complex where the antibodies recognize different epitopes/residues of the complex.

The disclosure further provides pharmaceutical compositions used as a medicament suitable for administration in human and non-human subjects. One or more antibodies that selectively binds a LTBP1-TGFβ1 complex and/or a LTBP3-TGFβ1 complex can be formulated or admixed with a pharmaceutically acceptable carrier (excipient), including, for example, a buffer, to form a pharmaceutical composition. Such formulations may be used for the treatment of a disease or disorder that involves TGFβ signaling. In some embodiments, such disease or disorder associated with TGFβ signaling involves one or more contexts, i.e., the TGFβ is associated with a particular type or types of presenting molecules. In some embodiments, such context occurs in a cell type-specific and/or tissue-specific manner. In some embodiments, for example, such context-dependent action of TGFβ signaling is mediated in part via GARP, LRRC33, LTBP1 and/or LTBP3.

In some embodiments, the antibody of the present disclosure binds selectively to a single context of TGFβ, such that the antibody binds TGFβ in a complex with LTBP presenting molecules, e.g., LTBP1 and/or LTBP3. Thus, such pharmaceutical compositions may be administered to patients for alleviating a TGFβ-related indication (e.g., fibrosis) associated with TGFβ1 activation/release from LTBP1 and/or LTBP3.

A pharmaceutically “acceptable” carrier (excipient) means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more than one antibody that selectively binds a LTBP1-TGFβ1 complex and/or a LTBP3-TGFβ1 complex, where the antibodies recognize different epitopes/residues of the LTBP1-TGFβ1 complex and/or LTBP3-TGFβ1 complex.

The pharmaceutical compositions to be used in the present methods can comprise pharmaceutically acceptable carriers, excipients, or stabilizers in the form of lyophilized formulations or aqueous solutions (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In some examples, the pharmaceutical composition described herein comprises liposomes containing an antibody that selectively binds a LTBP1-TGFβ1 complex and/or a LTBP3-TGFβ1 complex, which can be prepared by any suitable method, such as described in Epstein et al., Proc. Natl. Acad. Sci. USA 82:3688 (1985); Hwang et al. Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies that selectively bind a LTBP1-TGFβ1 complex and/or a LTBP3-TGFβ1 complex may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 mg to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. TWEEEN™ 20, 40, 60, 80 or 85) and other sorbitans (e.g. SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPSYN™, INFONUTROL™, LIPOFUNDIN™ and LIPIPHYSAN™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody that selectively binds a LTBP1-TGFβ1 complex and/or a LTBP3-TGFβ1 complex with INTRALIPID™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### TGFβ Inhibitors Suitable for Therapeutic Use

According to aspects described herein, the disclosure provides improved TGFβ inhibitors and methods of choosing therapeutically effective TGFβ inhibitors that can selectively target matrix-associated proTGFβ complexes. The isoform-selective inhibitors of TGFβ1 activation have advantageous features for the treatment of fibrosis, e.g., lung fibrosis (e.g., idiopathic pulmonary fibrosis (IPF)), kidney fibrosis (e.g., Alport syndrome), or muscle fibrosis (e.g., muscle fibrosis in Duchene's muscular dystrophy (DMD), which has clinical and preclinical evidence for LTBP involvement). The isoform-selective inhibitors of TGFβ1 activation have advantageous features for the treatment of fibrosis when the fibrotic condition involves extracellular matrix dysregulation.

The methods disclosed and claimed herein are based on in vivo data from preclinical fibrosis models, which demonstrate surprisingly effective therapeutic results such as reduction in the amount of collagen present in a fibrotic tissue, reduction in the amount of new collagen synthesis, and/or reduction in the amount of phosphorylated Smad2 in a fibrotic tissue. Moreover, the disclosure provides therapeutic dosing strategies, including a loading/maintenance dosing strategy demonstrated to be surprisingly therapeutically effective in vivo. The methods described herein provide a strong basis for a dosage determination and administration schedule for the isoform-selective inhibitors of TGFβ1 activation, which can be confirmed further in human clinical trials.

Use of Inhibitors that Selectively Bind a LTBP1/3-TGFβ1 Complex

The inhibitors, e.g., antibodies and antigen binding fragments thereof, described herein that selectively bind a LTBP1/3-TGFβ1 complex can be used in a wide variety of applications in which modulation of TGFβ1 activity associated with LTBP1 or LTBP3 is desired.

In one embodiment, the disclosure provides a method of inhibiting TGFβ1 activation by exposing a LTBP1-TGFβ1 complex and/or a LTBP3-TGFβ1 complex to an inhibitor, e.g., antibody, or antigen binding portion thereof, which selectively binds a LTBP1/3-TGFβ1 complex. The foregoing method can be performed in vitro, e.g., to inhibit TGFβ1 activation in cultured cells. The foregoing method can also be performed in vivo, e.g., in a subject in need of TGFβ1 inhibition, or in an animal model in which the effect of TGFβ1 inhibition is to be assessed.

Any inhibitor, e.g., an antibody, or an antigen binding fragment thereof, described herein which selectively binds a LTBP1-TGFβ1 complex and/or a LTBP3-TGFβ1 complex, and any pharmaceutical composition comprising such antibody, is suitable for use in the methods of the disclosure. For example, in one embodiment, the inhibitor, e.g., antibody, or antigen-binding portion thereof, selectively binds to a LTBP1-TGFβ1 complex and a LTBP3-TGFβ1 complex, but does not bind to one or more targets selected from LTBP1 alone, mature TGFβ1 alone, a GARP-TGFβ1 complex, a LRRC33-TGFβ1 complex, and combinations thereof. Exemplary inhibitor, e.g., antibodies, can inhibit the release of mature TGFβ1 from a LTBP1-proTGFβ1 complex and/or a LTBP3-proTGFβ1 complex, without inhibiting the release of mature TGFβ1 from a GARP-proTGFβ1 complex and/or a LRRC33-proTGFβ1 complex.

The antibody, or antigen-binding portion thereof, can, in some embodiments, bind a LTBP1-proTGFβ1 complex and/or a LTBP3-proTGFβ1 complex with a dissociation constant (K_{D}) of about 10-6 M or less, 10-7 M or less, 10-8 M or less. In one embodiment, the antibody, or antigen binding portion thereof, comprises at least one (e.g., one, two, or three) heavy chain CDRs shown in Table 5, and/or at least one (e.g., one, two, three) light chain CDRs shown in Table 5. In an exemplary embodiment, the antibody, or antigen binding portion thereof, comprises a heavy chain variable region comprising SEQ ID NO:7, and/or a light chain variable region comprising SEQ ID NO:8. Antibodies and antigen binding fragment thereof which bind the same epitope as the foregoing antibodies, and/or which compete for binding with the foregoing antibodies to LTBP1/3-proTGFβ1, are also useful in the methods described herein. Additional features of the antibodies, or antigen-binding fragment thereof, that are suitable for practicing the methods of the disclosure are described herein.

In one embodiment, contacting a LTBP1-TGFβ1 complex and/or a LTBP3-TGFβ1 complex with the inhibitor, e.g., antibody, or antigen binding portion thereof, inhibits the release of mature TGFβ1 from the LTBP1-TGFβ1 complex and/or the LTBP3-TGFβ1 complex. In one embodiment, said contacting does not inhibit the release of mature TGFβ1 from presenting molecules other than LTBP1 and LTBP3. For example, exposing a GARP-TGFβ1 complex or a LRRC33-TGFβ1 complex to a context-specific inhibitor, e.g., antibody, that selectively binds LTBP1/3-TGFβ1 but does not bind TGFβ1 in the context of GARP or LRRC33 will not inhibit the release of mature TGFβ1 from the GARP-TGFβ1 complex or the LRRC33-TGFβ1 complex. LTBP1 and LTBP3 are generally deposited in the extracellular matrix. Accordingly, in one embodiment, complexes comprising LTBP1-TGFβ1 and/or LTBP3-TGFβ1 are associated with the extracellular matrix, e.g., bound to the extracellular matrix. In some embodiments, the LTBP1/3-TGFβ1 complexes are bound to extracellular matrix comprising fibrillin, and/or a protein containing an RGD motif.

The disclosure also provides a method of reducing TGFβ1 activation in a subject, by administering to the subject an inhibitor, e.g., antibody, or antigen binding portion thereof, which selectively binds a LTBP1/3-TGFβ1 complex, as described herein. Any antibody, or antigen binding portion thereof, described herein which selectively binds a LTBP1-TGFβ1 complex and/or a LTBP3-TGFβ1 complex, and any pharmaceutical composition comprising such antibody, is suitable for use in the methods of the disclosure.

Exemplary LTBP1/3 inhibitors, e.g., antibodies, bind a LTBP1/3-TGFβ1 complex, and inhibit TGFβ1 activation in a context-specific manner, by inhibiting release of TGFβ1 presented by LTBP1 and LTBP3, without inhibiting release of TGFβ1 presented by GARP and/or LRRC33. Such antibodies are useful for blocking a particular subset of TGFβ1 activity in vivo. In one embodiment, the context-specific antibodies provided herein can be used to inhibit TGFβ1 localized to the extracellular matrix.

In another embodiment, the context-specific antibodies can inhibit TGFβ1 without modulating TGFβ1-associated immune activity or immune response, which is primarily mediated by TGFβ1 presented by GARP and LRRC33. In another embodiment, the context-specific antibodies can be used to inhibit TGFβ1 activity associated with the extracellular matrix (e.g., LTBP1-associated TGFβ1 activity and LTBP3-associated TGFβ1 activity) without modulating TGFβ1 activity associated with hematopoietic cells, e.g., hematopoietic cells that express GARP and/or LRRC33.

Possible Effects of TGFβ2 and/or TGFβ3 Inhibition

Traditional approach to TGFβ inhibition has been to block multiple isoforms of TGFβ (i.e., TGFβ1, TGFβ2 and TGFβ3, or at least two of the three isoforms), based at least on the assumption that it would be necessary to do so in order to achieve efficacy, given that all three isoforms signal through the same receptors. However, Applicant's recent observations in murine fibrosis models suggest detrimental effects of TGFβ3 inhibition in tissues with dysregulated ECM, raising the possibility that role of TGFβ3 expands beyond homeostasis. Applicant previously found that TGFβ1-selective inhibitors are capable of mitigating fibrosis in multiple preclinical models, including mouse liver fibrosis model where both the TGFβ1 and TGFβ3 isoforms are expressed in fibrotic liver tissue, albeit in discrete cell types. Surprisingly, inhibition of TGFβ3 promoted pro-fibrotic phenotypes. The exacerbation of fibrosis was observed when the TGFβ3 inhibitor was used alone. Furthermore, when used in combination with a TGFβ1-selective inhibitor, the TGFβ3-selective inhibitor attenuated the anti-fibrotic effect of the TGFβ1 inhibitor, indicating that TGFβ3 may counteract the anti-fibrotic effects of TGFβ1 and that inhibition of TGFβ3 is in fact detrimental for the treatment of fibrosis. These observations point to isoform-selective inhibition of TGFβ1 as the preferred approach in treating fibrosis.

In addition to the possible concerns of inhibiting TGFβ3 addressed above, Takahashi et al., (Nat Metab. 2019, 1(2): 291-303) recently reported a beneficial role of TGFβ2 in regulating metabolism. The authors identified TGFβ2 as an exercise-induced adipokine, which stimulated glucose and fatty acid uptake in vitro, as well as tissue glucose uptake in vivo; which improved metabolism in obese mice; and, which reduced high fat diet-induced inflammation. Moreover, the authors observed that lactate, a metabolite released from muscle during exercise, stimulated TGFβ2 expression in human adipocytes and that a lactate-lowering agent reduced circulating TGFβ2 levels and reduced exercise-stimulated improvements in glucose tolerance. These observations suggest that therapeutic use of a TGFβ inhibitor with inhibitory activity towards the TGFβ2 isoform may be harmful, at least in the metabolic aspect.

Accordingly, the present disclosure provides a TGFβ inhibitor for use in the treatment of a TGFβ-related indication (e.g., fibrosis) in a subject, wherein, the TGFβ inhibitor not only selectively inhibits TGFβ1 but also in a context-specific manner, e.g., TGFβ1 associated with LTBP1 and/or LTBP3 in the extracellular matrix, where it can provide anti-fibrotic effect without further aggravating inflammation of the tissue. In some embodiments, the subject benefits from improved metabolism, wherein optionally, the subject has or is at risk of developing a metabolic disease, such as obesity, high fat diet-induced inflammation, glucose or insulin dysregulation (e.g., type 1 diabetes and type 2 diabetes). In some embodiments, the TGFβ-related indication is a fibrotic disorder involving inflammation. In preferred embodiments, the subject suffers from a condition in which regulatory T cells serve a positive role, such as dampening overstimulation of the immune system or preventing or attenuating autoimmunity. In some embodiments, LTBP-selective TGFβ1 inhibitors are used in conjunction with a genotoxic therapy (such as radiation therapy and chemotherapy), or in a subject exposed to a genotoxic agent (such as radiation/irradiation and carcinogens), aimed to inhibit ROS-induced TGFβ1 activation without depleting Tregs and immunosuppressive macrophages which are beneficial for reducing treatment-induced inflammation.

Possible effects of TGFβ2 and/or TGFβ3 inhibition

Traditional approach to TGFβ inhibition has been to block multiple isoforms of TGFβ (i.e., TGFβ1, TGFβ2 and TGFβ3, or at least two of the three isoforms), based at least on the assumption that it would be necessary to do so in order to achieve efficacy, given that all three isoforms signal through the same receptors. However, Applicant's recent observations in murine fibrosis models suggest detrimental effects of TGFβ3 inhibition in tissues with dysregulated ECM, raising the possibility that role of TGFβ3 expands beyond homeostasis. Applicant previously found that TGFβ1-selective inhibitors are capable of mitigating fibrosis in multiple preclinical models, including mouse liver fibrosis model where both the TGFβ1 and TGFβ3 isoforms are expressed in fibrotic liver tissue, albeit in discrete cell types. Surprisingly, inhibition of TGFβ3 promoted pro-fibrotic phenotypes. The exacerbation of fibrosis was observed when the TGFβ3 inhibitor was used alone. Furthermore, when used in combination with a TGFβ1-selective inhibitor, the TGFβ3-selective inhibitor attenuated the anti-fibrotic effect of the TGFβ1 inhibitor, indicating that TGFβ3 may counteract the anti-fibrotic effects of TGFβ1 and that inhibition of TGFβ3 is in fact detrimental for the treatment of fibrosis. These observations point to isoform-selective inhibition of TGFβ1 as the preferred approach in treating fibrosis.

Clinical Applications of Context-Specific Inhibitors of L TBP1-Associated and/or LTBP3-Associated TGFβ1

Applicant previously described so-called “context-independent” inhibitors of TGFβ1 (see, for example: PCT/US2017/021972, PCT/US2018/012601, PCT/US2019/041390 and PCT/US2019/041373) which may be useful for treating various diseases and disorders involving TGFβ1 dysregulation, including, but are not limited to, cancer and fibrosis. Unlike traditional TGFβ1 antagonists, these context-independent TGFβ1 inhibitors are capable of selectively targeting the TGFβ1 isoform. Within the multifaceted biological functions driven by the TGFβ1 isoform, however, the context-independent inhibitors do not discriminate among tissue-specific (thus context-specific) proTGFβ1 complexes, such that such inhibitors are capable of binding and thereby inhibiting release of mature growth factor from any of the presenting molecule-proTGFβ1 complexes (e.g., LTBP1-proTGFβ1, LTBP3-proTGFβ1, GARP-proTGFβ1 and LRRC33-proTGFβ1).

Based at least in part on the recognition that it may be advantageous to provide even greater selectivity in targeting only a subset of TGFβ1 activities, context-selective inhibitors of the present disclosure have been generated. It is contemplated that by further fine-tuning particular biological contexts in which to inhibit TGFβ1 function, greater safety and efficacy may be achieved in a subset of disease conditions or patient populations. Specifically, the inventors of the present disclosure have recognized that in certain conditions, systemic perturbation of immune regulation may be particularly undesirable. Because TGFβ1 plays an important role in mediating immune response and maintaining immune homeostasis, broad inhibition of TGFβ1 activities effectuated in a context-independent manner may lead to unwanted side effects without justifiable benefits. In these circumstances, it is envisaged that it is advantageous to specifically target and inhibit matrix-associated TGFβ1 function using a context-selective inhibitor, such as those encompassed herein, which does not inhibit the immune components of TGFβ1 function. Thus, this approach may be particularly advantageous in situations where regulatory T cells and/or immunosuppressive macrophages (such as M2-type) serve a protective role to reduce inflammation and/or autoimmunity. In some embodiments, the inflammation is triggered by a tissue repair response. In some embodiments, the inflammation is triggered by oxidative stress associated with reactive oxygen species (ROS).

Accordingly, the context-specific antibodies can be used to inhibit LTBP1/3-associated TGFβ1 activity in applications in which TGFβ1 activation in the context of LTBP1 or LTBP3 is desirable, and in which TGFβ1 activation in the context of GARP and/or or LRRC33 is detrimental.

Rationale for the Development of Matrix-Targeted TGFβ1 Inhibitors that do not Inhibit GARP-Associated TGFβ1

The disclosure includes therapeutic use of potent context-specific inhibitors of LTBP1-associated and/or LTBP3-associated TGFβ1. Such inhibitors therefore are capable of specifically targeting the ECM-associated latent TGFβ1 complexes (e.g., LTBP1-proTGFβ1 and/or LTBP3-proTGFβ1) thereby inhibiting the release of mature TGFβ1 growth factor from the latent complex at disease environments, e.g., fibrotic tissues. Such inhibitors preferably show no significant binding activities towards a GARP-proTGFβ1 complex, thereby minimizing unwanted systemic immune modulations. To that end, use of TGFβ1 inhibitors that selectively and potently target extracellular matrix-associated TGFβ1 (e.g., LTBP1-proTGFβ1 and LTBP3-proTGFβ1 complexes) is considered advantageous as a therapy to treat fibrosis involving inflammation.

At least three rationales for supporting potential benefits of a TGFβ1 inhibitor that does not target the GARP-proTGFβ1 complex expressed on regulatory T cells are discussed below.

First, GARP-expressing T regulatory cells are a component of the immune system that suppress or dampen immune responses of other cells. This notion may be referred to as “tolerance.” This is an important “self-check” built into the immune system to prevent excessive reactions that in some situations can result in life-threatening conditions, such as sepsis, cytokine release syndrome and cytokine storm. TGFβ1 inhibition therapies that exert Treg-inhibitory effects may, therefore, pose certain risk when the normal Treg function is impaired, particularly for a prolonged duration of time, e.g., therapeutic regimen involving treatment of six months or longer, and chronic treatment that is administered for an indefinite period of time. For this reason, patients in need of TGFβ1 inhibition therapies, particularly to avoid the risk of eliciting autoimmunity, may benefit from TGFβ1 inhibitors that do not directly perturb the normal Treg function. For example, patient populations in need of a long-term TGFβ1 inhibition therapy may include those with genetic or congenital conditions, such as DMD, CF and others. In addition, patient populations that suffer from conditions that include inflammation may benefit from a context-specific inhibitor that does not perturb the GARP/Treg function so as to minimize the risk of exacerbating the existing inflammatory conditions.

Second, increasing evidence points to a link between disproportionate Th17/Treg ratios and pathologies involving inflammation and/or fibrosis. It is generally accepted that the differentiation of the two cell types, Th17 and Treg, is negatively regulated with an inverse relationship. TGFβ1 appears to be a master gatekeeper of this process, such that, TGFβ1 exposure promotes naïve T cells to differentiate into Foxp3+ Tregs, whereas TGFβ1 in combination with IL-6, promotes naïve T cells to differentiate into RORγt+Th17 cells instead. In addition, once differentiated, these cell populations negatively regulate each other.

Lines of evidence suggest that an imbalance in Th17/Treg ratios correlates with the pathogenesis and/or progression of fibrotic conditions involving chronic inflammation, or severity thereof.

For example, Shoukry et al. reported that Th17 cytokines drive liver fibrosis by regulating TGFβ signaling. The authors examined ex vivo the frequency of Th17 and Treg populations in liver biopsy samples and found that increased Th17/Treg ratio correlated with advanced fibrosis, as compared to moderate fibrosis or healthy tissue samples. Consistent with the observation, a strong bias towards Th17 cytokines, IL-22 in particular, was also detected in fibrotic livers. These data suggest that increased Th17/Treg ratios lead to an imbalance in pro-fibrotic Th17 cytokines, which correlate with severity of liver fibrosis.

Similarly, Rau et al. reported that progression from NAFLD to NASH is marked by a higher frequency of Th17 cells in the liver and an increased Th17/Treg ratio in peripheral blood and in the liver (J. Immunol 2016, 196: 97-105).

Similar inverse correlations of Th17 and Treg populations are observed in other diseases.

For example, increased muscle expression of IL-17 has been reported in patients with Duchenne muscular dystrophy (DMD), which is a condition that manifests chronic inflammation. De Pasquale et al. (Neurology 78(17): 1309-14) found that DMD muscle biopsy samples contained higher levels of IL-17 (a Th17 marker) and lower levels of Foxp3 (a Treg marker) mRNA compared to control. Elevations in other proinflammatory cytokines, such as TNF-α and MCP-1, were also observed and were found to be associated with worse clinical outcome of patients. The authors concluded that the data point to a possible pathogenic role of IL-17.

Similarly, Jamshidian et al. (J Neuroimmunol 2013, 262(1-2): 106-12) reported biased Treg/Th17 balance away from regulatory toward inflammatory phenotype in patients with relapsed multiple sclerosis and its correlation with severity of clinical symptoms.

A role of regulatory T cells is also implicated in the pathogenesis of cystic fibrosis (CF). In particular, CF lungs affected by the disease are associated with exaggerated Th17 and Th2 cell responses, indicative of a classic inflammatory phenotype, but also with a deficiency in numbers or function (i.e., impairment) of Treg cells (McGuire (2015) Am J Respir Crit Care Med 191(8): 866-8).

Furthermore, Zhuang et al. (Scientific Reports (2017) 7: 40141) found imbalance of Th17/Treg cells in patients with acute anterior uveitis (anterior segment intraocular inflammation with the positive of human class I major histocompatibility complex), in which both a marked increase in Th17 cells and a marked decrease in Treg cells were seen.

Taken together, the inventors of the present disclosure recognized that what appears to be a common feature in these various diseases associated with elevated Th17/Treg ratios is that the patient suffers from a fibrotic condition accompanied by an inflammatory component.

Thus, it is envisaged in the present disclosure that TGFβ1 inhibition therapy that spares the Treg/GARP-arm of the TGFβ1 function may be particularly advantageous for an effective treatment of diseases characterized by an elevated level of Th17/Treg ratios. In this way, the context-selective inhibitors of TGFβ1 according to the disclosure are aimed to avoid more systemic effects of TGFβ1 inhibition that may interfere with Treg function, which may lead to exacerbation of existing fibrotic/inflammatory conditions in patients. Thus, the isoform-specific, matrix-targeted TGFβ1 inhibitors described herein are used in a method for treating a patient who has or at risk of developing a fibrotic disorder that comprises inflammation. In some embodiments, the patient has an elevated Th17-to-Treg cell ratio. In some embodiments, the elevated Th17/Treg ratio may be predominantly caused by an increased number of Th17 cells, while in other embodiments, the elevated Th17/Treg ratio may be predominantly caused by a decreased number of Treg cells in the patient (or a biological sample collected from the patient). Yet in further embodiments, the elevated Th17/Treg ratio may be caused by a combination of an increased number of Th17 cells and a decreased number of Treg cells. In some embodiments, elevated levels of IL-17 and/or IL-22 detected in patients (or measured in samples collected from the patients) are also indicative of fibrotic conditions accompanied by chronic inflammation. Such patients may be therefore selected as candidates for receiving a context-selective TGFβ1 inhibitor therapy disclosed herein.

The third line of reasoning for keeping the GARP-TGFβ1 axis intact in a TGFβ1 inhibition therapy relates to the benefit of maintaining normal Treg function. As mentioned, GARP is expressed on the cell surface of Tregs and are thought to play a role in TGFβ1-mediated immunomodulation. Because Tregs are indispensable for immune homeostasis and the prevention of autoimmunity, unnecessary perturbation of which may put certain patient populations at higher risk of, for example, infections (reviewed, for example, by: Richert-Spuhler and Lund (2015) Prog Mol Biol Transl Sci. 136:217-243).

And finally, a further line of reasoning for keeping the GARP-TGFβ1 axis intact in a TGFβ1 inhibition therapy is that regulatory T cells function as a “break” to modulate or dampen over-reactive immune response. The discovery of Foxp3 as the master regulator of Treg cell development and function was critical for the understanding of Treg cell biology. Inactivating mutations in Foxp3 result in the spontaneous development of severe autoimmunity with a scurfy phenotype in mice and IPEX syndrome (‘immune dysregulation, polyendocrinopathy, enteropathy, X-linked’) in humans (see Dominguez-Villear and Haler, Nature Immunology 19, 665-673, 2018). Thus, it raises the possibility that TGFβ1 therapy that elicits inhibitory effects of the Treg/GARP arm of TGFβ1 function, especially in a prolonged treatment, may cause or exacerbate autoimmune response.

Increasing evidence suggests that Tregs not only act to dampen overexuberant effector immune responses, they also have the ability to potentiate appropriate immune responses to pathogens, by participating in pathogen clearance and protection of the host from collateral damage. Such diverse function of Treg cells is particularly apparent in delicate tissues such as the lung, which is constantly exposed to an external environment from which a variety of pathogens and other foreign components (e.g., viral pathogens, bacterial pathogens, fungal pathogens, and allergens) may gain access to host cells.

For example, influenza virus infection elicits a strong proinflammatory cytokine response with abundance immune cell infiltration. In acute and/or severe infections, such response can cause serious sequelae in susceptible individuals. Tregs provide a mechanism for dampening viral infection-associated pathology by controlling the magnitude of immune response in the host. Indeed, pathogen-exposed Tregs retain protective effects in adoptive transfer. Moreover, such adoptive transfer of primed Tregs have been shown to ameliorate influenza virus-associated morbidity and to prolong survival in severe immunocompromised animal models.

Accordingly, the disclosure provides use of an ECM-targeted, context-selective TGFβ1 inhibitor (e.g., LTBP1-selective or LTBP1/3-selective inhibitors of TGFβ1 activation inhibitors) for the treatment of a disease that involves matrix-associated TGFβ1 dysregulation in a subject. The subject is suffering from or at risk of an infection. The infection can be viral infections (e.g., influenza virus, respiratory syncytial virus or RSV, human immunodeficiency virus or HIV, MARS, SARS, herpes simplex virus or HSV, hepatitis A virus or HAV, hepatitis B virus or HBV, hepatitis C virus or HCV, CMV, Dengue virus, lymphocytic choriomeningitis virus, and West Nile virus), bacterial infections (meningitis, Mycobacterium tuberculosis, Listeria monocytogenes, Citrobacter rodentium, Salmonella, and E. coli), and/or fungal infections (e.g., Candida, Pneumocytis, Aspergillus, Cryptococcus, and Coccidioides).

Typically, high-risk or at-risk populations (individuals that are considered particularly susceptible to severe infections or infection-triggered responses) include pediatric populations (infants, young children, e.g., human individuals under the age of 7); elderly populations (those who are 65 years or older); those with compromised immune system due to medical condition, health status, life styles such as smoking, and/or medications with immunosuppressive effects, etc.

For example, certain medications cause weakened immunity, such as chemotherapy, therapies that target hematopoietic cells such as CD33 therapy, steroids, immunosuppressants, and statins.

In some embodiments, high-risk or at-risk populations are those with existing medical conditions, such as those with chronic infections such as HIV, those with bone marrow transplantation, pre-diabetic individuals, diabetic individuals, those with autoimmune disorders such as RA, asthma and allergy.

Thus, matrix-targeted, context-selective TGFβ1 inhibitors encompassed herein may be particularly advantageous for treating patients who require a long-term or chronic TGFβ1 therapy since in these scenarios it is beneficial to avoid impairment of immune homeostasis and the normal immune function that provides the ability to respond effectively to possible infections caused by a variety of pathogens such as those listed above.

Accordingly, antibodies, and antigen binding fragment thereof, that selectively bind LTBP-TGFβ1 (e.g., LTBP1-TGFβ1 and LTBP3-TGFβ1), and that do not inhibit TGFβ1 in the context of the immune-associated TGFβ1 presenters GARP and LRRC33, are therapeutic candidates for the treatment of fibrotic indications such as organ fibrosis, and are aimed to avoid TGFβ-related global immune activation. In one embodiment, the context-specific antibodies can be used to inhibit LTBP1/3-associated TGFβ1 activity in applications in which TGFβ1-mediated immune suppression is beneficial, e.g., in a subject who has received a transplant, who is a candidate for receiving a transplant, or who is expected to receive a transplant. In some embodiments, the subject has an advanced stage fibrosis and/or a bone marrow disease.

The foregoing methods can be used to treat a subject having a condition for which inhibition or reduction in LTBP-associated TGFβ1 activity is beneficial. For example, the subject may have or be at risk for developing a disorder in which extracellular matrix-associated TGFβ1 activity has been implicated.

Integrin-mediated activation of latent TGFβ1 in the extracellular matrix is a key contributor to fibrosis. Without wishing to be bound by theory, it is presently understood that integrins, including αVβ6 and αVβ8, can trigger the release of TGFβ1 from presenting molecules including LTBP1 and LTBP3. Inhibiting release of TGFβ1 in this context can reduce or eliminate fibrosis, and/or symptoms associated therewith.

As described, LTBP1 and LTBP3 are produced and are deposited extracellularly as components of the ECM, where they can “present” a proTGFβ1 complex (latent, inactive precursor of TGFβ1) within the ECM. Upon stimulation, the LTBP1/3-proTGFβ1 complex releases the TGFβ1 growth factor (the active, mature form of growth factor) which in turn is thought to be involved in the regulation of the local tissue microenvironment, such as ECM maintenance/remodeling and the process of fibrosis, possibly by responding to various cytokines, chemokines and growth factors, and by interacting with other ECM components, such as fibronectin, Fibrillin, collagen, elastin, and matrix metallopeptidases (MMPs).

In the normal wound healing process that occurs in response to an injury, for example, TGFβ is thought to facilitate granular tissue formation, angiogenesis, and collagen synthesis and production. TGFβ signaling is also implicated in abnormal tissue fibrogenesis (i.e., fibrosis), which results in formation of excess fibrous connective tissue in an organ or tissue in a reparative or reactive process characterized by the pathological accumulation of extracellular matrix (ECM) components, such as collagens. In these and other situations, the TGFβ axis may affect further aspects (in addition to fibrotic aspect), such as inflammation, recruitment and phenotypic switch of various cell types, which may be mediated by its interaction with one or more of the other presenting molecules, such as GARP/LRRC32 and LRRC33. In certain instances, it is advantageous to preferentially inhibit the LTBP1/3-context of TGFβ1 activation, without significantly inhibiting one or more of the other contexts of TGFβ1 activation, in situations where ECM-associated TGFβ1 that drives fibrosis is to be selectively inhibited.

Reactive oxygen species (ROS) has been reported to mediate TGFβ1-induced activation of fibroblasts. A conserved methionine residue present only on the TGFβ1 isoform, and not on TGFβ2 and TGFβ3 isoforms, may mediate this process. ROS-induced TGFβ1 activation has been reported to cause an increased level of alpha-smooth muscle actin (α-SMA), which is a myofibroblast marker (reviewed, for example, in Zhao et al., Signal Transduction and Targeted Therapy (2022) 7:206). Kraaij et al reported that macrophage-derived ROS caused Treg induction which protected mice from autoimmune conditions such as arthritis (PNAS (2010), 107(41): 17686-17691). These observations raise a possible benefit of sparing TGFβ1 signaling associated with LRRC33-expressing macrophages.

### Therapeutic Use of LTBP-Selective TGFβ1 Inhibitors

According to the present disclosure, a LTBP-selective TGFβ1 inhibitor is used in the treatment of tissue or organ lesions caused by tissue-damaging agents, such as chemical agents, toxic metabolites, hypoxia, ROS, and metabolic dysregulation. Repeated, prolonged, or chronic exposure to such tissue-damaging agents can cause progressive deterioration of the affected organ, typically characterized by initial repair responses (e.g., immune responses) followed by fibrogenesis that accompany hardening of the tissue, and ultimately leading to organ failure. It is therefore contemplated that the LTBP-selective approach of TGFβ1 inhibition described herein may serve dual purposes of i) normalizing the ECM function by reducing collagen deposits (e.g., anti-fibrotic effect); and, ii) maintaining the immunosuppressive axis of the TGFβ1 pathway (e.g., GARP-proTGFβ1 and LRRC33-proTGFβ1).

Accordingly, in one embodiment, the disclosure provides a method of reducing TGFβ1 activation in a subject having, or at risk of developing, a fibrotic disorder by administering to the subject an antibody, or antigen binding portion thereof, which selectively binds a LTBP1/3-TGFβ1 complex, as described herein. In another embodiment, the disclosure provides a method of treating a fibrotic disorder by administering to the subject an antibody, or antigen binding portion thereof, which selectively binds a LTBP1/3-TGFβ1 complex, as described herein. Such methods are particularly advantageous for the treatment of a disease involving both fibrosis and inflammation, such as those included below. With any of the therapeutic use disclosed herein, in preferred embodiments, the LTBP-selective TGFβ1 inhibitor for use is Ab42 or a variant thereof. In other embodiments, the LTBP-selective TGFβ1 inhibitor for use is an antibody or antigen-binding fragment thereof that competes antigen binding with Ab42, wherein optionally, the antibody or the fragment binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex, wherein the antibody is not Ab42. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain), wherein the antibody is not Ab42. In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In one embodiment, the subject has organ fibrosis, for example, kidney fibrosis (e.g., fibrosis associated with chronic kidney disease (CKD)), liver fibrosis (e.g., fibrosis associated with nonalcoholic steatohepatitis (NASH)), lung fibrosis (e.g., idiopathic pulmonary fibrosis (IPF)), cardiac fibrosis, and/or skin fibrosis (e.g., scleroderma). In one embodiment, the subject has desmoplasia.

In some embodiments, the subject can have advanced organ fibrosis. For example, the subject may be in need of an organ transplant. In one embodiment, the subject may be in need of an organ transplant, and the compounds and compositions described herein are administered to prevent allograft fibrosis from developing in the subject following receipt of the transplant.

A recent study examined whether inhibiting integrin αVβ6 could prevent TGFβ-mediated allograft fibrosis after kidney transplantation (Lo et al., Am. J. Transplant. (2013), 13:3085-3093). Surprisingly, animals treated with an inhibitory anti-αVβ6 antibody experienced a significant decrease in rejection-free survival compared to placebo animals. The authors conclude that this result cautions against TGFβ inhibition in kidney transplantation, because the immunosuppressive properties of TGFβ help prevent allograft rejection. The inhibitors, e.g., antibodies, and antigen binding fragment thereof, described herein advantageously inhibit activation of TGFβ1 presented by LTBP1 or LTBP3 in the extracellular matrix, but do not inhibit activation of TGFβ1 presented by GARP or LRRC33 on immune cells. Accordingly, the context-specific LTBP1/3-TGFβ1 inhibitors, e.g., antibodies, described herein can prevent or reduce allograft fibrosis, without eliminating the immunosuppressive properties of TGFβ1 that are useful for preventing allograft rejection. Accordingly, in one aspect, the disclosure provides a method for treating a fibrotic disorder in a subject, comprising administering to the subject a therapeutically effective amount of an inhibitor of TGFβ1 signaling, wherein the inhibitor is a selective inhibitor of ECM-associated TGFβ1; and, wherein the subject benefits from suppressed immunity. In one embodiment, the subject has a fibrotic condition and would benefit from an allograft transplant, or has received an allograft transplant.

Additional fibrotic conditions for which antibodies and/or compositions of the present disclosure may be used therapeutically include, but are not limited to, lung indications (e.g. idiopathic pulmonary fibrosis (IPF), chronic obstructive pulmonary disorder (COPD), allergic asthma, cystic fibrosis (CF), acute lung injury, eosinophilic esophagitis, pulmonary arterial hypertension and chemical gas-injury), kidney indications (e.g., diabetic glomerulosclerosis, focal segmental glomeruloclerosis (FSGS), chronic kidney disease, fibrosis associated with kidney transplantation and chronic rejection, IgA nephropathy, and hemolytic uremic syndrome), liver fibrosis (e.g., non-alcoholic steatohepatitis (NASH), chronic viral hepatitis, parasitemia, inborn errors of metabolism, toxin-mediated fibrosis, such as alcohol fibrosis, non-alcoholic steatohepatitis-hepatocellular carcinoma (NASH-HCC), primary biliary cirrhosis, and sclerosing cholangitis), cardiovascular fibrosis (e.g., cardiomyopathy, hypertrophic cardiomyopathy, atherosclerosis and restenosis,) systemic sclerosis, skin fibrosis (e.g. skin fibrosis in systemic sclerosis, diffuse cutaneous systemic sclerosis, scleroderma, pathological skin scarring, keloid, post-surgical scarring, scar revision surgery, radiation-induced scarring and chronic wounds), eye-related conditions such as subretinal fibrosis, uveitis syndrome, uveitis associated with idiopathic retroperitoneal fibrosis, extraocular muscle fibrosis, eye diseases associated with the major histocompatibility complex (MHC class I) or histocompatibility antigens, and cancers or secondary fibrosis (e.g. myelofibrosis, head and neck cancer, M7 acute megakaryoblastic leukemia and mucositis). Other diseases, disorders or conditions related to fibrosis that may be treated using compounds and/or compositions of the present disclosure, include, but are not limited to Marfan's syndrome, stiff skin syndrome, scleroderma, rheumatoid arthritis, bone marrow fibrosis, Crohn's disease, ulcerative colitis, systemic lupus erythematosus, muscular dystrophy, (such as DMD), Dupuytren's contracture, Camurati-Engelmann disease, neural scarring, dementia, proliferative vitreoretinopathy, corneal injury, complications after glaucoma drainage surgery, and multiple sclerosis (MS). Many such fibrotic indications are also associated with inflammation of the affected tissue(s), indicating involvement of an immune component. Such inflammation may be accompanied by aberrant immune cell populations, such as increased numbers of Th17 cells, reduced numbers of Treg cells, and/or both. In each case, the affected patient may exhibit increased Th17/Treg cell ratios.

In another aspect, the disclosure provides a method of selecting an isoform-specific TGFβ1 inhibitor for the treatment of a fibrotic disorder in a subject, comprising: (a) determining whether the subject manifests clinical presentations including fibrosis and one or more of the following: (i) inflammation; (ii) immune suppression; (iii) proliferative dysregulation; (iv) need for an allograft transplant; (v) at risk of severe infection; (vi) in need of a long-term TGFβ1 inhibition therapy; and (vii) manifestation of an autoimmune conditions(s); and (b) selecting an isoform-specific, context-dependent TGFβ1 inhibitor or an isoform-specific, context-independent TGFβ1 inhibitor for treatment of the fibrotic disorder based on the clinical presentations determined in step (a).

In another aspect, the disclosure provides a method of treating a subject having a fibrotic disorder, comprising (a) selecting a treatment regimen comprising an isoform-specific TGFβ1 inhibitor for the subject, said selection comprising (i) determining whether the fibrotic disorder manifests clinical presentations including fibrosis and one or more of the following: inflammation, immune suppression, proliferative dysregulation, and need for an allograft transplant; and (ii) selecting a treatment regimen comprising an isoform-specific, context-dependent TGFβ1 inhibitor or an isoform-specific, context-independent TGFβ1 inhibitor, based on the clinical presentations determined in step (i); and (b) administering the selected treatment regimen to the subject.

In one embodiment of the foregoing aspects, the fibrotic disorder manifests clinical presentations comprising fibrosis, inflammation, immune suppression, and proliferative dysregulation. In an exemplary embodiment, the fibrotic disorder is myelofibrosis, and the selected isoform-specific TGFβ1 inhibitor is an isoform-specific, context-independent TGFβ1 inhibitor.

In another embodiment, the fibrotic disorder manifests clinical presentations comprising fibrosis, inflammation, and need for an allograft transplant. In one embodiment, the fibrotic disorder manifests clinical presentations comprising fibrosis and inflammation. In another embodiment, the fibrotic disorder is a degenerative disease.

In one embodiment, the fibrotic disorder manifests clinical presentations comprising immune suppression and proliferative dysregulation. In an exemplary embodiment, the fibrotic disorder is associated with a solid tumor, and the selected isoform-specific TGFβ1 inhibitor is an isoform-specific LTBP1/3-specific inhibitor. In one embodiment, the solid tumor is a malignant tumor. In another embodiment, the tumor is a benign tumor. In one embodiment, the subject has desmoplasia, for example, pancreatic desmoplasia. In another embodiment, the subject has fibroids.

In another aspect, the disclosure provides a method of treating a subject having a fibrotic disorder with an isoform-specific, LTBP1/3-specific TGFβ1 inhibitor, comprising determining whether the fibrotic disorder manifests clinical presentations including fibrosis and the need for an allograft transplant; and administering an effective amount of an isoform-specific, LTBP1/3-specific TGFβ1 inhibitor to the subject if the fibrotic disorder manifests fibrosis and the need for an allograft transplant.

In one embodiment, the fibrotic disorder is an organ fibrosis, wherein optionally, the organ fibrosis is an advanced organ fibrosis. In a further embodiment, the organ fibrosis is selected from the group consisting of kidney fibrosis, liver fibrosis (hepatic fibrosis), lung fibrosis, cardiac fibrosis, pancreatic fibrosis, skin fibrosis, scleroderma (systemic sclerosis), muscle fibrosis, uterine fibrosis and endometriosis. In another further embodiment, the fibrotic disorder comprising chronic inflammation is a muscular dystrophy, multiple sclerosis (MS), or Cystic Fibrosis (CF). In a further embodiment, the muscular dystrophy is Duchenne muscular dystrophy (DMD). In another further embodiment, the MS comprises perivascular fibrosis. In a further embodiment, the lung fibrosis is idiopathic pulmonary fibrosis (IPF). In another further embodiment, the subject has chronic kidney disease (CKD). In another embodiment, the subject has nonalcoholic steatohepatitis (NASH).

In exemplary embodiments, the fibrotic disorder is fibrosis, Alport syndrome, scleroderma, fibroids, desmoplasia, amyotrophic lateral sclerosis (ALS), or Duchenne muscular dystrophy (DMD).

### Diseases Involving Endothelial-to-Mesenchymal Transition (EndMT)

Endothelial-to-mesenchymal transition (EndMT) is observed in vitro on exposure of endothelial cells to various tissue-damaging agents or conditions, such as TGFβ, high glucose concentrations, radiation and hypoxia. TGFβ is a key regulator of the endothelial-mesenchymal transition (EndMT) observed in normal development, such as heart formation. However, the same or similar phenomenon is also seen in many diseases, such as cancer stroma. In some disease processes, endothelial markers such as CD31 become downregulated upon TGFβ1 exposure and instead the expression of mesenchymal markers such as FSP-1, α-SMA and fibronectin becomes induced. Indeed, stromal CAFs may be derived from vascular endothelial cells. Thus, isoform-specific inhibitors of TGFβ1, such as those described herein, may be used to treat a disease that is initiated or driven by EndMT. It is contemplated that LTBP-selective approach to inhibiting the TGFβ1 signaling pathway is advantageous in controlling inflammation often associated with tissue/organ injury. In preferred embodiments, the isoform-selective activation inhibitor of TGFβ1 is Ab42, a derivative thereof, or an engineered molecule comprising an antigen-binding fragment thereof.

### Disease Involving Epithelial-to-Mesenchymal Transition (EMT)

EMT (epithelial mesenchymal transition) is the process by which epithelial cells with tight junctions switch to mesenchymal properties (phenotypes) such as loose cell-cell contacts. The process is observed in a number of normal biological processes as well as pathological situations, including embryogenesis, wound healing, cancer metastasis and fibrosis (reviewed in, for example, Shiga et al., (2015) “Cancer-Associated Fibroblasts: Their Characteristics and Their Roles in Tumor Growth.” Cancers, 7: 2443-2458). Generally, it is believed that EMT signals are induced mainly by TGFβ. In preferred embodiments, the isoform-selective activation inhibitor of TGFβ1 is Ab42, a derivative thereof, or an engineered molecule comprising an antigen-binding fragment thereof.

Epithelial cells have also been proposed to give rise to myofibroblasts by undergoing the process of EMT in several fibrotic tissues such as kidney, lung and in the liver. EMT takes place when epithelial cells lose their cuboidal shape, lose the expression of adherence and tight junction proteins, which leads to weak cell-cell contacts and reorganization of their actin cytoskeleton; while the cells acquire the expression of mesenchymal proteins (fibronectin, vimentin, N-cadherin), they adopt a fibroblast-like architecture favoring cell migration and invasion. EMT is induced by many growth factors, among them TGFβ being a very potent inducer, which regulate the expression and activity of several transcription factors known as EMT-TFs (Snail1/Snail, Snail2/Slug, ZEB1, ZEB2, Twist1/Twist and more) that are the responsible actors to execute the change in cell differentiation that is EMT. The gene and protein markers used to identify the generation of mesenchymal cells after EMT in the context of fibrosis are FSP1 (Fibroblast-specific protein 1), α-SMA and collagen I along with vimentin and desmin, whose expression increases concomitant with a reduction in levels of expression of epithelial markers (E-cadherin and certain cytokeratins). Cells that co-express epithelial and mesenchymal markers represent an intermediate stage of EMT (reviewed by, for example: Caja et al., Int. J. Mol. Sci. 2018, 19(5), 1294).

### Disease Involving Proteases

Activation of TGFβ from its latent complex may be triggered by integrin in a force-dependent manner, and/or by proteases. Evidence suggests that certain classes of proteases may be involved in the process, including but are not limited to Ser/Thr proteases such as Kallikreins, chemotrypsin, elastases, plasmin, thrombin, as well as zinc metalloproteases of ADAM family such as ADAM 10 and ADAM 17, as well as MMP family, such as MMP-2, MMP-9 and MMP-13. MMP-2 degrades the most abundant component of the basement membrane, Collagen IV, raising the possibility that it may play a role in ECM-associated TGFβ1 regulation. MMP-9 has been implicated to play a central role in tumor progression, angiogenesis, stromal remodeling and metastasis. Thus, protease-dependent activation of TGFβ1 in the ECM may be important for treating certain fibrotic diseases.

Kallikreins (KLKs) are trypsin- or chymotrypsin-like serine proteases that include plasma Kallikreins and tissue Kallikreins. The ECM plays a role in tissue homeostasis acting as a structural and signaling scaffold and barrier to suppress malignant outgrowth. KLKs may play a role in degrading ECM proteins and other components which may facilitate tumor expansion and invasion. For example, KLK1 is highly upregulated in certain breast cancers and can activate pro-MMP-2 and pro-MMP-9. KLK2 activates latent TGFβ1, rendering prostate cancer adjacent to fibroblasts permissive to cancer growth. KLK3 has been widely studied as a diagnostic marker for prostate cancer (PSA). KLK3 may directly activate TGFβ1 by processing plasminogen into plasmin, which proteolytically cleaves LAP. KLK6 may be a potential marker for Alzheimer's disease.

Known activators of TGFβ1, such as plasmin, TSP-1 and αVβ6 integrin, all interact directly with LAP. It is postulated that proteolytic cleavage of LAP may destabilize the LAP-TGFβ interaction, thereby releasing active TGFβ1. It has been suggested that the region containing 54-LSKLRL-59 (SEQ ID NO: 301) is important for maintaining TGFβ1 latency. Thus, agents (e.g., antibodies) that stabilize the interaction, or block the proteolytic cleavage of LAP may prevent TGFβ activation.

Many of these proteases associated with pathological conditions (e.g., cancer) function through distinct mechanisms of action. Thus, targeted inhibition of particular proteases, or combinations of proteases, may provide therapeutic benefits for the treatment of conditions involving the protease-TGFβ axis. Accordingly, it is contemplated that inhibitors (e.g., TGFβ1 antibodies) that selectively inhibit protease-induced activation of TGFβ1 may be advantageous in the treatment of such diseases (e.g., fibrosis and cancer). Similarly, selective inhibition of TGFβ1 activation by one protease over another protease may also be preferred, depending on the condition being treated. In preferred embodiments, the isoform-selective activation inhibitor of TGFβ1 is Ab42, a derivative thereof, or an engineered molecule comprising an antigen-binding fragment thereof.

Plasmin is a serine protease produced as a precursor form called Plasminogen. Upon release, Plasmin enters circulation and therefore is detected in serum. Elevated levels of Plasmin appear to correlate with cancer progression, possibly through mechanisms involving disruption of the extracellular matrix (e.g., basement membrane and stromal barriers) which facilitates tumor cell motility, invasion, and metastasis. Plasmin may also affect adhesion, proliferation, apoptosis, cancer nutrition, oxygen supply, formation of blood vessels, and activation of VEGF (Didiasova et al., Int. J. Mol. Sci, 2014, 15, 21229-21252). In addition, Plasmin may promote the migration of macrophages into the tumor microenvironment (Philips et al., Cancer Res. 2011 Nov. 1; 71(21):6676-83 and Choong et al., Clin. Orthop. Relat. Res. 2003, 415S, S46-S58). Indeed, tumor-associated macrophages (TAMs) are well characterized drivers of tumorigenesis through their ability to promote tumor growth, invasion, metastasis, and angiogenesis.

Plasmin activities have been primarily tied to the disruption of the ECM. However, there is mounting evidence that Plasmin also regulate downstream MMP and TGF beta activation. Specifically, Plasmin has been suggested to cause activation of TGF beta through proteolytic cleavage of the Latency Associated Peptide (LAP), which is derived from the N-terminal region of the TGF beta gene product (Horiguchi et al., J Biochem. 2012 October; 152(4):321-9), resulting in the release of active growth factor. Since TGFβ1 may promote cancer progression, this raises the possibility that plasmin-induced activation of TGFβ may at least in part mediate this process. In preferred embodiments, the isoform-selective activation inhibitor of TGFβ1 is Ab42, a derivative thereof, or an engineered molecule comprising an antigen-binding fragment thereof.

TGFβ1 has also been shown to regulate expression of uPA, which is a critical player in the conversion of Plasminogen into Plasmin (Santibanez, Juan F., ISRN Dermatology, 2013: 597927). uPA has independently been shown to promote cancer progression (e.g., adhesion, proliferation, and migration) by binding to its cell surface receptor (uPAR) and promoting conversion of Plasminogen into Plasmin. Moreover, studies have shown that expression of uPA and/or plasminogen activator inhibitor-1 (PAI-1) are predictors of poor prognosis in colorectal cancer (D. Q. Seetoo, et al., Journal of Surgical Oncology, vol. 82, no. 3, pp. 184-193, 2003), breast cancer (N. Harbeck et al., Clinical Breast Cancer, vol. 5, no. 5, pp. 348-352, 2004), and skin cancer (Santibanez, Juan F., ISRN Dermatology, 2013: 597927). Thus, without wishing to be bound by a particular theory, the interplay between Plasmin, TGFβ1, and uPA may create a positive feedback loop towards promoting cancer progression. Accordingly, inhibitors that selectively inhibit Plasmin-dependent TGFβ1 activation may be particularly suitable for the treatment of cancers reliant on the Plasmin/TGFβ1 signaling axis.

Thrombin may be involved in the activation of GARP-associated TGFβ1. Platelets are reported to express GARP-proTGFβ1. Therefore, thrombin may mediate TGFβ1 activation by targeting this axis in an integrin-independent manner.

In one aspect of the disclosure, the isoform-specific inhibitors of TGFβ1 described herein include inhibitors that can inhibit protease-dependent activation of TGFβ1. In some embodiments, the inhibitors can inhibit protease-dependent TGFβ1 activation in an integrin-independent manner. In some embodiments, such inhibitors can inhibit TGFβ1 activation irrespective of the mode of activation, e.g., inhibit both integrin-dependent activation and protease-dependent activation of TGFβ1. In some embodiments, the protease is selected from the group consisting of: serine proteases, such as Kallikreins, Chemotrypsin, Trypsin, Elastases, Plasmin, thrombin, as well as zinc metalloproteases (MMP family) such as MMP-2, MMP-9 and MMP-13.

In some embodiments, the inhibitors can inhibit Plasmin-induced activation of TGFβ1. In some embodiments, the inhibitors can inhibit Plasmin- and integrin-induced TGFβ1 activation. In some embodiments, the inhibitors are monoclonal antibodies that specifically bind TGFβ1. In some embodiments, the inhibitors can inhibit plasma kallikrein-induced activation of TGFβ1. In some embodiments, the inhibitors can inhibit plasma kallikrein-induced and Plasmin-induced activation of TGFβ1. In some embodiments, the inhibitors can inhibit Plasmin-induced, plasma kallikrein-induced and integrin-induced activation of TGFβ1. In some embodiments, the inhibitors do not significantly inhibit integrin-induced activation of TGFβ1 but inhibit protease-induced activation of TGFβ1. In some embodiments, the antibody is a monoclonal antibody that specifically binds proTGFβ1. In some embodiments, the antibody binds latent proTGFβ1 thereby inhibiting release of mature growth factor from the latent complex. In some embodiments, the inhibitor of TGFβ1 activation suitable for use in the method of inhibiting Plasmin-dependent activation of TGFβ1 is any one of the isoform-specific inhibitors disclosed herein. In some embodiments, the inhibitor is an antibody or fragment thereof that binds an epitope that comprises one or more amino acid residues of VKRKRIEA (SEQ ID NO: 9) within the α1 region of the LAP. In some embodiments, the inhibitor is an antibody or fragment thereof that binds an epitope that comprises one or more amino acid residues of DMELVKRKRIEAIR (SEQ ID NO: 46) within the α1 region of the LAP. In preferred embodiments, the isoform-selective activation inhibitor of TGFβ1 is Ab42, a derivative thereof, or an engineered molecule comprising an antigen-binding fragment thereof.

### Liver Fibrosis and Related Conditions

Liver fibrosis is often a result of chronic liver injury and is the leading cause of mortality from chronic liver diseases. Examples of chronic liver diseases include but are not limited to: chronic infection with hepatitis virus (e.g., HBV and HCV), NAFLD, alcoholic liver diseases, and autoimmune liver diseases. These conditions can progress to advanced liver fibrosis and eventually to liver cirrhosis. NAFLD is a spectrum of hepatic diseases associated with metabolic and cardiovascular disorders, such as obesity, insulin resistance, hypertension, dyslipidemia and type 2 diabetes. NAFLD is characterized by increased liver fat content, typically with a threshold of >5%, in the absence of significant alcohol consumption (defined as 30 grams/day and 20 grams/day for men and women, respectively). NAFLD can progress to nonalcoholic steatohepatitis (NASH), which can then progress to cirrhosis. However, main cause of morbimortality is associated with the cardiometabolic aspects of the liver disease. As noted previously, certain immune cells, including regulatory T cells, may impact disease progression from NAFLD to NASH, such that Tregs may serve a protective role. Thus, it is advantageous to avoid suppressing of Tregs.

Accordingly, a LTBP-selective TGFβ1 inhibitor (such as those described herein) is used in the treatment of a liver disease in a subject, wherein the treatment comprises administration of the LTBP-selective TGFβ1 inhibitor in an amount effective to treat the liver disease, wherein optionally the liver disease is selected from: non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and/or primary sclerosing cholangitis (PSC). In some embodiments, the subject has obesity, metabolic syndrome, and/or type 2 diabetis.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

Combination therapy or adjunct/add-on therapy may be considered to treat the liver disease, in which two or more therapeutic agents are used in conjunction to treat the disease, wherein the combination therapy or adjunct/add-on therapy comprises a LTBP-selective TGFβ1 inhibitor.

In some embodiments, the subject is further treated with one or more additional therapy/therapies aimed to address metabolic dysregulation associated with the liver disease. In some embodiments, the additional therapies comprise a myostatin pathway inhibitor. The myostatin pathway inhibitor may be any agent capable of suppressing myostatin signaling. In some embodiments, the myostatin pathway inhibitors useful for carrying out the present invention may be myostatin-selective inhibitors. In some embodiments, the inhibitors of the myostatin signaling pathway useful for carrying out the present invention may be myostatin non-selective inhibitors. In various embodiments, myostatin pathway inhibitor may include low molecular weight compounds (i.e., small molecules) that inhibit the pathway, as well as biologics, such as antibodies or antigen-binding fragments thereof, and engineered protein constructs that comprise ligand-binding domains (such as soluble receptor ligand traps, follistatin-based engineered constructs, and adnectins). In some embodiments, the myostatin pathway inhibitors are antibodies (e.g., antigen-binding fragments thereof or engineered constructs that comprise such fragments) that bind mature myostatin (also known as GDF-8 or GDF8). Such antibodies are typically referred to as neutralizing antibodies because they bind the growth factor thereby blocking its ability to bind endogenous receptors for activating the signaling pathway. In some embodiments, a myostatin pathway inhibitor binds a myostatin receptor thereby interfering with ligand binding. These include antibodies that bind the extracellular portion(s) of the receptor. Non-limiting examples of such antibodies include bimagrumab (BYM338) which binds ActRIIB. In preferred embodiments, the inhibitors of the myostatin signaling pathway useful for carrying out the invention are myostatin-selective inhibitors. In some embodiments, the myostatin-selective inhibitors are neutralizing antibodies that selectively bind mature myostatin (but not other growth factors such as GDF11 or Activin A). One example of such myostatin-selective antibody is trevogrumab, also known as REGN1033. In some embodiments, the myostatin-selective inhibitors are antibodies or antigen-binding fragments thereof that bind the pro/latent myostatin complex, thereby inhibiting activation (e.g., release) of mature myostatin. Such antibodies or antigen-binding fragments are referred to as “activation inhibitors” of myostatin. Non-limiting examples of myostatin-selective activation inhibitors include apitegromab (also known as SRK-015) and variants thereof, GYM329 and variants thereof, or a MST1032 variant as described in PCT/JP2015/006323.

Accordingly, a LTBP-selective TGFβ1 inhibitor (such as those described herein) is used in conjunction with a myostatin pathway inhibitor in the treatment of a liver disease in a subject, wherein the treatment comprises administration of the LTBP-selective TGFβ1 inhibitor in an amount effective to treat the liver disease, wherein optionally the liver disease is selected from: non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and/or primary sclerosing cholangitis (PSC), wherein optionally the subject has or is at risk of developing obesity, metabolic syndrome, and/or type 2 diabetes.

In some embodiments, the LTBP-selective TGFβ1 inhibitor (such as those described herein) is used in the treatment of a liver disease in a subject who is treated with a myostatin pathway inhibitor such as those listed above. In some embodiments, the myostatin pathway inhibitor (such as those listed above) is used in the treatment of a liver disease in a subject who is treated with a LTBP-selective TGFβ1 inhibitor (such as those described herein). In some embodiments, a combination of the LTBP-selective TGFβ1 inhibitor (such as those described herein) and the myostatin pathway inhibitor are used in the treatment of a liver disease in a subject in amounts effective to treat the disease. In preferred embodiments, the myostatin pathway inhibitor is a myostatin-selective inhibitor, wherein optionally the myostatin-selective inhibitor is an antibody or antigen-binding fragment thereof which binds the myostatin growth factor without binding GDF11 or Activin A, or, which binds pro/latent myostatin thereby inhibiting activation of myostatin (such as apitegromab and GYM329/RO7204239).

In some embodiments, the subject is further treated with one or more additional therapy/therapies aimed to address metabolic dysregulation associated with the liver disease. In some embodiments, the additional therapies comprise a GLP-1 pathway activator (such as GLP-1 receptor agonists). The GLP-1 pathway activator may be any agent capable of enhancing GLP-1 signaling. In preferred embodiments, the GLP-1 pathway activators useful for carrying out the present invention may be GLP-1 receptor agonists, such as GLP-1 analogs.

In some embodiments, the activators of the GLP-1 signaling pathway useful for carrying out the invention are GLP-1 receptor agonists, such as GLP-1 analogs. Endogenous GLP-1 is susceptible to degradation by peptidases and has a short half-life. Therefore, certain modifications can be introduced to the peptide hormone to generate GLP-1 analogs, aimed to prolong or stabilize its activity. In some embodiments, the GLP-1 analog may comprise the amino acid sequence EGTFTSD (SEQ ID NO: 116). In some embodiments, GLP-1 analogs comprise the amino acid sequence HXXGXFTXD (SEQ ID NO: 117), wherein X is any amino acid residue. Non-limiting examples of GLP-1 analogs include semaglutide, exenatide ER, liraglutide, lixisenatide, tirzepatide, dulaglutide, XW003, Noiiglutide, MED10382, and albiglutide. In some embodiments, the activators of the GLP-1 signaling pathway useful for carrying out the invention are inhibitors of peptidases responsible for the degradation of GLP-1. In some embodiments, the peptidase inhibitor inhibits dipeptidyl peptidase 4 (DPP-4) and/or neutral endopeptidase (NEP). In some embodiments, the activators of the GLP-1 signaling pathway useful for carrying out the invention are agents capable of modulating GLP-1 receptor trafficking (e.g., agents that cause slower internalization of the receptor); agents that increase expression of cell-surface GLP-1 receptors; agents that enhance ligand-induced activation of the GLP-1 receptor; agents that enhance pathways such as cAMP signaling that are downstream of the GLP-1 receptor, and/or agents that antagonize the phosphatase responsible for de-phosphorylating the GLP-1 receptor.

Non-limiting examples of GLP-1 analogs include albiglutide, taspoglutide, semaglutide, exenatide, BPI-3016, GW002, glutazumab, exendin-4, exenatide, GLP-1 (7-36)NH2, everestmab, liraglutide, lixisenatide, tirzepatide, XW003, Noiiglutide, MED10382, and dulaglutide. In one embodiment, the GLP-1 analog is liraglutide, which is currently approved for type 2 diabetes and obesity.

Liraglutide (Victoza®, Saxenda®, CAS Number 204656-20-2) is an acylated GLP-1 agonist derived from GLP-1(7-37). While wild-type GLP-1 has a plasma half-life of 1.5-2 minutes due to degradation by the enzyme dipeptidyl peptidase-4 (DPP-IV) and/or neutral endopeptidases, liraglutide is an acylated form of GLP-1 engineered to bind albumin when administered to a subject, thus greatly extending its half-life compared to GLP-1(7-37), with a plasma half-life of 13 hours. Liraglutide is also available in combination with insulin degludec under the name Xultophy®, Semaglutide (Ozempic®, Rybelsus®, Wegovy®, CAS Number 910463-68-2) is a modified GLP-1 peptide comprising two amino acid replacements: 2-aminoisobutyric acid replaces the alanine moiety at position 8 and arginine replaces the lysine moiety at position 34. The substitution at position 8 prevents breakdown by the enzyme DPP-IV. Semaglutide was approved by the USFDA in 2017. See, e.g., Novo Nordisk A/S. Ozempic (semaglutide) injection, for subcutaneous use; prescribing information; 2020.

Dulaglutide (Trulicity®, CAS Number 923950-08-7) comprises GLP-1(7-37) covalently linked to an Fc fragment of human IgG4. Dulaglutide is approved for adults with type 2 diabetes mellitus (combined with diet and exercise). See, e.g., Meece J., Adv Ther. 2017; 34:638-657.

Exenatide (Byetta®, Bydureon®, CAS Number 141758-74-9) is a synthetic form of the GLP-1 mimetic exendin-4 (a Gila monster saliva hormone) approved by the USFDA in 2005 for treatment of diabetes mellitus type 2 combined with diet and exercise.

Glutazumab is an antibody fusion protein engineered by linking the human GLP-1 derivative to a humanized GLP-1R antibody via a peptide linker. Li et al., Biochem Pharmacol. 2018 April; 150:46-53.

Lixisenatide (Lyxumia®, Adlyxin®, CAS Number 320367-13-3) is a GLP-1 mimetic approved for treatment of diabetes mellitus type 2 in conjunction with diet and exercise. See, e.g., Sanofi-Aventis US. Adlyxin (lixisenatide), prescribing information, 2016. Lixisenatide is also available in combination with insulin glargine under the name Soliqua®/Suliqua®.

Everestmab is a long-acting GLP-1/anti-GLP1R fusion protein comprising a mutated GLP-1(A8G) fused to tandem bispecific humanized GLP-1R targeting and albumin-binding nanobodies designed for treatment of diabetes mellitus type 2. Pan et al., ARTIFICIAL CELLS, NANOMEDICINE, AND BIOTECHNOLOGY 2020, VOL. 48, NO. 1, 854-866.

Albiglutide (Eperzan®, Tanzeum®, CAS Number 782500-75-8) is a GLP-1R agonist that was approved for type 2 diabetes mellitus. Its manufacture was discontinued in 2018 due to lack of sales. See, e.g., GlaxoSmithKline LLC. Tanzeum® (albiglutide) for injection, for subcutaneous use prescribing information; 2017.

Taspoglutide is a GLP-1R agonist that was previously under clinical investigation for treatment of diabetes mellitus type 2.

Tirzepatide (LY3298176) is a dual glucose-dependent insulinotropic polypeptide, GIP and GLP-1 receptor agonist that is under clinical investigation for the treatment of type 2 diabetes. It was made by engineering GLP-1 activity into the GIP sequence and has been shown to improve glycemic control and to reduce body weight (Rosenstock et al., Lancet (2020) 398:143-155).

BP3016 is a long-acting hGLP-1 analog comprising human GLP-1(7-37) with engineered resistance to DPP-IV cleavage. See, e.g., Pharmacol Res. 2017 August; 122:130-139.

XW003 is an acylated human glucagon-like peptide-1 (GLP-1) analog that has been shown to induce dose-dependent weight reductions in early clinical trials. The drug is currently in phase 2 stage of development for the treatment of obesity. See, e.g., ClinicalTrials.gov identifier NCT05111912.

Noiiglutide is a 40-mer exenatide analog, which acts as a glucagon-like peptide-1 (GLP-1) agonist. The drug is being developed by Jiangsu Hansoh Pharmaceutical for the treatment of obesity. It is currently in phase 2 clinical development. See, e.g., Cabri et al. Front Mol Biosci. 2021; 8: 697586.

MED10382 is an oxyntomodulin-like peptide with targeted GLP-1 and glucagon receptor activity. It is currently in phase 2 stage of clinical development for the treatment of obesity. See, e.g., Ambery et al. Lancet. 2018 Jun. 30; 391(10140):2607-2618.

According to the present disclosure, GLP-1 pathway activators include danuglipron (Pfizer), GLP-1 receptor agonist/GIP receptor antagonist combination such as AMG 133 (Amgen), mas receptor activators, e.g., Sarconeos, GLP-1/GIP combination such as tirzepatide (Eli Lilly), amylin/GLP-1 combination such as cagrilintide/semaglutide combination (Novo Nordisk), GLP-1/glucagon combination such as DD01 (Neuraly), GLP-1/glucagon combination such as ALT-801 (Altimmune), GLP-1/GIP such as CT-388 (Carmot), GLP-1/glucagon dual agonist such as 1B1362 (mazdutide) (LY-330567 (Innovent/Eli Lilly), danuglipron (PF-06882961) (Pfizer), setmelanotide (Rhythm), GLP-1/GIP/Glucagon triple receptor agonist such as LY3437943 (Eli Lilly) and a dual GIP/GLP-1 receptor agonist such as LY3298176 (Eli Lilly).

GLP-1 pathway activators further encompass agents that act on multiple pathways, including the GLP-1 pathway, such as metformin and derivatives thereof. Rena et al., Diabetologia 60:1577-85 (2017). Non-limiting examples of metaformin include Fortamet, Glucophage, Glucophage XR, Glumetza, Riomet, Obimet, Gluformin, Dianben, Diabex, Diaformin, Metsol, Siofor, Metforgamma and Glifor. GLP-1 pathway activators also include metformin-containing medications that include additional active agents. Examples include, but are not limited to, thiazolidinediones (glitazones) and rosiglitazone. In some embodiments, the GLP-1 pathway activator is FGF-2 or a peptide derived therefrom.

Accordingly, a LTBP-selective TGFβ1 inhibitor (such as those described herein) is used in conjunction with a GLP-1 pathway activator in the treatment of a liver disease in a subject, wherein the treatment comprises administration of the LTBP-selective TGFβ1 inhibitor and the GLP-1 pathway activator in amounts effective to treat the liver disease, wherein optionally the liver disease is selected from: non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and/or primary sclerosing cholangitis (PSC), wherein optionally the subject has or is at risk of developing obesity, metabolic syndrome, and/or type 2 diabetis.

In some embodiments, the LTBP-selective TGFβ1 inhibitor (such as those described herein) is used in the treatment of a liver disease in a subject who is treated with a GLP-1 pathway activator such as those listed above. In some embodiments, the GLP-1 pathway activator (such as those listed above) is used in the treatment of a liver disease in a subject who is treated with a LTBP-selective TGFβ1 inhibitor (such as those described herein). In some embodiments, a combination of the LTBP-selective TGFβ1 inhibitor (such as those described herein) and the GLP-1 pathway activator are used in the treatment of a liver disease in a subject in amounts effective to treat the disease. In preferred embodiments, the GLP-1 pathway activator is a GLP-1 analog which acts as a GLP-1 receptor agonist.

In some embodiments, triple combination/adjunct/add-on therapy is contemplated, in which a LTBP-selective TGFβ1 inhibitor, myostatin pathway inhibitor and a GLP-1 pathway activator are used in conjunction to treat the liver disease in the subject, wherein optionally the liver disease is NAFLD or NASH, wherein further optionally the subject has or is at risk of developing a metabolic disease such as obesity, metabolic syndrome and/or type 2 diabetes. In preferred embodiments, the myostatin pathway inhibitor is a myostatin-selective inhibitor. Examples of myostatin-selective inhibitors include nautralizing antibodies that bind myostatin but not GDF11 or Activin (such as REGN1033 and variants thereof), as well as activation inhibiting antibodies that bind pro/latent myostatin thereby inhibiting release of the growth factor from the latent complex (such as apitegromab, GYM329, and variants thereof). In further preferred embodiments, the GLP-1 pathway activator is a GLP-1 receptor agonist, such as GLP-1 analogs. Applicant has obtained preclinical mouse data showing that a combination of a myostatin-selective inhibitor and a GLP-1 receptor agonist (e.g., liraglutide) can achieve overall body weight loss, enhanced fat mass reduction, retention of lean mass, and overall enhancement of body composition, as compared to the GLP-1 receptor agonist alone, in a diet-induced obesity model. Based on these observations, together with the anti-fibrotic effects observed with LTBP-selective inhibition approach, it is contemplated herein that addition of a LTBP-selective TGFβ1 inhibitor to the myostatin inhibitor/GLP-1 combination may further provide anti-fibrotic effects in NASH, or potentially prevent or delay the disease progression from NAFLD to NASH in a subject, by addressing both the metabolic aspect and liver injury-induced fibrosis.

In some embodiments, the subject also receives a FGF21 receptor agonist or a FGF19 receptor agonist. Zhao et al., Signal Transduction and Targeted Therapy (2022) 7:206. In some embodiments, the subject also receives, or has received, one or more of the following therapeutics to treat the liver disease: Hydronidone, B1089-100, Efruxifermin, Pegbelfermin, Aldafermin, MK-3655, PRI-724, Selonsertib, CC-90001, Epeleuton, Elafibranor, Saroglitazar, Lanifibranor, Pemafibrate, ZSP0678, Obeticholic Acid, Cilofexor, Nidufexor, TERN-101, Vonafexor, EDP-305, Tropifexor, JKB-121, JKB-122, Semaglutide, Tirzepatide, Cotadutide, HM-15211, Resmetirom, VK2809, Cenicriviroc, Belapectin, GB1211, Azemiglitazone potassium, Deuterium-Stabilized (R)-Pioglitazone, Aramchol, PF-05221304, Firsocostat, ZSP1601, Epeleuton, PXL-770, ALS-L1023, Namodenoson, TVB-2640, LPCN 1144, HepaStem, BMS-986263, Foralumab, Elobixibat, Apararenone, PF-06835919, ARO-HSD, and CB4211.

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

### Renal (Kidney) Fibrosis and Related Conditions

Renal fibrosis is a progressive pathological feature of chronic kidney diseases (CKD) and may be associated with conditions such as diabetes, proteinuria, hypertension, glomerular disease and crystal deposition. Chronic kidney disease is defined based on the presence of either kidney damage or decreased kidney function for three or more months, irrespective of cause. Chronic kidney disease (CKD) is common in people with both type 1 and type 2 diabetes. It is defined by the presence of reduced glomerular filtration rate (GFR) and/or increased urinary albumin excretion for at least three months.

Patients with end-stage kidney disease (ESKD) are known to display a cellular immune dysfunction, such as micro-inflammation and atherogenesis which may be in part mediated by reduced Tregs in these patients. Indeed, in most, if not all, studies on inflammatory diseases, including acute and chronic renal disease, depletion of Tregs worsens the disease, whereas Treg supplementation offers robust protection (reviewed for example by Sharma & Kinsey (2017) Am J Physiol Renal Physiol 314: F679-F698).

For example, in acute kidney injury, Treg depletion worsened the inflammation, acute tubular necrosis and renal function assessed by plasma creatinine, whereas Treg transfer showed the protective effect of Tregs. Similarly, in cisplatin-induced renal injury model, Tregs were shown to protect from cisplatin-induced renal dysfunction and damage. By contrast, Treg depletion in wild type mice exacerbated the cisplatin-induced injury.

Role of Tregs in glomerulopathies have been reported. Autoimmune diseases such as systemic lupus erythematosus (SLE or lupus), Goodpasture's syndrome, IgA nephropathy (idiopathic), and others often have glomerular manifestations. SLE is a systemic autoimmune disease manifested as increased production of autoantibodies, immune complex deposition, and multiorgan inflammation. Kidneys are affected in over 50% of lupus patients, leading to glomerulonephritis, with a fifth of these patients proceeding to ESRD. Clinical and experimental studies show that Treg deficiency correlates with incidence and severity of lupus.

Diabetic kidney disease represents the leading cause of kidney failure (e.g., ESRD and mortality). In fact, about 1 in 3 adults with diabetes has CKD. Both type 1 and type 2 diabetes are affected. Tregs play a role in suppressing inflammation (e.g., in type 2 diabetes) and preventing severe autoimmune diseases (e.g., in type 1 diabetes).

Therefore, when the ability of Tregs to become activated, proliferate and/or be recruited to disease loci is impaired, tissue inflammation induced by various factors (e.g., ischemia reperfusion, nephrotoxic drugs, lupus nephritis, diabetic nephropathy, etc.) cannot be sufficiently suppressed, ultimately leading to end-stage renal disease. Advantageously, the LTBP-selective inhibitors of TGFβ1 activation can selectively target matrix-associated TGFβ1, sparing Tregs expressing cell-surface GARP.

Accordingly, the present disclosure includes therapeutic use of LTBP-selective TGFβ1 inhibitors for treating CKD. In some embodiments, a LTBP-selective TGFβ1 inhibitor is used in the treatment of CKD in a subject, wherein the treatment comprises administration of the LTBP-selective TGFβ1 inhibitor in an amount sufficient to treat CKD. In some embodiments, the subject has diabetes, wherein optionally the diabetes is type 2 diabetes or type 1 diabetes.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In some embodiments, CKD is associated with a genetic mutation. In some embodiments, the subject has Alport syndrome. Alport syndrome is a rare genetic disorder of specialized basement membranes in the kidney, ear, and eye and is caused by mutations in genes for type IV collagen (Col4A3, Col4A4 and Col4A5). Most affected individuals experience progressive loss of kidney function due in part to impaired renal filtration, which may lead to end-stage kidney disease. People with Alport syndrome also frequently develop sensorineural hearing loss in late childhood or early adolescence.

A 2021 report by Chimenz et al. (J Nephrol. 2021 December; 34(6):1915-1924) evaluated urinary (u) and serum (s) levels of tumor growth factor (TGF)-beta(β) and high mobility group box (HMGB)-1 in ALP patients with normal renal function, albuminuria and proteinuria. This group found that ALP patients had significantly higher levels of serum and urinary TGF-β1 compared to healthy subjects, and thus concluded that high levels of HMGB1 and TGF-β1 characterized ALP patients with normal renal function, highlighting the subclinical pro-fibrotic and inflammatory mechanisms triggered before the onset of proteinuria.

Currently there are limited treatment options for Alport syndrome, focused primarily on supportive care. These include, but are not limited to: angiotensin converting enzyme (ACE) inhibitors and angiotensin II type 1 receptor blockers (ARBs), and renal transplant.

In some embodiments, a LTBP-selective TGFβ1 inhibitor is used in the treatment of Alport syndrome in a subjec, wherein the treatment comprises administration of the TGFβ1 inhibitor in an amount effective to treat the disease, wherein optionally the subject is treated with an ACE inhibitor, an ARB and/or an endothelin antagonist.

In some embodiments, the subject suffering from kidney fibrosis or CKD may be further treated with a background therapy aimed to manage or treat diabetes. In some embodiments, the subject is on a background therapy containing a biguanide such as metformin or a derivative thereof. Non-limiting examples of metaformin include Fortamet, Glucophage, Glucophage XR, Glumetza, Riomet, Obimet, Gluformin, Dianben, Diabex, Diaformin, Metsol, Siofor, Metforgamma and Glifor, as well as metformin-containing medications that include additional active. Examples include, but are not limited to, thiazolidinediones (glitazones) and rosiglitazone. In some embodiments, the subject is on a medication for type 2 diabetes. The anti-diabetic medication may be an alpha-glucosidase inhibitor; a dipeptidyl peptidase-4 (DPP-4) inhibitor such as alogliptin, linagliptin, saxagliptin or sitagliptin; a sodium-glucose cotransporter-2 (SGLT-2) inhibitor such as dapagliflozin, canagliflozin, empagliflozin, or ertugliflozin; a sulfonylurea; or a thiazolidinedione.

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

A significant fraction of CKD patients suffer from anemia (i.e., anemia of chronic kidney disease). Anemia associated with CKD may in part be caused by myelophthisis, characterized by the presence of immature erythrocytes in the peripheral blood due to infiltration of the bone marrow by abnormal tissue. Myelophthisic anemia results from fibrosis and crowding out of the normal bone marrow owing to infiltration by non-hematopoietic or abnormal cells. Therefore, in addition to treating kidney fibrosis, LTBP-selective TGFβ1 inhibitors described herein may also treat fibrosis in the bone marrow and alleviate anemia. In addition, the patients may further receive a therapy aimed to address anemia.

### Lung Fibrosis and Related Conditions

Chronic respiratory diseases may be caused by various factors, including but are not limited to allergens, chemicals, radiation, microbial agents, and environmental particles. Prolonged exposure to such factors may result in lung fibrosis. In some embodiments, chronic respiratory diseases comprise pneumoconiosis. In some embodiments, chronic respiratory diseases comprise IPF. In some embodiments, chronic respiratory diseases are associated with a gene mutation. In some embodiments, chronic respiratory diseases comprise a chronic infection.

An inflammatory disease with dysregulated TGFb1 includes certain infections, e.g., viral infections and bacterial infections. In some embodiments, the viral infection is a severe acute respiratory syndrome, such as COVID19, which manifests an inflammatory response. Patients with severe acute respiratory syndrome may develop a pulmonary fibrosis. According to the present disclosure, LTBP-selective TGFb1 inhibitors may be used to treat such conditions.

Patients with acute respiratory distress syndrome who are mechanically ventilated often develop pulmonary fibrosis. It has been suggested that stress induced by mechanical ventilation may cause tissue damage that induces fibrogenesis, such as damage to the alveolar epithelium, biotrauma, etc, via epithelial-mesenchymal transition. Such exposure to mechanical stress could trigger wound/repair response in the lung tissue, involving inflammation. Therefore, it is contemplated that LTBP-selective approach to inhibiting TGFβ1 may be advantageous in treating the condition.

Radiation is a well-established therapy for thoracic neoplasms. However, the lung is a radiosensitive organ and radiation-induced lung injury (e.g., radiation-induced pneumonitis and pulmonary fibrosis) poses a treatment challenge. Regulatory T cells (Tregs) may play a key role in the pathogenesis of radiation-induced lung injury, a frequent and potentially life-threatening complication of thoracic radiotherapy. Therefore, use of LTBP-selective TGFβ1 inhibitors may provide an advantageous treatment option.

Accordingly, a LTBP-selective TGFβ1 inhibitor (such as Ab42) is used as an adjunct therapy in the treatment of radiation-induced lung injury in a subject who receives radiation therapy aimed to treat thoracic neoplasm (e.g., lung cancer, head and neck cancer, etc.).

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

### Skeletal Muscle Fibrosis and Related Conditions

Accumulating evidence indicates that TGFβ plays an important role in muscle homeostasis, repair, and regeneration. In skeletal muscle, TGFβ plays a variety of roles including inhibition of proliferation and differentiation, induction of atrophy, and development of fibrosis. The hallmark of DMD is excessive, and progressive, fibrosis. TGFβ1 has been implicated in human muscular dystrophies. Duchenne muscular dystrophy (DMD) is a severe, progressive, and ultimately fatal disease caused by the absence of dystrophin (Bushby, K., et al., Lancet Neurol, 2010. 9(1): p. 77-93). Lack of dystrophin results in increased susceptibility to contraction-induced injury, leading to continual muscle degeneration (Petrof, B. J., et al., Proc Natl Acad Sci USA, 1993. 90(8): p. 3710-4; Dellorusso, C., et al., J Muscle Res Cell Motil, 2001. 22(5): p. 467-75; Pratt, S. J., et al., Cell Mol Life Sci, 2015. 72(1): p. 153-64). Repeated rounds of repair contribute to chronic inflammation, fibrosis, exhaustion of the satellite cell pool, eventual loss of mobility and death (Bushby, K., et al., Lancet Neurol, 2010. 9(1): p. 77-93; McDonald, C. M., et al., Muscle Nerve, 2013. 48(3): p. 343-56). Expression of TGFβ1 is significantly increased in patients with DMD and correlates with the extent of fibrosis observed in these patients (Bernasconi, P., et al., J Clin Invest, 1995. 96(2): p. 1137-44; Chen, Y. W., et al., Neurology, 2005. 65(6): p. 826-34). Excessive ECM deposition has detrimental effects on the contractile properties of the muscle and can limit access to nutrition as the myofibers are isolated from their blood supply (Klingler, W., et al., Acta Myol, 2012. 31(3): p. 184-95). In human patients, there is a strong correlation between the extent of TGFβ1 upregulation and fibrosis, and a strong link between the extent of fibrosis and negative mobility outcomes.

For these reasons, multiple groups have previously evaluated TGFβ pathway inhibitors, such as neutralizing antibodies (pan-inhibitors of TGFβ1, 2, and 3 such as 1 D11), TGFβ receptor antagonists (e.g., Alk5 inhibitors and dominant-negative receptors) aimed to address fibrosis associated with DMD and observed some anti-fibrotic effects at least in preclinical models. None has to date succeeded in the clinic.

As alluded to above, apart from severe fibrosis, muscular dystrophies such as DMD manifest inflammation. While TGFβ inhibition may address the fibrotic feature of the disease by reducing excess ECM deposit such as collagen build-up, TGFβ also has immunosuppressive effects. Therefore, broad inhibition of this pathway may cause immune stimulation, which could exacerbate inflammation of the dystrophic muscle. Indeed, this notion is supported by literature. For example, Villalta et al. (Sci Transl Med. 6(258): 258ra142) reported that regulatory T cells suppressed muscle inflammation and injury in a murine muscular dystrophy model. Although Tregs were largely absent in the muscle of wild-type mice and normal human muscle, they were present in necrotic lesions, and depletion of Tregs exacerbated muscle injury and the severity of muscle inflammation. These findings suggest that Tregs modulate the progression of muscular dystrophy by suppressing inflammation in muscle associated with muscle fiber injury.

Applicant recognized the need for a therapy that addresses muscle fibrosis, while minimizing the risk of Treg inhibition for the treatment of muscle conditions involving both fibrosis and inflammation. Agents, such as monoclonal antibodies described herein, that selectively modulate LTBP-associated TGFβ1 signaling may be effective for treating damaged muscle fibers, such as in chronic/genetic muscular dystrophies, congenital fibrosis of ocular/extraocular muscles, and acute muscle injuries, without the toxicities associated with more broadly-acting TGFβ inhibitors.

Accordingly, the present disclosure provides methods for treating damaged muscle fibers using an agent that preferentially modulates a subset, but not all, of TGFβ effects in vivo. Such agents can selectively modulate TGFβ1 signaling (“isoform-specific modulation”) in a particular context, i.e., when presented by LTBP1 or LTBP3. In preferred embodiments, the agent is an antibody that selectively binds LTBP1/3-proTGFβ1 thereby inhibiting release of TGFβ1 from the latent complex, wherein optionally the antibody is Ab42 or a variant thereof. Alternatively, in some embodiments, the agent is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above).

Additionally or alternatively, in some embodiments, the agent is an antibody which is not Ab42 and competes binding with Ab42 and binds an epitope comprising one or more residues of the amino acid stretch YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In some embodiments, the LTBP-selective TGFβ1 inhibitor is used in conjunction with another therapy aimed to treat the muscle, such as dystrophin-directed therapy. Such therapies include, but are not limited to: exon skipping therapies (e.g., Golodirsen, Eteplirsen, Viltolarsen, Casimersen, SRP-5051, DS-514-1 b); stop codon readthrough therapies (e.g., Ataluren, NPC14/Arbekacin sulfate); gene addition therapies (e.g., PF-06939926, rAAVrh74.MHCK7, SGT-001); genome editing therapies (CRISPR-Cas9); protein replacement therapies (C1100/Ezutromid, rAAVrh74.MCK.GALGT2); myoblast transplantation (e.g., donor-derived myoblasts). Thus, the LTBP-selective TGFβ1 inhibitor is used in the treatment of DMD in a subject, wherein the treatment comprises administration of the LTBP-selective TGFβ1 inhibitor in an amount effective to treat DMD-associated inflammation and/or fibrosis, wherein the subject is treated with any one of the therapies listed above (e.g., dystrophin-directed therapies).

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

### Cardiac Fibrosis and Related Conditions

Cardiac fibrosis is often associated with cardiovascular disorders (e.g., atherosclerosis, hypertension, heart failure, pulmonary arterial hypertension, abnormal aortic aneurysm, left venticular hypertrophy, atrial fibriation, postischemic neovascularization, myocardititis and dilated cardiomyopathy and myocardial infarction) and manifests as either reactive interstitial fibrosis or replacement fibrosis. The latter typically occurs in response to ischemia, ischemia.reperfusion, inflammation, and/or toxic injury to the cardiac tissues. Ischemic injury triggers an inflammatory response that is essential for tissue repair, but excessive and prolonged or chronic immune responses can lead to cardiovascular diseases, including cardiac fibrosis. Regulatory T cells (Tregs) serve an important role in regulating self-tolerance and immune suppression thereby preventing excessive inflammation and participate in tissue repair following an ischemic event (reviewed in, for example, Zhuang & Feinberg (2020) J Mol Cell Cardiol, 147: 1-11).

Therefore, it is contemplated that LTBP-selective TGFβ1 inhibitors may be used in the treatment of cardiovascular disorders (e.g., cardiac fibrosis) in a subject in an amount effective to treat the disorder, wherein the cardiovascular disorder involves inflammation of the affected tissue.

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

### Systemic Sclerosis, Skin Fibrosis and Related Conditions

In some embodiments, the subject has scleroderma. Scleroderma is a chronic, autoimmune disease in which normal tissue is replaced with dense, thick fibrous tissue. Abnormal vasculature, inflammation, and fibrosis are main characteristics of the disease. Lung fibrosis is a major cause of mortality in patients with systemic sclerosis (SSc), a form of scleroderma. Even though characteristics of the disease have been well documented, the pathogenesis of scleroderma remains largely unknown. Fibrosis of the skin due to an accumulation of collagen is a common presenting finding of the disease, and the extent of skin involvement is one way in which scleroderma or SSc is classified.

Increased serum and tissue levels of TGFβ1 have been observed in patients with scleroderma suggesting its role in the pathogenesis of fibrosis associated with the disease. Dantas et al. (Disease Markers 14 Nov. 2016) found raised active TGFβ1 serum levels in SSc patients, and a significant association between active TGFβ1 serum levels and vascular (digital ulcers) and fibrotic (lung and skin) manifestations in SSc patients, suggesting that raised levels could be evaluated as a marker of more advanced disease.

Due to inherent challenges of studying scleroderma or SSc in patients, many inducible and genetic animal models have been developed for the study of initial events, genes, and other influences on manifestation of the disease. For example, tight skin 1 mice (Tsk1+/+) with a homozygous lethal mutation that causes thickened skin firmly bound to the subcutaneous tissue have proven useful for studying the efficacy of drugs that target fibrosis. Another animal model utilizes mice with double heterozygous deficiency of two transcription factors, Friend leukemia integration 1 (Fli1) and Kruppel-like factor 5 (KLF5), to mimic the epigenetic phenotype of SSc skin (Noda et al., Nat Commun. 2014; 5: 5797).

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (I); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

The inhibitors, e.g., antibodies, described herein can be administered to a subject in an amount effective to treat or reduce symptoms of fibrosis. The effective amount of such an inhibitor is an amount effective to achieve both therapeutic efficacy and clinical safety in the subject. In one embodiment, an effective amount is an amount effective to reduce TGFβ1 activity in the extracellular matrix. In another embodiment, an effective amount is an amount effective to reduce fibrosis in a subject. In another embodiment, the effective amount does not inhibit TGFβ31-mediated immune suppression. In some embodiments, such an inhibitor, e.g., antibody, is a context-specific inhibitor that can block activation of TGFβ1 that is mediated by an LTBP-containing, ECM-associated TGFβ1. In some embodiments, the LTBP is LTBP1 and/or LTBP3. Assays useful for determining the efficacy of the inhibitors, e.g., antibodies, and/or compositions of the present disclosure for the alteration of fibrosis include, but are not limited to, histological assays for counting fibroblasts and basic immunohistochemical analyses known in the art.

### Cardiovascular Diseases/Ischemia

Hypoxia (such as prolonged or chronic hypoxia) can trigger cardiac repair pathways that lead to scarring or cardiac fibrosis.

In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (1); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

In some embodiments, the subject also receives, or has received, one or more of Treg-enhancing agents, such as all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

### Other Diseases and Uses

In some embodiments, non-fibrotic conditions may be treated with the compositions comprising a LTBP-selective TGFβ1 inhibitor and/or methods described herein. In preferred embodiments, the TGFβ1 inhibitor is Ab42 or a variant thereof. Alternatively, in some embodiments, the TGFβ1 inhibitor is an antibody which is not Ab42 and competes binding with Ab42 and binds an epitope comprising one or more residues of the N-terminal stretch of amino acids DMELVKRKRIEAIR (SEQ ID NO: 46) within the so-called “alpha-1 helix” region of the prodomain of the latent proTGFβ1 complex. In some embodiments, the antibody or the fragment contacts one or more of the following residues within the prodomain: Asp27 (D); Leu30 (L); Arg33 (R); Ile36 (1); Glu37 (E) (shown in bold above). Additionally or alternatively, in some embodiments, the inhibitor is an antibody which is not Ab42 and competes binding with Ab42 and binds an epitope comprising one or more residues of the amino acid sequence YIDFRKDLGWK (SEQ ID NO: 93) within the TGFβ1 growth factor domain (e.g., the “finger-1” domain). In some embodiments, the antibody or fragment thereof contacts one or more of the following residues: Tyr289 (Y); Lys294 (K); Asp295 (D) (shown in bold above). In some embodiments, such antibody binds a conformational epitope comprising one or more residues of DMELVKRKRIEAIR (SEQ ID NO: 46) and one or more residues of YIDFRKDLGWK (SEQ ID NO: 93), wherein the antibody is not Ab42.

LTBP-selective TGFβ1 inhibitors are contemplated to be used in conjunction with a genotoxic therapy (such as radiation therapy and chemotherapy), or in a subject exposed to a genotoxic or tissue-damaging agent (such as radiation/irradiation and carcinogens). Such use is aimed to inhibit ROS-induced TGFβ1 activation without depleting Tregs and immunosuppressive macrophages which are beneficial for reducing treatment-induced or tissue-damaging agent-induced inflammation.

Genotoxic agents such as radiation therapy and chemotherapy can induce oxidative stress involving reactive oxygen species. This can also trigger a rapid immune response (e.g., inflammation) which can lead to tissue damage and/or scarring. Without being bound by a particular theory, it is believed that this process is at least in part mediated by ROS-induced activation of TGFβ1. Tregs are effective in dampening or preventing an excessive immune response and reducing fibrosis. At the same time, Tregs can promote polarization of macrophages into a more immunosuppressive phenotype. For these reasons, it is envisaged that it is beneficial to avoid the inhibition of GARP-associated and LRRC33-associated TGFβ1.

As cancer combination or adjunct therapy, the present disclosure provides a LTBP-selective TGFβ1 inhibitor for use in the treatment of cancer in a subject who receives a genotoxic therapy (e.g., radiation therapy and/or chemotherapy) wherein the treatment comprises administration of an effective amount of the LTBP-selective TGFβ1 inhibitor to treat the cancer. In some embodiments, the cancer comprises a solid tumor. In some embodiments, the cancer is a blood cancer. In some embodiments, the cancer is myelofibrosis, wherein optionally the myelofibrosis is primary myelofibrosis (PMF). In some embodiments, the LTBP-selective TGFβ1 inhibitor and the genotoxic therapy (e.g., radiation therapy and/or chemotherapy) are used in combination for the treatment of cancer in a subject in amounts effective to treat the cancer. In some embodiments, the LTBP-selective TGFβ1 inhibitor is administered to the subject prior to the subject receiving the genotoxic therapy.

In various embodiments, the cancer to be treated with the LTBP-selective TGFβ1 inhibitor may be carcinoma, wherein optionally the carcinoma is a basal cell carcinoma, squamous cell carcinoma, transitional cell carcinoma, renal cell carcinoma, adenocarcinoma. In some embodiments, the basal cell carcinoma is basal cell carcinoma of the skin. In some embodiments, the squamous cell carcinoma (SCC) is squamous cell carcinoma of the skin (cutaneous SCC), SCC of the lung, SCC of the esophagus, SCC of the head and neck. In some embodiments, the transitional cell carcinoma is a transitional cell carcinoma of the kidney. In some embodiments, the adenocarcinoma is breast adenocarcinoma, colorectal adenocarcinoma, lung adenocarcinoma, pancreatic adenocarcinoma, or prostate adenocarcinoma. Preferably, the LTBP-selective TGFβ1 inhibitor is used in conjunction with a genotoxic therapy, wherein optionally the genotoxic therapy comprises radiation therapy and/or chemotherapy.

In various embodiments, the cancer to be treated with the LTBP-selective TGFβ1 inhibitor is: uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), head and neck squamous cell carcinoma (HNSC), glioblastoma multiforme (GMB), esophageal carcinoma (ESCA), colon adenocarcinoma (COAD), breast invasive carcinoma (BRCA), or bladder urothelial carcinoma (BLCA).

Antibodies or antigen-binding fragments thereof encompassed by the present disclosure may be used in the treatment of cancer, including, without limitation: myelofibrosis, melanoma, adjuvant melanoma, renal cell carcinoma (RCC), including clear cell RCC, papillary RCC, chromophobe RCC, collecting duct RCC, or unclassified RCC, bladder cancer, colorectal cancer (CRC) (e.g., microsatellite-stable CRC, mismatch repair deficient colorectal cancer), colon cancer, rectal cancer, anal cancer, breast cancer, triple-negative breast cancer (TNBC), HER2-negative breast cancer, HER2-positive breast cancer, BRCA-mutated breast cancer, hematologic malignancies, non-small cell carcinoma, non-small cell lung cancer/carcinoma (NSCLC), small cell lung cancer/carcinoma (SCLC), extensive-stage small cell lung cancer (ES-SCLC), lymphoma (classical Hodgkin's and non-Hodgkin's), primary mediastinal large B-cell lymphoma (PMBCL), T-cell lymphoma, diffuse large B-cell lymphoma, histiocytic sarcoma, follicular dendritic cell sarcoma, interdigitating dendritic cell sarcoma, myeloma, chronic lymphocytic leukemia (CLL), acute myeloid leukemia (AML), small lymphocytic lymphoma (SLL), head and neck cancer (e.g., head and neck squamous cell cancer), urothelial cancer e.g., metastatic urothelial carcinoma), merkel cell carcinoma (e.g., metastatic merkel cell carcinoma), merkel cell skin cancer, cancer with high microsatellite instability (MSI-H), cancer with mismatch repair deficiency (dMMR), tumor mutation burden high cancer, mesothelioma (e.g., malignant pleural mesothelioma), gastric cancer, gastroesophageal junction cancer (GEJ), gastric adenocarcinoma, neuroendocrine tumors, gastrointestinal stromal tumors (GIST), gastric cardia adenocarcinoma, renal cancer, biliary cancer, cholangiocarcinoma, pancreatic cancer, prostate cancer, adenocarcinoma, squamous cell carcinoma, non-squamous cell carcinoma, cutaneous squamous cell carcinoma (CSCC), ovarian cancer, endometrial cancer, fallopian tube cancer, cervical cancer, peritoneal cancer, stomach cancer, brain cancers, malignant glioma, glioblastoma, gliosarcoma, neuroblastoma, thyroid cancer, adrenocortical carcinoma, oral intra-epithelial neoplasia, esophageal cancer, nasal cavity and paranasal sinus squamous cell carcinoma, nasopharynx carcinoma, salivary gland cancer, liver cancer, basal cell carcinoma; and hepatocellular cancer (HCC). However, any cancer (e.g., patients with such cancer) in which TGFβ1 is overexpressed or is at least a predominant isoform, as determined by, for example biopsy, may be treated with an isoform-selective inhibitor of TGFβ1 in accordance with the present disclosure.

In some embodiments, isoform-specific inhibitors of TGFβ1 described herein can be used to treat or prevent heterotopic ossification. Heterotopic ossification (HO) is a diverse pathologic process, defined as the formation of extraskeletal bone in muscle and soft tissues. The classic presentation of nongenetic HO is in young adults with a clear history of local trauma or surgery. HO is well documented to occur at increased frequency with certain predisposing conditions, including orthopedic surgery, bone fracture or dislocation, high-energy extremity trauma, traumatic brain and spinal cord injury and other neurologic disorders, and severe burns. See., e.g., Meyers et al., JBMR Plus. 2019 April; 3(4): e10172, incorporated by reference in its entirety herein.

In some embodiments, isoform-specific inhibitors of TGFβ1 described herein can be used to treat or prevent osteogenesis imperfecta. Osteogenesis imperfecta (01) is a group of genetic disorders that mainly affect the bones. There are at least 19 recognized forms of osteogenesis imperfecta, designated type I through type XIX. Several types are distinguished by their signs and symptoms, although their characteristic features overlap. Increasingly, genetic causes are used to define rarer forms of osteogenesis imperfecta. Type I (also known as classic non-deforming osteogenesis imperfecta with blue sclera) is the mildest form of osteogenesis imperfecta. Type II (also known as prenatally lethal osteogenesis imperfecta) is the most severe. Other types of this condition, including types III (progressively deforming osteogenesis imperfecta) and IV (common variable osteogenesis imperfecta with normal sclera), have signs and symptoms that fall somewhere between these two extremes. The milder forms of osteogenesis imperfecta, including type I, are characterized by bone fractures during childhood and adolescence that often result from minor trauma, such as falling while learning to walk. Other types of osteogenesis imperfecta are more severe, causing frequent bone fractures that are present at birth and result from little or no trauma. Additional features of these types can include blue sclera of the eyes, short stature, scoliosis, joint deformities (contractures), hearing loss, respiratory problems, and a disorder of tooth development called dentin genesis imperfecta. The most severe forms of osteogenesis imperfecta, particularly type II, can include an abnormally small, fragile rib cage and underdeveloped lungs.

In the treatment of various conditions discussed herein where Tregs serve a beneficial (e.g., protective) function, LTBP-selective TGFβ1 inhibitors such as those disclosed herein may be used in conjunction with one or more Treg-enhancing agents which are capable of promoting or enhancing the maintenance, proliferation, recruitment and/or function of Tregs. Non-limiting examples of Treg-enhancing agents include: all-trans retinoic acid (ATRA), vitamin D3, indoleamine-pyrrole 2,3-dioxygenase (IDO), short-chain fatty acids such as butyrate, and AKT/mTOR pathway inhibitors, such as rapamycin. Because Tregs dampen inflammation and/or alleviate or prevent autoimmune responses, Treg-enhancing agents may help regulate an immune response, while the LTBP-selective TGFβ1 inhibitors can inhibit TGFβ1 activation in the affected tissue or organ without significantly suppressing immunosuppressive Tregs and macrophages.

### Administration

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, inhibitors, e.g., antibodies, or antigen binding fragment thereof, that selectively bind a LTBP1-TGFβ1 complex and/or a LTBP3-TGFβ1 complex can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a TGFβ-related indication, such as those noted above. A subject having a TGFβ-related indication can be identified by routine medical examination, e.g., laboratory tests, organ functional tests, CT scans, or ultrasounds. A subject suspected of having any of such indication might show one or more symptoms of the indication. A subject at risk for the indication can be a subject having one or more of the risk factors for that indication.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present disclosure, the intended purpose may be to inhibit TGFβ-1 activation in vivo, to achieve clinically meaningful outcome associated with the TGFβ-1 inhibition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a TGFβ-related indication. Alternatively, sustained continuous release formulations of an antibody that selectively binds a LTBP1-TGFβ1 complex and/or a LTBP3-TGFβ1 complex may be appropriate. Various formulations and devices for achieving sustained release would be apparent to the skilled artisan and are within the scope of this disclosure.

In one example, dosages for an inhibitor, e.g., antibody, that selectively binds a LTBP1-TGFβ1 complex and/or a LTBP3-TGFβ1 complex as described herein may be determined empirically in individuals who have been given one or more administration(s) of the inhibitor. Individuals are given incremental dosages of the inhibitor. To assess efficacy, an indicator of the TGFβ-related indication can be followed. For example, methods for measuring for myofiber damage, myofiber repair, inflammation levels in muscle, and/or fibrosis levels in muscle are well known to one of ordinary skill in the art.

The present disclosure encompasses the recognition that agents capable of modulating the activation step of TGFβs in an isoform-specific manner, and a context-specific manner, may provide improved safety profiles when used as a medicament. Accordingly, the disclosure includes inhibitors, e.g., antibodies and antigen-binding fragments thereof, that selectively bind and inhibit activation of TGFβ1, but not TGFβ2 or TGFβ3, thereby conferring specific inhibition of the TGFβ1 signaling in vivo while minimizing unwanted side effects from affecting TGFβ2 and/or TGFβ3 signaling. Likewise, the disclosure includes inhibitors, e.g., antibodies and antigen-binding fragments thereof, that selectively inhibit activation of TGFβ1 presented by LTBP1 and/or LTBP3, but not TGFβ1 presented by GARP or LRRC33, thereby conferring specific inhibition of LTBP1/3-associated TGFβ1 signaling in vivo while minimizing unwanted side effects caused by modulation of GARP-associated TGFβ1 and/or LRRC33-associated TGFβ1.

In some embodiments, the inhibitors, e.g., antibodies, or antigen binding fragment thereof, as described herein, are not toxic when administered to a subject. In some embodiments, the inhibitors, e.g., antibodies, or antigen binding fragment thereof, as described herein, exhibit reduced toxicity when administered to a subject as compared to an antibody that binds to both TGFβ1 and TGFβ2. In some embodiments, the inhibitors, e.g., antibodies, or antigen binding fragment thereof, as described herein, exhibit reduced toxicity when administered to a subject as compared to an inhibitor that binds to both TGFβ1 and TGFβ3. In some embodiments, the inhibitors, e.g., antibodies, or antigen binding fragment thereof, as described herein, exhibit reduced toxicity when administered to a subject as compared to an inhibitor that binds to TGFβ1, TGFβ2 and TGFβ3.

Generally for administration of any of the inhibitors, e.g., antibodies, described herein, an initial candidate dosage can be about 0.5-30 mg/kg per dose, e.g., about 0.5 mg/kg, about 1 mg/kg, about 2 mg/kg, about 5 mg/kg, about 10 mg/kg, about 15 mg/kg, about 20 mg/kg, about 30 mg/kg per dose. In some embodiments, frequency of administration may be adjusted to, for example, once a week, every two weeks, every three weeks, every four weeks, every month, every six weeks, every two months, every three months, every four months, every five months, every six months, etc. For repeated administrations over several days or longer, depending on the condition, the treatment is sustained until a desired suppression of symptoms occurs or until sufficient therapeutic levels are achieved to alleviate a TGFβ-related indication, or a symptom thereof.

According to some embodiments, at least one TGFβ inhibitor, e.g., antibody or antigen-binding portion thereof, described herein, is administered as one or more initial loading dose(s) followed by one or more maintenance or treatment doses. The term “loading dose” refers to a first dose(s) of TGFβ inhibitor, e.g., antibody or antigen-binding portion thereof, which is initially used to treat a subject who may benefit from TGFβ inhibition in vivo. The loading dose may be larger in comparison to the subsequent maintenance or treatment dose(s). The loading dose can be a single dose or, alternatively, a set of doses. For example, a 160 mg loading dose may be administered as a single 160 mg dose, as two doses of 80 mg each, or four doses of 40 mg each. According to some embodiments, a loading dose is subsequently followed by administration of smaller doses of TGFβ inhibitor, e.g., antibody or antigen-binding portion thereof, described herein, e.g., the treatment or maintenance dose(s).

The term “maintenance dose” or “treatment dose” is the amount of TGFβ inhibitor, e.g., antibody or antigen-binding portion thereof, described herein, taken by a subject to maintain or continue a desired therapeutic effect. A maintenance dose can be a single dose or, alternatively, a set of doses. A maintenance dose is administered during the treatment or maintenance phase of therapy. In one embodiment, a maintenance dose(s) is smaller than the induction dose(s) and may be equal to each other when administered in succession.

The term “treatment phase” or “maintenance phase,” as used herein, refers to a period of treatment comprising administration of a TGFβ inhibitor, e.g., antibody or antigen-binding portion thereof, described herein, to a subject in order to maintain a desired therapeutic effect, e.g., improved symptoms associated with a TGFβ-related indication as described herein.

According to some embodiments, the loading dose is administered via intravenous administration, and the maintenance dose is administered by subcutaneous administration. For example, in an exemplary embodiment, a loading dose approach may be applicable in the clinic if the pharmacologically active dose is fairly high and it is desirable to reach a high serum exposure in a short amount of time. Accordingly, an intravenous loading dose could be administered in a medical center, followed by subcutaneous administration of maintenance doses if the antibody half-life is sufficiently long, the antibody is potent enough, and it can be formulated for subcutaneous application.

In some embodiments, an initial loading dose might be first administered, followed by a lower maintenance dose. For example, an initial loading dose might be administered once at a dosage of about 30 mg/kg to about 90 mg/kg intravenously or subcutaneously, followed by a maintenance dosage of about 10 mg/kg to about 30 mg/kg intravenously or subcutaneously.

In one embodiment, the antibody, or antigen-binding fragment thereof, is administered to the subject at a dosage of between 0.1 and 30 mg/kg, between 0.5 and 30 mg/kg, between 1 and 30 mg/kg, between 5 and 30 mg/kg, between 10 and 30 mg/kg, between 15 and 30 mg/kg, between 20 and 30 mg/kg, between 25 and 30 mg/kg, between 0.1 and 25 mg/kg, between 0.5 and 25 mg/kg, between 1 and 25 mg/kg, between 5 and 25 mg/kg, between 10 and 25 mg/kg, between 15 and 25 mg/kg, between 20 and 25 mg/kg, between 0.1 and 20 mg/kg, between 0.5 and 20 mg/kg, between 1 and 20 mg/kg, between 5.0 and 20 mg/kg, between 10 and 20 mg/kg, between 15 and 20 mg/kg, between 0.1 and 15 mg/kg, between 0.5 and 15 mg/kg, between 1 and 15 mg/kg, between 5 and 15 mg/kg, between 10 and 15 mg/kg, between 5.0 and 20 mg/kg, between 10 and 20 mg/kg, between 15 and 20 mg/kg, between 0.1 and 10 mg/kg, between 0.5 and 10 mg/kg, between 1 and 10 mg/kg, between 5 and 10 mg/kg, optionally, wherein the subject is administered the antibody, or antigen-binding portion thereof, twice a week, once a week, once every 2 weeks, once every 3 weeks, once a month, or every other month.

An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used.

Pharmacokinetics experiments have shown that the serum concentration of an inhibitor, e.g., antibody, disclosed herein (e.g., SR-AB42) remains stable for at least 7 days after administration to a preclinical animal model (e.g., a mouse model). Without wishing to be bound by any particular theory, this stability post-administration may be advantageous since the antibody may be administered less frequently while maintaining a clinically effective serum concentration in the subject to whom the antibody is administered (e.g., a human subject). In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 30.00 mg/kg (e.g., about 1, 2, 5, 10, 15, 20 or 30 mg/kg) may be administered. The particular dosage regimen, e.g., dose, timing and repetition, will depend on the particular individual and that individual's medical history, as well as the properties of the individual agents (such as the half-life of the agent, and other relevant considerations).

Suitable dosage (e.g., efficacious amount) may be determined by the level of serum concentration (exposure) of the antibody that can be achieved in a patient, based on the pharmacokinetic profile of the particular antibody. For example, about 10-350 μg of the antibody exposure per mL of serum/blood may represent a therapeutically effective amount of the antibody. Once minimally efficacious dosage or serum exposure is determined, dosing regimen may be adjusted to achieve sufficient amount of the antibody.

For the purpose of the present disclosure, the appropriate dosage of an inhibitor, e.g., antibody or antigen-binding fragment thereof, that selectively binds a LTBP1-TGFβ1 complex and/or a LTBP3-TGFβ1 complex will depend on the specific antibody (or compositions thereof) employed, the type and severity of the indication, whether the antibody is administered for preventive or therapeutic purposes, previous therapy, the patient's clinical history and response to the inhibitor, and the discretion of the attending physician. In some embodiments, a clinician will administer an inhibitor, e.g., antibody, that selectively binds a LTBP1-TGFβ1 complex and/or a LTBP3-TGFβ1 complex, until a dosage is reached that achieves the desired result. Administration of an inhibitor, e.g., antibody, that selectively binds a LTBP1-TGFβ1 complex and/or a LTBP3-TGFβ1 complex can be continuous or intermittent, depending, for example, upon the recipient's physiological condition, whether the purpose of the administration is therapeutic or prophylactic, and other factors known to skilled practitioners. The administration of an inhibitor, e.g., antibody, that selectively binds a LTBP1-TGFβ1 complex and/or a LTBP3-TGFβ1 complex may be essentially continuous over a preselected period of time or may be in a series of spaced dose, e.g., either before, during, or after developing a TGFβ-related indication.

As used herein, the term “treating” refers to the application or administration of a composition including one or more active agents to a subject, who has a TGFβ-related indication, a symptom of the indication, or a predisposition toward the indication, with the purpose to cure, heal, alleviate, relieve, alter, remedy, ameliorate, improve, or affect the indication, the symptom of the indication, or the predisposition toward the indication.

Alleviating a TGFβ-related indication with an inhibitor, e.g., antibody, that selectively binds a LTBP1-TGFβ1 complex and/or a LTBP3-TGFβ1 complex includes delaying the development or progression of the indication, or reducing indication's severity. Alleviating the indication does not necessarily require curative results. As used therein, “delaying” the development of an indication associated with a TGFβ-related indication means to defer, hinder, slow, retard, stabilize, and/or postpone progression of the indication. This delay can be of varying lengths of time, depending on the history of the indication and/or individuals being treated. A method that “delays” or alleviates the development of an indication, or delays the onset of the indication, is a method that reduces probability of developing one or more symptoms of the indication in a given time frame and/or reduces extent of the symptoms in a given time frame, when compared to not using the method. Such comparisons are typically based on clinical studies, using a number of subjects sufficient to give a statistically significant result.

### Selection of a TGFβ1 Inhibitor

The present disclosure includes specific inhibitors of ECM-associated TGFβ1 activation, e.g., antibodies, and antigen-binding fragment thereof, that selectively bind a LTBP1/3-TGFβ1 complex, and which inhibit activation of TGFβ1 presented in the context of LTBP1 or LTBP3. Context-specific antibodies that selectively bind a GARP-TGFβ1 complex and inhibit activation of TGFβ1 presented in the context of GARP have recently been described in WO 2018/013939. Isoform-specific, context-independent TGFβ1 inhibitors have also been described, which bind pro/latent TGFβ1 presented by LTBP1/3, GARP, or LRRC33, and inhibit the release of mature TGFβ1 from the presenting molecule complex (see, e.g., WO 2017/156500, WO 2018/129329, WO 2020/014460 and WO 2020/014473. Indeed, this approach has been more recently followed by other groups (see, for example, WO 2021/039945)). The entire contents of each of the foregoing applications are incorporated herein by reference.

The present disclosure encompasses insights into selection of “the right TGFβ1 inhibitor” for “the right patient” to treat a disease condition with certain clinical features. In one aspect, the present disclosure provides use of preferred TGFβ1 inhibitors suitable for a particular patient population with fibrotic conditions. Accordingly, the disclosure includes use of an LTBP1/LTBP3-proTGFβ1 inhibitor in the treatment of a fibrotic condition in a subject, wherein the subject benefits from immunosuppression. This is based on the notion that at least a subset of TGFβ1 activities involves immune regulation which is mediated by GARP-associated and/or LRRC33-associated TGFβ1. Thus, the disclosure includes the recognition that use of TGFβ1 inhibitors that also affect the immune aspect of TGFβ1 effects may be detrimental for treating patients with fibrotic conditions where immunostimulation may cause exacerbation of the disease. The disclosure therefore aims at least in part to provide means of selectively inhibiting TGFβ1 effects within the ECM context (e.g., LTBP-associated) while sparing TGFβ1 effects associated with non-ECM contexts (e.g., immune cells, leukocytes, etc. expressing GARP or LRRC33 on cell surface), so as to prevent unwanted immunostimulation.

In some embodiments, patient populations who benefit from both: i) inhibition of TGFβ1 signaling, and, ii) immunosuppression, include those who suffer from a severe or late stage organ fibrosis and who are to receive an allograft organ transplant. The severe or late-stage organ fibrosis may be associated with IPF, CKD, and/or NASH. Such patients may have already received other therapies for treating the fibrotic disease, yet which may have failed to sufficiently treat or manage the condition. Attending physicians may determine that remaining treatment options may include allograft transplantation. Such patients may be placed in a wait list for an available organ for transplantation. Such patients may be treated with an immunosuppressant. A selective inhibitor of LTBP1/LTBP3-presented TGFβ1 activation, which does not inhibit GARP-presented TGFβ1 activation, can be used to treat such patients, without raising risk of triggering immunostimulation mediated by effector T cells. Similarly, following the transplantation, such patients may continue to receive the selective inhibitor of LTBP1/LTBP3-presented TGFβ1 activation to avoid risk of an organ rejection.

In some embodiments, patient populations who benefit from both: i) inhibition of TGFβ1 signaling, and, ii) immunosuppression, include those who suffer from a fibrotic disorder and who have an inflammatory or autoimmune condition.

In some embodiments, the inflammatory or autoimmune condition is associated with the fibrosis. Non-limiting examples of inflammatory or autoimmune conditions associated with fibrosis include muscular dystrophy, such as DMD.

In other embodiments, where patient populations who benefit from both: i) inhibition of TGFβ1 signaling, and, ii) immunosuppression, include those who suffer from a fibrotic disease and who have an inflammatory or autoimmune condition that is not directly associated with the fibrosis, but rather a discrete disorder.

Such inflammatory or autoimmune conditions, whether or not directly associated with the underlining fibrotic disease or separate condition(s), may be caused by or associated with imbalance of regulatory T cells (Treg) in human autoimmune diseases. For example, such disorders that are linked to Treg dysregulation include, but are not limited to: Juvenile idiopathic arthritis; Rheumatoid arthritis (RA); Spondyloarthritis; Psoriatic arthritis; HCV mixed cryoglobulinaemia; cryoglobulinaemia; Multiple sclerosis; Autoimmune liver disease; Systemic lupus erythematodes; Immune-mediated diabetes; Myasthenia gravis; Primary Sjögren syndrome; Kawasaki disease; and, Inflammatory bowel disease (IBD).

Thus, LTBP1/3-sepective inhibitors of TGFβ1 signaling, such as those described herein, can be used to treat patients who suffer from a fibrotic condition and inflammatory or autoimmune condition such as one or more of the disorders listed above. The LTBP1/3-sepective inhibitors of TGFβ1 signaling used accordingly can treat or alleviate TGFβ1-dependent fibrosis in the ECM, while sparing immune-associated TGFβ1 signaling.

Accordingly, related methods of the disclosure include methods for selecting an appropriate TGFβ1 inhibitor for treating a fibrotic disorder, based on the clinical manifestations of the fibrotic disorder in a subject. In one embodiment, the disclosure provides a method of selecting an isoform-specific TGFβ1 inhibitor for treatment of a fibrotic disorder in a subject. The method comprises (a) determining whether the fibrotic disorder manifests clinical presentations including fibrosis and one or more of inflammation, immune suppression, proliferative dysregulation, and need for an allograft transplant, and (b) selecting an isoform-specific, context-dependent TGFβ1 inhibitor or an isoform-specific, context-independent TGFβ1 inhibitor for treatment of the fibrotic disorder based on the clinical presentations determined in step (a). In another embodiment, the disclosure provides a method of treating a subject having a fibrotic disorder, comprising selecting a treatment regimen including an isoform-specific TGFβ1 inhibitor for the subject, and administering the selected treatment regimen to the subject, wherein the selection comprises (a) determining whether the fibrotic disorder manifests clinical presentations including fibrosis and one or more of the following: inflammation, immune suppression, proliferative dysregulation, and need for an allograft transplant; and (b) selecting a treatment regimen comprising an isoform-specific, context-dependent TGFβ1 inhibitor or an isoform-specific, context-independent TGFβ1 inhibitor, based on the clinical presentations determined in step (a).

Subjects afflicted with fibrotic disorders can display a wide range of symptoms, in addition to fibrosis. The specific combination of clinical manifestations in a subject can guide the selection of an appropriate TGFβ1-inhibitory treatment regimen. For example, a context-independent, isoform-specific TGFβ1 inhibitor can be used to treat the subject if the subject's clinical manifestations indicate a need for inhibition of TGFβ1, without modulating the activity of TGFβ2 or TGFβ3. A treatment regimen including a LTBP context-specific inhibitor can be used to treat the subject if the subject's clinical manifestations indicate that inhibition of TGFβ1 in the extracellular matrix would be beneficial. A LTBP context-specific inhibitor is also advantageous if the subject's clinical manifestations indicate that stimulation of immune effector cells is undesirable. A GARP context-specific inhibitor can be used to treat the subject if the subject's clinical manifestations indicate that blocking the activation/release of TGFβ1 on regulatory T cells (Treg cells) would be beneficial, e.g., to prevent Treg cells from suppressing effector T cell activity. A LRRC33 context-specific inhibitor can be used to treat the subject if the subject's clinical manifestations indicate that blocking the activation/release of TGFβ1 on myeloid cells, monocytes, macrophages, dendritic cells and/or microglia would be beneficial, e.g., to reverse or reduce immune suppression in the subject.

By way of example, a subject having a fibrotic disorder may display clinical manifestations including fibrosis, inflammation, immune suppression, and proliferative dysregulation. Fibrotic disorders which commonly present with the foregoing combination of symptoms include, e.g., myelofibrosis. In this embodiment, an isoform-specific, context-independent TGFβ1 inhibitor can be selected for treating the subject.

A subject having a fibrotic disorder may display clinical manifestations including fibrosis, inflammation (e.g., autoimmune disorders), and need for an allograft transplant. Fibrotic disorders which commonly present with the foregoing combination of symptoms include, e.g., organ fibrosis, such as kidney fibrosis (e.g., fibrosis associated with chronic kidney disease), liver fibrosis (e.g., fibrosis associated with nonalcoholic steatohepatitis (NASH)), scleroderma, and lung fibrosis (e.g., fibrosis associated with idiopathic pulmonary fibrosis (IPF)). In this embodiment, a context-specific LTBP1/3-specific inhibitor is selected for treating the subject.

In another example, a subject having a fibrotic disorder may display clinical manifestations including fibrosis and inflammation. Fibrotic disorders which commonly present with the foregoing combination of symptoms include, e.g., scleroderma. In this embodiment, a context-specific LTBP1/3-specific inhibitor is selected for treating the subject. Additional fibrotic disorders which commonly present with the foregoing combination of symptoms include, e.g., degenerative diseases, such as muscular dystrophy, e.g., Duchenne muscular dystrophy (DMD). In this embodiment, a context-specific LTBP1/3-specific inhibitor is selected for treating the subject.

A subject having a fibrotic disorder may display clinical manifestations including immune suppression and proliferative dysregulation. Fibrotic disorders which commonly present with the foregoing combination of symptoms include, e.g., solid tumors. In some embodiments, the solid tumor is a malignant tumor. In other embodiments, the solid tumor is a benign tumor. In an exemplary embodiment, the subject has desmoplasia (e.g., pancreatic desmoplasia). In some embodiments, patients may have a solid tumor that has been assessed as “inoperable” or not suitable for surgical resection. Thus, in some embodiments, patients are not candidates for surgical resection of the tumor. However, TGFβ1 inhibition therapy comprising a context-selective TGFβ1 inhibitor of the present disclosure may reverse such non-candidate patients to be more suited for receiving a surgery.

In some embodiments, subjects having a solid tumor are poorly responsive to cancer therapy (e.g., the tumor is resistant to the cancer therapy), such as chemotherapy, radiation therapy, CAR-T therapy and checkpoint inhibitor therapy. TGFβ1 inhibition therapy comprising a context-selective TGFβ1 inhibitor of the present disclosure may at least in part reverse the resistance to render the patient more responsive to the cancer therapy. In some embodiments, a combination therapy comprising both the context-selective TGFβ1 inhibition therapy and the cancer therapy may synergistically treat the cancer. In some embodiments, the context-selective TGFβ1 inhibition therapy administered in conjunction with the cancer therapy may reduce the required dosage of the cancer therapy to produce equivalent or improved clinical effects.

In another exemplary embodiment, the subject has fibroids. In the foregoing embodiments, in which the fibrotic disorder displays clinical manifestations including immune suppression and proliferative dysregulation, a context-specific LTBP1/3-specific inhibitor and/or a context-specific GARP-specific inhibitor are selected for treating the subject.

In another aspect, the disclosure provides a method of treating a subject having a fibrotic disorder with an isoform-specific, LTBP1/3-specific TGFβ1 inhibitor, by selecting a subject having a fibrotic disorder manifesting clinical presentations including fibrosis and the need for an allograft transplant, and administering an effective amount of an isoform-specific, LTBP1/3-specific TGFβ1 inhibitor to the subject. In one embodiment, the method comprises determining whether the fibrotic disorder manifests clinical presentations including fibrosis and the need for an allograft transplant. The LTBP1/3-specific TGFβ1 inhibitor is administered to the subject if the subject exhibits symptoms including fibrosis and the need for an allograft transplant.

In another aspect, the disclosure provides a method of treating a subject having a fibrotic disorder with an isoform-specific, context-independent TGFβ1 inhibitor, by selecting a subject having a fibrotic disorder manifesting clinical presentations including fibrosis, immune suppression, and/or proliferative dysregulation, and administering an effective amount of an isoform-specific, context-independent TGFβ1 inhibitor to the subject. In one embodiment, the method comprises determining whether the fibrotic disorder manifests clinical presentations including fibrosis, immune suppression, and/or proliferative dysregulation. The isoform-specific, context-independent TGFβ1 inhibitor is administered to the subject if the subject inhibits symptoms including fibrosis, immune suppression, and/or proliferative dysregulation.

Clinical manifestations including inflammation, immune suppression, proliferative dysregulation, and/or the need for an allograft transplant can be determined in a subject having a fibrotic disorder using methods and practices known in the art. Such methods include, for example, physical examination and standard diagnostic tests. In one embodiment, inflammation can be assessed by determining if a subject displays an elevated level of inflammatory biomarkers in plasma, blood, or serum. Such inflammatory biomarkers include, for example, C-reactive protein, interleukin 1 (IL-1), interleukin 6 (IL-6), tumor necrosis factor α (TNF-α), or combinations thereof. Blood tests including erythrocyte sedimentation rate (ESR) and plasma viscosity (PV) can also indicate the presence of inflammation in a subject with a fibrotic disorder. In another embodiment, immune suppression can be assessed by determining the number and composition of a subject's blood cells, e.g., T cells, B cells, NK cells, monocytes, macrophages, etc. Immune suppression can also be assessed by determining if the subject is taking or has a history of taking immunosuppressant medications, or determining if the subject has a condition associated with immune suppression (e.g., hematological malignancies, HIV/AIDS, etc.). In another embodiment, proliferative dysregulation can be assessed using standard tests including blood tests, biopsy, and/or imaging procedures such as CT scan, ultrasound, and MRI. Other standard tests for diagnosing cancer (e.g., biomarker tests, etc.) can also be used to assess proliferative dysregulation. The need for an allograft transplant can be determined by a clinician using standard procedures. In one embodiment, the loss or partial loss of organ function, or an increased likelihood of loss of organ function, indicates the need for a transplant.

As mentioned, the present disclosure provides selective targeting of the ECM-associated TGFβ1 complexes enabled by the use of antibodies that are capable of specifically binding LTBP-presented TGFβ1 precursors. While some antibodies of the present disclosure are capable of binding and inhibiting both LTBP1- and LTBP3-associated proTGFβ1 complexes, others show even greater selectivity in that they only bind either LTBP1-proTGFβ1 or LTBP3-proTGFβ1.

The disclosure therefore encompasses the recognition that certain patient populations may benefit from TGFβ1 inhibition therapy comprising a context-selective inhibitor that is specific to LTBP1-proTGFβ1.

LTBP1 and LTBP3 are both components of the ECM, where they can display or “present” a latent TGFβ1 precursor complex. Some observations from expression studies raise the possibility that deletion, ablation or functional inhibition of LTBP3 may cause certain toxicities. LTBP3−/− mice (as well as some human mutations) have short stature, as well as bone and dental anomalies. These phenotypes are likely associated with disruptions in development, however, but it is possible that LTBP3 plays a role in homeostasis of these tissues in adults (expression in adult bone is reported). Based on these observations, in certain clinical situations (where the disease manifests in a tissue known to express LTBP3 and associated with toxicities) or in certain patient populations, such as pediatric patients who are still in active development, it may be advisable to avoid potential toxicities of LTBP3-related inhibition. Loss of LTBP1 function does appear to be sufficient to protect against at least some forms of fibrosis, as LTBP1−/− KO mice are protected against liver fibrosis (induced by bile duct ligation). Taken together, these data raise the possibility that LTBP1-specific TGFβ1 inhibition could have a superior safety profile as compared to LTBP1/3-TGFβ1 inhibitors in certain situations.

### Combination Therapies

The disclosure further encompasses pharmaceutical compositions and related methods used as combination therapies for treating subjects who may benefit from TGFβ1 inhibition in vivo. In any of these embodiments, such subjects may receive combination therapies that include a first composition comprising at least one TGFβ1 inhibitor, e.g., antibody or antigen-binding portion thereof, described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. To give but one example, the first composition may treat a disease or condition associated with TGFβ1 signaling, while the second composition may treat inflammation or fibrosis associated with the same disease, etc. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

Accordingly, the disclosure provides pharmaceutical compositions and methods for use in combination therapies for the reduction of TGFβ1 protein activation and the treatment or prevention of diseases or conditions associated with TGFβ1 signaling, as described herein. Accordingly, the methods or the pharmaceutical compositions further comprise a second therapy. In some embodiments, the second therapy may be useful in treating or preventing diseases or conditions associated with TGFβ1 signaling. The second therapy may diminish or treat at least one symptom(s) associated with the targeted disease. The first and second therapies may exert their biological effects by similar or unrelated mechanisms of action; or either one or both of the first and second therapies may exert their biological effects by a multiplicity of mechanisms of action.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

In one embodiment, the inhibitors, e.g., antibodies, described herein that selectively binds a LTBP1-TGFβ1 complex and/or a LTBP3-TGFβ1 complex can be administered with another agent that inhibits TGFβ1 activity. For example, the second agent can be another context-specific TGFβ1 inhibitor. In one embodiment, the combination therapy comprises (i) an inhibitor, e.g., antibody or antigen binding portion thereof, that selectively binds a LTBP1-TGFβ1 complex and/or a LTBP3-TGFβ1 complex, and (ii) an inhibitor, e.g., antibody or antigen binding portion thereof, that selectively binds a GARP-TGFβ1 complex. In another embodiment, the combination therapy comprises (i) an inhibitor, e.g., antibody or antigen binding portion thereof, that selectively binds a LTBP1-TGFβ1 complex and/or a LTBP3-TGFβ1 complex, and (ii) an inhibitor, e.g., antibody or antigen binding portion thereof, that selectively binds a LRRC33-TGFβ1 complex. Context-specific antibodies that selectively bind LRRC33-TGFβ1 are described, for example, in U.S. 62/503,785, and context-specific antibodies that selectively bind GARP-TGFβ1 are described, above. The entire contents of the foregoing applications are incorporated by reference herein. In one embodiment, the combination therapy comprises (i) an inhibitor, e.g., antibody or antigen binding portion thereof, that selectively binds a LTBP1-TGFβ1 complex and/or a LTBP3-TGFβ1 complex, and (ii) an context-independent inhibitor, e.g., antibody or antigen binding portion thereof, that selectively binds pro/latent TGFβ1 in a complex with a presenting molecule (e.g., LTBP, GARP, and/or LRRC33). Context-independent inhibitors of TGFβ1 are described, for example, in PCT/US2017/21972, the entire contents of which are incorporated herein by reference.

The one or more anti-TGFβ1 inhibitors, e.g., antibodies, or antigen binding fragment thereof, of the disclosure may be used in combination with one or more additional therapeutic agents. Examples of the additional therapeutic agents which can be used with an anti-TGFβ antibody of the disclosure include, but are not limited to, a myostatin inhibitor, a VEGF agonist, an IGF1 agonist, an FXR agonist, a CCR2 inhibitor, a CCR5 inhibitor, a dual CCR2/CCR5 inhibitor, a lysyl oxidase-like-2 inhibitor, an ASK1 inhibitor, an Acetyl-CoA Carboxylase (ACC) inhibitor, a β8 kinase inhibitor, Pirfenidone, Nintedanib, a GDF11 inhibitor, and the like.

In some embodiments, the additional agent is a checkpoint inhibitor. In some embodiments, the additional agent is selected from the group consisting of a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an oncolytic virus, and a PARP inhibitor. In some embodiments, the additional therapy is radiation. In some embodiments, the additional agent is a chemotherapeutic agent. In some embodiments, the chemotherapeutic agent is Taxol. In some embodiments, the additional agent is an anti-inflammatory agent. In some embodiments, the additional agent inhibits the process of monocyte/macrophage recruitment and/or tissue infiltration. In some embodiments, the additional agent is an inhibitor of hepatic stellate cell activation. In some embodiments, the additional agent is a chemokine receptor antagonist, e.g., CCR2 antagonists and CCR5 antagonists. In some embodiments, such chemokine receptor antagonist is a dual specific antagonist, such as a CCR2/CCR5 antagonist. In some embodiments, the additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that binds a pro/latent myostatin complex and blocks activation of myostatin. In some embodiments, the monoclonal antibody that binds a pro/latent myostatin complex and blocks activation of myostatin does not bind free, mature myostatin.

For known genetic disorders, a combination therapy or adjunct therapy may include a gene therapy or RNA-based therapy aimed to normalize the gene expression of the affected gene. For example, an LTBP-selective inhibitor of TGFβ1 activation provided herein (such as Ab42) may be used in the treatment of a muscular dystrophy such as DMD in a patient, wherein the patient is further treated with a gene therapy or RNA-based therapy aimed to increase the level of functional protein affected by the genetic disease (e.g., dystrophin). Similarly, an LTBP-selective inhibitor of TGFβ1 activation provided herein (such as Ab42) may be used in the treatment of Alport syndrome in a patient, wherein the patient is further treated with a gene therapy or RNA-based therapy aimed to increase the level of functional protein affected by the genetic disease (e.g., collagen)

Such combination therapies may advantageously utilize lower dosages of the administered therapeutic agents, thus avoiding possible toxicities or complications associated with the various monotherapies.

Diagnostic and/or Prognostic Biomarkers

A number of biomarkers have been described, alternations of which are indicative of or correlate with a or the progression of a disease. In some embodiments, the biomarkers are cellular markers. In some embodiments, such disease-associated biomarkers are useful for the diagnosis and/or monitoring of the disease progression as well as effectiveness of therapy (e.g., patients' responsiveness to the therapy). These biomarkers include a number of fibrotic markers, as well as cellular markers.

In some embodiments, useful biomarkers include fibrotic markers. These include, without limitation: TGFβ1, PAI-1 (also known as Serpine1), MCP-1 (also known as CCL2), Coil a1, Col3a1, FN1, CTGF, α-SMA, ACTA2, Timp1, Mmp8, and Mmp9. In some embodiments, useful biomarkers are serum markers (e.g., proteins or fragments found and detected in serum samples).

In some embodiments, an effective amount of the inhibitor may normalize, as compared to control, the levels of multiple inflammatory or fibrotic serum biomarkers as assessed following the start of the therapy, at, for example, 12-36 weeks. In some embodiments, inflammatory or fibrotic biomarkers may be used to assess severity of NAFLD (by measure levels of hepatic steatosis), select patients for treatment, and/or monitor disease progression or treatment response. For example, blood biomarkers and panels may include, but are not limited to:
i) the Fatty liver index (BMI, waist circumference, serum triglycerides, and gamma-glutamyltransferase (GGT);
ii) the Hepatic steatosis index (serum aspartate aminotransferase (AST):alanine aminotransferase (ALT) ratio, BMI, gender, and presence of diabetes mellitus);
iii) the NAFLD liver fat score (serum ALT, HDL cholesterol, triglicerides, haemoglobin A_{1c} and leukocyte count);
iv) the SteatoTest (BioPredictive) (serum levels of total bilirubin, GGT, α2-macroglobin, haptoglobin, ALT, apolipoprotein AI, total cholesterol, triglycerides, glucose (adjusted for age and gender) and BMI); and
v) the NAFLD ridge score (serum levels of ALT, HDL cholesterol, triglycerides, haemoglobin A_{1c}, leukocyte count, and comorbidity data (and the presence of hypertension)).

In some embodiments, imaging biomarkers can be used to assess levels of hepatic steatosis. For example, imaging biomarkers may include but are not limited to: ultrasonography, controlled attenuation parameter (CAP), MRI-estimated proton density fat fraction (MRI-PDFF), and magnetic resonance spectroscopy (MRS).

Liver biopsies are the current standard for diagnosis NASH, however, variability among pathologists limits the effectiveness of such diagnostic method. Accordingly, use of the Fatty Liver Inhibition of Progression (FLIP) algorithm (comprising histological steatosis, activity and fibrosis scores) may be used to improve the consistency of NASH diagnosis by biopsy. Moreover, many noninvasive biomarkers may also be useful for diagnosing and monitoring disease. Accordingly, in some embodiments, inflammatory or fibrotic biomarkers may be used to assess severity of NASH, select patients for treatment, and/or monitor disease progression or treatment response. Blood biomarkers may include:
i) apoptosis markers, such as CK18 fragments, total cytokeratin and sFAS;
ii) inflammatory markers, such as CRP, TNF, IL-8, and CXCL10;
iii) lipid oxidation products, such as 11-HETE, 9-HODE, 13-HODE, 12-oxo-ODE, LA-13-HODE (oxNASHscore), and 11,12-diHETrE;
iv) lysosomal enzymes, such as cathepsin D; and
v) combination panels, such as NASHTest (BioPredictive) and NASH Diagnostics Panel (comprising, presence of diabetes mellitus, sex, BMI, and serum levels of triglyceride, CK18 fragments, and total CK18).

In some embodiments, biomarkers and related panels may be useful in diagnosis levels of fibrosis and/or cirrhosis, select patients for treatment, and/or monitor disease progression or treatment response. For example, noninvasive tests of liver fibrosis and cirrhosis include, but are not limited to: AST:ALT ratio, AST:platelet ratio index, fibrosis-4 index (age, AST, ALT, and platelet count), NAFLD fibrosis score (age, BMI, impaired fasting glucose and/or diabetes, AST ALT, platelet count, and albumin), BARD score (AST, ALT, BMI, and diabetes).

Specific fibrosis markers and panels may also be useful, and include, but are not limited to: hyaluronic acid; PIIPNP; Pro-C3; TIMP1; Laminin; enhanced liver fibrosis (ELF) panel (PIINP, hyaluronic acid, TIMP1); FibroTest (GGT, total bilirubin, α2m, apolipoprotein AI, and haptoglobin); and FibroMeter NAFLD (body weight, prothrombin index, ALT, AST, ferritin, and fasting glucose). Imaging biomarkers for liver fibrosis may include, but are not limited to: FibroScan (TE), point shear wave elastography (pSWE) (aka acoustic radiation force impulse (ARFI)), 2D-3D SWE, magnetic resonance elastography (MRE), and multiparameteric MRI.

In some embodiments, serum levels of liver enzymes such as alkaline phosphatase (ALP), alanine aminotransferase (ALT), aspartate tramsaminase (AST), or G-glutamyl transferase (GGT) may be measured as indicators of fibrosis in the liver.

In some embodiments, genetic and genomic biomarkers may be useful in assessing NAFLD risk and severity, which include the assessment of various SNPs, cell-free ncRNAs, and miRNAs. A comprehensive review of known genetic and genomic biomarkers, as well as the above-discussed blood biomarkers, panels, imaging biomarkers, and tests are summarized in VWS Wong et al., Nat Rev Gastroenterol Hepatol. 2018 August; 15(8):461-478; the contents of which are incorporated herein by reference in its entirety.

Assays for Detecting a LTBP1-TGFβ1 Complex and/or a LTBP3-TGFβ1 Complex

In some embodiments, methods and compositions provided herein relate to a method for detecting a LTBP1-TGFβ1 complex and/or a LTBP3-TGFβ1 complex in a sample obtained from a subject. As used herein, a “subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, poultry, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a patient or a healthy volunteer.

In some embodiments, a method for detecting a LTBP1-TGFβ1 complex and/or a LTBP3-TGFβ1 complex in a sample obtained from a subject involves (a) contacting the sample with an antibody that selectively binds a LTBP1-TGFβ1 complex and/or a LTBP3-TGFβ1 complex under conditions suitable for binding of the antibody to the antigen, if the antigen is present in the sample, thereby forming binding complexes; and (b) determining the level of the antibody bound to the antigen (e.g., determining the level of the binding complexes).

In one embodiment, a screening assay that utilizes biotinylated latent TGFβ1 complexes immobilized onto a surface is utilized, which allows for the activation of latent TGFβ by integrins, e.g., by providing a tether. Other, non-integrin activators could also be tested in that system. A readout can be measured through reporter cells or other TGFβ3-dependent cellular responses.

### Diagnostics and Monitoring

According to some embodiments, therapeutic methods that include TGFβ1 inhibition therapy may comprise diagnosis of a TGFβ1 indication and/or selection of patients likely to respond to such therapy. Additionally, patients who receive the TGFβ1 inhibitor may be monitored for therapeutic effects of the treatment, which typically involves measuring one or more suitable parameters which are indicative of the condition and which can be measured (e.g., assayed) before and after the treatment and evaluating treatment-related changes in the parameters. For example, such parameters may include levels of biomarkers present in biological samples collected from the patients. Biomarkers may be RNA-based, protein-based, cell-based and/or tissue-based. For example, genes that are overexpressed in certain disease conditions may serve as the biomarkers to diagnose and/or monitor the disease or response to the therapy. Cell-surface proteins of disease-associated cell populations may serve as biomarkers. Such methods may include the direct measurements of disease parameters indicative of the extent of the particular disease, such as extent of fibrosis. Any suitable sampling methods may be employed, such as serum/blood samples, biopsies, and imaging. The biopsy may include tissue biopsies and liquid biopsies.

### Circulating/Circulatory Latent-TGFβ

According to the present disclosure, circulating latent TGFβ may serve as a target engagement biomarker. Where an activation inhibitor is selected as a therapeutic candidate, for example, such biomarker may be employed to evaluate or confirm in vivo target engagement by monitoring the levels of circulating latent TGFβ before and after administration. In various embodiments, the present disclosure provides methods of treating a TGFβ-related disorder, comprising monitoring the level of circulating latent TGFβ (e.g., TGFβ1) in a sample obtained from a patient (e.g., in the blood, e.g., plasma and/or serum, of a patient) receiving a TGFβ inhibitor. The level of circulating latent TGFβ may be monitored alone or in conjunction with one or more of the biomarkers disclosed herein (e.g., MDSCs). In certain embodiments, the TGFβ inhibitor may be administered alone or in conjunction with an additional therapy. In some embodiments, the treatment may be administered to a subject afflicted with a fibrotic disorder (e.g., organ fibrosis). In some embodiments, the TGFβ inhibitor is a TGFβ1-selective antibody or antigen-binding fragment thereof described herein. In some embodiments, the TGFβ inhibitor is an isoform-non-selective TGFβ inhibitor (such as low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors). In some embodiments, the TGFβ inhibitor is an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In various embodiments, circulating latent TGFβ (e.g., latent TGFβ1) may be measured in a sample obtained from a subject (e.g., whole blood or a blood component). In various embodiments, the circulating latent TGFβ levels (e.g., latent TGFβ1) may be measured within 1, 2, 3, 4, 5, 6, 7, 8, 10, 12, 14, 16, 18, 21, 25, 28, 30, 35, 40, 45, 48, 50, or 56 days following administration of the TGFβ inhibitor to a subject, e.g., up to 7 days after administration of a therapeutic dose of a TGFβ inhibitor. In some embodiments, the circulating latent TGFβ levels (e.g., latent TGFβ1) may be measured by any method known in the art (e.g., ELISA).

In various embodiments, a method of treating a fibrotic disorder or other TGF-β elated disorder comprises administering a TGFβ inhibitor (e.g., an anti-TGFβ1 antibody, e.g., an isoform-selective activation inhibitor of TGFβ1 such as Ab42, or derivatives thereof,) to a patient in need thereof and confirming the level of target engagement by the inhibitor. In some embodiments, determining the level of target engagement comprises determining the levels of circulating latent TGFβ (e.g., TGFβ1) in a sample obtained from a patient (e.g., in the blood or a blood component of a patient) receiving the TGFβ inhibitor. In some embodiments, an increase in circulating latent TGFβ (e.g., TGFβ1) after administration of the TGF inhibitor indicates target engagement. In some embodiments, an increase in circulating latent TGFβ (e.g., TGFβ1) of at least 2-fold, at least 3-fold, at least 4-fold, at least 5-fold, at least 6-fold, at least 7-fold, at least 8-fold, at least 9-fold, at least 10-fold, or more, after administration of the TGF inhibitor indicates target engagement. In various embodiments, the present disclosure also provides methods of using circulating latent TGFβ levels (e.g., TGFβ1 levels) as a predictive biomarker, i.e., to predict therapeutic response, as well as for informing further treatment decisions (e.g., by continuing treatment if an increase is observed).

In one aspect of the current disclosure, levels of circulating latent TGFβ are determined to inform treatment and predict therapeutic efficacy in subjects administered a TGFβ inhibitor such as a TGFβ1-selective inhibitor described herein (e.g., Ab42, or derivatives thereof). In certain embodiments, a TGFβ inhibitor (e.g., an isoform-selective activation inhibitor of TGFβ1 such as Ab42, or derivatives thereof) is administered alone or concurrently (e.g., simultaneously), separately, or sequentially with an additional therapy, such that the amount of TGFβ1 inhibition administered is sufficient to increase the levels of circulating latent-TGFβ (e.g., latent TGFβ1) as compared to baseline circulating latent-TGFβ levels. Circulating latent-TGFβ levels may be measured prior to or after each treatment such that an increase in circulating latent-TGFβ levels (e.g., latent TGFβ1) following the treatment indicates therapeutic efficacy. For instance, circulating latent-TGFβ levels (e.g., latent TGFβ1) may be measured prior to and after the administration of a TGFβ inhibitor and an increase in circulating latent-TGFβ levels (e.g., latent TGFβ1) following the treatment predicts therapeutic efficacy. In some embodiments, treatment is continued if an increase is detected. In certain embodiments, circulating latent-TGFβ levels may be measured prior to and following administration of a first dose of a TGFβ inhibitor such as a TGFβ1 inhibitor described herein (e.g., an isoform-selective activation inhibitor of TGFβ1 such as Ab42, or derivatives thereof), and an increase in circulating latent-TGFβ levels (e.g., latent TGFβ1) following the administration predicts therapeutic efficacy and further warrants administration of a second or more dose(s) of the TGFβ inhibitor. In some embodiments, circulating latent-TGFβ levels (e.g., latent TGFβ1) may be measured prior to and after a combination treatment of TGFβ inhibitor such as a TGFβ1-selective inhibitor (e.g., an isoform-selective activation inhibitor of TGFβ1 such as Ab42, or derivatives thereof, and an additional therapy, administered concurrently (e.g., simultaneously), separately, or sequentially, and a change in circulating latent-TGFβ levels following the treatment predicts therapeutic efficacy. In some embodiments, treatment is continued if an increase is detected. In some embodiments, the increase in circulating latent-TGFβ levels following a combination treatment may warrant continuation of treatment.

In various embodiments, the current disclosure encompasses a method of treating a TGFβ-related disorder, e.g., a fibrotic disorder, comprising administering a therapeutically effective amount of a TGFβ inhibitor (e.g., an isoform-selective activation inhibitor of TGFβ1 such as Ab42, or derivatives thereof), to a subject having a TGFβ-related disorder, wherein the therapeutically effective amount is an amount sufficient to increase the level of circulating latent TGFβ (e.g., latent TGFβ1). In certain embodiments, the TGFβ inhibitor is a TGFβ activation inhibitor. In certain embodiments, the TGFβ inhibitor is a TGFβ1 inhibitor. In some embodiments, the TGFβ inhibitor is an isoform-selective activation inhibitor of TGFβ1 such as Ab42, or derivatives thereof. In certain embodiments, the circulating latent TGFβ is latent TGFβ1. In some embodiments, the therapeutically effective amount of the TGFβ inhibitor (e.g., an isoform-selective activation inhibitor of TGFβ1 such as Ab42, or derivatives thereof) is between 0.1-30 mg/kg per dose. In some embodiments, the TGFβ inhibitor is dosed weekly, every 2 weeks, every 3 weeks, every 4 weeks, monthly, every 6 weeks, every 8 weeks, bi-monthly, every 10 weeks, every 12 weeks, every 3 months, every 4 months, every 6 months, every 8 months, every 10 months, or once a year.

In various embodiments, circulating latent TGFβ (e.g., latent TGFβ1) may be measured in a sample obtained from a subject (e.g., whole blood or a blood component). In some embodiments, circulating latent TGFβ1 may be measured using an enzyme-linked immunosorbent assay (ELISA) that measures total free TGFβ1 after acid treatment. Analysis of latent TGF-β1 by TGF-β1 ELISA first requires dissociation of TGF-β1 from the latent complex, e.g. by acidification of samples. The ELISA then measures total TGF-β1, equivalent to dissociated latent TGF-β1, in addition to any free TGF-β1 present prior to acidification, which is known to be only a small fraction of circulating TGFβ1. In certain embodiments, the level of circulating latent TGFβ (e.g., latent TGFβ1) following administration of a TGFβ inhibitor is increased by at least two-fold (e.g., at least 2-fold, 3-fold, 4-fold, 5-fold, 6-fold, 7-fold, 8-fold, 9-fold and 10-fold) as compared to circulating latent TGFβ levels prior to the administration.

In certain embodiments, circulating total TGFβ levels (e.g., latent and mature TGFβ1) may be used to monitor target engagement and pharmacological activity of a TGFβ inhibitor in a subject receiving a TGFβ inhibitor therapy (e.g., an isoform-selective activation inhibitor of TGFβ1 such as Ab42, or derivatives thereof). In certain embodiments, circulating total TGFβ levels (e.g., latent and mature TGFβ1 levels) may be measured prior to and after administration of a first dose of TGFβ inhibitor such that an increase in circulating TGFβ levels of at least two-fold (e.g. at least 2-fold, 3-fold, 4-fold, 5-fold, 6-fold, 7-fold, 8-fold, 9-fold, 10-fold, or more) in following the administration indicates target engagement (e.g., binding of the TGFβ inhibitor to human large latent proTGFβ1 complex). In certain embodiments, circulating latent TGFβ levels (e.g., latent TGFβ1) may be measured prior to and after administration of a first dose of TGFβ inhibitor such that an increase in circulating latent TGFβ levels (e.g., latent TGFβ1) following the administration indicates therapeutic efficacy. In certain embodiments, treatment is continued if an increase in circulating latent-TGFβ levels (e.g., latent TGFβ1) following administration of a TGFβ inhibitor is detected. In some embodiments, total TGFβ1 in a blood sample (e.g., serum) comprises both latent and mature forms, the former of which representing vast majority of circulatory TGFβ1. Acid treatment of the sample releases mature growth factor from the latent complex. Well-known assays such as ELISA can then be used to measure the amount of free TGFβ1. Alternatively, reagents such as antibodies that specifically bind the latent form of TGFβ1 may be employed to specifically measure circulatory latent TGFβ1. In some embodiments, circulating latent-TGFβ levels (e.g., latent TGFβ1) may be measured prior to and after administration of a first dose of a TGFβ inhibitor, and an increase in circulating latent-TGFβ levels (e.g., latent TGFβ1) after the administration indicates target engagement and/or treatment response, and/or further warrants administration of a second or more dose(s) of the TGFβ inhibitor. In another embodiment, circulating latent-TGFβ levels may be measured prior to and after administration of a first dose of a TGFβ inhibitor such as a TGFβ31-selective inhibitor, and an increase in circulating latent-TGFβ levels after the administration indicates target engagement and/or treatment response, and/or further warrants continuation of treatment. In various embodiments, a TGFβ inhibitor such as a TGFβ1-selective inhibitor, an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In some embodiments, the processing of blood to serum activates platelets and leads to release of large pools of latent TGFβ1, which confounds the measurements. Therefore, according to some embodiments, it is preferable to measure circulating levels of TGFβ1 in plasma instead of in serum. Accordingly, in preferred embodiments, circulating TGFβ (e.g., circulating latent-TGFβ levels (e.g., latent TGFβ1)) is measured in plasma samples collected from the subject.

### Cell-Based Assays for Measuring TGFβ Activation

Activation of TGFβ (and inhibition thereof by a TGFβ test inhibitor, such as an antibody) may be measured by any suitable method known in the art. For example, integrin-mediated activation of TGFβ can be utilized in a cell-based assay, such as the “CAGA12” luciferase assay, described in more detail herein.

The development of novel context-dependent cell-based assays of TGFβ1 activation is described in U.S. Provisional Patent Application No. 62/538,476 and the corresponding International Application Pub. No. WO 2019/023661, each of which are incorporated by reference in their entirety herein. Previous assay formats could not differentiate between the activation of proTGFβ1 presented by endogenous presenting molecules and the activation of proTGFβ1 presented by exogenous LTBPs. By directly transfecting integrin-expressing cells, the novel assays disclosed in International Application Pub. No. WO 2019/023661, and used herein, establish a window between endogenous presenter-proTGFβ1 activity and exogenous LTBP-proTGFβ1 activity.

As opposed to GARP- or LRRC33-proTGFβ1 complexes, which are presented on the surface of cells, LTBP-proTGFβ1 complexes are embedded in the extracellular matrix. Thus, the assay plate coating is an important component of the assay when assessing activation of proTGFβ1 in complex with LTBP (e.g., LTBP1/3). In this regard, it has been shown, fibronectin and fibrillin are two ECM components that appear to be critical for LTBP association with the matrix and activation of latent TGFβ (Robertson et al., Matrix Biol. 2015 September; 47:44-53). For example, LTBP3 ECM-incorporation appears to be dependent on fibrillin expression in both in vitro and in vivo models (Zilberberg et al., J Cell Physiol. 2012; 227(12):3828-3836).

On the other hand, LTBP1 has been shown to interact with fibrillin microfibrils and fibronectin via its C- and N-termini, respectively (Dallas et al., J Biol Chem. 2005; 280(19):18871-18880; Fontana et al., FASEB J. 2005; 19(13):1798-1808; and Kantola et al., Exp Cell Res. 2008; 314(13):2488-2500). Moreover, in the absence of fibrillin, LTBP1 still co-localizes with fibronectin fibers (Robertson et al., Matrix Biol. 2015 September; 47:44-53). LTBP1 has also been shown to interact with ADAMTSL2 and 3 (Sengle et al., PloS Genet. 2012; 8(1):e1002425), IGFBP3 (Gui and Murphy, Mol Cell Biochem. 2003; 250(1-2):189-195), fibulin-4 (Massam-Wu et al., J Cell Sci. 2010 Sep. 1; 123(Pt 17):3006-18), and heparin (Chen et al., J Biol Chem. 2007; 282(36):26418-26430).

Beyond traditional ECM components/proteins, tissue transglutaminase (TG2) may also play a critical role in TGFβ localization in the ECM. TG2 is known to catalyze inter- and intramolecular isopeptide bonds which cross-link ECM fibrils, effectively stiffening the ECM and protecting the ECM from proteolytic degradation (Benn et al., Current Opinion in Biomedical Engineering., 2019, https://doi.org/10.1016/j.cobme.2019.06.003). Moreover, TG2 can cross-link LTBP1 to the ECM, thus promoting a matrix reservoir of TGFβ (Nunes et al., J Cell Biol. 1997; 136(5):1151-1163).

Accordingly, the N-terminus of LTBPs may be covalently bound to the ECM via an isopeptide bond, the formation of which may be catalyzed by transglutaminases. The structural integrity of the ECM is believed to be important in mediating LTBP-associated TGFβ1 activity. For example, stiffness of the matrix can significantly affect TGFβ1 activation. In addition, incorporating fibronectin and/or fibrillin in the scaffold may significantly increase the LTBP-mediated TGFβ1 activation. Similarly, presence of fibronectin and/or fibrillin in LTBP assays (e.g., cell-based potency assays) may increase an assay window.

Accordingly, a cell-based assay for measuring TGFβ1 activation may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ3-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al., (1994) Anal Biochem. 216(2): 276-84, incorporated herein by reference.

In some embodiments, the assay comprises a single-cell system, in which each of the aforementioned assay components are is provided from the same source (e.g., the same cell). In some embodiments, such cell-based assay comprises a single-cell reporter assay, in which the three assay components are expressed by a single clone, preferably as a stably transfected cell line. In some embodiments, the cell line is an LN229 cell line, which is a human glioblastoma cell line. In some embodiments, the cell line is stably transfected with proTGFb1, αvβ8 integrin, TGFβ receptors, and CAGA12 promoter operatively linked to a reporter gene, preferably the luciferase gene. In preferred embodiments, proTGFβ1 expression is driven by an inducible promoter, wherein optionally the inducible promoter is regulated by tetracycline. In preferred embodiments, the inducible promoter is doxycycline-inducible.

In some embodiments, the assay comprises a two-cell system, in which two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and a transfected cell). When cells are used as the source of one or more of the assay components, such components of the assay may be endogenous to the cell, stably expressed in the cell, transiently transfected, or any combination thereof. In some embodiments, the assay is performed in a tissue culture plate or dish. In some embodiments, the tissue culture plate or dish is coated with a component of the extracellular matrix (ECM). In some embodiments, the tissue culture plate or dish is coated with fibronectin and/or fibrillin. In some embodiments, the cell-based assay further comprises a fourth component comprising a source of TG2. In some embodiments, the TG2 component is provided from the same, or different, source as any one of the above-mentioned components.

A skilled artisan could readily adapt such assays to various suitable configurations. For instance, a variety of sources of TGFβ may be considered. In some embodiments, the source of TGFβ is a cell that expresses and deposits TGFβ (e.g., a primary cell, a propagated cell, an immortalized cell or cell line, etc.). In some embodiments, the source of TGFβ is purified and/or recombinant TGFβ immobilized in the assay system using suitable means. In some embodiments, TGFβ immobilized in the assay system is presented within an extracellular matrix (ECM) composition on the assay plate, with or without de-cellularization, which mimics fibroblast-originated TGFβ. In some embodiments, TGFβ is presented on the cell surface of a cell used in the assay. Additionally, a presenting molecule of choice may be included in the assay system to provide suitable latent-TGFβ complex. One of ordinary skill in the art can readily determine which presenting molecule(s) may be present or expressed in certain cells or cell types. Using such assay systems, relative changes in TGFβ activation in the presence or absence of a test agent (such as an antibody) may be readily measured to evaluate the effects of the test agent on TGFβ activation in vitro.

Such cell-based assays may be modified or tailored in a number of ways depending on the TGFβ isoform being studied, the type of latent complex (e.g., presenting molecule), and the like. In some embodiments, a cell known to express integrin capable of activating TGFβ may be used as the source of integrin in the assay. Such cells include SW480/β6 cells (e.g., clone 1E7). In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest (such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out (e.g., luciferase activity) is detected using suitable means (e.g., for luciferase-expressing reporter cell lines, the Bright-Glo reagent (Promega) can be used). In some embodiments, Luciferase fluorescence may be detected using a BioTek (Synergy H1) plate reader, with autogain settings.

Kits for Use in Alleviating Diseases/Disorders Associated with LTBP1/3-TGF3

The present disclosure also provides kits for use in alleviating diseases/disorders associated with a TGFβ-related indication. Such kits can include one or more containers comprising an inhibitor, e.g., antibody, or antigen binding portion thereof, that selectively binds to a LTBP1-TGFβ1 complex and/or a LTBP3-TGFβ1 complex, e.g., any of those described herein.

In some embodiments, the kit can comprise instructions for use in accordance with any of the methods described herein. The included instructions can comprise a description of administration of the inhibitor, e.g., antibody, or antigen binding portion thereof, that selectively binds a LTBP1-TGFβ1 complex and/or a LTBP3-TGFβ1 complex to treat, delay the onset, or alleviate a target disease as those described herein. The kit may further comprise a description of selecting an individual suitable for treatment based on identifying whether that individual has the target disease. In still other embodiments, the instructions comprise a description of administering an antibody, or antigen binding portion thereof, to an individual at risk of the target disease.

The instructions relating to the use of inhibitors, e.g., antibodies, or antigen binding fragment thereof, that selectively bind a LTBP1-TGFβ1 complex and/or a LTBP3-TGFβ1 complex generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable. The label or package insert can indicate that the composition is used for treating, delaying the onset and/or alleviating a disease or disorder associated with a TGFβ-related indication. Instructions may be provided for practicing any of the methods described herein.

The kits of this disclosure can be provided in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an inhibitor, e.g., antibody, or antigen binding portion thereof, that selectively binds a LTBP1-TGFβ1 complex and/or a LTBP3-TGFβ1 complex, as described herein.

Kits may optionally provide additional components such as buffers and interpretive information. Normally, the kit comprises a container and a label or package insert(s) on or associated with the container. In some embodiments, the disclosure provides articles of manufacture comprising contents of the kits described above.

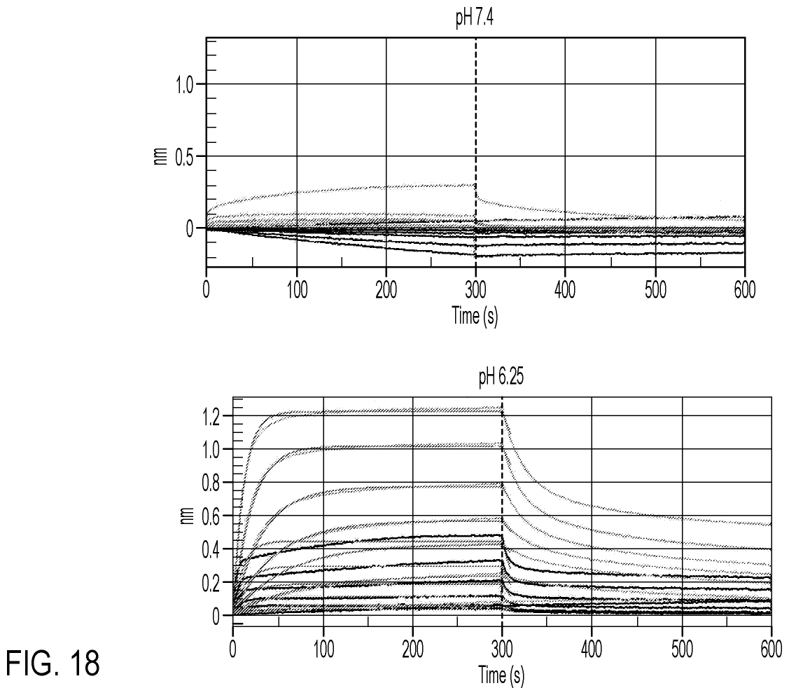

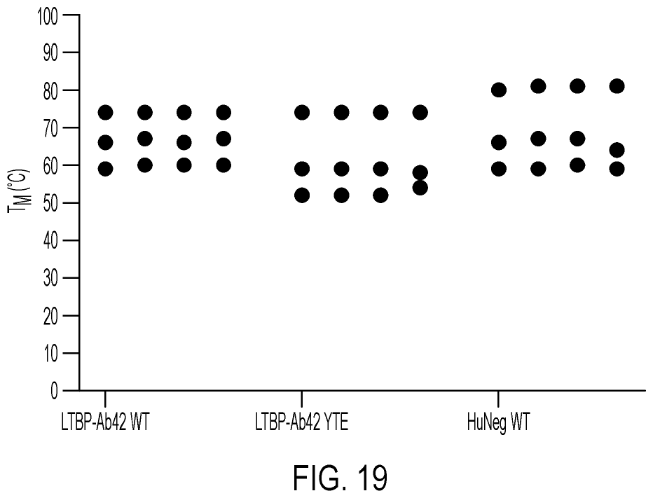

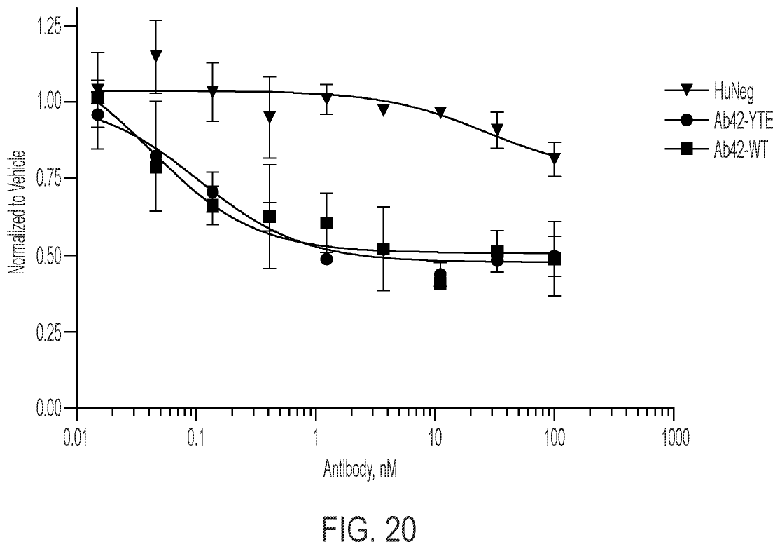

Ab42-YTE characterization. In summary, (1) Ab42-YTE transient expression resulted in similar titer compared to the parental antibody. (2) Both Ab42 and Ab42-YTE antibodies tolerated 3× freeze thaw without aggregation. (3) The Ab42-YTE antibody shows enhanced binding to human FcRn by Octet® measurements. See FIG. 18. (4) The melting temperature of Ab42-YTE is decreased compared with Ab42. See FIG. 19.

The present disclosure is further illustrated by the following examples, which are not intended to be limiting in any way. The entire contents of all references, patents and published patent applications cited throughout this application, as well as the Figures, are hereby incorporated herein by reference.

### Example 1: Effects of LTBP-TGFβ1 Antibodies on Adenine-Induced Kidney Injury and Fibrosis in a Rat Model

The objective of this study was to evaluate the effects of therapeutically administered Ab42 on adenine-induced kidney injury and fibrosis in Sprague-Dawley rats. Ab42 is described in WO 2020/160291 A2, incorporated by reference in its entirety herein. This antibody binds and inhibits LTBP1-presented proTGFβ or LTBP3-presented proTGFβ at high affinities (at least nanomolar range) but does not bind and inhibit immune cell-associated TGFβ, e.g., GARP- or LRRC33-presented proTGFβ1, or the binding is below meaningful levels, e.g., at least fifty times affinities for the LTBP complexes over GARP or LRRC33 complex.

As shown in FIG. 2, Ab42 human Fab binding to human TGFβ1 complexes was highly specific for (1) LTBP1-proTGFβ, (2) LTBP3-proTGFβ, and had picomolar monovalent affinities as measured by BIACORE®. Ab42 has ˜100 fold binding selectivity for human LTBP complexes of TGFβ1 compared to GARP or LRRC33 complexes. The binding profile of Ab42 has been shown to be similar for human and cyno antigens, and the data have been consistent when comparing fab (monovalent) to mab (divalent).

This antibody can selectively inhibit activation of TGFβ in a context-dependent manner, such that they selectively bind, thereby inhibiting the TGFβ signaling axis associated with the extracellular matrix, including selective inhibitors of matrix-associated, e.g., LTBP1-associated or LTBP3-associated or both, TGFβ activation. The inhibitors do not inhibit activation of TGFβ1 associated with immune cell function, mediated by GARP or LRRC33. The improved antibodies encompassed by this disclosure have affinities towards human LTBP1-proTGFβ1 or human LTBP3-proTGFβ1 in at least a nanomolar range, i.e., 1×10⁻⁹M to 10×10⁻⁹M. Such antibodies also have affinities towards murine LTBP1-proTGFβ1 or murine LTBP3-proTGFβ1 in at least a nanomolar range, i.e., 1×10⁻⁹M to 10×10⁻⁹M.

Animals were enrolled to groups in a balanced design such that an approximately equal number of animals were enrolled per group.

Three endpoints initially measured the efficacy of Ab42: (1) kidney function (reduction of plasma blood urea nitrogen, creatinine, etc.); (2) hydroxyproline levels; and (3) picosirius red (PSR) staining. In all measurements, the inventors observed efficacy with all the inhibitory antibodies. Note that (2) hydroxyproline levels and (3) picosirius red staining both measure collagens, the accumulation of which is a hallmark of fibrosis. The former (2) hydroxyproline levels is more reflective of new collagen synthesis. The latter (3) picosirius red staining detects some collagens present in the tissue. The inventors saw clearer effects in (2) hydroxyproline levels and (3) less clear, but still significant, effects in picosirius red staining, which is detecting both the existing and accumulated collagen.

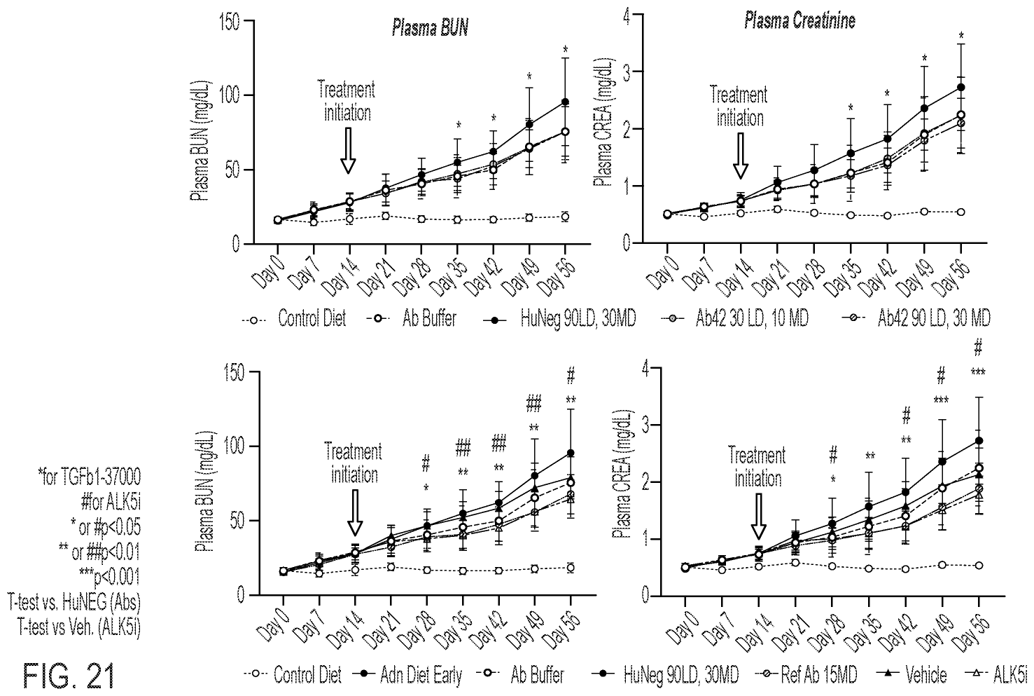

(1) Kidney function. Ab42 reduced plasma blood urea nitrogen and creatinine in the efficacy analysis. In Sprague Dawley rats, an adenine diet induced increased levels of plasma blood urea nitrogen (BUN) and creatinine over analysis duration (FIG. 21). Thus, kidney fibrosis was induced by adenine diet. The inventors observed a significant reduction in levels of plasma blood urea nitrogen and creatinine with Ab42 treatment starting on day 35. The two positive controls small molecule inhibitors of TGFβ type I (ALK5) receptor (ALK5i) and a context-independent reference antibody showed decrease in blood urea nitrogen and creatinine starting at day 28. No significant reduction was observed with Ab1 D11 and Ab46.

Ab1 D11 was used as a pan-TGFβ positive control antibody. Ab1 D11 is TGF beta-1,2,3 Monoclonal Antibody (1 D11), mouse Anti-TGF-β1, 2, 3 Monoclonal Antibody (THERMOFISHER® Product #MA5-23795).

A context-independent reference antibody, also described in WO 2020/160291 A2, incorporated he reference in its entirety herein, was used as a positive control.

Ab42 monovalent (Fab) affinities

| Human antigen complex | Ab42 K_{D} (nM) |
|---|---|
| LTBP1-proTGFβ1 | 0.38 |
| LTBP3-proTGFβ1 | 0.57 |
| GARP-proTGFβ1 | 144.61 |
| LRRC33-proTGFβ1 | 134.25 |

Methods: Animals: Male, Sprague Dawley (SD) rats, weighing 275-300 g at arrival. The model afforded functional as well as fibrotic readouts. The dosing strategy used a loading dose and a maintenance dose. The method was to administer 0.25% adenine for two weeks, then collect Group 1 after two weeks. Then, the rats were randomized using body weight. Rats were enrolled to groups in a balanced design such that an approximately equal number of animals were enrolled per group. Serum creatinine and blood urea nitrogen (BUN) were assayed using standard clinical chemistry methods. Next, the rats received a loading dose of 0.25% adenine+treatment for six weeks. Following, the maintenance dose was twice weekly (BIW) by intraperitoneal administration. Testing for the serum exposure from Ab42 was weekly at trough levels. The rats were tested for creatinine and blood urea nitrogen (BUN) using standard clinical chemistry assays and measured from plasma, which was collected weekly from the rats. Fixed blood was taken weekly at the same time as serum or plasma collection. The last Ab dose was given on day 54, two days before necropsy.
Frequency: Twice/week.
Time points (days): 14 (pre-dose), 21, 28, 35, 42, 49, and 56.

### Dosing Schedule

Loading Dose: Day 14.

### Maintenance Dose: Days 18, 21, 25, 28, 32, 35, 39, 42, 46, 49, and 54.

### Sample Collection

Groups 1 & 3-10: Days 21, 28, 35, 42, and 49 (immediately prior to next dose).

Groups 1 & 3-10: Day 56 (two days post-final dose).

Other tests performed included picosirius red staining, Col1 immunohistochemistry [using anti-Collagen I antibody [COL-1], Abcam, Cambridge, CB2 0AX, UK (catalogue #ab6308)], and pSmad2 immunohistochemistry (kidney-cortex).

Material for Ab42 pharmacokinetics/pharmacodynamics analysis. Immediately after the rats were sacrificed, tissue was taken from the rats and snap-frozen tissue for hydroxyproline testing, TGFβ testing, and testing for other markers. The table below summarizes the design of the Rat adenine efficacy analysis study described herein.

Study Design
Rat adenine efficacy analysis design

| | | | Loading | Maintenance | | No. of |
|---|---|---|---|---|---|---|
| Group | | | dose | dose | Dose | animals |
| no. | Diet | Test material | (mg/kg) | (mg/kg) | regimen | (males) |
| 1 | 0.25% | No treatment | N/A | | N/A | 10 |
| | adenine | | | | | |
| 2 | 0.25% | Antibody buffer | N/A | | 2x/IP | 10 |
| | adenine | | | | weekly | |
| 3 | Chow | No treatment | N/A | | N/A | 10 |
| 4 | 0.25% | HuNeg-rIgG1 | 90 | 30 | 2x/IP | 15 |
| 5 | adenine | Ab1D11 | N/A | 5 | weekly | 15 |
| 6 | | context- | N/A | 15 | | 15 |
| | | independent | | | | |
| | | reference | | | | |
| | | antibody | | | | |
| 7 | | Ab42 | 90 | 30 | | 15 |
| 8 | | Ab42 | 30 | 10 | | 15 |
| 11 | | Vehicle | N/A | | BID days | 15 |
| 12 | | GS-490470 | N/A | 1.0 | 1-56 | 15 |

(2) Hydroxyproline reduction was observed with TGFβ1 specific inhibitors. Immediately after the rats were sacrificed, tissue for hydroxyproline testing was taken from the rats and snap-frozen. Hydroxyproline levels were determined in pulverized kidney tissue.

Hydroxyproline levels are indicative of the fibrotic phenotype. The efficacy analysis used 30 mg/kg/week. In WO 2020/160291 A2, incorporated by reference in its entirety herein, the inventors had tested 30 mg/kg/week and saw reduced hydroxyproline levels and p-Smad2/3 in choline-deficient high fat diet (CDHFD) mice and Alport syndrome mice.

In subsequent analyses, the inventors tested an adenine-induced kidney fibrosis model and saw efficacy as well. Here, much lower doses of Ab42 were tested (as low as 3 mg/kg) and effects were still observed.

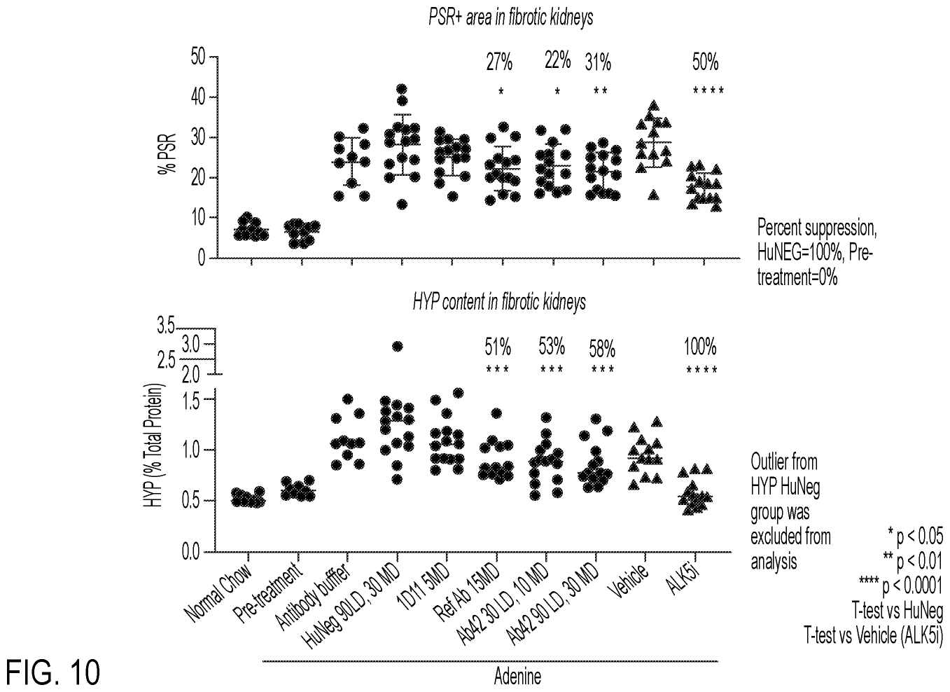

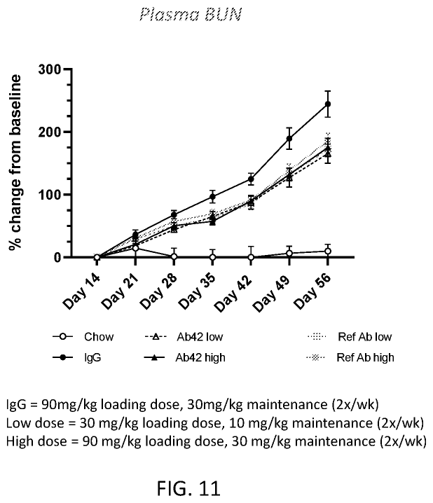

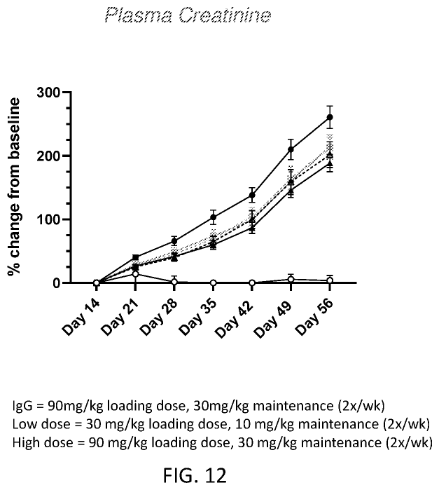

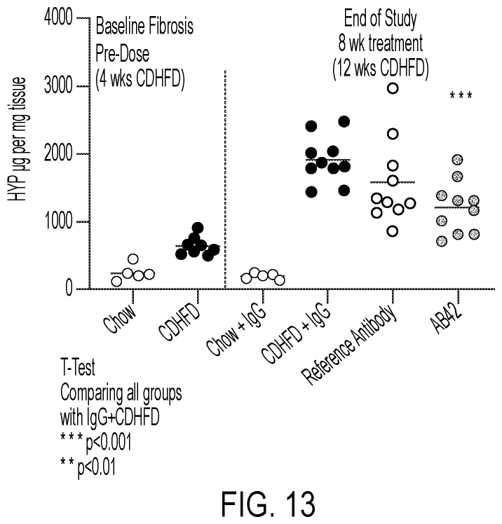

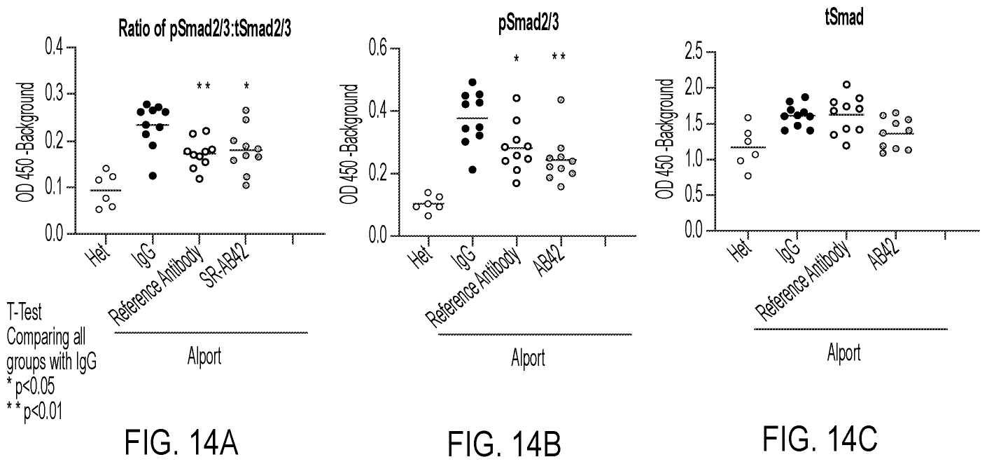

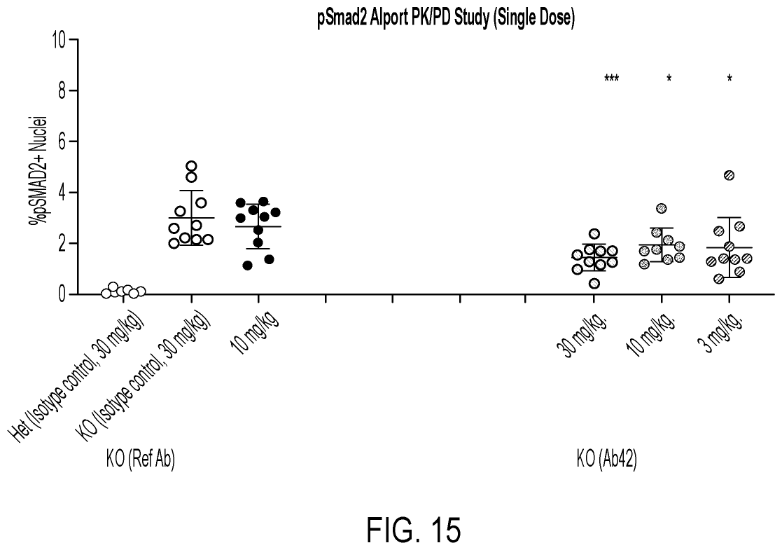

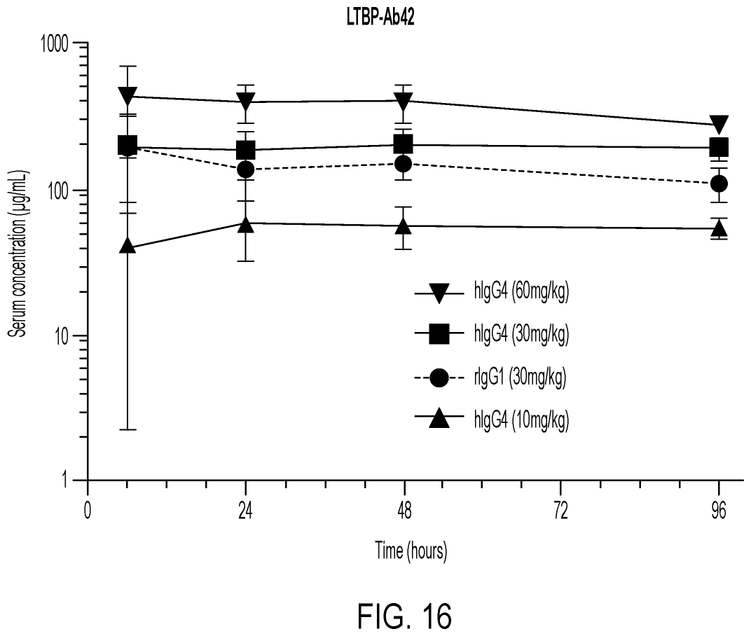

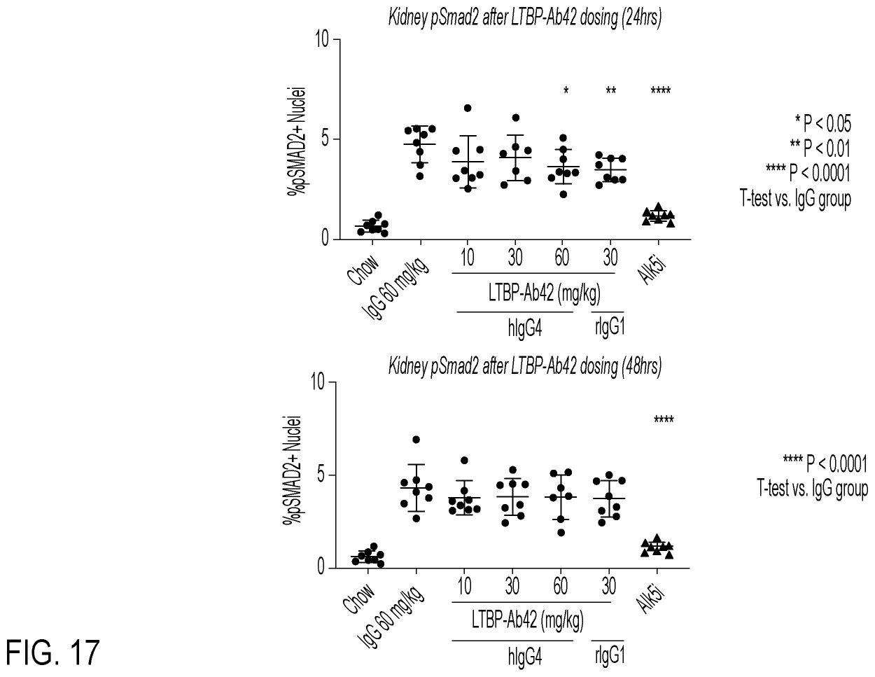

Rats on the adenine diet over seven weeks showed an increase in kidney hydroxyproline content. A significant reduction of hydroxyproline in all TGFβ1 selective antibody treated groups and in ALK5i groups. These results are shown in FIG. 10.

(3) Picrosirius red. Picrosirius red staining was also carried out; however the results were not conclusive and further testing will be carried out.

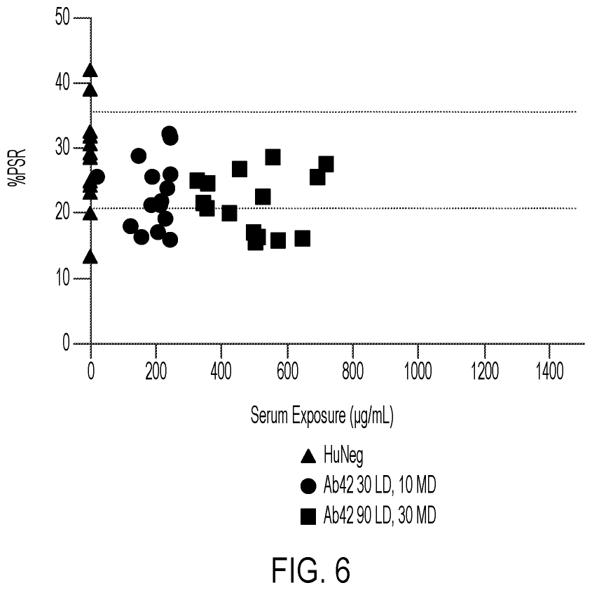

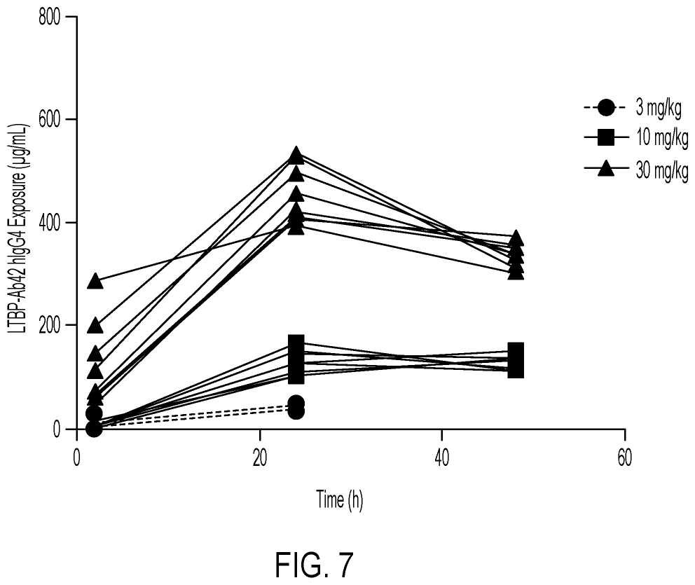

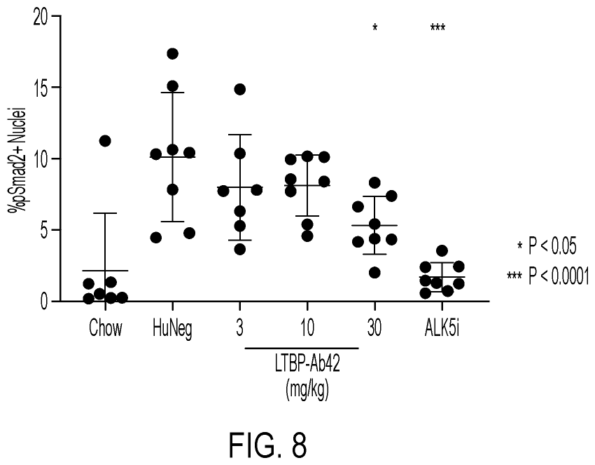

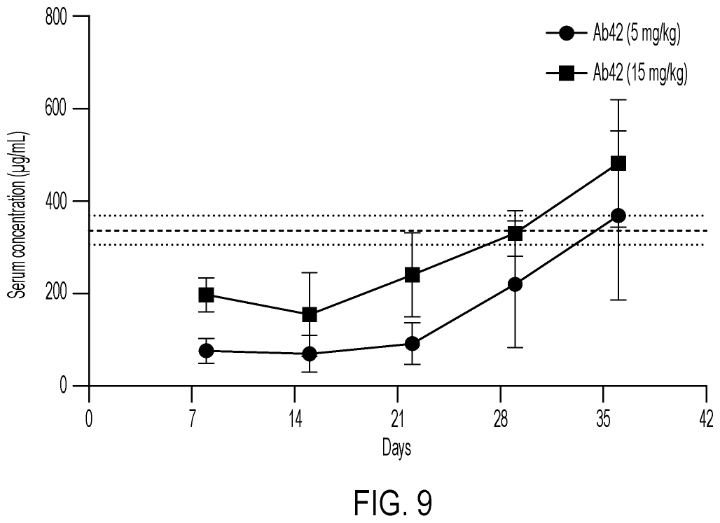

The inventors generated a picosirius red quantitation analysis from an idiopathic pulmonary fibrosis-precision cut lung slice (IPF-PCLS) analysis. An example of picosirius red-based in vitro binding assay is provided in EXAMPLE 9 of WO 2020/160291 A2, incorporated by reference in its entirety herein. Fab fragments of Ab42 and other antibodies which are activation inhibitors of TGFβ1 were used in this EXAMPLE. As illustrated in the EXAMPLE, these antibodies have sub-nanomolar K_{D} and “OFF” rates that are ≤5×10⁴/seconds). A picosirius red/serum exposure correlation for Ab42. See FIG. 6.

Significance by unpaired T-test compared to HuNeg

| picosirius red staining | p-value |
|---|---|
| context-independent reference | 0.0202 |
| antibody 15 mg/kg | |
| Ab42 10 mg/kg | 0.0354 |
| Ab42 30 mg/kg | 0.0068 |

The inventors also compared Alk5i administration to vehicle. The statistical significance was maintained.

| Group | N/group Animal # | Compound and Dose |
|---|---|---|
| 6 | n = 15 (217-231) | context-independent reference |
| | | antibody 15 mg/kg |
| 7 | n = 15 (232-246) | Ab42 30-10 mg/kg |
| 8 | n = 15 (247-261) | Ab42 90-30 mg/kg |

### Results

An adenine diet over 8 weeks induced kidney damage and fibrosis in rats (data not shown). During the course of the study, average body weight and food consumption in the treatment groups did not significantly change. As shown in FIG. 10, an anti-fibrotic response was achieved by selectively targeting LTBP-TGFβ1 complexes with Ab42, administered at a loading dose of 30 mg/kg and maintenance dose of 10 mg/kg and at loading dose of 90 mg/kg and maintenance dose of 30 mg/kg. FIG. 10 (top) is a graph that shows quantitation of picosirius red (PSR) staining of collagen fibers (by percent (%)) in fibrotic kidneys. FIG. 10 (bottom) is a graph that shows quantitation of hydroxyproline (HYP) content as a measurement of collagen levels (by percent (%)) in fibrotic kidneys. An adenine diet induced increase in kidney PSR staining and in HYP content. As shown in FIG. 10, there was a significant reduction of fibrosis with Ab42, a LTBP/TGFβ1-specific antibody, and efficacy was observed at both dose levels, An antifibrotic response was observed with TGFβ1 context-independent inhibitors and ALK5i. Treatment with second pan-TGFβ positive control 1 D11-rIgG1 was not antifibrotic.

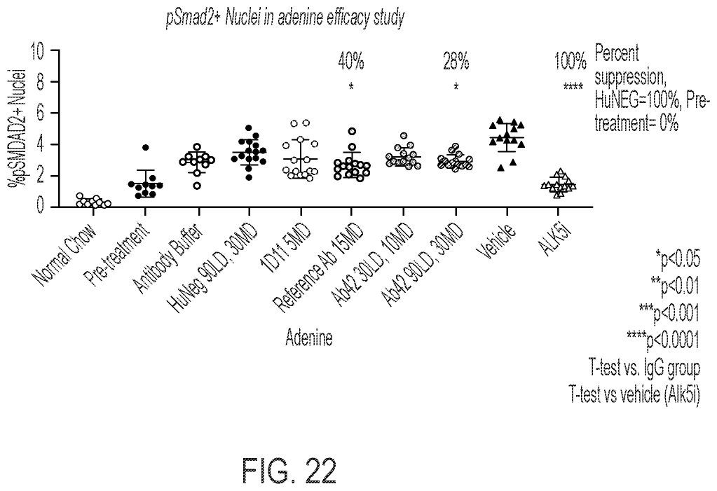

An immunohistochemical (IHC) analysis was carried out to determine the amount of phosphorylated Smad2 (active Smad2) in fibrotic kidney samples after treatment with Ab42 (FIG. 22). pSmad2-positive nuclei were assayed at 48 hours after antibody treatment. There was a partial suppression of pSmad2 by Ab42 in efficacy study, which was similar to the partial reduction of pSmad2 observed in the adenine PK/PD study (shown below). Treatment with the second pan-TGFβ positive control 1 D11-rIgG1 was not antifibrotic. These results indicated that alternative PD marker(s) for Ab42 would be useful.

### PK Simulation Provided Rationale for Loading Dose Approach in Rat Adenine Efficacy Study

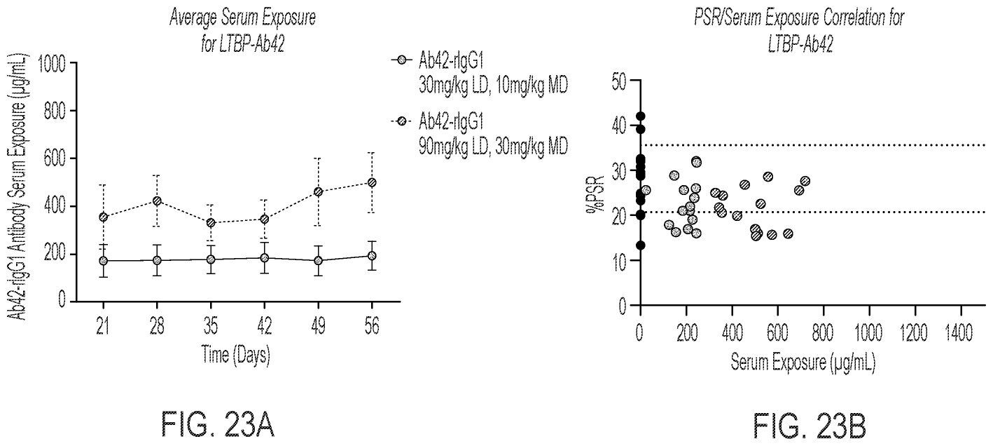

Dose accumulation in previous adenine studies required three weeks of treatment. Several rounds of dosing were required to achieve dose “stacking” in previous adenine repeat-dose efficacy studies using Ab42. Based on PK simulation (not shown), it was found that the loading dose approach would help achieve the serum exposure that was needed to engage the target within first week of dosing. Thus, a loading dose strategy was used to achieve consistent serum exposure over treatment duration. Based on this strategy, the average serum exposure for Ab42 is shown in FIG. 23A. The PSR/serum exposure correlation for Ab42 is shown in FIG. 23B. These results demonstrated that Ab42 serum exposures of ˜200 ug/mL can achieve significant reduction in PSR in fibrotic kidneys.

Taken together, the results from these studies demonstrated that targeting LTBP-TGFβ1 complexes with Ab42 elicited an anti-fibrotic response. An anti-fibrotic response was achieved with Ab42 in rat adenine (kidney) model. The picrosirius red staining, hydroxyproline, and decline in kidney function (plasma blood urea nitrogen and creatinine) significantly improved. These in vivo pharmacological results constitute proof-of-biology that targeting LTBP-TGFβ1 complexes elicits an anti-fibrotic response.

Using these results as guidance, administering Ab42 can result in evidence of anti-fibrotic response in idiopathic pulmonary fibrosis (IPF) precision cut lung slices (for the lung acquisition phase), as described in Example 10.

### Example 2: Rat Adenine Pharmacokinetics/Pharmacodynamics (PK/PD) Analysis

The objective of this study was to evaluate the effects of therapeutically administered context-specific TGFβ1 antibodies on adenine-induced kidney injury and fibrosis in Sprague-Dawley rats. The results described herein demonstrated that the rat adenine kidney model provided biological evidence that targeting LTBP-TGFβ1 complexes is anti-fibrotic.

Male Sprague-Dawley rats (275-300 g in weight on arrival) were put on an adenine diet for 8 weeks. Each test group of animals consisted of 3 groups of 8 rats (total of 24 rats). After 8 weeks, the animals were randomized based on body weight, serum BUN, and serum creatinine. Animals were given a single dose of antibody on Day 1, at a dosage as in indicated in the Study Design, below. Endpoints were phosphoSmad2 immunohistochemistry (pSmad2-IHC) hydroxyproline assay (HYP) and antibody exposure. During the course of the study, average body weight and food consumption in the treatment groups did not significantly change (not shown). Further, it was confirmed that all animals had a similar course of disease progression by assessment of hydroxyproline levels (not shown). The study design is shown in the Table below.

Study Design

| | |
|---|---|
| Diet: | Control Diet: 0.9% Ca, 0.6% P/Envigo/TD.170417. |
| | 0.25% Adenine diet: Envigo/TD.180493. |
| Doses/Route/ | Control diet: p.o./Ad lib day 0 through endpoint. 0.25% |
| Frequency: | Adenine diet: p.o./Ad lib day 0 through endpoint. |
| | p.o. = oral route of administration. |
| Dose | Control diet: Ad lib access. 0.25% Adenine diet: Ad lib |
| Volume: | access. |
| Compound | Control diet: 20 kg (pellet form). 0.25% Adenine diet: |
| Requirement: | 210 kg (pellet form). |

Time course: The rat tissue (kidney-cortex) collection was a serum/blood collection @6, 24, 48, and 96 hours.

The small molecule inhibitors of TGFβ type I (ALK5) receptor (ALK5i) were dosed two hours pre-necropsy.

Study Design

| | | | | | Tissue | |
|---|---|---|---|---|---|---|
| | | Therapeutic | Dose level | Dosing | Collection | No. |
| Group | Diet | agent | (mg/kg) | frequency | (hours) | animals |
| 1 | Chow | N/A | N/A | N/A | 96 | 8 |
| 2 | 0.25% | HuNeg-hIgG4 | 60 | Day 1 | 24, 48, 96 | 3 × 8 (24) |
| 3 | Adenine | Ab42-hIgG4 | 60 | Day 1 | 24, 48, 96 | 3 × 8 (24) |
| 4 | | Ab42-hIgG4 | 30 | Day 1 | 24, 48, 96 | 3 × 8 (24) |
| 5 | | Ab42-hIgG4 | 10 | Day 1 | 24, 48, 96 | 3 × 8 (24) |
| 6 | | Ab42-rlgG1 | 30 | Day 1 | 24, 48, 96 | 3 × 8 (24) |
| 10 | | GS-490470 | 10 | Once | 2-hour pre- | 8 |
| | | | | | necropsy | |

### Results

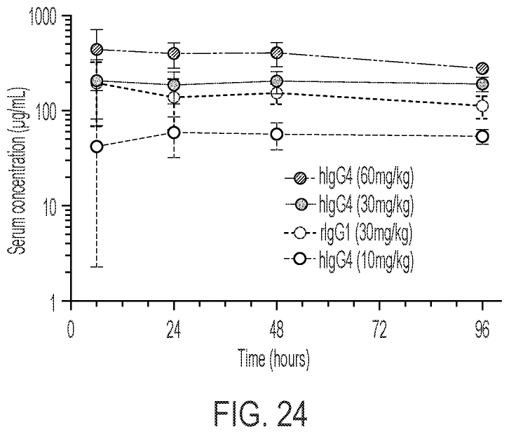

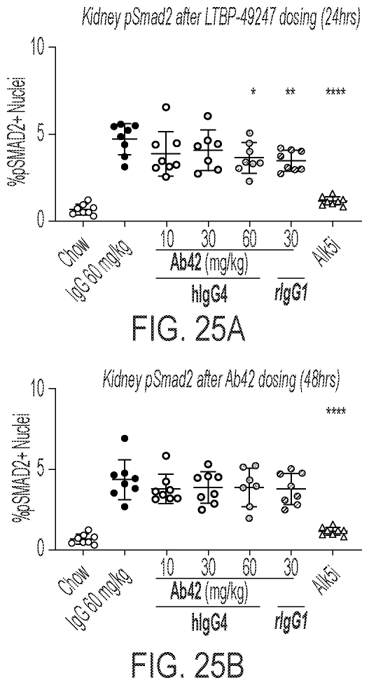

As shown in FIG. 24, exposure with Ab42 was dose-proportional and maintained across all time points. FIGS. 25A and 25B show the results of immunohistochemical (IHC) analysis to determine the amount of phosphorylated Smad2 (active Smad2) in fibrotic kidney samples after treatment with Ab42. FIGS. 25A and 25B show that LTBP-selective inhibition was not sufficient to suppress pSMAD using the methods tested here. One hypothesis is that only a few cells, or a subset of cells, are responsible for driving fibrosis and/or there is a temporal factor involved in driving fibrosis, that is not captured in a one-time-point data.

This is in contrast to when a context independent TGFβ1 inhibitor was employed, there was a significant reduction of pSmad2-positive nuclei. Thus, Ab42 differentiates from context-independent TGFβ1 inhibition in vivo, even at high serum exposure. It can be concluded from these results that context-independent TGFβ1 inhibition is required to substantially suppress Smad2 phosphorylation. Similar efficacy observed compared to context independent TGFβ1 and ALK5i suggesting that fibrotic response is driven by LTBP presented TGFβ1.

### Minimal Effective Serum Exposure

The results herein indicated that minimal effective serum exposure was significantly lower than in previous rodent fibrosis models. In Alport kidneys, the minimal effective serum exposure of Ab42 was approximately 13 μg/mL. In the rat adenine kidney model, the minimal effective serum exposure of Ab42 was approximately 200 μg/mL. In the rat CDHFD liver model, the minimal effective serum exposure of Ab42 was approximately 340 μg/mL.

### Example 3: Rat Kidney Gene Expression Analysis from Adenine Efficacy Study

Experimental groups correspond to those in Example 1, and all tissue samples were obtained from the same. All cortical tissue from the right kidney was harvested and immediately flash-frozen in liquid nitrogen, and stored at −80° C. Approximately 200 mg of powdered tissue was analyzed.

### Materials:

MAGMAX™ mirVana™ Total RNA Isolation Kit: ThermoFisher® (Cat #A27828)
High Capacity cDNA Reverse Transcription Kit: Applied Biosystems by Thermo Fisher Scientific® (Cat #4368813)
TaqMan Fast Advanced Master Mix: Applied Biosystems by Thermo Fisher Scientific® (Cat #4444557)
QuantStudio 6 Flex from Applied Biosystems by Life Technologies®

The gene panel used was as follows:

| | Thermo Cat# | | Thermo Cat# |
|---|---|---|---|
| ACTB | Rn00667869_m1 | IL-6 | Rn01410330_m1 |
| HPRT | Rn01527840_m1 | LOXL2 | Rn01466080_m1 |
| CCL2/MCP1 | Rn00580555_m1 | LRRC32/GARP | Rn01510975_m1 |
| COL1a1 | Rn01463848_m1 | LTBP1 | Rn00572098_m1 |
| COL3a1 | Rn01437681_m1 | LTBP3 | Rn01501879_m1 |
| CTGF | Rn01537279_g1 | MMP2 | Rn01538170_m1 |
| CD68 | Rn01495634_g1 | MMP9 | Rn00579162_m1 |
| TIMP1 | Rn01430873_g1 | NRROS/LRRC33 | Rn01439286_m1 |
| LCN2 | Rn00590612_m1 | SERPINE1/PAI-1 | Rn01481341_m1 |
| HAVCR1/ | Rn00597703_m1 | TGFβ1 | Rn00572010_m1 |
| KIM1 | | | |
| TNFa | Rn01525859_g1 | TGFβ2 | Rn00676060_m1 |
| FN1 | Rn00569575_m1 | TGFβ3 | Rn00565937_m1 |
| IL-1b | Rn00580432_m1 | THBS1 | Rn01513693_m1 |

RNA Isolation: RNA isolation was prepared according to manufacturer's (ThermoFisher®) protocol as follows:
tissue samples were lysed
size of tissue (in mg) to be homogenized was determined amount of Lysis Binding Mix needed to homogenize the tissue was determined
Lysis Binding Mix was prepared
tissue added to the prepared Lysis Binding Mix.
tissue sample homogenized using standard homogenization procedures.
Bind the RNA to the RNA Binding Beads
Vortexed the lysates and transferred 100 μL to a separate well in a MAGMAX™ Express-96 Deep Well Plate.
(Optional) Added 10 μL of chloroform to each well.
Covered the plate and shake.
Added 100 μL of isopropanol to each sample, covered the plate, and shake.
Added 20 μL of the prepared Binding Beads Mix to each sample and shake.
Proceeded immediately to “Wash, rebind, and elute the RNA”.
Set up the processing plates
Washed, rebinded, and eluted the RNA
Ensured that the instrument was set up for processing with the deep well magnetic head and selected the program on the instrument.
Started the run and loaded the prepared processing plates in their positions when prompted by the instrument.
Loaded the sample plate (containing lysate, isopropanol, and Binding Beads Mix) at position 1 when prompted by the instrument.
When prompted by the instrument (30-35 minutes after the initial start):
Removed the DNase Plate from the instrument.
Added 50 μL of Rebinding Buffer and 100 μL of isopropanol to each sample well. Added Rebinding Buffer and isopropanol immediately after the prompt, to prevent excessive drying of any beads that are still captured on the Tip Comb.
Loaded the DNase Plate back onto the instrument, and pressed Start.
At the end of the run (approximately 45 minutes after the initial start), removed the Elution Plate from the instrument and sealed immediately with a new MICROAMP™ Clear Adhesive Film.
   cDNA Synthesis:
Pipetted 14 ul RNA samples into designated wells of 96 well semi-skirted PCR plate.
Prepared+RT Reaction Mix according to manufacturer instructions and number of samples.
Pipetted 14 ul prepared+RT Reaction into each sample well; total volume of 28 ul.
Plate was sealed and centrifuged at 2,000×g for 2 min.
Plate was placed into thermocycler and reaction conditions were set as follows:

| 25° C. | 37° C. | 85° C. | 4° C. |
|---|---|---|---|
| 5 min | 120 min incubation | min | ∞ |

Plate was stored at −20 C
   qPCR:
Prepare designated genes according to TagMan® Fast Advanced Master Mix manufacturer instructions.
9 ul of master mix/probe was added per well, in duplicates, into MICROAMP™ Optical 96-w reaction plate.
1 ul cDNA sample was added to designated wells. Plate was sealed and centrifuged at 2,000×g for 1 min.
Plate was run on QuantStudio 6 Flex; reaction conditions as follows:

| Hold | Cycles (40 cycles) |
|---|---|
| 20 sec @ 95° C. | 1 sec @ 95° C. 20 sec @ 60° C. |

### Results

TGFβ3-related genes analyzed included TGFβ1, TGFβ2, TGFβ3, LTBP1, LTBP3, LRRC32 (GARP), LRRC33, Serpine1 (PAI-1), and Thbs1 (thrombospondin). Fibrotic genes analyzed included Coil a1, Fn1, Mmp2, Mmp9, Col3a1, Loxl2, Ctgf, Timp1, and Kim1. Inflammatory genes analyzed included CD68, MCP-1 (CCL2), IL-6, TNFα, IL-1β, and NGAL. Gene expression was tested at 10, 30 and 60 mg/kg at 24, 48 and 96 hours after Ab42 treatment.

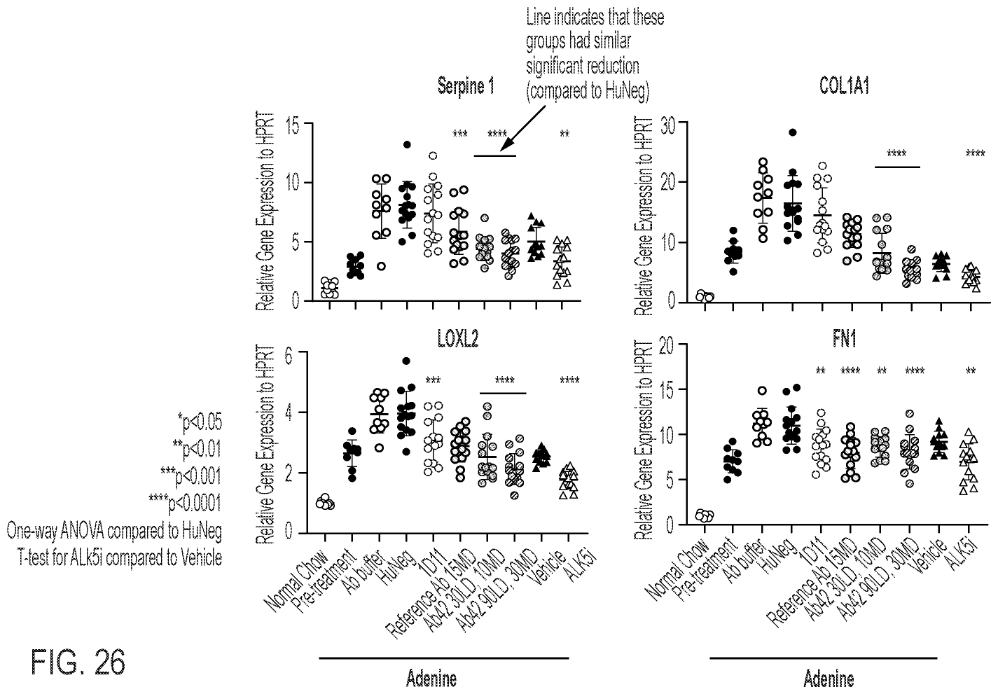

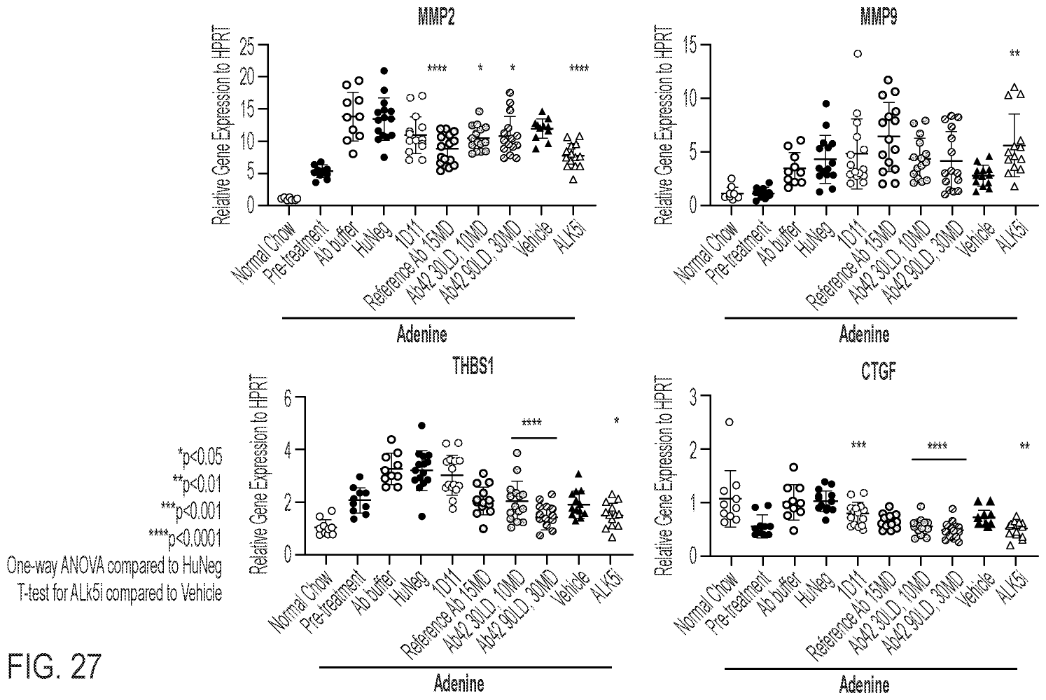

It was found that there was a significant reduction of TGFβ related and pro-fibrotic genes with Ab42 treatment. As shown in FIG. 26 and FIG. 27, treatment with Ab42 reduced TGFβ related genes Serpine 1 and Thrombospondin 1 (Thbs1), respectively. There was a strong induction of profibrotic gene expression (e.g., col1a1, Fn1, Loxl2, ctgf, MMP2 and MMP9, shown in FIG. 27) in the adenine model, and a reduction of pro-fibrotic genes was seen with treatment with LTBP-TGFβ1 specific inhibitor Ab42 at the tested doses.

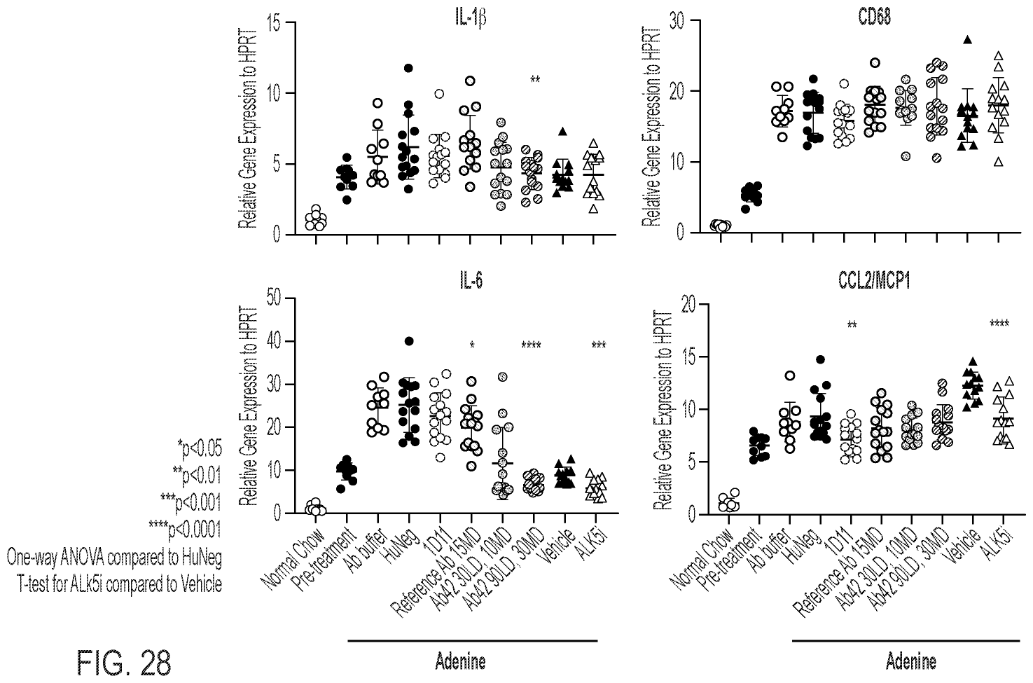

Upregulation of pro-inflammatory genes was measured in damaged kidneys (FIG. 28). As shown in FIG. 28, treatment with Ab42 reduced II-1b, II-6, and Tnfα (not shown) gene expression. CD68 is a macrophage marker accumulation of which indicates inflammation. Work by Meng et al. (Cell Death and Disease (2016) 7 e2495, incorporated by reference in its entirety herein) suggested that accumulation of CD68+macrophages in fibrotic kidney may be associated with macrophage-to-miofibroblast transition which leads to increased collagen deposit. Results demonstrated that there was no effect on Cd68 mRNA levels by Ab42, indicating that selective targeting and inhibition of LTBP-associated TGFβ31 did not cause macrophage accumulation at the site of fibrosis.

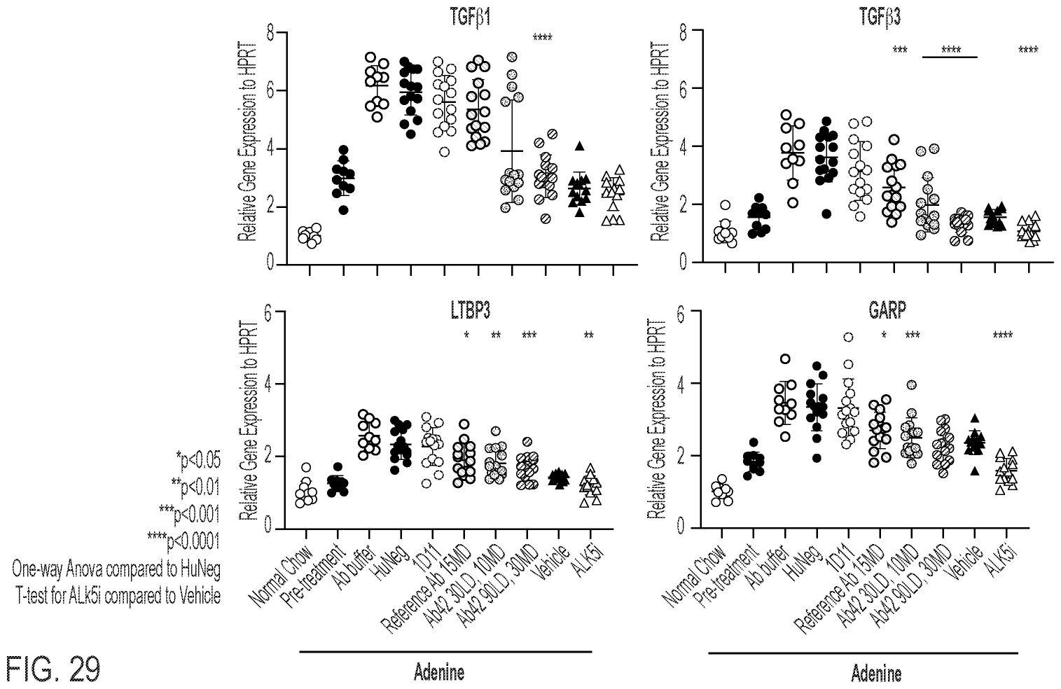

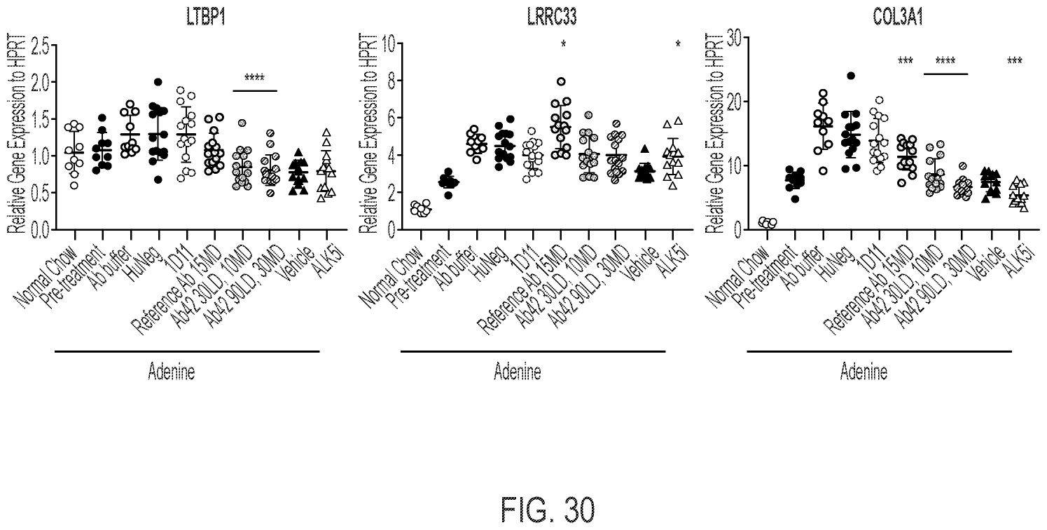

A reduction of TGFβ ligands and presenting molecules was observed with Ab42 treatment (FIG. 29). As shown in FIG. 29, there was a significant reduction of TNFα and TGFβ ligands with Ab42 treatment. Interestingly, TNFα gene expression was increased upon treatment with pan-TGFβ inhibitor (1 D11) but did not significantly increase upon treatment with an isoform-selective TGFβ1 inhibitor alone, demonstrating that Ab42 does not exacerbate inflammatory gene expression to the same degree as pan-TGFβ inhibitors. A significant reduction of Ltbp1 and Col3a1 genes (FIG. 30) Ltbp3 and Garp genes was also found after multiple treatments with Ab42.

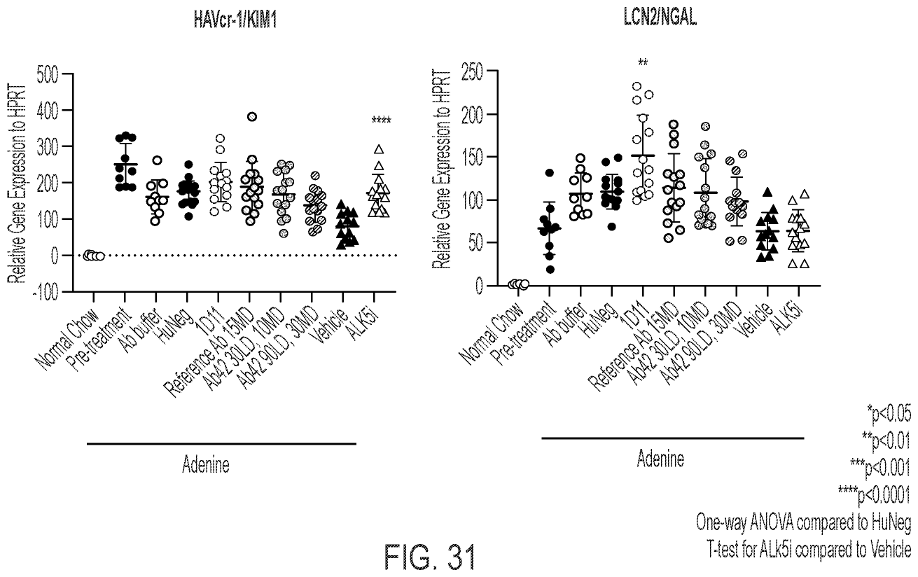

Unexpectedly, neither of the pro-fibrotic genes Havcr/KIM1 or Lcn2/NGAL were reduced by treatment with Ab42 at any of the doses (FIG. 31).

### Example 4: Rat Kidney Gene Expression Analysis from Adenine PK/PD Study

Experimental groups correspond to those in Example 2, and all tissue samples were obtained from the same. All cortical tissue from the right kidney was harvested and immediately flash-frozen in liquid nitrogen, and stored at −80° C. Approximately 200 mg of powdered tissue was analyzed.

Methods are carried out as described in Example 3, above.

### Results

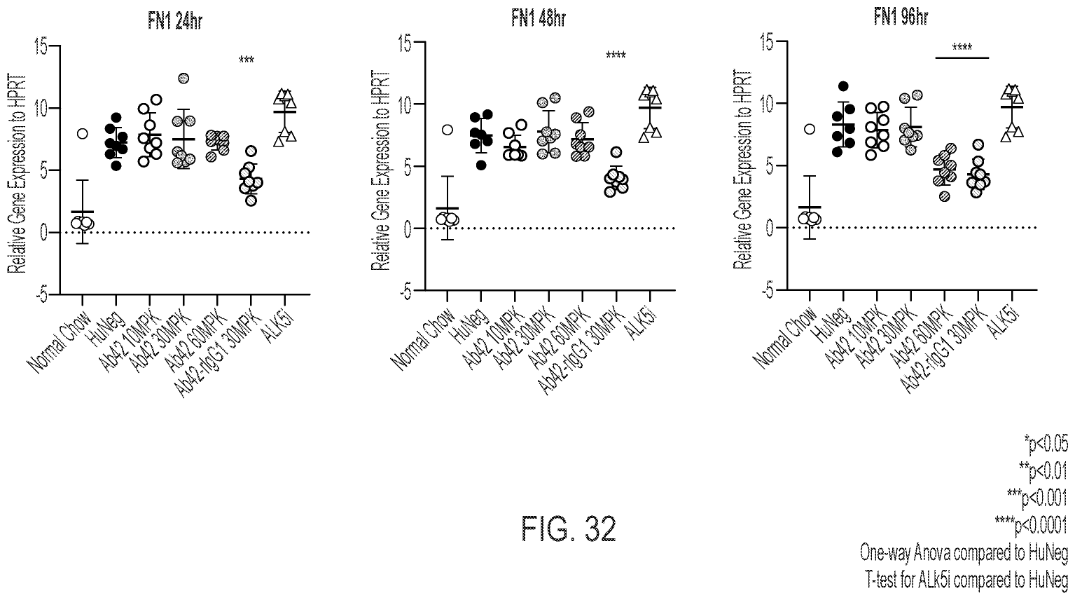

FIG. 32 shows consistent suppression of Fn1 gene with Ab42. Treatment with Ab42 at 30 mg/kg showed suppression of Fn1 at all timepoints. There was no reduction by Ab42 at 30 mg/kg but significant reduction with 60 mg/kg Ab42. There was no effect observed with ALK5i.

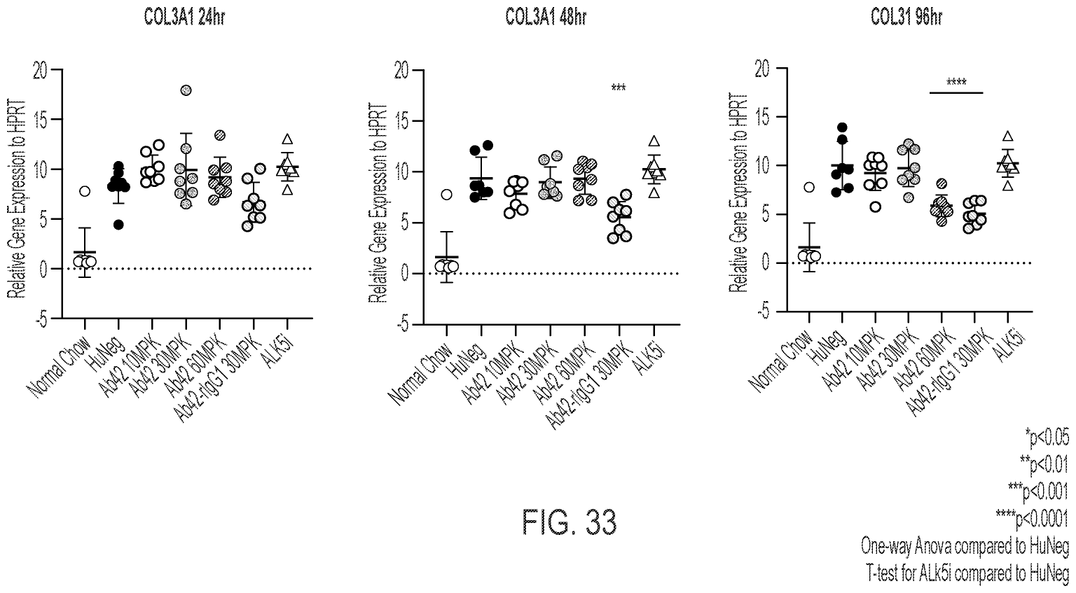

FIG. 33 shows reduction of Col3a1 with the treatment of Ab42 at 48 and 96 hrs. Treatment with Ab42 at 30 mg/kg showed suppression of Col3a1 at 48 hrs and 96 hrs. Only at 96 hrs does Ab42 at 60 mg/kg reduce Col3a1 gene expression. No reduction was observed with any timepoints for Coil a1 with Ab42 (not shown). There was no effect observed with ALK5i.

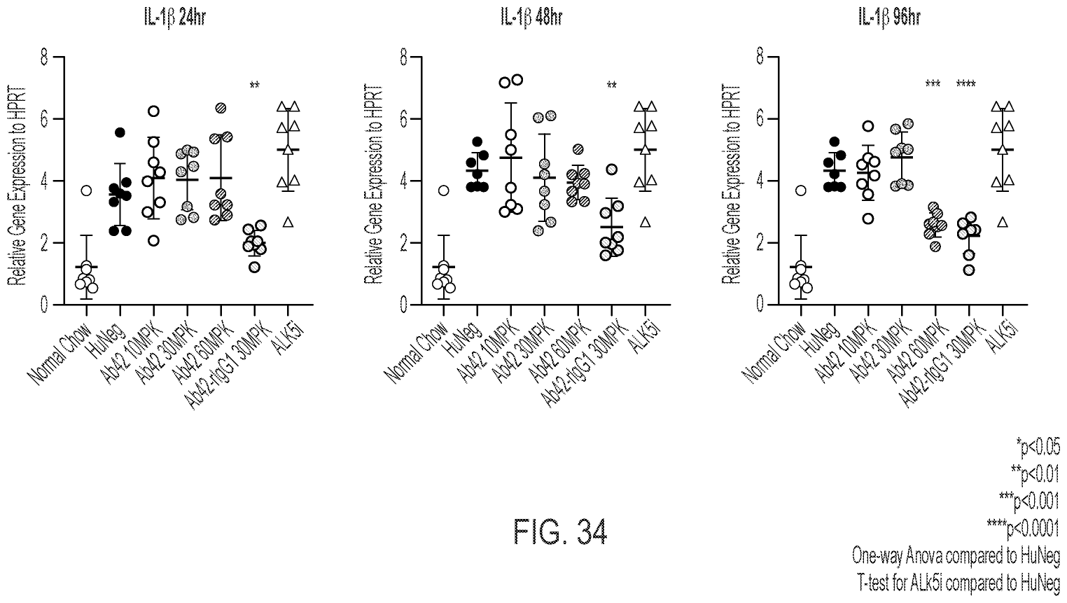

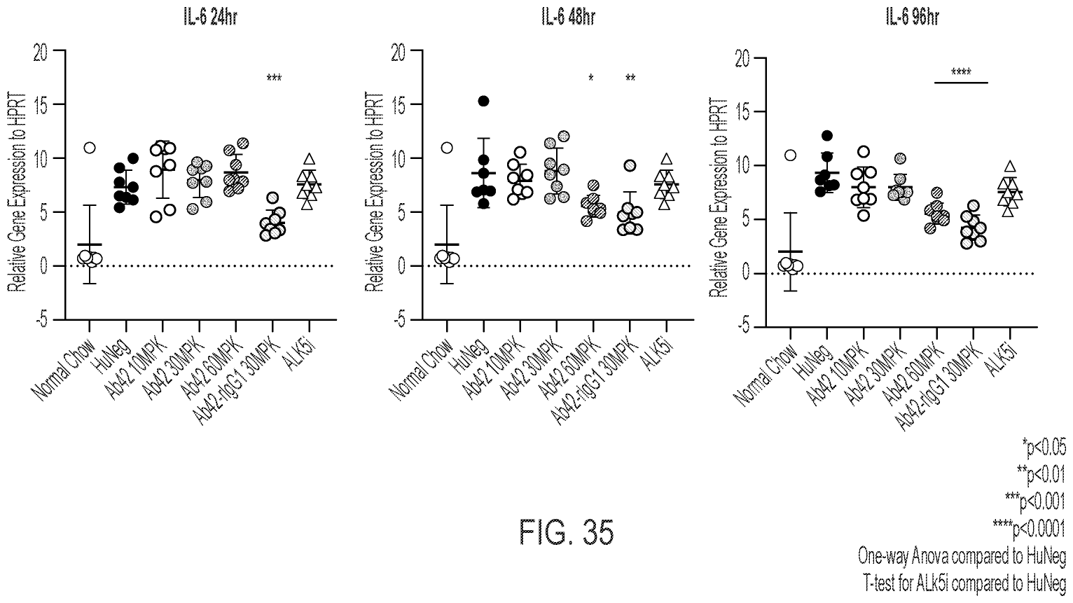

A reduction in pro-inflammatory genes was observed with II-6 and II-1b (FIGS. 34 and 35, respectively). This suggested that targeting the LTBP-TGFβ1 complexes in the ECM may affect surrounding inflammatory cells.

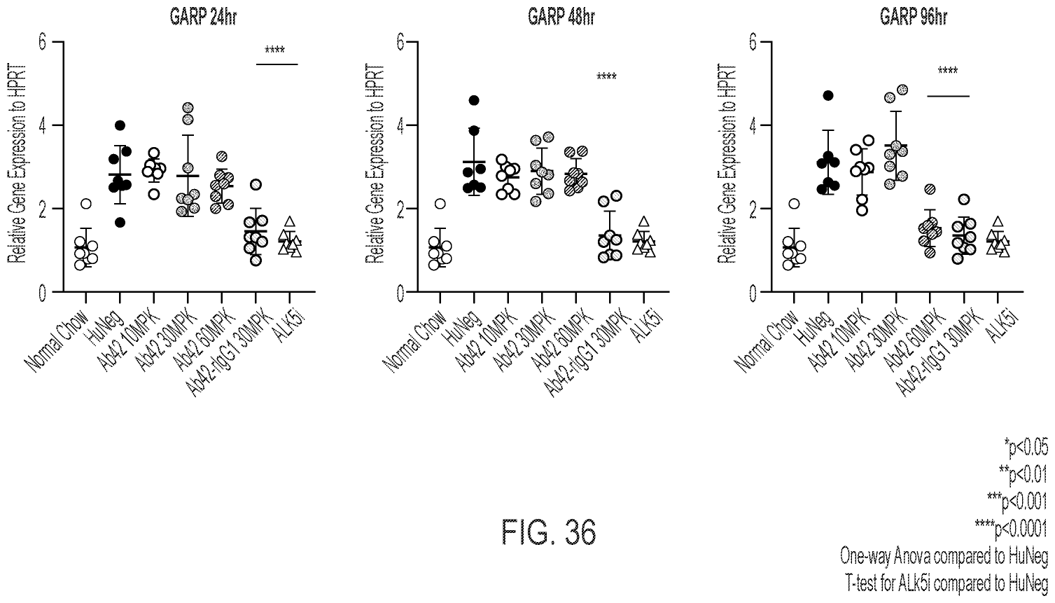

FIG. 36 shows that there was a reduction of GARP gene After Single Dose of Ab42. Treatment with Ab42-IgG1 at 30 mg/kg showed suppression of Garp at all timepoints. Similar to previous that 60 mg/kg of Ab42-IgG4 reduced Garp at 96 hrs. GARP is expressed not only on activated Tregs but also endothelial cells. Potentially Ab42 could be inhibiting Garp gene expression in endothelial cells.

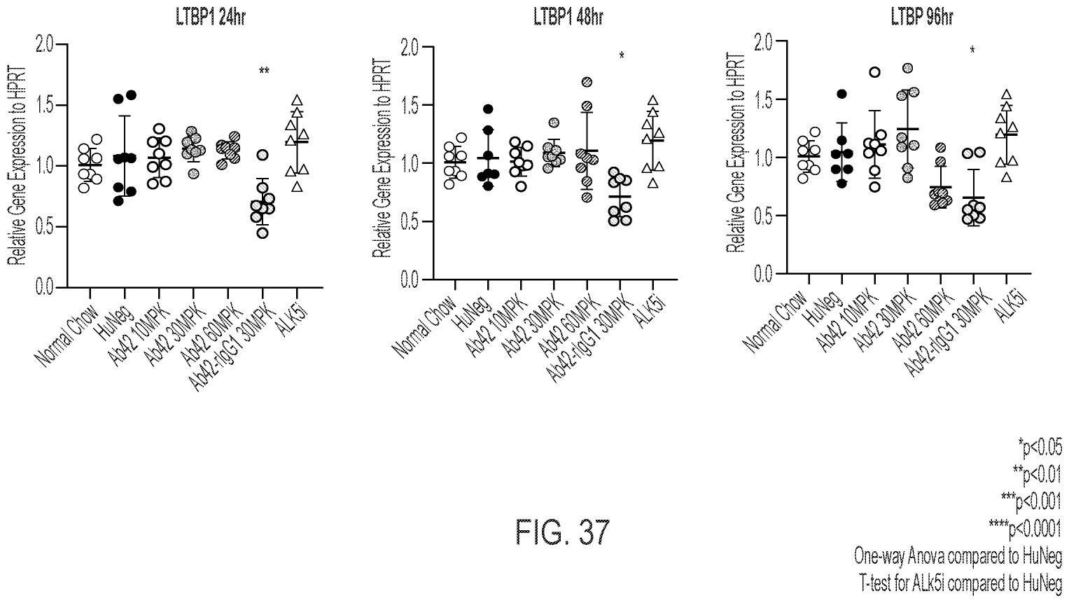

FIG. 37 shows that there was a reduction of Ltbp3 gene with treatment of Ab42. Treatment with Ab42 at 30 mg/kg showed suppression of Ltbp3 at all timepoints. There was no induction of Ltbp1 gene when compared to normal chow (not shown).

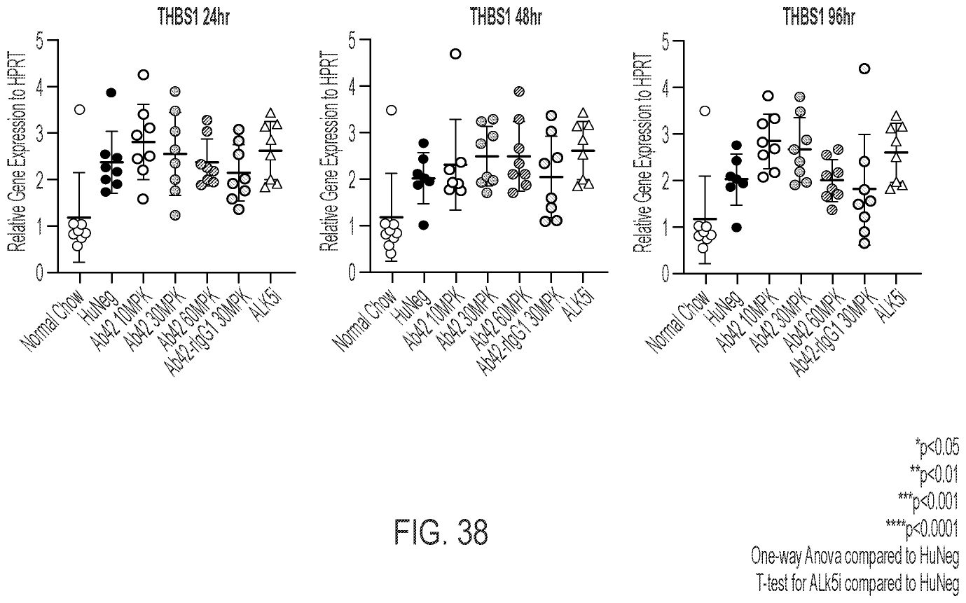

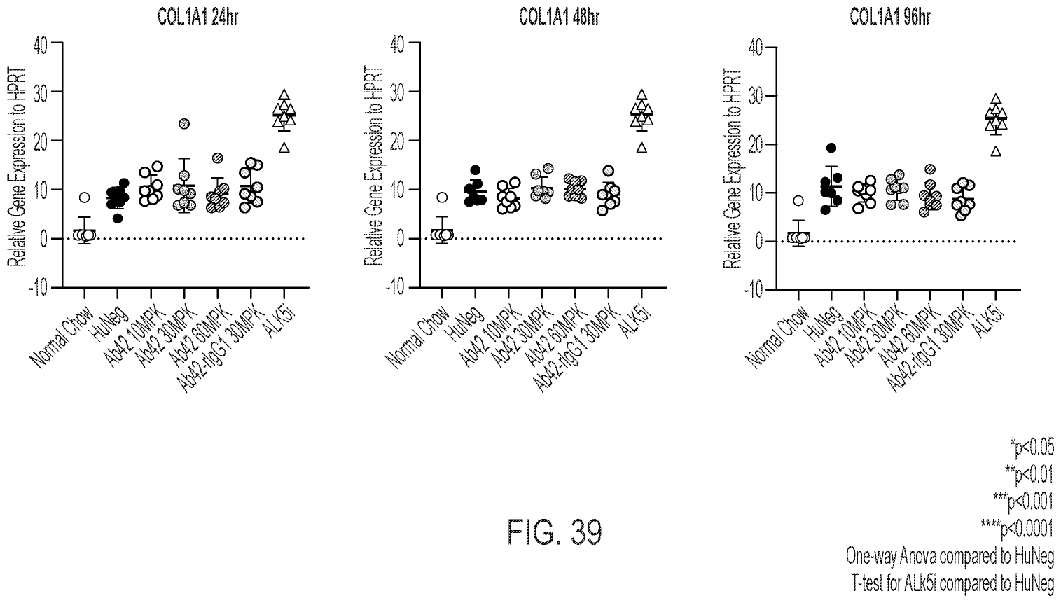

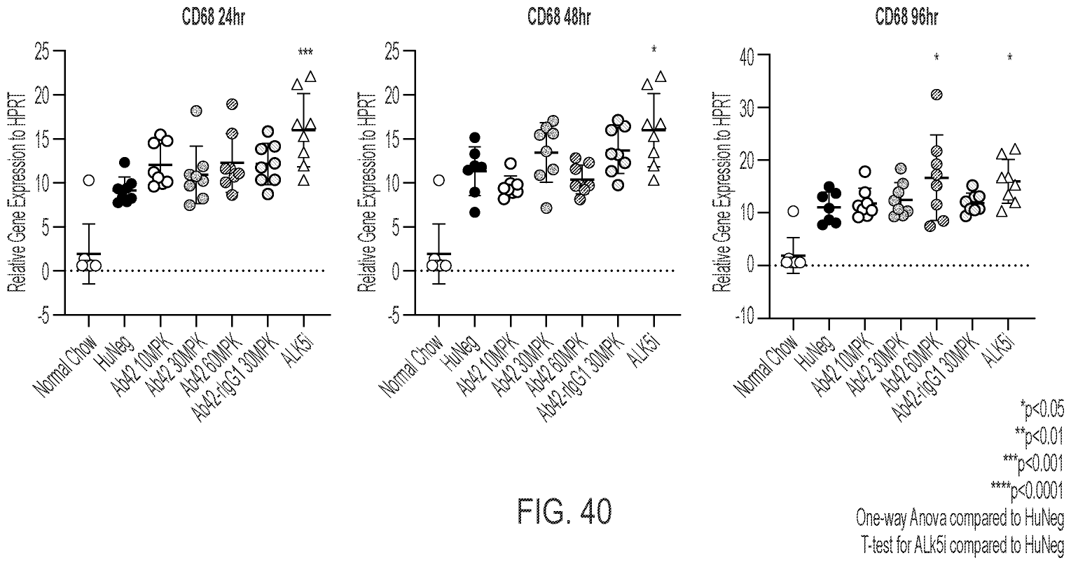

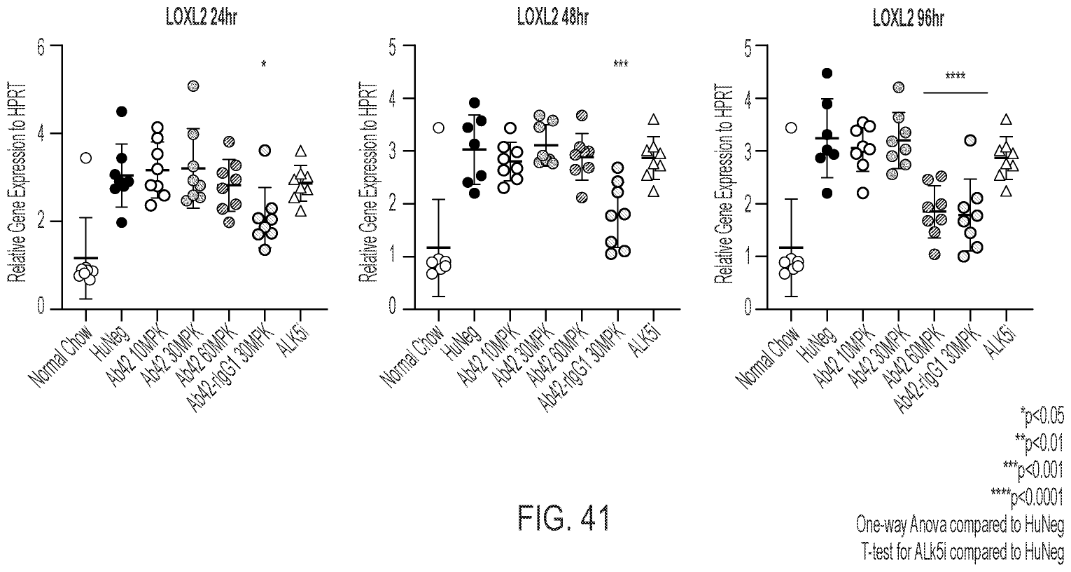

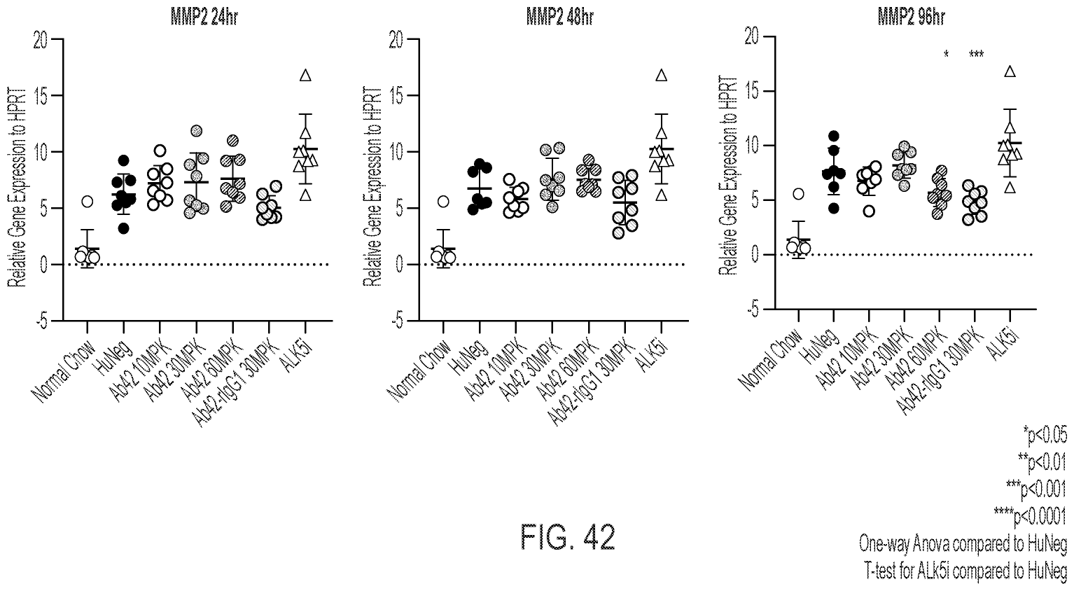

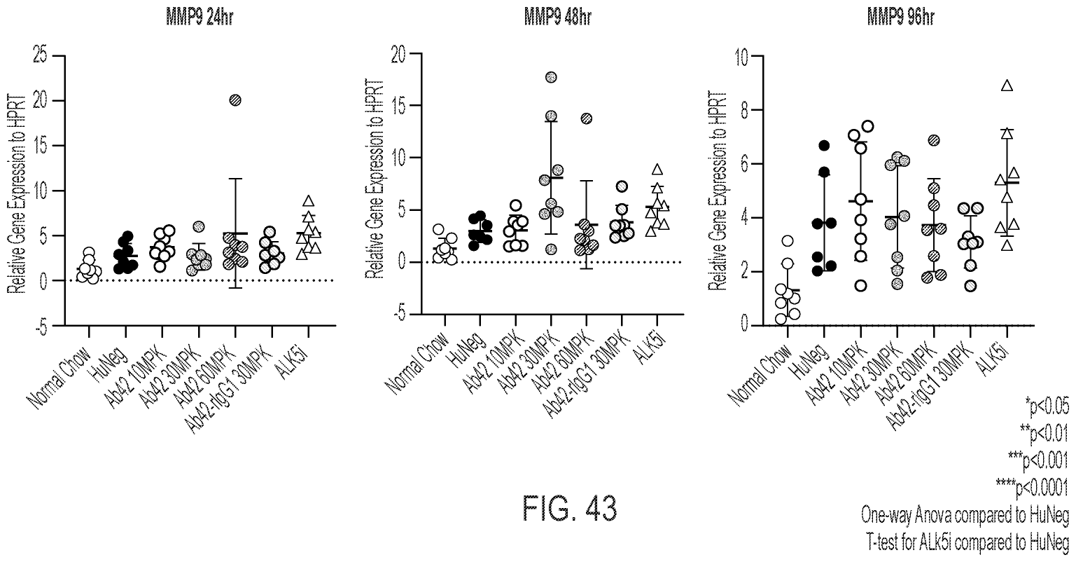

Surprisingly, LTBP-selective inhibition did not significantly affect Thbs (FIG. 38), Col1a1 (FIG. 39), CD68 (FIG. 40) Col3A1, NGAL, lox12 (FIG. 41), MMP2 (FIG. 42), MMP9 (FIG. 43), Serpine1, Timp1, and TGFβ1, 2 and 3 and LTBP1 expression with the exception of the highest dose tested, 60 mg/kg, at 96 hours.

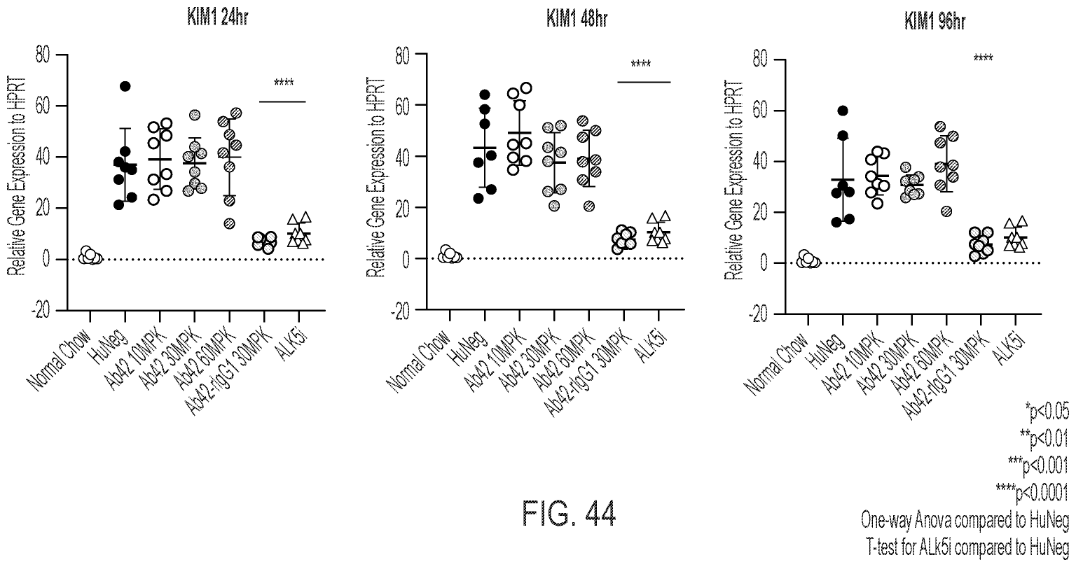

Unexpectedly, a pro-fibrotic gene, KIM1, was not affected by LTBP-selective inhibition at all doses and time-points tested (FIG. 44), in contrast to what was observed with a context-independent inhibitor, where KIM1 gene expression was almost reduced to baseline.

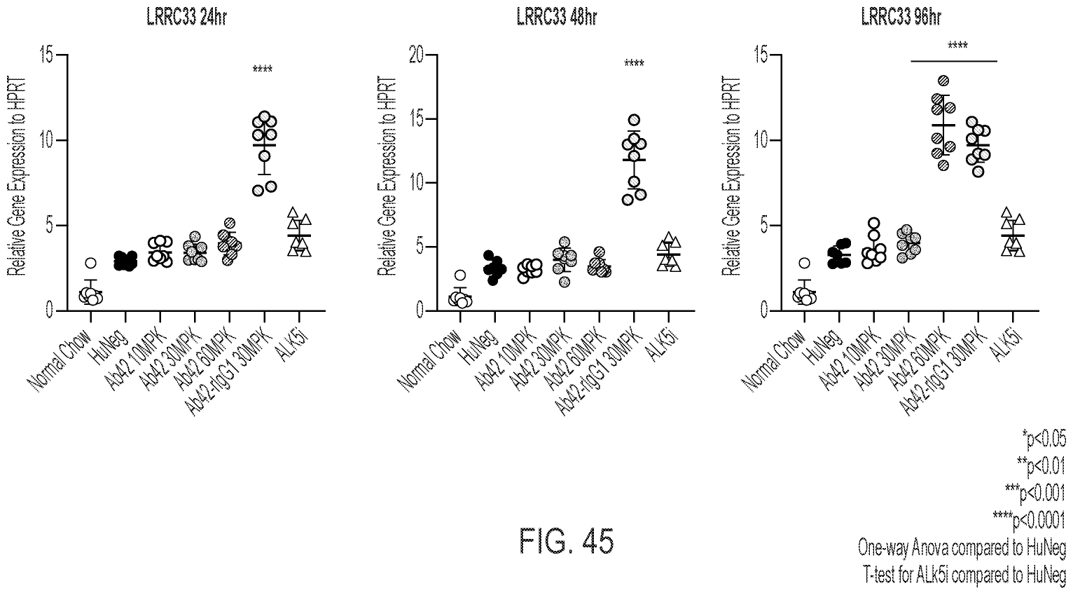

Context-independent inhibition was also shown to increase expression of LRRC33 (FIG. 45), whereas pan-inhibition did not. Additionally, it was demonstrated that LTBP-selective inhibition did not demonstrate significant changes in LRRC33 expression, in contrast with a reference antibody that inhibited TGFβ1 in a context-independent manner.

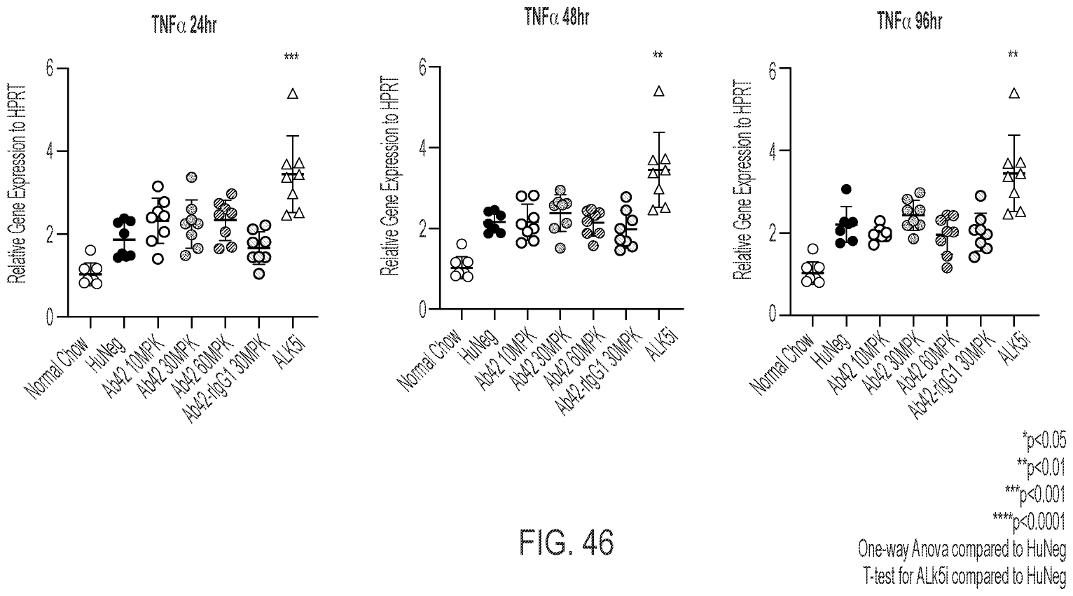

Finally, it was demonstrated that LTBP-selective inhibition did not exacerbate inflammatory markers such as TNFα (FIG. 46), as compared to a pan-TGFβ inhibitor, which did increase TNFα expression.

### Example 5: Determination of Safety Profile in Sub-Chronic Mouse Toxicology Analysis

A previous twelve-week mouse toxicology analysis with a context-independent reference antibody showed treatment-related, non-adverse findings with TGFβ1 inhibition. This analysis was used to provisionally determine a maximally tolerated dose.

The inventors are currently performing a thirteen-week, repeat-dose, non-GLP, sub-chronic mouse toxicology analysis as a risk assessment for hyperplastic pathology.

Drugs broadly targeting TGFβ (pan-TGFβ inhibitors) demonstrated dose-limiting toxicities in preclinical analyses, including heart, ovaries, and epithelial toxicities.

Goal: A further study was performed to determine if selectively targeting the LTBP-TGFβ1 complex leads to an improved safety profile, e.g., in the peritoneum (stomach, pancreas and kidneys), heart, and ovaries.
Design: Species: CD-1 mice (8-10-week-old; 30-40 g)
CD-1 mice selected as tox species based on substantial historical data
Class effect-related findings have been documented in CD-1 mice
Group Size. n=16/sex
Route: IP
Dosing Frequency: 3 times/week
TK Time points:
Day 1:1, 4, 24, 72, 96 and 168 hours.
Day 29: predose, 4 and 24 hours.
Day 85: predose, 1, 4, 24, 96 and 168 hours.
Endpoints: Clinical observations, body weight, food consumption, ophthalmic observations.

Organ weights and gross pathology.

Clinical and microscopic pathology (hearts serial sectioning to capture all 4 valves as described by O'Brien et al. (2011)), incorporated by reference in its entirety herein. Study design is shown in the Table below:

Study Design

| | | Dose mg/kg | Number of | | Number of | |
|---|---|---|---|---|---|---|
| | T.A | (3X | Animals in Tox | | Animals in TK | |
| Group | (hIgG4) | weekly) | Males | Females | Males | Females |
| 1 | Vehicle | 0 | 16 | 16 | 3 | 3 |
| 2 | Ab42 | 10 | 16 | 16 | 24 | 24 |
| 3 | Ab42 | 30 | 16 | 16 | 24 | 24 |
| 4 | Ab42 | 100 | 16 | 16 | 24 | 24 |

### Example 6: Ab42 Dose Exposure Data

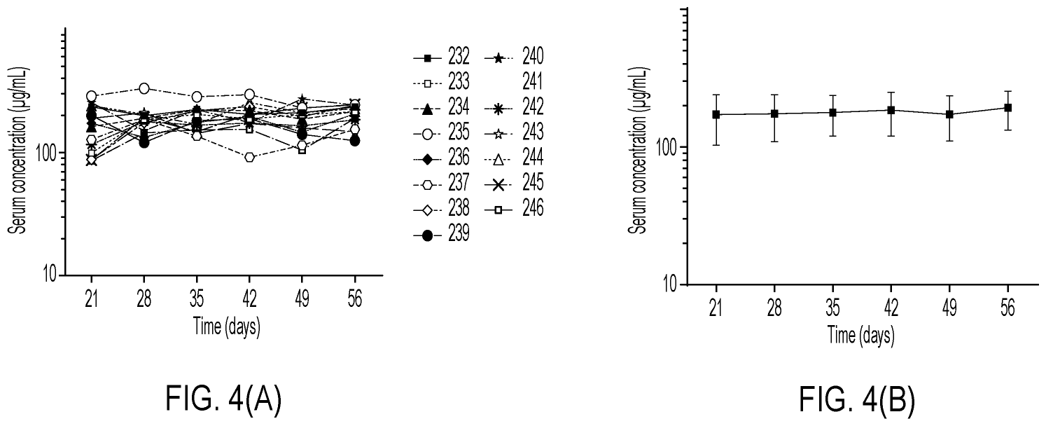

Variability in exposure in between animals. See FIG. 4 for results of 10 mg/kg.

In the case of average animal exposure, dose response is not observed with time, with exposure of 171 μg/mL at twenty-one days and exposure of 193 μg/mL at fifty-six days.

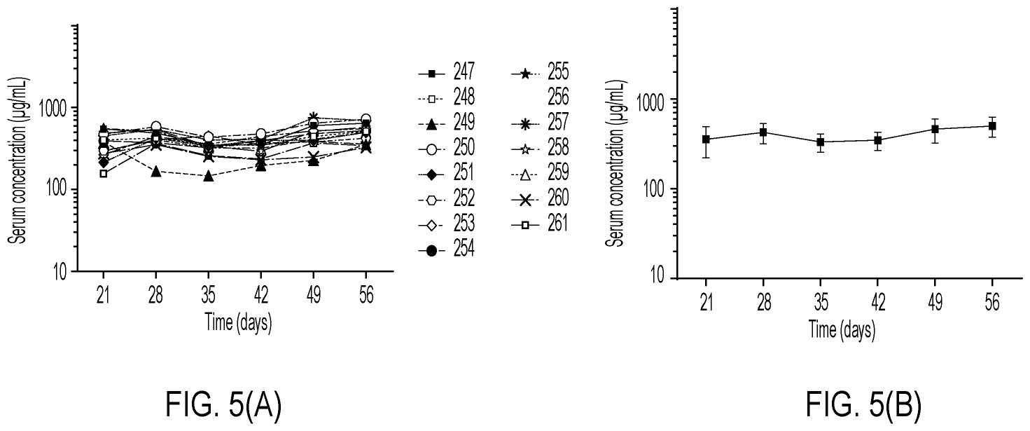

Variability in exposure in between animals. See FIG. 5 for results of 30 mg/kg.

In case of average animal exposure, an increase dose response was observed over time, with a lower exposure at twenty-one days (˜355 μg/mL) and a higher exposure at fifty-six days (˜500 μg/mL).

### Example 7: Pharmacokinetics Simulation Provides Rationale for Loading Dose Approach in Rat Adenine Efficacy Analysis. Serum Exposures that are Efficacious in the Rat Adenine Model can be Clinically Feasible

In a previous pharmacokinetics simulation analysis, Ab42 required serum exposures of ˜340 μg/mL to reduce pSmad2 in CDHFD (liver) single dose analysis. See WO 2020/160291 A2, incorporated by reference in its entirety herein. These results suggested that a once every 2 weeks (Q2W) intravenous (IV) dosing regimen was required to achieve ˜400 μg/mL serum exposure.

Several rounds of dosing were required to achieve dose “stacking” in previous adenine repeat-dose efficacy analysis.

Possibly due to target-mediated drug disposition (TMDD), the phenomenon in which a drug binds with high affinity to its pharmacological target site (such as a receptor) to such an extent that this affects its pharmacokinetic characteristics.

A recent adenine efficacy study demonstrated that pharmacologically active exposure may be lower. Serum exposures of ˜200 μg/mL led to anti-fibrotic efficacy, as measured by picrosirius red staining and hydroxyproline assays.

A well-behaved antibody (t_{1/2}=twenty-eight days) can be dosed subcutaneous QW to reach 164 μg/mL steady state serum exposure. An extended half-life may enable a loading dose approach combined with subcutaneous maintenance dosing. Based on pharmacokinetics simulation, loading dose approach achieves the serum exposures needed to engage the target within first week of dosing.

### Example 8: Antibody Discovery and In Vitro Pharmacology. H/DX-MS Data Elucidating the Epitope of Ab42

H/DX-MS provides insights into the binding region of lead antibodies for context-dependent TGFβbinding.

The H/DX-MS workflow can be followed using the mass spectroscopy technology performed at the University of Massachusetts, Amherst. Using these methods, human LTBP1-ProTGFβ1 showed good peptide coverage.

A deuterium uptake heat map was produced to show the flexibility profile of human LTBP1-ProTGFβ1 across the region of coverage. An Ab42 Fab shows strong H/DX protection within the α1 helix of the LAP of proTGFβ1. The binding region of Ab42 in the α1 helix within the LAP of pro-TGFβ1 shows sequence diversity. See WO 2020/160291 A2, incorporated by reference in its entirety herein.

The Ab42 binding profile. Ab42 binds the pro-TGFβ1 C4S region only, with no observed binding to proTGFβ2, proTGFβ3, or TGFβ1 growth factor only. Ab42 exhibits greater than 100-fold specificity for LTBP presented pro-TGFβ1 over GARP and LRRC33 complexes. Ab42 binds with similar low nM K_{D} to both pro-TGFβ1 and LTBP(1/2)-pro-TGFβ1. These binding data suggested that the epitope of the antibody is likely within the pro-TGFβ1, consistent with H/DX-MS data for Ab42.

### Example 9: Inhibition of TGFβ Signaling by LTBP-TGFβ1 Antibodies in a Genetic Model of Alport Syndrome

Alport Syndrome (Mouse) Kidney Fibrosis Pharmacokinetics/Pharmacodynamics Analysis

The murine Col4a3−/− model is an established genetic model of autosomal recessive Alport syndrome. Alport mice lack a functional collagen 4A3 gene (Col4A3−/−) and therefore cannot form normal type IV collagen trimers, which require a3, a4, and a5 chains. Col4a3−/− mice develop fibrosis in the kidney consistent with renal fibrosis in human patients, including interstitial fibrosis and tubular atrophy, and Col4a3−/− mice develop end-stage renal disease (ESRD) between 10 and 30 weeks of age, depending on the genetic background of the mouse. The structural and functional manifestation of renal pathology in Col4a3−/− mice, combined with the progression to ESRD make Col4a3−/− mice an ideal model to understand kidney fibrosis. Previous reports point to the importance of the TGFβ signaling pathway in this process, and treatment with either αvβ6 integrin, a known activator of TGFβ, or with a TGFβ ligand trap has been reported to prevent renal fibrosis and inflammation in Alport mice (Hahm et al., (2007) The American Journal of Pathology, 170(1): 110-125).

Ab42 was tested for its ability to inhibit or mitigate renal fibrosis in Alport mice as follows.

F1 offspring from Col4a3+/−males on a 129/Sv genetic background crossed to Col4a3+/−females on a C57/B16 genetic were employed for the study. These mice typically exhibit proteinuria by 4-5 weeks old and typically progress to ESRD by 14-15 weeks old, providing a good therapeutic window for testing efficacy of treatment.

It is well documented that TGFβ receptor activation leads to a downstream signaling cascade of intracellular events, including phosphorylation of SMAD2/3. Therefore, the ability of Ab42 antibody treatment to inhibit TGFβ signaling may be assessed in kidney lysate samples by measuring relative phosphorylation levels of SMAD2/3 by immunohistochemistry (IHC) according to the manufacturer's instructions.

Targeting latent TGFβs via its latency-associated peptide (LAP) or prodomain creates multiple “handles” for selectivity.

Eleven-week-old mice were treated with a single dose of antibody. Mouse tissue was collected after forty-eight hours. Treatment with Ab42 significantly reduce pSmad2 in Alport syndrome mouse kidneys (not shown).

### Example 10: Testing of LTBP-TGFβ1 Antibodies in Human Idiopathic Pulmonary Fibrosis (IPF) Precision-Cut Lung Slices

Precision Cut Lung Slices (PCLuS) from explanted human IPF lung tissue are physiologically and structurally representative of the tissue architecture and cellular composition of the diseased lung. Testing potential therapeutic targets and interrogating mechanisms underpinning disease pathophysiology in human PCLuS allows the assessment of their effectiveness and relevance to the clinical situation, overcoming many of the limitations of widely employed in vivo rodent models and in vitro 2D cell culture methodologies.

To date, the main problems with using human lung PCLuS has been the loss of metabolic and structural viability of the tissue within a relatively short timeframe (reported to be as little as 8 hrs in some studies) and the use of poorly characterised PCLuS from “normal” tissue collected from patients undergoing tumor resection. The IPF model is an ex-vivo model, where PCLuS are prepared from human donor IPF lungs, and maintain viability and functionality for at least 7 days ex-vivo. These IPF-PCLuS maintain their inflammatory and fibrotic phenotype in ex-vivo culture and these responses can be attenuated by Pirfenidone and Nintedanib, first-generation approved IPF therapies. This approach is expected to have better translatability to human disease because the extracellular matrix architecture is maintained during ex vivo culture of tissue slices. In this model, the progression of fibrosis in culture is driven by TGFβ signaling.

The following studies were carried out to determine if selectively targeting LTBP-TGFβ1 complexes elicits an anti-fibrotic response in human IPF tissue slices.

### PCLuS Preparation and Culture

PCLuS were prepared from biopsy confirmed, explanted IPF human lung tissue collected at the time of lung transplantation. All PCLS were sectioned from one area of the lung, then pooled into a single petri-dish and then loaded into culture plates (n=2). PCLS were rested for 48 hours to allow the post-slicing stress period to elapse before experimentation. Treatment started with test articles as shown in the table below, after 48 hrs of resting period. Media was collected every 24 hrs to measure soluble proteins over time and replenished with new media plus test articles.

All PCLuS were harvested at 168 hours.

### Study Groups

Ten (10) different groups with n=6 human PCLuS were investigated as shown below. PCLuS were prepared from n=2 biopsy confirmed, explanted human IPF lung.

Study Design

| Group | T.A. | Doses | Duration | | No. |
|---|---|---|---|---|---|
| 1 | N.A. | N.A. | 48 | hours | 6 |
| 2 | N.A. | N.A. | 48-168 | hours | |

| 3 | Vehicle | N.A. | |
|---|---|---|---|
| 4 | Pirfenidone | 2.5 | mM |
| 5 | Nintedanib | 2.5 | μM |
| 6 | ALK5i | 10 | μM |
| 7 | αvβ6 inhib. (SMI) | 1 | μM |
| 8 | HuNeg | 100 | μg/mL |
| 9 | Ab42 | 3, 30, 100 | μg/mL |
| 10 | Ab1D11 | 30 | μg/mL |

### Harvest and Analysis

PCLuS harvest: Cell culture supernatant (n=6 per group) was collected daily and snap frozen for quantification of the soluble outputs listed below. At harvest, n=2 PCLuS were snap frozen for RNA isolation and qPCR analysis, n=2 PCLuSwere Formalin fixed, paraffin embedded (FFPE) for IHC and n=2 PCLuS were used for a resazurin assay to measure cell viability.

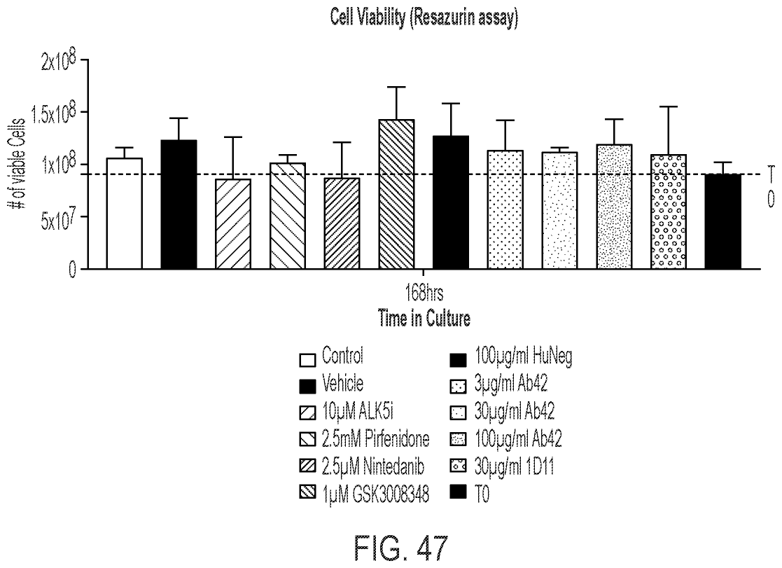

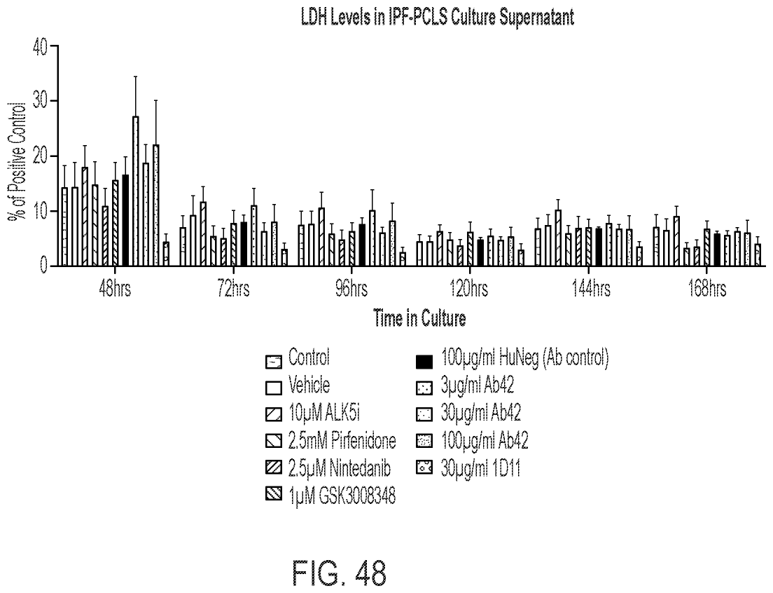

Viability: Cell viability was assayed using either resazurin assay at endpoint or measuring lactate dehydrogenase levels in supernatant at each timepoint. T0 tissue was collected before start of treatment. It was found that over 168 hours there was overall similar viability (FIG. 47) and low LDH (FIG. 48X) levels across groups, which suggested that tissue was healthy throughout the study. Levels of LDH in the cell culture supernatants (48 hrs, 72 hrs, 96 hrs, 120 hrs, 144 hrs & 168 hrs) were quantified using an LDH assay kit (Sigma). Metabolic activity of PCLuS were quantified by resazurin assay.

Soluble outputs analysis: Levels of collagen 1a1, Fibronectin, TIMP1, MMP7 and Hyaluronic Acid (HA) in the cell culture supernatants were quantified using R&D Duoset® ELISA kits. Levels of total TGF-β1/TGF-β2/TGF-β3 in the cell culture supernatants were quantified using a U-plex TGF-β Combo multiplex ELISA (Meso Scale Discovery®).

Formalin fixed, paraffin embedded blocks: an additional n=3 FFPE blocks containing macroscopically normal tissue from an adjacent area of the IPF lung were provided.

RNA Analysis: Total RNA extraction from PCLuS was performed on all samples (n=2 pooled PCLuS per group, per donor) including TO PCLuS).

The endpoints were as follows:
Measurement of soluble proteins: Collagen 1, fibronectin, TIMP1, MMP7, hyaluronic acid, and TGFβ ligands

Assays on fixed lung tissue: PSR and pSmad2.

### Results

Gene analysis was performed on samples from two separate donors. While further experiments will be performed using a larger sample size, from these results, it can be concluded that Ab42 showed stronger reduction in profibrotic soluble factors in donor 1 than in donor 2. ALK5i, Pirfenidone, and Nintedanib showed more consistency with reducing soluble factors. One prediction that can be made from these results is that Ab42 may have potential anti-fibrotic effect in the IPF lung.

### Example 11: Human Clinical Trial to Test the Efficacy of a Combination Treatment for Idiopathic Pulmonary Fibrosis, Comprising the Administration of Pirfenidone, Nintedanib, and Ab42

The results provided in the specification above are a good foundation for persons having ordinary skill in the medical art to conduct a series of human clinical trials to confirm the safety and efficacy of a combination therapy comprising the administration of pirfenidone, nintedanib, and Ab42 to treat idiopathic pulmonary fibrosis in humans.

### Example 12: Alport Dose Response PK/PD Test for Ab42-Reanalysis

Dose at eleven-week-old mice. Takedown after forty-eight hours.

Collection: Kidney: for immunohistochemistry/pSmad ELISA assays.

Serum collection at twenty-four hours to determine antibody exposure at 1 mg/kg.

Study Design

| Group | Genotype | Ab (hIgG4) | Dose (mg/kg) | Frequency | No. |
|---|---|---|---|---|---|
| 1 | Het | HuNeg | 30 | 1 | 6 |
| 2 | KO | HuNeg | 30 | 1 | 10 |
| 3 | KO | context- | 10 | 1 | 10 |
| | | independent | | | |
| | | reference | | | |
| | | antibody | | | |
| 8 | KO | Ab42 | 30 | 1 | 10 |
| 9 | KO | Ab42 | 10 | 1 | 10 |
| 10 | KO | Ab42 | 3 | 1 | 10 |

Re-stain of study removes batch artifact. The inventors re-stain and re-analysis shows uniform batch consistency. Significant pSmad2 suppression was seen in Ab42 treatments by t-test and ANOVA. For Ab42, serum exposures of ˜13 μg/mL is sufficient to reduce pSmad2. This result suggests that the Alport dose response model might be driven by LTBP-TGFβ1 complexes.

The inventors observed a reduction of GARP and inflammatory genes after a single dose of Ab42, including CCL2, IL-1 b, and IL-6. Ab42 at 30 mg/kg showed reduction at all timepoints. Ab42 at 60 mg/kg showed reduction at ninety-six hours. The inventors performed a re-analysis of the raw qPCR data and saw the same results.

### Example 13: Gene Expression Re-Analysis from Adenine PK/PD Test

A small subset of the earlier results above were taken for assay and RNA integrity check. The localization, intensity matched prior immunohistochemistry results.

Study Design

| | | | Dose | Dosing | Tissue | |
|---|---|---|---|---|---|---|
| | | | level | fre- | Collec- | No. |
| Group | Diet | TA | (mg/kg) | quency | tion (hr) | animals |
| 1 | Chow | N/A | N/A | N/A | 96 | 8 |
| 2 | 0.25% | HuNeg | 60 | Day 1 | 24, 48, 96 | 3 × 8 (24) |
| 3 | Adenine | Ab42 | 60 | Day 1 | 24, 48, 96 | 3 × 8 (24) |
| 4 | | Ab42 | 30 | Day 1 | 24, 48, 96 | 3 × 8 (24) |
| 5 | | Ab42 | 10 | Day 1 | 24, 48, 96 | 3 × 8 (24) |
| 6 | | Ab42 | 30 | Day 1 | 24, 48, 96 | 3 × 8 (24) |
| 10 | | GS- | 10 | Once | 2 hr pre- | 8 |
| | | 490470 | | | necropsy | |

qPCR analysis showed a reduction of GARP after a single dose of Ab42.

GARP in situ hybridization results did not show observable reduction in fibrotic regions.

In situ hybridization results are being measured independently as well as relative to pSmad2 localization.

Expression levels are being compared against qPCR data to confirm earlier observations.

Notably, Ab42 did not cause exacerbation of TNFα gene expression, which is an inflammatory marker. Contrast this to ALK5 inhibitor, which is a potent panTGFß blocker, which exacerbated expression of that gene as compared to the controls in diseased animals.

### Example 14: A 13-Week Toxicity and Toxicokinetic Study of Ab42 Human IgG4 by Intraperitoneal Administration in Mice

The objectives of this study were to determine the potential toxicity of Ab42 Human IgG4, when given via intraperitoneal injection 3 times weekly for up to 13 weeks (up to 39 total doses) to mice.

Crl:CD1 (ICR; Charles River) mice were administered 0 (vehicle control), 10, 30 or 100 mg/kg/dose Ab42Human IgG4 by intraperitoneal injection 3 times per week for up to 13 weeks (up to 39 doses).

Three main study animals administered 30 mg/kg/dose were found dead on Day 27 or 48. The probable cause of death for Animal 3009 was iatrogenic hemorrhage into the abdominal cavity (i.e., hemoabdomen) from an intraperitoneal injection. A cause of death could not be determined for Animals 3007 and 3010.

No test article-related differences in organ weight parameters were noted. Increased thymus weights (absolute and relative to body and brain weights) in animals administered 10 mg/kg/dose were of uncertain relationship to the test article because of the intragroup variation and lack of a dose relationship or microscopic correlate.

No test article-related macroscopic findings were noted.

Test article-related microscopic findings were limited to an increased incidence and/or severity of minimal to marked mixed cell inflammation in the Harderian gland in females administered 30 mg/kg/dose and males administered 100 mg/kg/dose. Harderian gland inflammation was often associated with minimal degeneration/necrosis and the male with marked mixed cell inflammation had more pronounced (moderate) degeneration/necrosis with loss of glandular tissue. Moderate or marked mixed cell inflammation in the Harderian glands was considered adverse in mice; however, humans do not have a Harderian gland. An increased incidence and/or severity of leukocytic infiltrates attributed to intraperitoneal injections in test article-treated animals was suggestive of an increased inflammatory response to the antibody test article compared with the vehicle.

### Example 15: Determination of the Pharmacokinetics of Ab42 hlgG4 and Ab42 hlgG4-YTE Mutant Following a Single Dose Intravenous Administration to Monkeys

A study was carried out to determine the pharmacokinetics of Ab42 hlgG4 and Ab42 hlgG4-YTE mutant following a single dose intravenous administration to cynomolgus monkeys.

A YTE mutation in the antibody Fc region has previously resulted in increased antibody serum half-lives, ˜2-5 fold longer half-life in humans, based on the results of two different antibodies published by others.

Ab42 with the YTE mutation (Ab42-YTE) has been generated. The expression titers have been found to be similar to Ab42, with a similar potency of Ab42-YTE to Ab42, which was confirmed by CAGA assay.

Study Design: the study design is shown in the table below. There were 6 groups of animals (Cynomolgus monkey (females): Age 2-5 years (˜2.5 to 5 kg), 3 animals per group. Test agent (T.A) was given intravenously (IV) as a bolus injection.

Study Design

| Group | T.A. (hIgG4) | Dose (mg/kg; IV) | Number of Animals |
|---|---|---|---|
| 1 | Ab42 | 0.3 | 3 |
| 2 | Ab42 | 1 | 3 |
| 3 | Ab42 | 3 | 3 |
| 4 | Ab42 | 10 | 3 |
| 5 | Ab42-YTE | 0.3 | 3 |
| 6 | Ab42-YTE | 10 | 3 |

Sample collection was carried out at the time points shown in the Table below.

| Days | | ADA |
|---|---|---|
| 0 | Interim | |
| 0.04 (1 hour) | Analysis 1 | |
| 0.17 (4 hour) | | |
| 0.33 (8 hour) | | |
| 1 | | |
| 3 | | ✓ |
| 5 | | |
| 7 | | ✓ |
| 10 | | |
| 14 | | ✓ |
| 21 | Interim | ✓ |
| 28 | Analysis 2 | ✓ |
| 35 | | ✓ |
| 42 | | ✓ |
| 49* | Final | ✓ |
| 56* | Analysis | ✓ |

*Groups 5 and 6 only

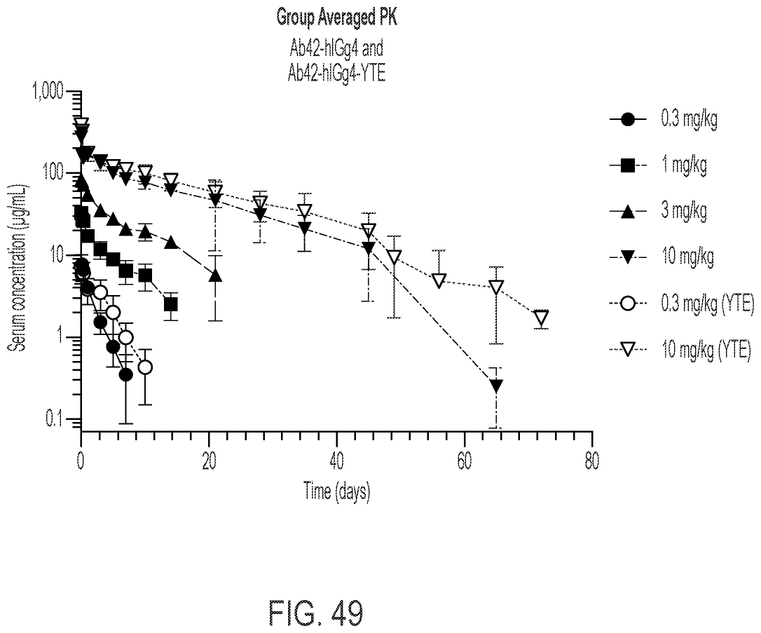

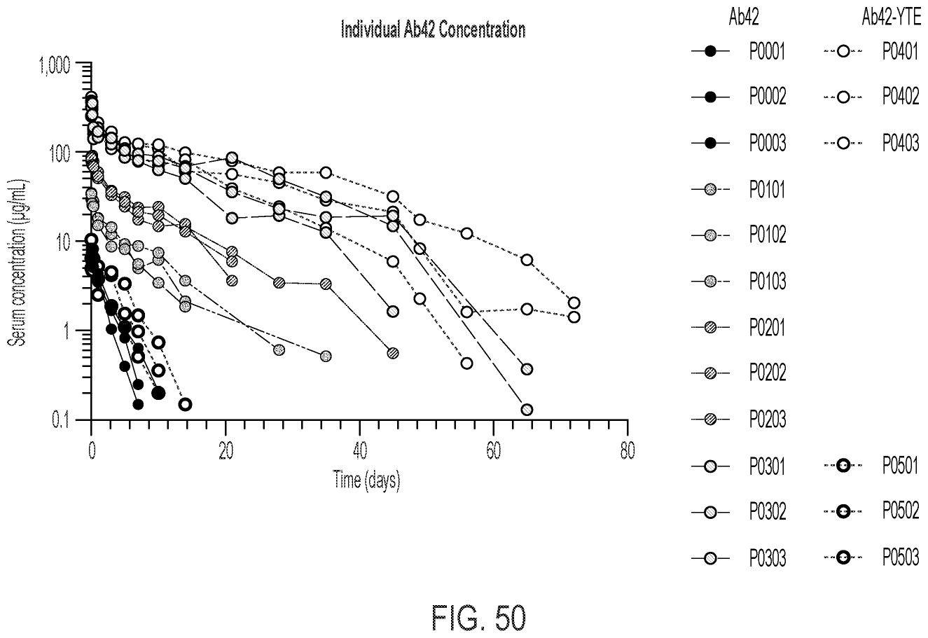

FIG. 49 shows group averaged PK data up to day 72. As seen in FIG. 49, there was a slight difference in apparent half-life between the WT and YTE test article. Comparing WT and YTE at the 0.3 and 10 mg/kg doses, there was a clear extension of half-life. FIG. 50 shows PK data for individual animals. Again, comparing WT and YTE at the 0.3 and 10 mg/kg doses, there was overall a clear extension of half-life.

According to some embodiments, thr YTE mutation does not affect antigen binding affinity.

### REFERENCES

A person having ordinary skill in the biomedical art can use these patents, patent applications, and scientific references as guidance to predictable results when making and using the invention.

### PATENT LITERATURE

WO 2020/160291 A2 discloses the antibodies characterized by at least all of their six CDR sequences.

WO 2019/023661 A1 (Scholar Rock Inc.), published Jan. 31, 2019, discloses inhibitory antibodies binding to human LTBP1-proTGFβ1 complex or human LTBP3-proTGFβ1 complex that do not bind human GARP-proTGFβ1 complex and their use for fibrosis treatment. The '661 patent publication discloses inhibitory antibodies binding to human LTBP1-proTGFβ1 complex or human LTBP3-proTGFβ1 complex which do not bind human GARP-proTGFβ1 complex. The '661 patent publication also discloses methods for antibody generation and selection, antibodies designated identical to antibodies having the identical designation of the present application.

WO 2018/129329 A1 (Scholar Rock Inc.), published Jul. 12, 2018, discloses isoform-specific inhibitor antibodies targeting proTGFβ1 complexes and use thereof for treatment of fibrosis or proliferative diseases. The antibodies designated Ab-1, Ab-2 and Ab-3 are shown to bind to LTBP1-proTGFβ, LTBP3-proTGFβ, GARP-proTGFβ and LRRC33-proTGFβ with different affinities, i.e., preferential binding to one complex over the others. See, e.g., Examples and Table 10. The '329 patent publication discloses isoform-specific inhibitor antibodies targeting both ECM-associated TGFβ1 binding (LTBP1-proTGFβ1 or LTBP3-proTGFβ1) and immune cell-associated TGFβ1 (GARP-TGFβ1, LR RC33-TGFβ1). The '329 patent publication further discloses the use thereof for treatment of fibrosis and proliferative diseases.

WO 2017/156500 A1 (Scholar Rock Inc.), published Sep. 14, 2017, relates to isoform specific inhibition of TGFβ1, refers to antibodies that bind to one up to all complexes of GARP-TGFβ1, LRRC33-TGFβ1, LTBP1-TGFβ1, and LTBP3-TGFβ1, thereby targeting pro/latent complex, and discloses their use for treatment purposes. The '500 patent publication shows that inhibitory antibodies Ab1 and a context-independent reference antibody do not bind to mature TGFβ1, TGFβ2, or TGFβ3, but bind to LTBP1-proTGFβ1, LTBP3-proTGFβ3, GARP-proTGFβ1, and LRRC33-proTGRbeta1. The antibodies have therapeutic utility alone or in combination with anti-PD-1. To overcome toxicities associated with TGFβ inhibition, isoform-specific inhibition of TGFβ1 are to be generated and used in therapy. See, e.g., page 3, first paragraph. The antibodies directed to TGFβ are also present in a particular context. See, e.g., page 4-page 5, bridging paragraph and page 5, last paragraph-page 8, 2nd paragraph). The '500 patent publication further teaches how to obtain such antibodies. See, e.g., page 73 and Examples. The specific disclosed antibodies Ab1 and a context-independent reference antibody are shown not to bind mature TGFβ1, TGFβ2, TGFβ3 and bind to GARP-proTGFβ1, L TBP1-proTGFβ1, L TBP3-proTGFβ3 and LRRC33-proTGFβ1.

WO 2016/161410 discloses neutralizing antibodies that bind both TGFβ1 and TGFβ2 (i.e., TGFβ1/2 inhibitors).

WO 2014/182676 A2 (Scholar Rock Inc.), published Nov. 13, 2014, discloses an antibody, which can be humanized (see, e.g., paragraph [167]), directed to LTBP1-proTGFβ complex (see, e.g., Table 17, paragraphs [558]-[559] and Example 18) and used for treatment of fibrosis (see, e.g., claim 40). The '676 patent publication discloses the screening of antibodies directed to proTGFβ complexed with LTBP1, which do not bind to free TGFβ1 and have inhibitory activity (“ability to decrease TGFβ signaling) as well as use thereof for treatment of, e.g. fibrosis. The '676 patent publication discloses the screening of antibodies directed to proTGFβ complexed with LTBP1 which do not bind to free TGFβ1 and have inhibitory activity (“ability to decrease TGFβ signaling) as well as use thereof for treatment of, e.g., fibrosis.

WO 2011/102483 A1 (Riken), published Aug. 25, 2011, discloses antibodies binding to LAP (=proTGFβ). The BP-873 antibody binds to peptides present in proTGFβ1 but not to fragments of proTGFβ2 and does not bind mature TGFβ1. It is not clear from the '483 patent publication whether the antibody binds to proTGFβ complex, i.e., whether formation of complex prevent its binding.

WO 2006/116002 provides neutralizing antibodies that bind both TGFβ1 and TGFβ3 (i.e., TGFβ1/3 inhibitors),

### Non-Patent Literature

Aluwihare et al. (2009) J. Cell Sci., 122(Pt 2), 227-32. Mice that lack both β6 and b8 integrins recapitulate all essential phenotypes of TGFβ1 and TGFβ3 knockout mice, including multiorgan inflammation and cleft palate, confirming the essential role of these two integrins for TGFβ1 activation in development and homeostasis.
Anderton et al. (2011) Toxicology Pathology, 39: 916-24, reported that small molecule inhibitors of TGFβ type I (ALK5) receptor induced heart valve lesions characterized by hemorrhage, inflammation, degeneration and proliferation of valvular interstitial cells in a preclinical animal model. The toxicity was observed in all heart valves at all doses tested.
Bird et al. (1988) Science, 242, 423-426 discloses single-chain Fv (scFv) molecules.
Brennan et al. (2018) mAbs, 10:1, 1-17. The prevailing view of the field continues to be that it is advantageous to inhibit multiple isoforms of TGFβ to achieve therapeutic effects, and that to accomplish toxicity management by “careful dosing regimen.”
Derynck & Zhang (2003) Nature, 425(6958), 577-84. SMAD-independent TGFβ signaling pathways were described, e.g., in cancer or in the aortic lesions of Marfan mice.
Dong et al., Nat Struct Mol Biol, 2014. 21(12), 1091-6. αV integrins bind the RGD sequence present in TGFβ1 and TGFβ1 LAPs with high affinity.
Ezzie et al. (2011) Am. J. Respir. Cell Mol. Biol., 44(4), 556-61. Mechanisms of TGFβ1 activation were implicated, including thrombospondin-1 and activation by proteases such as matrix metalloproteinases (MMPs), cathepsin D or kallikrein. Most of these studies were performed in vitro using purified proteins. There is less evidence for the role of these molecules from in vivo studies. Knockout of thrombospondin-1 recapitulates some aspects of the TGFβ1^{-/-} phenotype in some tissues but is not protective in bleomycin-induced lung fibrosis, known to be TGFβ3-dependent.
Frazier et al. (2007) Toxicology Pathology, 35: 284-295 reported that administration of the small molecule inhibitor of TGFβ type I (ALK5) receptor GW788388 induced physeal dysplasia in rats.
Holm et al. (2011) Science, 332(6027), 358-61). SMAD-independent TGFβ signaling pathways were described, e.g., in cancer or in the aortic lesions of Marfan mice.
Horan et al. (2008) Am J Respir Crit Care Med, 177(1), 56-65. The αvβ6 blockade with an antibody or loss of integrin β6 expression suppresses bleomycin-induced lung fibrosis and radiation-induced fibrosis.
Huston et al. (1988) Proc. Nat'l. Acad. Sci., USA 85, 5879-5883 discloses single-chain Fv (scFv) molecules.
Janssens et al. (2006) J. Med. Genet., 43(1), 1-11. Camurati-Engelman disease results in bone dysplasia due to an autosomal dominant mutation in the TGFβ1 gene, leading to constitutive activation of TGFβ1 signaling.
John et al. (2020) “Translational pharmacology of an inhaled small molecule αvβ6 integrin inhibitor for idiopathic pulmonary fibrosis.” Nature Communications, 11, 11, Article number 4659.
Lacouture et al. (2015) Cancer Immunol. Immunother., 64, 437-46. Fresolimumab (GC1008), a “pan” TGFβ antibody capable of neutralizing all human isoforms of TGFβ, was reported to induce an epithelial hyperplasia of the gingiva, bladder, and of the nasal turbinate epithelium after multiple administrations in studies with cynomolgus macaques. The most notable adverse reaction to fresolimumab includes the induction of cutaneous keratoacanthomas and/or squamous cell carcinomas in human cancer patients
Lonning et al. (2011) Current Pharmaceutical Biotechnology, 12, 2176-89. Fresolimumab (GC1008), a “pan” TGFβ antibody capable of neutralizing all human isoforms of TGFβ, was reported to induce an epithelial hyperplasia of the gingiva, bladder, and of the nasal turbinate epithelium after multiple administrations in studies with cynomolgus macaques. The most notable adverse reaction to fresolimumab includes the induction of cutaneous keratoacanthomas and/or squamous cell carcinomas in human cancer patients
Massague et al. (2005) Genes Dev., 19(23), 2783-810. Ligand-induced oligomerization of TGFβRI/II triggers the phosphorylation of SMAD transcription factors, resulting in the transcription of target genes, such as Coil a1, Col3a1, ACTA2, and SERPINE1.
Munger et al. (1999) Cell, 96(3), 319-28. Several studies have shed light on the mechanisms of TGFβ1 activation. Three integrins, αvβ6, αvβ8, and αvβ1 were demonstrated to be key activators of latent TGFβ1. The αvβ6 blockade with an antibody or loss of integrin β6 expression suppresses bleomycin-induced lung fibrosis and radiation-induced fibrosis.
Rabat et al. (1987; 1991) Sequences of Proteins of Immunological Interest (National Institutes of Health, Bethesda, Md.) describes a system that not only provides an unambiguous residue numbering system applicable to any variable region of an antibody, but also provides precise residue boundaries defining the three CDRs on each of the heavy and light chains.
Reed et al. (2015) Sci. Transl. Med., 7(288), 288ra79. Several studies have shed light on the mechanisms of TGFβ1 activation. Three integrins, αvβ6, αvβ8, and avp1 were demonstrated to be key activators of latent TGFβ1. The importance of integrin-dependent TGFβ1 activation in disease has also been well validated. A small molecular inhibitor of avp1 protects against bleomycin-induced lung fibrosis and carbon tetrachloride-induced liver fibrosis.
Richert-Spuhler & Lund (2015) Prog. Mol. Biol. Transl. Sci., 136, 217-243. A review showing potential side effects stemming from perturbation of normal Treg function in maintaining immune homeostasis
Robertson et al. (2015) Matrix Biol., 47, 44-53. Fibroblasts secrete latent TGFβ1 tethered to latent TGFβ3-binding proteins (LTBPs), which then associate with proteins in the extracellular matrix (ECM), i.e., fibronectin, fibrillin-1, to link latent TGFβ to the ECM.
Rudikoff et al. (1982) “Single amino acid substitution altering antigen-binding specificity.” Proceedings of The National Academy of Sciences, 79(6), 1979-1983 (Mar. 1, 1982). D5 teaches that all of the CDR sequences are important for antibodies binding to its target antigen.
Sela-Culang et al. (2013) “The structural basis of antibody-antigen recognition.” Frontiers in Immunology, 4: Article 302, p. 1-13, teaches that the widely assumed role of CDRs as the only determinants of antibody binding may be an oversimplification, as some position in the CDR sequences were shown to never participate in antibody binding, and some non-CDR residues often contribute critically to antibody-antigen interaction. Sela-Culang also teaches that framework region residues may play an important role in antigen binding, as evidenced by the observation that grafting only the CDRs during antibody humanization usually results in a significant drop or complete loss of binding. The binding affinity can be retained by back-mutating some of the framework region residues to the original sequence, emphasizing their role in antigen binding (p. 7, 1st column, 3rd paragraph). Sela-Culang further teaches that some framework region residues contact antigen and are therefore part of the antigen-binding site, while other framework region residues are far from the CD Rs in sequence but are near it in the 3-D structure (p. 7, paragraph spanning columns 1 and 2). There are also framework region residues that affect antigen binding but are not in contact with antigen, but affect antigen binding indirectly, and these residues are believed to provide a structural support for the CDRs, enabling them to adopt the correct conformation and orientation, or by directing and maintaining the relative orientation of the heavy chain variable region vs the light chain variable region, and thus the orientation of the CD Rs relative to each other (p. 7, 2nd column, 2nd-3rd paragraphs).
Shi et al. (2011) Nature, 474(7351), 343-9. Structural insights into the activation mechanism of TGFβ1 have enabled more specific approaches to TGFβ inhibition. The structure of latent TGFβ1 illuminates how integrins enable release of active TGFβ1 from the latent complex: the covalent link of latent TGFβ1 to its presenting molecule anchors latent TGFβ1, either to the ECM through LTBPs, or to the cytoskeleton through GARP or LRRC33. Integrin binding to the RGD sequence results in a force-dependent change in the structure of LAP, allowing active TGFβ1 to be released and bind nearby receptors.
Shoukry et al. (2017) J. Immunol., 198 (1 Supplement), 197.12. Evidence suggests that an alteration in the Th17/Treg ratio leads to an imbalance in pro-fibrotic Th17 cytokines, which correlate with severity of fibrosis, such as liver fibrosis.
Stauber et al. (2014) J. Clin. Practice 4:3, reported that a chronic, over 3-month administration of the inhibitor of TGFβ receptor I kinase, LY2157299, which was investigated for certain cancer treatments, caused multiple organ toxicities involving the cardiovascular, gastrointestinal, immune, bone/cartilage, reproductive, and renal systems, in rats and dogs.
Stevenson et al., 2013, Oncolmmunology, 2:8, e26218. Fresolimumab (GC1008), a “pan” TGFβ antibody capable of neutralizing all human isoforms of TGFβ, was reported to induce an epithelial hyperplasia of the gingiva, bladder, and of the nasal turbinate epithelium after multiple administrations in studies with cynomolgus macaques. The most notable adverse reaction to fresolimumab includes the induction of cutaneous keratoacanthomas and/or squamous cell carcinomas in human cancer patients. Additional evidence from a clinical trial suggests that in some cases this antibody may accelerate tumor progression.
Travis & Sheppard (2014) Annu. Rev. Immunol., 32, 51-82. Several studies have shed light on the mechanisms of TGFβ1 activation. Three integrins, αvβ6, αvβ8, and αvβ1 were demonstrated to be key activators of latent TGFβ1.
Van Laer et al. (2014) Adv. Exp. Med. Biol., 802: p. 95-105. Patients with Loeys/Dietz syndrome carry autosomal dominant mutations in components of the TGFβ signaling pathway, which cause aortic aneurism, hypertelorism, and bifid uvula.
Wang et al. (2012) Mol. Biol. Cell., 23(6), 1129-39 and T.A. Springer, Int. BMP Conference (2016). On the surface of activated regulatory T cells latent TGFβ1 is covalently linked to the transmembrane protein GARP (glycoprotein-A repetitions predominant protein (GARP), and a protein closely related to GARP, LRRC33 (leucine-rich repeat-containing protein 33), serves as a presenting molecule for TGFβ1 on the surface of monocytes, macrophages and microglia. Key for integrin-dependent activation of latent TGFβ1 is the covalent tether to presenting molecules. Disruption of the disulfide bonds between GARP and TGFβ1 LAP by mutagenesis does not impair complex formation, but completely abolishes TGFβ1 activation by αvβ6.
Ward et al. (1989) Nature, 341, 544-546 discloses dAb fragments.
Worthington, Klementowicz, & Travis (2011) Trends Biochem. Sci., 36(1), 47-54. Knockout of candidate proteases did not result in a TGFβ1 phenotype. This could be explained by redundancies or by these mechanisms being critical in specific diseases rather than development and homeostasis.
Yang et al. (2007) J. Cell Biol., 176(6), 787-93. Transgenic mice with a mutation in the TGFβ1 RGD site that prevents integrin binding, but not secretion, phenocopy the TGFβ1−/− mouse.

### TEXTBOOKS AND TECHNICAL REFERENCES

Current Protocols in Immunology (CPI), Coligan et al., eds. (John Wiley and Sons, Inc., 2003).
Current Protocols in Molecular Biology (CPMB), Ausubel, ed. (John Wiley and Sons, 2014).
Current Protocols in Protein Science (CPPS), Coligan et al., ed, (John Wiley and Sons, Inc., 2005).
Immunology, Werner Luttmann (ed.) (Elsevier 2006).
Janeway's Immunobiology, Kenneth Murphy, Allan Mowat, Casey Weaver (eds.) (Taylor & Francis Limited, 2014).
Laboratory Methods in Enzymology: DNA, Jon Lorsch (ed.) (Elsevier, 2013).
Lewin's Genes XI (Jones & Bartlett Publishers, 2014).
Molecular Biology and Biotechnology: A Comprehensive Desk Reference, Robert A. Meyers (ed.) (VCH Publishers, Inc., 1995).
Molecular Cloning: A Laboratory Manual, 4th ed., Michael Richard Green and Joseph Sambrook, (Cold Spring Harbor, N.Y., Cold Spring Harbor Laboratory Press, 2012).
The Encyclopedia of Molecular Cell Biology and Molecular Medicine, Robert S. Porter et al. (eds.) (Blackwell Science Ltd., 1999-2012).
The Merck Manual of Diagnosis and Therapy, 19^{th} edition (Merck Sharp & Dohme Corp., 2018).
Pharmaceutical Sciences 23^{rd} edition (Elsevier, 2020).

### List of Certain Embodiments

1. A method of treating a fibrotic condition, comprising the steps of:
   (a) selecting a TGFβ inhibitor for the treatment of fibrosis,
      (1) wherein the TGFβ inhibitor (A) specifically binds a LTBP1-proTGFβ complex; (B) does not bind a human GARP-proTGFβ complex; (C) does not bind a human LRRC33-proTGFβ complex; and (D) does not bind mature TGFβ1, mature TGFβ2 or mature TGFβ3; and
      (2) wherein the TGFβ inhibitor is selected using an assay to measure the amount of a marker, wherein the amount of the marker is indicative of the treatment of fibrosis, with the proviso that the marker is not pSmad2; and
   (b) providing the selected TGFβ inhibitor for administration to a subject in need of treatment for the fibrotic condition, wherein the fibrotic condition is treatable by administration of an isoform-specific, context-selective TGFβ1 inhibitors that selectively target matrix-associated TGFβ1 activation but not immune cell-associated TGFβ1 activation.
2. The method of embodiment 1, wherein the selected antibody comprises (1) CDR-H1 comprising the amino acid sequence FTFRSYVMH (SEQ ID NO: 1); (2) CDR-H2 comprising the amino acid sequence VISHEGSLKYYADSVKG (SEQ ID NO: 2); (3) CDR-H3 comprising the amino acid sequence ARPRIAARRGGFGY (SEQ ID NO: 3); (4) CDR-L1 comprising the amino acid sequence TRSSGNIDNNYVQ (SEQ ID NO: 4); (5) CDR-L2 comprising the amino acid sequence EDNQRPS (SEQ ID NO: 5); and (6) CDR-L3 comprising the amino acid sequence QSYDYDTQGVV (SEQ ID NO: 6).
3. The method of embodiment 1, wherein the selected antibody comprises an antibody or antigen-binding fragment thereof competes or cross-competes with an antibody having a heavy chain variable region sequence as set forth in SEQ ID NO: 7 and light chain variable region sequence as set forth in SEQ ID NO: 8.
4. The method of embodiment 1, wherein the selected antibody comprises either the LTBP-specific antibody Ab42 or the LTBP-specific antibody Ab42-YTE.
5. The method of embodiment 1, wherein the fibrotic condition is pulmonary fibrosis (PF).
6. The method of embodiment 1, wherein the fibrotic condition is idiopathic pulmonary fibrosis (IPF).
7. The method of embodiment 1, wherein the fibrotic condition is kidney fibrosis.
8. The method of embodiment 7, wherein the kidney fibrosis is associated with CKD.
9. The method of embodiment 7, wherein the kidney fibrosis is associated with Alport syndrome, wherein further optionally the subject is treated with a gene therapy or an RNA-based therapy.
10. The method of embodiment 1, wherein the fibrotic condition is muscle fibrosis.
11. The method of embodiment 10, wherein the muscle fibrosis is associated with a muscular dystrophy, wherein optionally the muscular dystrophy is DMD, wherein further optionally the subject is treated with a gene therapy or RNA-based therapy.
12. The method of embodiment 8, wherein the muscle fibrosis is associated with muscular atrophy, wherein optionally the muscular atrophy is SMA.
13. The method of embodiment 1, wherein the fibrotic condition is a fibrosis of the connective tissue, wherein optionally the fibrosis is scleroderma.
14. The method of embodiment 1, further wherein the selected TGFβ inhibitor is for administration to a subject at a time overlapping the time that the subject is being treated with pirfenidone and/or nintedanib.
15. The method of embodiment 1, further wherein the selected TGFβ inhibitor is for administration to a subject after a time that the subject is being treated with pirfenidone and nintedanib.
16. An antibody for use in treating a fibrotic condition, wherein the antibody is selected among the TGFβ inhibitors that (A) specifically bind a LTBP1-proTGFβ complex; (B) does not bind a human GARP-proTGFβ complex; (C) does not bind a human LRRC33-proTGFβ complex; and (D) does not bind mature TGFβ1, mature TGFβ2 or mature TGFβ3; wherein the TGFβ inhibitor is selected using an assay to measure the amount of a marker, wherein the amount of the marker is indicative of the treatment of fibrosis, with the proviso that the marker is not pSmad2.
17. An LTBP-selective inhibitor of TGFβ1 activation for use in the treatment of a fibrotic condition is a subject, wherein the LTBP-selective inhibitor of TGFβ1 activation is an antibody or antigen-binding fragment thereof that binds an epitope in the α-1-helix region of proTGFβ1, wherein optionally the epitope comprises one or more amino acid residues of VKRKRIEA (SEQ ID NO: 9), wherein further optionally the antibody is not Ab42.
18. The LTBP-selective inhibitor of TGFβ1 activation for use according to embodiment 17, wherein the fibrotic condition is NASH, NAFLD, liver fibrosis, chronic kidney disease (CKD), kidney fibrosis, lung disease, lung fibrosis, COPD, IPF, scleroderma, or a muscular dystrophy.
19. The LTBP-selective inhibitor of TGFβ1 activation for use according to embodiment 17 or 18, wherein the treatment comprises subcutaneous administration of a composition comprising the LTBP-selective inhibitor of TGFβ1 activation.
20. A combination therapy for use in treating a fibrotic condition, wherein the therapy comprises the combination of pirfenidone, nintedanib, and an antibody of embodiment 16.
21. The combination therapy of embodiment 20, wherein the fibrotic condition is idiopathic pulmonary fibrosis.
22. The combination therapy of embodiment 20, wherein the antibody of claim 16 is for administration to a subject at a time overlapping the time that the subject is being treated with pirfenidone and nintedanib.
23. The combination therapy of embodiment 20, wherein the antibody of claim 16 is for administration to a subject after a time that the subject is being treated with pirfenidone and nintedanib.
24. A composition comprising an antibody or an antigen-binding fragment thereof that binds a human LTBP1-proTGFβ1 complex and/or human LTBP3-proTGFβ1 complex, wherein the antibody or the fragment binds an epitope comprising one or more amino acid residues of DMELVKRKRIEAIR (SEQ ID NO: 46) within the prodomain of the complex, wherein optionally the epitope further comprises at least one additional amino acid residue within the growth factor domain of the complex, and wherein the antibody does not have the same six CDR sequences as Ab42, and wherein optionally the antibody or the fragment has a monovalent IC50 value of 1 nM or less as measured by a cell-based potency assay as described in WO 2020/160291.
25. The composition of embodiment 24, wherein the antibody or the fragment contacts one or more of the following residues within the prodomain: Asp27 (D); Leu30 (L); Arg33 (R); Ile36 (I); Glu37 (E).
26. The composition of embodiment 24 or 25, wherein the epitope binds the Finger-1 domain within the growth factor, wherein optionally the epitope comprises one or more amino acid residues of YIDFRKDLGWK (SEQ ID NO: 93) within the Finger-1 domain, wherein further optionally the antibody or the fragment contacts one or more of the following residues: Tyr289 (Y); Lys294 (K); Asp295 (D).
27. The composition of any one of embodiments 24-26, wherein the antibody or the fragment binds to a recombinant human LTBP1-proTGFβ1 complex and/or LTBP3-proTGFβ1 complex with at least 50-fold greater affinity/affinities (50-fold lower KD values) than to a recombinant human GARP-proTGFβ1 complex and/or LRRC33-proTGFβ1 complex.
28. A method for manufacturing a pharmaceutical composition comprising the antibody or the fragment according to any one of embodiments 24-27, wherein the method comprises producing a recombinant antibody or a protein construct comprising the antigen-binding fragment of the antibody in a large-scale cell culture comprising a culture volume of 500 L or larger, purifying the recombinant antibody or the protein construct from the cell culture, and formulating into a pharmaceutical composition comprising at least one excipient.
29. An LTBP-selective inhibitor of TGFβ1 activation for use in the treatment of chronic kidney disease (CKD) in a subject, wherein the treatment comprises administration of an LTBP-selective inhibitor which is an antibody that does not bind GARP-proTGFβ1, in an amount effective to treat CKD, wherein optionally the subject is further treated with a myostatin-selective inhibitor.
30. A myostatin-selective inhibitor for use in the treatment of CKD in a subject, wherein the treatment comprises administration of a myostatin-selective inhibitor to the subject in an amount effective to treat CKD, wherein optionally the subject is further treated with an LTBP-selective inhibitor of TGFβ1 activation.
31. An LTBP-selective inhibitor and a myostatin-selective inhibitor for use in the treatment of CKD in a subject, wherein the treatment comprises administration of an LTBP-selective inhibitor and a myostatin-selective inhibitor to the subject in amounts effective to treat CKD.
32. The LTBP-selective inhibitor and/or the myostatin-selective inhibitor for use according to any one of embodiments 29-31, wherein the CKD is associated with iron deficiency, wherein optionally the subject is treated with an iron-enhancing therapy.

What is claimed is:

1. A LTBP-selective TGFβ1 inhibitor for use in the treatment of an inflammatory fibrotic disease in a subject, wherein the treatment comprises administration of an effective amount of a LTBP-selective TGFβ1 inhibitor to the subject, wherein the LTBP-selective TGFβ1 inhibitor is:
a) an antibody or antigen-binding fragment thereof comprising the following six CDRs: (1) CDR-H1 comprising the amino acid sequence FTFRSYVMH (SEQ ID NO: 1); (2) CDR-H2 comprising the amino acid sequence VISHEGSLKYYADSVKG (SEQ ID NO: 2); (3) CDR-H3 comprising the amino acid sequence ARPRIAARRGGFGY (SEQ ID NO: 3); (4) CDR-L1 comprising the amino acid sequence TRSSGNIDNNYVQ (SEQ ID NO: 4); (5) CDR-L2 comprising the amino acid sequence EDNQRPS (SEQ ID NO: 5); and (6) CDR-L3 comprising the amino acid sequence QSYDYDTQGVV (SEQ ID NO: 6);
b) an antibody that is not Ab42 and comprises a VH and a VL each of which has at least 90% sequence identity to SEQ ID NO: 7 and SEQ ID NO: 8, respectively, and binds human LTBP1-proTGFβ1 and human LTBP3-proTGFβ1 with a K_{D} of 10 nM or less as measured by a surface plasmon resonance (SPR)-based assay (such as BIACORE);
c) an antibody that is not Ab42 and competes for antigen binding with SR42 and binds an epitope comprising one or more amino acid residues of the sequence DMELVKRKRIEAIR (SEQ ID NO: 46), wherein optionally the antibody contacts one or more of the residues: Asp27 (D); Leu30 (L); Arg33 (R); Ile36 (I); Glu37 (E); or,
d) an antibody that is not Ab42 and competes for antigen binding with SR42 and binds an epitope comprising one or more amino acid residues of the sequence YIDFRKDLGWK (SEQ ID NO: 93), wherein optionally the antibody contacts one or more of the residues: Tyr289 (Y); Lys294 (K); Asp295 (D);
wherein the antibody binds recombinant human LTBP1-proTGFβ1 and/or recombinant human LTBP3-proTGFβ1 with at least 50-fold greater affinities over recombinant human GARP-proTGFβ1 as measured in an in vitro binding assay.

2. The LTBP-selective TGFβ1 inhibitor for use according to claim 1, wherein the inflammatory fibrotic disease is a viral infection, wherein optionally the viral infection is a severe acute respiratory syndrome; wherein further optionally the severe acute respiratory syndrome is SARS-CoV (e.g., SARS-CoV1) or COVID19 (SARS-CoV2).

3. The LTBP-selective TGFβ1 inhibitor for use according to claim 1, wherein the inflammatory fibrotic disease is a chronic kidney disease (CKD), a chronic lung disease, a chronic liver disease, diabetes, muscle disease, systemic sclerosis, cancer, and/or genotoxic therapy-induced inflammation.

4. The LTBP-selective TGFβ1 inhibitor for use according to claim 3, wherein the chronic kidney disease is diabetic nephropathy, renal fibrosis, and/or Alport syndrome.

5. The LTBP-selective TGFβ1 inhibitor for use according to claim 3, wherein the chronic liver disease is non-alcoholic fatty liver disease (NAFLD), e.g., non-alcoholic fatty liver (NAFL) and non-alcoholic steatohepatitis (NASH), which may include: noncirrhotic NASH with liver fibrosis, liver cirrhosis, NASH with compensated cirrhosis, NASH with decompensated cirrhosis, liver inflammation with fibrosis, liver inflammation without fibrosis; stage 2 and 3 liver fibrosis, stage 4 fibrosis (NASH cirrhosis or cirrhotic NASH with fibrosis), primary biliary cholangitis (PBC) (formerly known as primary biliary cirrhosis), and/or primary sclerosing cholangitis (PSC), wherein optionally the subject has or is at risk of developing obesity, metabolic syndrome, and/or type 2 diabetes.

6. The LTBP-selective TGFβ1 inhibitor for use according to claim 5, wherein the LTBP-selective TGFβ1 inhibitor is used in conjunction with another therapy comprising a myostatin inhibitor and/or a GLP-1 receptor agonist.

7. The LTBP-selective TGFβ1 inhibitor for use according to claim 6, wherein the myostatin inhibitor is a myostatin-selective inhibitor, wherein optionally the myostatin-selective inhibitor is apitegromab, GYM329, trevogromab, or a variant thereof.

8. The LTBP-selective TGFβ1 inhibitor for use according to claim 6, wherein the myostatin inhibitor is a non-selective myostatin inhibitor, wherein optionally the non-selective myostatin inhibitor is an antibody that binds ActRII (e.g., bimagromab), a ligand trap, or an anti-myostatin Adnectin (e.g., BMS-986089.

9. The LTBP-selective TGFβ1 inhibitor for use according to claim 3, wherein the diabetes is type 1 diabetes or type 2 diabetes.

10. The LTBP-selective TGFβ1 inhibitor for use according to claim 3, wherein the chronic lung disease is IPF, infection, and/or radiation-induced pulmonary fibrosis.

11. The LTBP-selective TGFβ1 inhibitor for use according to claim 3, wherein the muscle disease is a dystrophy, wherein optionally the dystrophy is DMD, wherein further optionally the subject is treated with a dystrophin-directed therapy.

12. The LTBP-selective TGFβ1 inhibitor for use according to claim 3, wherein the cancer comprises a fibrotic solid tumor (e.g., desmoplasia).

13. The LTBP-selective TGFβ1 inhibitor for use according to any one of the previous claims, wherein the subject is treated with a genotoxic therapy, wherein the genotoxic therapy-induced inflammation is radiation therapy-induced inflammation or chemotherapy-induced inflammation, wherein optionally, the subject has cancer selected from:
uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), head and neck squamous cell carcinoma (HNSC), glioblastoma multiforme (GMB), esophageal carcinoma (ESCA), colon adenocarcinoma (COAD), breast invasive carcinoma (BRCA), bladder urothelial carcinoma (BLCA), myelofibrosis, melanoma, adjuvant melanoma, renal cell carcinoma (RCC) (e.g., clear cell RCC, papillary RCC, chromophobe RCC, collecting duct RCC, and unclassified RCC), bladder cancer, colorectal cancer (CRC) (e.g., microsatellite-stable CRC, mismatch repair deficient colorectal cancer), colon cancer, rectal cancer, anal cancer, breast cancer, triple-negative breast cancer (TNBC), HER2-negative breast cancer, HER2-positive breast cancer, BRCA-mutated breast cancer, hematologic malignancies, non-small cell carcinoma, non-small cell lung cancer/carcinoma (NSCLC), small cell lung cancer/carcinoma (SCLC), extensive-stage small cell lung cancer (ES-SCLC), lymphoma (classical Hodgkin's and non-Hodgkin's), primary mediastinal large B-cell lymphoma (PMBCL), T-cell lymphoma, diffuse large B-cell lymphoma, histiocytic sarcoma, follicular dendritic cell sarcoma, interdigitating dendritic cell sarcoma, myeloma, chronic lymphocytic leukemia (CLL), acute myeloid leukemia (AML), small lymphocytic lymphoma (SLL), head and neck cancer (e.g., head and neck squamous cell cancer), urothelial cancer e.g., metastatic urothelial carcinoma), merkel cell carcinoma (e.g., metastatic merkel cell carcinoma), merkel cell skin cancer, cancer with high microsatellite instability (MSI-H), cancer with mismatch repair deficiency (dMMR), tumor mutation burden high cancer, mesothelioma (e.g., malignant pleural mesothelioma), gastric cancer, gastroesophageal junction cancer (GEJ), gastric adenocarcinoma, neuroendocrine tumors, gastrointestinal stromal tumors (GIST), gastric cardia adenocarcinoma, renal cancer, biliary cancer, cholangiocarcinoma, pancreatic cancer, prostate cancer, adenocarcinoma, squamous cell carcinoma, non-squamous cell carcinoma, cutaneous squamous cell carcinoma (CSCC), ovarian cancer, endometrial cancer, fallopian tube cancer, cervical cancer, peritoneal cancer, stomach cancer, brain cancers, malignant glioma, glioblastoma, gliosarcoma, neuroblastoma, thyroid cancer, adrenocortical carcinoma, oral intra-epithelial neoplasia, esophageal cancer, nasal cavity and paranasal sinus squamous cell carcinoma, nasopharynx carcinoma, salivary gland cancer, liver cancer, basal cell carcinoma, and hepatocellular cancer (HCC).

14. The LTBP-selective TGFβ1 inhibitor for use according to any one of the preceding claims, wherein the subject is treated with a Treg-enhancing agent, wherein optionally the Treg-enhancing agent comprises all-trans retinoic acid (ATRA), vitamin D3, indoleamine-pyrrole 2,3-dioxygenase (IDO), short-chain fatty acids (e.g., butyrate), and/or AKT/mTOR pathway inhibitors such as rapamycin.

15. The LTBP-selective TGFβ1 inhibitor for use according to any one of the preceding claims, wherein the LTBP-selective TGFβ1 inhibitor is administered in an amount sufficient to achieve a serum concentration of about 100-300 μg/mL.

16. A pharmaceutical composition comprising an antibody or an antigen-binding fragment thereof that binds recombinant human LTBP1-proTGFβ1 and/or recombinant human LTBP3-proTGFβ1 with a K_{D} of 10 nM or less as measured by a SPR-based assay (such as Biacore),
wherein the antibody is not Ab42,
wherein the antibody competes for antigen binding with SR42 and binds an epitope comprising one or more amino acid residues of the sequence DMELVKRKRIEAIR (SEQ ID NO: 46), wherein optionally the antibody contacts one or more of the residues: Asp27 (D); Leu30 (L); Arg33 (R); Ile36 (I); Glu37 (E).

17. A pharmaceutical composition comprising an antibody or an antigen-binding fragment thereof that binds recombinant human LTBP1-proTGFβ1 and/or recombinant human LTBP3-proTGFβ1 with a K_{D} of 10 nM or less as measured by a SPR-based assay (such as Biacore),
wherein the antibody is not Ab42;
wherein the antibody competes for antigen binding with SR42 and binds an epitope comprising one or more amino acid residues of the sequence YIDFRKDLGWK (SEQ ID NO: 93), wherein optionally the antibody contacts one or more of the residues: Tyr289 (Y); Lys294 (K); Asp295 (D).


SEQUENCE LISTING (93 sequences)

SEQ ID NO: 1 - protein, 9 aa, synthetic construct
  REGION 1..9: note = Synthetic: CDRH1 sequence
FTFRSYVMH

SEQ ID NO: 2 - protein, 17 aa, synthetic construct
  REGION 1..17: note = Synthetic: CDRH2 sequence
VISHEGSLKYYADSVKG

SEQ ID NO: 3 - protein, 14 aa, synthetic construct
  REGION 1..14: note = Synthetic: CDRH3 sequence
ARPRIAARRGGFGY

SEQ ID NO: 4 - protein, 13 aa, synthetic construct
  REGION 1..13: note = Synthetic: CDRL1 sequence
TRSSGNIDNNYVQ

SEQ ID NO: 5 - protein, 7 aa, synthetic construct
  REGION 1..7: note = Synthetic: CDRL2 sequence
EDNQRPS

SEQ ID NO: 6 - protein, 11 aa, synthetic construct
  REGION 1..11: note = Synthetic: CDR-L3
QSYDYDTQGVV

SEQ ID NO: 7 - protein, 121 aa, synthetic construct
  REGION 1..121: note = Synthetic: HCVR of antibody AB42
QVQLVESGGGVVQPGRSLRLSCAASGFTFRSYVMHWVRQAPGKGLEWVAVISHEGSLKYYADSVKGRFTISRDNSKNTLYLQMNSLRAEDTAVYYCARPRIAARRGGFGYWGQGTLVTVSS

SEQ ID NO: 8 - protein, 112 aa, synthetic construct
  REGION 1..112: note = Synthetic: LCVR of antibody AB42
NFMLTQPHSVSESPGKTVTISCTRSSGNIDNNYVQWYQQRPGSSPTTVIYEDNQRPSGVPDRFSGSIDSSSNSASLTISGLKTEDEADYYCQSYDYDTQGVVFGGGTKLTVL

SEQ ID NO: 9 - protein, 8 aa, synthetic construct
  REGION 1..8: note = Synthetic: alpha1-helix region of the prodomain of proTGF-beta-1
VKRKRIEA

SEQ ID NO: 10 - protein, 447 aa, synthetic construct
  REGION 1..447: note = Synthetic: Sequence of Ab42-YTE huG4
QVQLVESGGGVVQPGRSLRLSCAASGFTFRSYVMHWVRQAPGKGLEWVAVISHEGSLKYYADSVKGRFTISRDNSKNTLYLQMNSLRAEDTAVYYCARPRIAARRGGFGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 218 aa, synthetic construct
  REGION 1..218: note = Synthetic: Sequence of Ab42-YTE huLambda
NFMLTQPHSVSESPGKTVTISCTRSSGNIDNNYVQWYQQRPGSSPTTVIYEDNQRPSGVPDRFSGSIDSSSNSASLTISGLKTEDEADYYCQSYDYDTQGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 447 aa, synthetic construct; identical to SEQ ID NO: 10
  REGION 1..447: note = Synthetic: Sequence of Ab42-YTE huG4

SEQ ID NO: 13 - protein, 218 aa, synthetic construct; identical to SEQ ID NO: 11
  REGION 1..218: note = Synthetic: Sequence of Ab42-YTE huLambda

SEQ ID NO: 14 - protein, 360 aa, synthetic construct
  REGION 1..360: note = Synthetic: proTGF-beta-1 D2G
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 15 - protein, 360 aa, synthetic construct
  REGION 1..360: note = Synthetic: proTGF-beta-1 C4S D2G
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 16 - protein, 395 aa, synthetic construct
  REGION 1..395: note = Synthetic: proTGF-beta-2
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 17 - protein, 395 aa, synthetic construct
  REGION 1..395: note = Synthetic: proTGF-beta-2 C5S
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 18 - protein, 394 aa, synthetic construct
  REGION 1..394: note = Synthetic: proTGF-beta-2 C5S D2G
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 19 - protein, 394 aa, synthetic construct
  REGION 1..394: note = Synthetic: proTGF-beta-2 D2G
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 20 - protein, 392 aa, synthetic construct
  REGION 1..392: note = Synthetic: proTGF-beta-3
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 21 - protein, 392 aa, synthetic construct
  REGION 1..392: note = Synthetic: proTGF-beta-3 C7S
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 22 - protein, 391 aa, synthetic construct
  REGION 1..391: note = Synthetic: proTGF-beta-3 C7S D2G
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 23 - protein, 391 aa, synthetic construct
  REGION 1..391: note = Synthetic: proTGF-beta-3 D2G
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 24 - protein, 361 aa, Mus musculus
  REGION 1..361: note = misc_feature - proTGF-beta-1
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 25 - protein, 361 aa, Macaca fascicularis
  REGION 1..361: note = misc_feature - proTGF-beta-1
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 249 aa, Mus musculus
  REGION 1..249: note = misc_feature - TGF-beta-1 LAP C4S
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 27 - protein, 249 aa, Macaca fascicularis
  REGION 1..249: note = misc_feature - TGF-beta-1 LAP C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRR

SEQ ID NO: 28 - protein, 360 aa, Mus musculus
  REGION 1..360: note = misc_feature - proTGF-beta-1 C4S D2G
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHGALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 29 - protein, 361 aa, Mus musculus
  REGION 1..361: note = misc_feature - proTGF-beta-1 C4S
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 30 - protein, 361 aa, Macaca fascicularis
  REGION 1..361: note = misc_feature - proTGF-beta-1 C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 31 - protein, 360 aa, Macaca fascicularis
  REGION 1..360: note = misc_feature - proTGF-beta-1 C4S D2G
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 32 - protein, 1375 aa, Homo sapiens
  REGION 1..1375: note = misc_feature - LTBP1S
NHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGVGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 33 - protein, 1373 aa, Macaca fascicularis
  REGION 1..1373: note = misc_feature - LTBP1S
NHTGRIKVVFTPSICKVTCTKGSCQNSCEKGNTTTLISENGHAADTLTATNFRVVLCHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYKFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCTNGDCSNLEGSYMCSCHKGYTRTPDHKHCKDIDECQQGNLCVNGQCKNTEGSFRCTCGQGYQLSAAKDQCEDIDECQHHHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVEQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPMKDSDDYAQLCNIPVTGRRQPYGRDALVDFSEQYAPEADPYFIQDRFLNSFEELQAEECGILNGCENGRCVRVQEGYTCDCFDGYHLDTAKMTCVDVNECDELNNRMSLCKNAKCINTEGSYKCLCLPGYVPSDKPNYCTPLNTALNLEKDSDLE

SEQ ID NO: 34 - protein, 1374 aa, Mus musculus
  REGION 1..1374: note = misc_feature - LTBP1S
NHTGRIKVVFTPSICKVTCTKGNCQNSCQKGNTTTLISENGHAADTLTATNFRVVICHLPCMNGGQCSSRDKCQCPPNFTGKLCQIPVLGASMPKLYQHAQQQGKALGSHVIHSTHTLPLTMTSQQGVKVKFPPNIVNIHVKHPPEASVQIHQVSRIDSPGGQKVKEAQPGQSQVSYQGLPVQKTQTVHSTYSHQQLIPHVYPVAAKTQLGRCFQETIGSQCGKALPGLSKQEDCCGTVGTSWGFNKCQKCPKKQSYHGYTQMMECLQGYKRVNNTFCQDINECQLQGVCPNGECLNTMGSYRCSCKMGFGPDPTFSSCVPDPPVISEEKGPCYRLVSPGRHCMHPLSVHLTKQICCCSVGKAWGPHCEKCPLPGTAAFKEICPGGMGYTVSGVHRRRPIHQHIGKEAVYVKPKNTQPVAKSTHPPPLPAKEEPVEALTSSWEHGPRGAEPEVVTAPPEKEIPSLDQEKTRLEPGQPQLSPGVSTIHLHPQFPVVVEKTSPPVPVEVAPEASTSSASQVIAPTQVTEINECTVNPDICGAGHCINLPVRYTCICYEGYKFSEQLRKCVDIDECAQVRHLCSQGRCENTEGSFLCVCPAGFMASEEGTNCIDVDECLRPDMCRDGRCINTAGAFRCEYCDSGYRMSRRGYCEDIDECLKPSTCPEEQCVNTPGSYQCVPCTEGFRGWNGQCLDVDECLQPKVCTNGSCTNLEGSYMCSCHRGYSPTPDHRHCQDIDECQQGNLCMNGQCRNTDGSFRCTCGQGYQLSAAKDQCEDIDECEHHHLCSHGQCRNTEGSFQCVCNQGYRASVLGDHCEDINECLEDSSVCQGGDCINTAGSYDCTCPDGFQLNDNKGCQDINECAQPGLCGSHGECLNTQGSFHCVCEQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRVTEDSGVDRQPREEKKECYYNLNDASLCDNVLAPNVTKQECCCTSGAGWGDNCEIFPCPVQGTAEFTEMCPRGKGLVPAGESSYDTGGENYKDADECLLFGEEICKNGYCLNTQPGYECYCKQGTYYDPVKLQCFDMDECQDPNSCIDGQCVNTEGSYNCFCTHPMVLDASEKRCVQPTESNEQIEETDVYQDLCWEHLSEEYVCSRPLVGKQTTYTECCCLYGEAWGMQCALCPMKDSDDYAQLCNIPVTGRRRPYGRDALVDFSEQYGPETDPYFIQDRFLNSFEELQAEECGILNGCENGRCVRVQEGYTCDCFDGYHLDMAKMTCVDVNECSELNNRMSLCKNAKCINTEGSYKCLCLPGYIPSDKPNYCTPLNSALNLDKESDLE

SEQ ID NO: 35 - protein, 1260 aa, Homo sapiens
  REGION 1..1260: note = misc_feature - LTBP3
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRTGALSTGALPPLAPEGDSVASKHAIYAVQVIADPPGPGEGPPAQHAAFLVPLGPGQISAEVQAPPPVVNVRVHHPPEASVQVHRIESSNAESAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPTDGTAAFKEICPAGKGYHILTSHQTLTIQGESDFSLFLHPDGPPKPQQLPESPSQAPPPEDTEEERGVTTDSPVSEERSVQQSHPTATTTPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCLDVDECEAGDVCDNGICSNTPGSFQCQCLSGYHLSRDRSHCEDIDECDFPAACIGGDCINTNGSYRCLCPQGHRLVGGRKCQDIDECSQDPSLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGSEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPLAGPALTFDDCCCRQGRGWGAQCRPCPPRGAGSHCPTSQSESNSFWDTSPLLLGKPPRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 36 - protein, 1260 aa, Macaca fascicularis
  REGION 1..1260: note = misc_feature - LTBP3
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRAGALSTGALPPLAPEGDSVASKHAIYAVQVIADPPGPGEGPPAQHAAFLVPLGPGQISAEVQAPPPVVNVRVHHPPEASVQVHRIESSNAEGAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPADGTAAFKEICPAGKGYHILTSHQTLTIQGESDFSLFLHPDGPPKPQQLPESPSQAPPPEDTEEERGVTTDSPVSEERSVQQSHPTATTSPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCVDVDECEAGDVCDNGICTNTPGSFQCQCLSGYHLSRDRSHCEDIDECDFPAACIGGDCINTNGSYRCLCPQGHRLVGGRKCQDIDECTQDPGLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGAEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPQAGPALTFDDCCCRQGRGWGAQCRPCPPRGAGSQCPTSQSESNSFWDTSPLLLGKPRRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 37 - protein, 1228 aa, Mus musculus
  REGION 1..1228: note = misc_feature - LTBP3
GPAGERGTGGGGALARERFKVVFAPVICKRTCLKGQCRDSCQQGSNMTLIGENGHSTDTLTGSAFRVVVCPLPCMNGGQCSSRNQCLCPPDFTGRFCQVPAAGTGAGTGSSGPGLARTGAMSTGPLPPLAPEGESVASKHAIYAVQVIADPPGPGEGPPAQHAAFLVPLGPGQISAEVQAPPPVVNVRVHHPPEASVQVHRIEGPNAEGPASSQHLLPHPKPPHPRPPTQKPLGRCFQDTLPKQPCGSNPLPGLTKQEDCCGSIGTAWGQSKCHKCPQLQYTGVQKPVPVRGEVGADCPQGYKRLNSTHCQDINECAMPGNVCHGDCLNNPGSYRCVCPPGHSLGPLAAQCIADKPEEKSLCFRLVSTEHQCQHPLTTRLTRQLCCCSVGKAWGARCQRCPADGTAAFKEICPGKGYHILTSHQTLTIQGESDFSLFLHPDGPPKPQQLPESPSRAPPLEDTEEERGVTMDPPVSEERSVQQSHPTTTTSPPRPYPELISRPSPPTFHRFLPDLPPSRSAVEIAPTQVTETDECRLNQNICGHGQCVPGPSDYSCHCNAGYRSHPQHRYCVDVNECEAEPCGPGKGICMNTGGSYNCHCNRGYRLHVGAGGRSCVDLNECAKPHLCGDGGFCINFPGHYKCNCYPGYRLKASRPPICEDIDECRDPSTCPDGKCENKPGSFKCIACQPGYRSQGGGACRDVNECSEGTPCSPGWCENLPGSYRCTCAQYEPAQDGLSCIDVDECEAGKVCQDGICTNTPGSFQCQCLSGYHLSRDRSRCEDIDECDFPAACIGGDCINTNGSYRCLCPLGHRLVGGRKCKKDIDECSQDPGLCLPHACENLQGSYVCVCDEGFTLTQDQHGCEEVEQPHHKKECYLNFDDTVFCDSVLATNVTQQECCCSLGAGWGDHCEIYPCPVYSSAEFHSLVPDGKRLHSGQQHCELCIPAHRDIDECILFGAEICKEGKCVNTQPGYECYCKQGFYYDGNLLECVDVDECLDESNCRNGVCENTRGGYRCACTPPAEYSPAQAQCLIPERWSTPQRDVKCAGASEERTACVWGPWAGPALTFDDCCCRQPRLGTQCRPCPPRGTGSQCPTSQSESNSFWDTSPLLLGKSPRDEDSSEEDSDECRCVSGRCVPRPGGAVCECPGGFQLDASRARCVDIDECRELNQRGLLCKSERCVNTSGSFRCVCKAGFTRSRPHGPACLSAAADDAAIAHTSVIDHRGYFH

SEQ ID NO: 38 - protein, 645 aa, synthetic construct
  REGION 1..645: note = Synthetic: GARP
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 39 - protein, 610 aa, synthetic construct
  REGION 1..610: note = Synthetic: sGARP
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNIN

SEQ ID NO: 40 - protein, 646 aa, Mus musculus
  REGION 1..646: note = misc_feature - GARP mouse
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 41 - protein, 611 aa, Mus musculus
  REGION 1..611: note = misc_feature - sGARP mouse
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVN

SEQ ID NO: 42 - protein, 692 aa, Homo sapiens
  REGION 1..692: note = misc_feature - LRRC33 (also known as NRROS; Uniprot Accession No. Q86YC3)
MELLPLWLCLGFHFLTVGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLYLVLILPSCLTLLVACTVIVLTFKKPLLQVIKSRCHWSSVY

SEQ ID NO: 43 - protein, 660 aa, synthetic construct
  REGION 1..660: note = Synthetic: soluble LRRC33 (sLRRC33)
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLHHHHHH

SEQ ID NO: 44 - protein, 689 aa, synthetic construct
  REGION 1..689: note = Synthetic: Human LRRC33-GARP chimera
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLIIILTFILVSAILLTTLAACCCVRRQKFNQQYKA

SEQ ID NO: 45 - protein, 5 aa, synthetic construct
  REGION 1..5: note = Synthetic: IgGl-like hinge sequence
CPPCP

SEQ ID NO: 46 - protein, 14 aa, synthetic construct
  REGION 1..14: note = Synthetic: epitope
DMELVKRKRIEAIR

SEQ ID NO: 47 - protein, 249 aa, synthetic construct
  REGION 1..249: note = Synthetic: Latency Associated Peptide (LAP)(prodomain)
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 48 - protein, 45 aa, synthetic construct
  REGION 1..45: note = Synthetic: Straight Jacket
LSTCKTIDMELVKRKRIEAIRGQILSKLRLASPPSQGEVPPGPLP

SEQ ID NO: 49 - protein, 112 aa, synthetic construct
  REGION 1..112: note = Synthetic: Growth Factor Domain
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 50 - protein, 4 aa, synthetic construct
  REGION 1..4: note = Synthetic: Furin cleavage site
RHRR

SEQ ID NO: 51 - protein, 204 aa, synthetic construct
  REGION 1..204: note = Synthetic: Arm
EAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 52 - protein, 29 aa, synthetic construct
  REGION 1..29: note = Synthetic: Finger-1
CVRQLYIDFRKDLGWKWIHEPKGYHANFC

SEQ ID NO: 53 - protein, 35 aa, synthetic construct
  REGION 1..35: note = Synthetic: Finger-2
CVPQALEPLPIVYYVGRKPKVEQLSNMIVRSCKCS

SEQ ID NO: 54 - protein, 15 aa, synthetic construct
  REGION 1..15: note = Synthetic: Latency Lasso
LASPPSQGEVPPGPL

SEQ ID NO: 55 - protein, 27 aa, synthetic construct
  REGION 1..27: note = Synthetic: Extended Latency Lasso
LASPPSQGEVPPGPLPEAVLALYNSTR

SEQ ID NO: 56 - protein, 29 aa, synthetic construct
  REGION 1..29: note = Synthetic: Alpha-1 Helix
LSTCKTIDMELVKRKRIEAIRGQILSKLR

SEQ ID NO: 57 - protein, 10 aa, synthetic construct
  REGION 1..10: note = Synthetic: Alpha-2 Helix
AVLALYNSTR

SEQ ID NO: 58 - protein, 20 aa, synthetic construct
  REGION 1..20: note = Synthetic: Trigger Loop
NGFTTGRRGDLATIHGMNRP

SEQ ID NO: 59 - protein, 13 aa, synthetic construct
  REGION 1..13: note = Synthetic: Bowtie
CSCDSRDNTLQVD

SEQ ID NO: 60 - protein, 361 aa, synthetic construct
  REGION 1..361: note = Synthetic: TGF-beta-1
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 61 - protein, 395 aa, synthetic construct; identical to SEQ ID NO: 16
  REGION 1..395: note = Synthetic: TGF-beta-2

SEQ ID NO: 62 - protein, 392 aa, synthetic construct; identical to SEQ ID NO: 20
  REGION 1..392: note = Synthetic: TGF-beta-3

SEQ ID NO: 63 - protein, 361 aa, synthetic construct; identical to SEQ ID NO: 60
  REGION 1..361: note = Synthetic: proTGF-beta-1

SEQ ID NO: 64 - protein, 361 aa, synthetic construct
  REGION 1..361: note = Synthetic: proTGF-beta-1 C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 65 - protein, 13 aa, synthetic construct
  REGION 1..13: note = Synthetic: exemplary linker
ASTKGPSVFPLAP

SEQ ID NO: 66 - protein, 6 aa, synthetic construct
  REGION 1..6: note = Synthetic: exemplary linker
ASTKGP

SEQ ID NO: 67 - protein, 12 aa, synthetic construct
  REGION 1..12: note = Synthetic: exemplary linker
TVAAPSVFIFPP

SEQ ID NO: 68 - protein, 5 aa, synthetic construct
  REGION 1..5: note = Synthetic: exemplary linker
TVAAP

SEQ ID NO: 69 - protein, 16 aa, synthetic construct
  REGION 1..16: note = Synthetic: exemplary linker
AKTTPKLEEGEFSEAR

SEQ ID NO: 70 - protein, 17 aa, synthetic construct
  REGION 1..17: note = Synthetic: exemplary linker
AKTTPKLEEGEFSEARV

SEQ ID NO: 71 - protein, 9 aa, synthetic construct
  REGION 1..9: note = Synthetic: exemplary linker
AKTTPKLGG

SEQ ID NO: 72 - protein, 10 aa, synthetic construct
  REGION 1..10: note = Synthetic: exemplary linker
SAKTTPKLGG

SEQ ID NO: 73 - protein, 6 aa, synthetic construct
  REGION 1..6: note = Synthetic: exemplary linker
SAKTTP

SEQ ID NO: 74 - protein, 6 aa, synthetic construct
  REGION 1..6: note = Synthetic: exemplary linker
RADAAP

SEQ ID NO: 75 - protein, 9 aa, synthetic construct
  REGION 1..9: note = Synthetic: exemplary linker
RADAAPTVS

SEQ ID NO: 76 - protein, 12 aa, synthetic construct
  REGION 1..12: note = Synthetic: exemplary linker
RADAAAAGGPGS

SEQ ID NO: 77 - protein, 27 aa, synthetic construct
  REGION 1..27: note = Synthetic: exemplary linker
RADAAAAGGGGSGGGGSGGGGSGGGGS

SEQ ID NO: 78 - protein, 18 aa, synthetic construct
  REGION 1..18: note = Synthetic: exemplary linker
SAKTTPKLEEGEFSEARV

SEQ ID NO: 79 - protein, 5 aa, synthetic construct
  REGION 1..5: note = Synthetic: exemplary linker
ADAAP

SEQ ID NO: 80 - protein, 12 aa, synthetic construct
  REGION 1..12: note = Synthetic: exemplary linker
ADAAPTVSIFPP

SEQ ID NO: 81 - protein, 6 aa, synthetic construct
  REGION 1..6: note = Synthetic: exemplary linker
QPKAAP

SEQ ID NO: 82 - protein, 13 aa, synthetic construct
  REGION 1..13: note = Synthetic: exemplary linker
QPKAAPSVTLFPP

SEQ ID NO: 83 - protein, 6 aa, synthetic construct
  REGION 1..6: note = Synthetic: exemplary linker
AKTTPP

SEQ ID NO: 84 - protein, 13 aa, synthetic construct
  REGION 1..13: note = Synthetic: exemplary linker
AKTTPPSVTPLAP

SEQ ID NO: 85 - protein, 6 aa, synthetic construct
  REGION 1..6: note = Synthetic: exemplary linker
AKTTAP

SEQ ID NO: 86 - protein, 13 aa, synthetic construct
  REGION 1..13: note = Synthetic: exemplary linker
AKTTAPSVYPLAP

SEQ ID NO: 87 - protein, 15 aa, synthetic construct
  REGION 1..15: note = Synthetic: exemplary linker
GGGGSGGGGSGGGGS

SEQ ID NO: 88 - protein, 15 aa, synthetic construct
  REGION 1..15: note = Synthetic: exemplary linker
GENKVEYAPALMALS

SEQ ID NO: 89 - protein, 15 aa, synthetic construct
  REGION 1..15: note = Synthetic: exemplary linker
GPAKELTPLKEAKVS

SEQ ID NO: 90 - protein, 15 aa, synthetic construct
  REGION 1..15: note = Synthetic: exemplary linker
GHEAAAVMQVQYPAS

SEQ ID NO: 91 - protein, 24 aa, synthetic construct
  REGION 1..24: note = Synthetic: exemplary linker
TVAAPSVFIFPPTVAAPSVFIFPP

SEQ ID NO: 92 - protein, 26 aa, synthetic construct
  REGION 1..26: note = Synthetic: exemplary linker
ASTKGPSVFPLAPASTKGPSVFPLAP

SEQ ID NO: 93 - protein, 11 aa, synthetic construct
  REGION 1..11: note = Synthetic: epitope
YIDFRKDLGWK